(19) United States
(12) Patent Application Publication
(10) Pub. No.: US 2010/0035863 A1
(43) Pub. Date: Feb. 11, 2010

(54) 2 Amino-Pyrimidine Derivatives As H4 Receptor Antagonists, Processes For Preparing Them And Their Use In Pharmaceutical Compositions

(75) Inventors: Gilles Raphy, Stortford (GB); Robert John Watson, Suffolk (GB); Duncan Hannah, Suffolk (GB); Cecile Pegurier, Forest (BE); Isabelle Ortmans, Woluwe-Saint-Lambert (BE); Christopher James Lock, Cambridge (GE); Roland Laurence Knight, St. Edmund's (GB); David Alan Owen, Cambridge (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Brussels (BE)

(21) Appl. No.: 12/440,899

(22) PCT Filed: Sep. 11, 2007
(86) PCT No.: PCT/EP2007/007898

§ 371 Date: Aug. 3, 2009

(30) Foreign Application Priority Data
Sep. 12, 2006 (GB) 0617966.7
Sep. 19, 2006 (EP) 06019518.7

(51) Int. Cl. A61K 31/5513; C07D 403/04; A61K 31/497; A61K 31/4985

(52) U.S. Cl. 514/218; 544/295; 514/252.14; 540/575; 514/249

(57) ABSTRACT

The present invention concerns novel 2 amino pyrimidine derivatives of formula (I), processes for preparing them, pharmaceutical compositions containing them and their use as harmaceuticals.

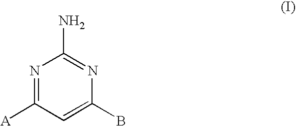

The present invention concerns novel 2 amino-pyrimidine derivatives, processes for preparing them, pharmaceutical compositions containing them and their use as pharmaceuticals.

### BACKGROUND

Histamine was isolated and identified by Windhaus & Vogt (1907) and demonstrated to exert a wide range of physiological effects (Dale & Laidlaw, 1910). Histamine is produced from cellular stores, such as mast cells, basophils, enterochromaffin like cells and within histaminergic neurons, but can also be synthesised by the enzyme, histidine decarboxylase, and released from a number of different cell types. Several haematopoietic cell populations possess this enzymatic activity. The actions of histamine are mediated by members of the G-protein coupled receptor superfamily. To date four histamine receptor subtypes have been identified and characterised. The H₁-, H₂-, and H₃-receptor were defined on the basis of quantitative receptor pharmacology using selective receptor antagonists and their physiological effects are well characterised (see Hill et al., 1997). However, some actions of histamine and other histamine receptor agonists, such as calcium mobilisation in human eosinophils (Raible et al., 1994) were concluded to be mediated by receptor, which was distinct from the above known subtypes as judged by agonist potency orders and antagonist affinity estimates. Subsequently, a number of groups (Oda et al. 2000; Nakamura et al. 2000; Zhu et al. 2001; Nguyen et al. 2001; Morse et al. 2001; Liu et al. 2001; Coge et al 2001; O'Reilly et al. 2002) identified and characterised a novel histamine receptor, which was termed the H₄-receptor. The gene encoding this receptor is located on chromosome 18q11.2 and encodes a 390 amino acid receptor, which is expressed predominantly on cells of immune origin. The amino acid sequence of human H₄-receptor is most closely related to the human H₃-receptor sharing 35-43% sequence identity at the protein level and increasing to 58% in the transmembrane domains. Sequence identity with the H₁- and H₂-receptor subtypes is between 18-31%. The H₄-receptor has subsequently been cloned in a number of species; mouse, rat, guinea pig, porcine and monkey. With the exception of the monkey H₄-receptor, which is highly homologous to the human receptor (>90%, Oda et al. 2005), the homology across the remaining species is between 65-72% (Oda et al. 2002; Liu et al. 2001). The expression profile of this receptor is consistent across species, being present in haematopoietic cells, including eosinophils, mast cells, basophils, T-lymphocytes and dendritic cells. In addition, low positive signals have been detected in brain, lung and liver. This relatively restricted expression suggests a potential role in inflammation, haematopoiesis and immunity.

To date a number of inflammatory actions of the H₄-receptor have been described: in vitro actions, calcium mobilisation and chemotaxis of murine mast cells (Hofstra et al. 2003) and eosinophils (Buckland et al., 2003; Ling et al., 2004), upregulation of adhesion molecules, CD11 b/CD18 (Mac1) and CD54 on eosinophils (Buckland et al. 2003; Ling et al. 2004) and reduction in pro-inflammatory cytokine profiles following TLR ligand stimulation of dendritic cells (Dunford et al. 2006); in vivo actions, histamine-induced mast cell recruitment (Thurmond et al., 2004), neutrophil infiltration in a mouse zymosan-induced peritonitis model (Thurmond et al. 2004) and zymosan-induced neutrophilia to the pleural cavity (Takeshita et al. 2003), eosinophil recruitment (Dunford et al. 2006; Douglas et al., 2006) and mediating itch/puritis (Bell et al. 2004).

On this basis histamine H₄-receptor antagonists and inverse agonists may be used for the prophylaxis and treatment of different kinds of diseases and disorders such as: respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal disfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

EP1437348 discloses 2,4-diamino-6-methyl-pyrimidines of formula

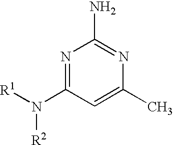

as cosmetics for the use in active deodorants and pharmaceuticals for the use of treating acne and greasy skins or flakes of scurf. EP1437348 discloses compounds 4-methyl-6-(4-methyl-[1,4]diazepan-1-yl pyrimidin-2-ylamine and 4-methyl-6-(4-methyl-piperazin-1-yl)-pyrimidine-2-ylamine. Chem. Therapeutics 1965, (1), 26-31 describes the synthesis of compound 4-methyl-6-(4-methyl-piperazin-1-yl)-pyrimidine-2-ylamine.

Chimica Therapeutica 1971, 6(2), 105-8 describes the synthesis of compound 2-amino-4-methyl-6-(1-piperazinyl)-pyrimidine.

Journal of Heterocyclic Chemistry 2005, 42(7), 1289-1295 describes the synthesis of compound 2-amino-4-(ethyl-1-piperazinyl)-6-methylpyrimidine.

Compound 2-amino-4-methyl-6-(4-(1-methylethyl )-1-piperazinylpyrimidine is part of Interchim Intermediates, a chemical library.

Chemical & Pharmaceutical Bulletin 1986, 34(10), 4150-4165 describes the synthesis of compound 2-amino-4-(4-ethyl-1-piperazinyl)-6-propylpyrimidine.

WO2005/054239 discloses pyrimidine derivatives of formula

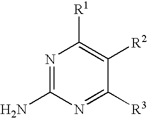

as H₄ receptor antagonists.

WO2005/014556 discloses pyrimidines of formula

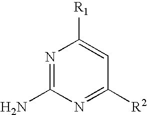

as H₄ receptor antagonists.

There still is a need for H₄ receptor antagonists, it has now surprisingly been found that some novel analogs of 2-amino pyrimidines demonstrate therapeutic properties in this field.

In one aspect, the invention provides a compound having formula I or pharmaceutically acceptable salts thereof or stereoisomeric forms thereof, and the geometrical isomers, enantiomers, diastereoisomers, and pharmaceutically acceptable salts thereof

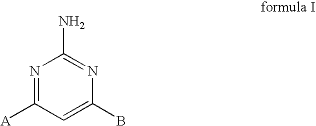

* represents the point of attachment to the rest of the molecule
wherein A is a group of formula II

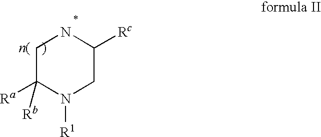

wherein
n is 1 or 2;
R¹ is hydrogen or is unsubstituted C₁₋₃ alkyl groups or is C₃₋₅ unsubstituted cycloalkyl;
R^{a} is hydrogen or is unsubstituted C₁₋₄ alkyl groups;
R^{b} is hydrogen or is unsubstituted C₁₋₃ alkyl groups;
R^{c} is hydrogen or is unsubstituted C₁₋₃ alkyl groups;
or A is a group of formula III

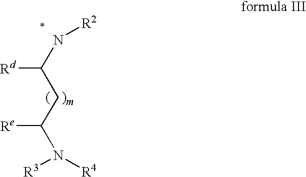

wherein
m is 0, 1 or 2;
R² is hydrogen or is unsubstituted C₁₋₃ alkyl group;
R³ is hydrogen or is unsubstituted C₁₋₃ alkyl group;
R⁴ is hydrogen or is unsubstituted C₁₋₃ alkyl group;
R^{d} is hydrogen or is unsubstituted C₁₋₃ alkyl group;
R^{e} is hydrogen or is unsubstituted C₁₋₃ alkyl group;
or A is a group of formula IV

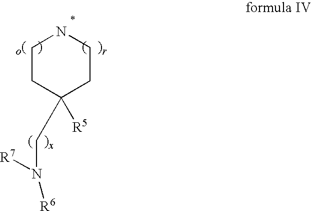

wherein
o is 0 or 1;
r is 0, 1 or 2;
x is 0 or 1;
R⁵ is hydrogen or is unsubstituted C₁₋₃ alkyl group;
R⁶ is hydrogen or is unsubstituted C₁₋₃ alkyl group;
R⁷ is hydrogen or is unsubstituted C₁₋₃ alkyl group;
or A is a group of formula V

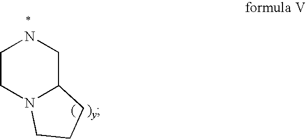

wherein
y is 1 or 2;
or A is a group of formula VI

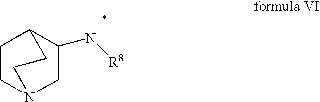

wherein
R⁸ is hydrogen or is unsubstituted C₁₋₃ alkyl group;
or A is a group of formula VII

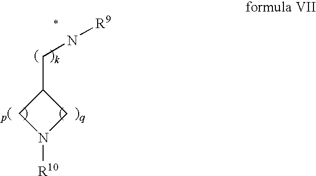

wherein
k is 0 or 1;
p is 1 or 2 or 3;
q is 0 or 1 or 2;
R⁹ is hydrogen or is unsubstituted C₁₋₃ alkyl group;
R¹⁰ is hydrogen or is unsubstituted C₁₋₃ alkyl group;
or A is a group of formula VIII

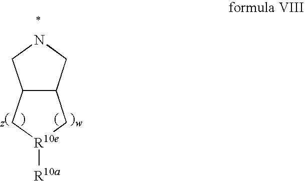

wherein
z is 0, 1, 2 or 3;
w is 0 or 1;
R^{10a} is hydrogen or is unsubstituted C₁₋₃ alkyl group or is NH₂; and R^{10e} is a CH group;
or
R^{10a} is hydrogen or is unsubstituted C₁₋₃ alkyl group; and R^{10e} is N;
or A is a group of formula XII

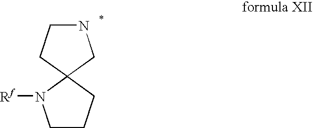

wherein
R^{f} is hydrogen or is unsubstituted C₁₋₃ alkyl group;
or A is a group of formula XIII

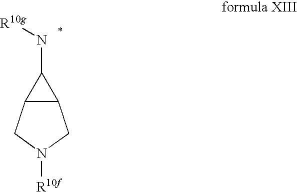

wherein
R^{10f} is hydrogen or is unsubstituted C₁₋₃ alkyl group;
R^{10g} is hydrogen or is unsubstituted C₁₋₃ alkyl group;
or A is a group of formula XIV

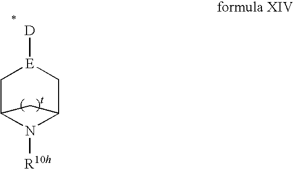

wherein
D is NH; and E is CH; or
D is direct bond; and E is CH or N;
t is 1, 2 or 3;
R^{10h} is hydrogen or is unsubstituted C₁₋₃ alkyl group;
and

wherein

B is defined as C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl;

or B is defined as C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups;

or B is defined as C₂₋₇ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups;

or B is defined as C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl;

or B is defined as C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

or B is a group of formula IX

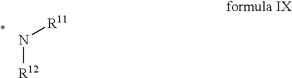

wherein

R¹¹ is hydrogen and R¹² is C₅₋₁₀ cycloalkyl (mono- or polycyclic) optionally substituted by 1 to 3 C₁₋₄ alkyl groups, or by aryl, or by an amide moiety, or fused to an aryl;

or R¹¹ is hydrogen and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups and R¹² is C₃₋₁₀ cycloalkyl (mono- or polycyclic) optionally substituted by 1 to 3 C₁₋₄ alkyl groups;

or R¹ 1 is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

or B is a group of formula X

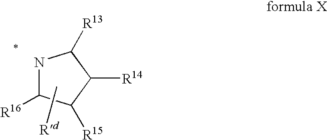

wherein

R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen;

R¹⁻⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens;

R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups;

R¹⁶ is hydrogen or can form together with R¹³ a C₁₋₃ alkylene chain;

R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups;

d is 0 to 2;
or B is a group of formula XI

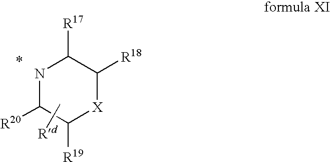

wherein

R¹⁷ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by nitrile, or by aryl optionally substituted by 1 to 3 C₁₋₄ alkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl, or can form together with R¹⁸ a benzene ring fused to the nitrogen heterocycle;

R¹⁸ is hydrogen or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1-3 C₁₋₄ alkyl groups or can form together with R²¹ a benzene ring fused to the nitrogen heterocycle, in which case R²² is not present;

R¹⁹ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups or or can form together with R¹⁷ a C₁₋₃ alkylene chain;

R²⁰ is hydrogen or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups, or can form together with R¹⁷ a C₁₋₃ alkylene chain;

X is CR²¹ R²², or is NR²³ or is C₂₋₃ alkylene chain;

R²¹ is hydrogen, or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or is C₁₋₇ alkyl groups (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups or is C₁₋₃ alkoxy group;

R²² is hydrogen, or is hydroxyl, or is C₁₋₃ alkoxy, or is C₁₋₃ haloalkyl groups;

R²³ is hydrogen, or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by 1 to 3 C₁₋₄ alkyl groups, or is aryl optionally substituted by a heterocycle (aromatic or nonaromatic) optionally substituted by a ketone moiety or is aryl optionally substituted by a heterocycle (aromatic or nonaromatic) optionally substituted by nitrile or is aryl optionally substituted by a heterocycle (aromatic or nonaromatic) optionally substituted by 1 to 3 C₁₋₄ alkyl groups;

R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups;

d is 0 to 2;

except:

4-[3-(Methylamino)azetidin-1-yl]-6(4-methylpiperidin-1-yl)pyrimidin-2,4-diamine;

N⁴-Bicyclo[1.1.1]pent-1-yl-6[(3R)-3-methylamino)pyrolidin-1-yl]pyrimidine-2,4-diamine;

4-[3-(Methylamino)azetidin-1-yl]-6-piperidin-1-yl)pyrimidin-2-diamine;

4-[(4-ethyl-1-piperazinyl)-6-propyl]pyrimidin-2-amine.

The term “alkyl”, as used herein, refers to saturated, monovalent or divalent hydrocarbon radicals having linear or branched moieties and containing 1-7 carbon atoms. Alkyl groups may optionally be substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by nitrile, or by aryl optionally substituted by 1 to 3 C₁₋₄ alkyl groups, or by a heterocycle (aromatic or nonaromatic), or by aryl optionally substituted by a heterocycle (aromatic or nonaromatic) optionally substituted by 1 to 3 C₁₋₄ alkyl groups, or by aryl optionally substituted by a heterocycle (aromatic or nonaromatic) optionally substituted by nitrile. One methylene group, of the alkyl, can be replaced by oxygen.

Usually alkyl groups in the present case are methoxymethyl, propyl, tert-butyl, methyl, 1-phenylethyl, 1,3-dioxalan-2-yl-ethyl, 2-phenylethyl, cyclopentylmethyl, ethyl, iso-propyl, 1-methylpentyl, 1-ethylpropyl, iso-butyl, cyclohexylmethyl. Preferred alkyl groups are methyl, ethyl, tert-butyl, iso-butyl, 1-ethylpropyl, 1-methylpentyl, 1-phenylethyl, cyclohexylmethyl, iso-propyl, cyclopentylmethyl. More preferred alkyl groups are methyl, cyclohexylmethyl, cyclopentylmethyl.

The term “alkenyl”, as used herein refers to monovalent or divalent hydrocarbon radicals having 2 to 6 carbon atoms, derived from a saturated alkyl,as described above, having at least a double bond. C₂₋₆ alkenyl groups can be in Z or E configuration. The preferred configuration is E. Alkenyl groups may optionally be substituted by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups, or by a heterocycle (aromatic or nonaromatic). Usually alkenyl groups are (1E) 3,3 dimethyklbuty-1-en, (E)-2-cyclopropylvinyl, (E)-2-phenylvinyl. Preferred alkenyl group is (1E) 3,3 dimethyklbuty-1-en.

The term “cycloalkyl”, as used herein, refers to a monovalent or divalent group of 3 to 10 carbon atoms, derived from a saturated cyclic hydrocarbon. Cycloalkyl groups can be monocyclic or polycyclic and can optionally be substituted by 1 to 3 C₁₋₄ alkyl groups, as defined above, or 1-3 halogens, or 1 or 2 C₁₋₃ alkoxy, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by 1-3 halogens, or by aryl optionally substituted by 1 to 3 C₁₋₄ alkyl groups or can be fused to an aryl.

Usually cycloalkyl groups, in the present case, are 2,6,6-trimethylbicyclo[3.1.1]hept-3-yl, N-phenylcyclohexanecarboxamide, N-methylcyclohexanecarboxamide, N-cyclopropylcyclohexanecarboxamide, N-tert-butylcyclohexanecarboxamide, N-(4-methoxyphenyl)cyclohexanecarboxamide, cyclohexyl, 2,3-dihydro-1H-inden-2-yl, adamant-2-yl, (1R*,2R*,4S*)-bicyclo[2.2.1]hept-2-yl, cyclopentyl, cyclopropyl 1,2,3,4-tetrahydronaphthalen-2-yl, cycloheptyl, (1R*,2S*,4S*)-bicyclo[2.2.1]hept-2-yl, bicyclo[2.2.1]hept-2-yl, (1R; 4R) 1,7,7-trimethylbicyclo[2.2.1]hept-2-yl), adamant-1-yl, 1,3,3-trimethylbicyclo[2.2.1]hept-2-yl, [exo-bicyclo[2.2.1]hept-2-yl.

Preferred cycloalkyl groups, in the present case are, cyclohexyl, adamant-1-yl, 1,3,3-trimethylbicyclo[2.2.1]hept-2-yl, adamant-2-yl, (1R; 4R) 1,7,7-trimethylbicyclo[2.2.1]hept-2-yl), cyclopentyl, cycloheptyl, (1R*,2S*,4S*)-bicyclo[2.2.1]hept-2-yl, bicyclo[2.2.1]hept-2-yl, (1 R*,2R*,4S*)-bicyclo[2.2.1]hept-2-yl, cyclopropyl, 1,2,3,4-tetrahydronaphthalen-2-yl, 2,3-dihydro-1H-inden-2-yl, [exo-bicyclo[2.2.1]hept-2-yl, [(1R*,2S*,4S*)-bicyclo[2.2.1]hept-2-yl.

More preferred cycloalkyl groups, in the present case are, [(1R*,2S*,4S*)-bicyclo[2.2.1]hept-2-yl, cyclohexyl, [exo-bicyclo[2.2.1]hept-2-yl, (1 R*,2R*,4S*)-bicyclo[2.2.1]hept-2-yl, cyclopentyl,adamant-2-yl.

The term “cycloalkenyl”, as used herein, refers to a monovalent or divalent group of 5 to 10 carbon atoms, derived from a saturated cycloalkyl having one double bond. Cycloalkenyl groups can be monocyclic or polycyclic. Cycloalkenyl groups can be substituted by 1 to 3 C₁₋₄ alkyl groups, or by 1-3 halogens, or by 1 or 2 C₁₋₃ alkoxy, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by 1-3 halogens, or by aryl optionally substituted by 1 to 3 C₁₋₄ alkyl groups. Usually cycloalkenyl groups in the present case are, cyclohex-1-en, cyclohept-1-en, cyclohex-1-en, 4-methylcyclohex-1-en. Preferred cycloalkenyl group in the present case is cyclohex-1-en.

The term “halogen”, as used herein, refers to an atom of Cl, Br, F, I. Usually, halogens are Cl, F.

The term C₁₋₃ “alkylene”, as used herein, refers to a saturated, divalent hydrocarbon moieties containing 1 to 3 carbon atoms, preferably 1 to 2 carbon atoms. Usually alkylene groups are methylene, ethylene.

The term C₁₋₃ “alkoxy”, as used herein, refers to a group of formula —O R²⁴ wherein R²⁴ is an alkyl as defined above, containing 1 to 3 carbon atoms. Usually C₁₋₃ alkoxy group is methoxy.

The term C₁₋₃ “haloalkyl”, as used herein, refers to a C₁₋₃ alkyl group, as defined above, substituted by 1 to 3 halogens. Usually the alkyl group is methyl and the halogen is fluoro. Usually “haloalkyl” group is trifluoromethyl.

The term C₁₋₃ “haloalkoxy”, as used herein, refers to a C₁₋₃ alkoxy group, as defined above, substituted by 1 to 3 halogens. Usually the haloalkoxy group is trifluoromethoxy.

The term “nitrile”, as used herein, refers to a group of formula —CN.

The term “ketone”, as used herein, refers to a group of formula —C(O) R²⁵, wherein R²⁵ is C₁₋₃ alkyl as defined above or an aryl, optionally substituted by 1-3 halogens, by 1 or 2 C₁₋₃ alkoxy groups, by 1 or 2 C₁₋₃ haloalkyl groups, by 1 or 2 C₁₋₃ haloalkoxy groups as defined above.

The term “aryl” as used herein, refers to an organic moiety derived from an aromatic hydrocarbon consisting of a ring or multiple rings, containing 6 to 10 carbon atoms by removal of one hydrogen atom, which can optionally be substituted by 1 to 3 C₁₋₄ alkyl groups, by 1-3 halogens, by 1 or 2 C₁₋₃ alkoxy groups, by 1 or 2 C₁₋₃ haloalkyl groups, by 1 or 2 C₁₋₃ haloalkoxy groups, by nitrile, by a heterocycle (aromatic or nonaromatic) optionally substituted by a ketone moiety, by a heterocycle (aromatic or nonaromatic) optionally substituted by nitrile, by a heterocycle (aromatic or nonaromatic) optionally substituted by 1 to 3 C₁₋₄ alkyl groups, as defined above. The aryl moiety can be directly attached to the rest of the molecule (in the case of phenyl) or via a —CH₂-group (in the case of benzyl) or via an oxygen atom (in the case of phenoxy) or via a —O—CH₂— group (in the case of benzoxy). Usually, in the present case, aryl groups are phenyl, benzyl, 3-fluorophenyl, 4-fluorophenyl, 2-methoxyphenyl, 3-(trifluoromethyl)phenyl], 4-chlorophenoxy, 4-oxy)benzonitrile, 4-(trifluoromethyl)phenoxy, 4-fluorobenzyl, 4-chlorobenzyl, 4-fluorobenzyl)oxy], 4-methoxyphenyl), 2-fluorophenyl, 3-fluorophenyl), 2-(trifluoromethyl)phenyl, 2-methylphenyl, 4-chlorophenyl.

Preferred aryl groups are 2-methoxyphenyl, 4-chlorophenyl, phenyl.

The term “amide”, as used herein, refers to a group of formula —C(O)N—.

The term “hydroxyl”, as used herein, refers to a group of formula —OH.

The term “amino”, as used herein, refers to a group of formula —NH₂.

The term “alkylamino”, as used herein, refers to a group of formula —NHR²⁶, wherein R²⁶ is a C₁₋₃ alkyl group as defined above.

The term “dialkylamino”, as used herein, refers to a group of formula —NR²⁷R²⁸, wherein R²⁷ is as defined above and R²⁸ is a C₁₋₃ alkyl group as defined above. The term “heterocycle”, as used herein refers to a 3 to 10 membered ring, which can be aromatic or non -aromatic, containing at least one heteroatom selected from O or N or S or combinations of at least two thereof, interrupting the carbocyclic ring structure. The heterocyclic ring can be interrupted by —C═O. The S heteroatom can be oxidized. Heterocycles can be monocyclic or polycyclic. Heterocycles can optionally be substituted by 1 to 3 C₁₋₄ alkyl, amino, nitrile, alkylamino, dialkylamino, 1 to 3 halogens, C₁₋₃ alkoxy, ketone groups, dialkylamido groups, optionally substituted aryl groups, as defined above. Usually in the present case heterocycles groups are 4-methylpiperazin-1-yl), (4-phenylpiperidin-1-yl, 4-benzylpiperidin-1-yl, 4-tert-butylpiperidin-1-yl, 3-fluorophenyl)piperidin-1-yl, 4-methoxypiperidin-1-yl, 4-fluorophenyl)piperazin-1-yl, 2-methoxyphenyl)piperazin-1-yl, 4-(trifluoromethyl)piperidin-1-yl, 3-(trifluoromethyl)phenyl]piperazin-1-yl, (2S)-2-(methoxymethyl)pyrrolidin-1-yl, 2-propylpyrrolidin-1-yl, 2-tert-butylpyrrolidin-1-yl, 2,6-dimethylpiperidin-1-yl), 4-(4-chlorophenoxy)piperidin-1-yl, piperidin-4-yl}oxy)benzonitrile, 3-[4-(trifluoromethyl)phenoxy]pyrrolidin-1-yl, 4-(4-fluorobenzyl)piperidin-1-yl, 4-(4-chlorobenzyl)piperidin-1-yl, 4-fluorobenzyl)oxy]pyrrolidin-1-yl} 1-acetylpiperidin-4-yl, N⁴-[(1R,2S*,4S*)-bicyclo[2.2.1]hept-2-yl, 1,3-dihydro-2H-isoindol-2-yl, 3-(dimethylamino)pyrrolidin-1-yl, 4-(2-fluorophenyl)piperidin-1-yl], [3-(3-fluorophenyl)piperidin-1-yl], 4-[2-(trifluoromethyl)phenyl]piperidin-1-yl, 4-(2-methylphenyl)piperidin-1-yl, N⁴-(1-phenylpiperidin-4-yl, N⁴-(1-benzylpiperidin-4-yl (octahydro-2H-pyrido[1,2-a]pyrazin-2-yl), 2-(1,3-dioxolan-2-yl)ethyl, (4-cyclopropylpiperazin-1-yl), 4-isopropylpiperazin-1-yl), 3-methylpiperazin-1-yl, 3-amino-3-methylpyrrolidin-1-yl, 3-aminoazetidin-1-yl,3,3-dimethylpiperazin-1-yl), 3,4-dimethylpiperazin-1-yl, N⁴-(1-methylazetidin-3-yl), 1,7-diazaspiro[4.4]non-7-yl, (3aR*,6aS*)-hexahydropyrrolo[3,4-c]pyrrol-2(1H)-yl, (3aR*,6aS*)-5-methylhexahydropyrrolo[3,4-c]pyrrol-2(1H)-yl, (2R)-2-methylpiperazin-1-yl, [(1R*,5S*,6S*)-3-azabicyclo[3.1.0]hexan, (2S)-2,4-dimethylpiperazin-1-yl], 3-[(methylamino)methyl]azetidin-1-yl, (2R)-2,4-dimethylpiperazin-1-yl, N⁴-[(1R*,5S*,6S*)-3-azabicyclo[3.1.0]hex-6-yl, N⁴-[(1R*,5S*,6S*)-3-methyl-3-azabicyclo[3.1.0]hex-6-yl, 3-(diethylamino)azetidin-1-yl, N⁴-8-azabicyclo[3.2.1]oct-3-yl, N⁴-[(3-endo)-8-methyl-8-azabicyclo[3.2.1]oct-3-yl, N⁴-[(1-methylpiperidin-2-yl)methyl, (1R*,5S*)-8-azabicyclo[3.2.1]oct-8-yl, (tetrahydro-2H-pyran-4-yl, 4-(methylamino)piperidin-1-yl, 3-methyl-3,4-dihydroisoquinolin-2(1H)-yl), 6-azabicyclo[3.2.1]oct-6-yl, 4-azepan-1-yl, 4-chlorophenyl)piperidin-4-ol, 3-phenylpiperidin-1-yl), 7-azabicyclo[2.2.1]hept-7-yl, 1-azabicyclo[2.2.2]oct-3-yl, 4-ethylpiperazin-1-yl, 4-methyl-1,4-diazepan-1-yl, hexahydropyrrolo[1,2-a]pyrazin-2(1H)-yl, [3-(aminomethyl )azetidin-1-yl, (3R)-3-aminopyrrolidin-1-yl, (2S)-2-methylpiperazin-1-yl, (3R)-3-methylpiperazin-1-yl, (3S)-3-methylpiperazin-1-yl, (3S)-3-(methylamino)pyrrolidin-1-yl, (3S)-3-aminopyrrolidin-1-yl], 1-methylpiperidin-4-yl, 1-methylpyrrolidin-3-yl, piperazin-1-yl, 4-methylpiperidin-1-yl, 3,4-dihydroisoquinolin-2(1H)-yl, 4-(2-methoxyphenyl)piperidin-1-yl, 5-fluoro-1,3-dihydro-2H-isoindol-2-yl, [3-(ethylamino)azetidin-1-yl, (3S)-3-isobutylpiperazin-1-yl, 3-(methylamino)pyrrolidin-1-yl, 3,8-diazabicyclo[3.2.1]oct-3-yl, (3S)-3-isopropylpiperazin-1-yl, 3-ethylpiperazin-1-yl, 3-aminopyrrolidin-1-yl), (2-methylpyrrolidin-1-yl, (3-methylpiperazin-1-yl, (4aR*,7aR*)-octahydro-6H-pyrrolo[3,4-b]pyridin-6-yl), 3-(methylamino)azetidin-1-yl.

Preferred heterocycles are 3-aminopyrrolidin-1-yl, (2-methylpyrrolidin-1-yl, (3-methylpiperazin-1-yl, (4aR*,7aR*)-octahydro-6H-pyrrolo[3,4-b]pyridin-6-yl), 3-(methylamino)azetidin-1-yl, methylpiperazin-1-yl, 1,3-dihydro-2H-isoindol-2-yl, piperazin-1-yl, 4-methylpiperidin-1-yl, 3,4-dihydroisoquinolin-2(1H)-yl, 4-(2-methoxyphenyl)piperidin-1-yl, 5-fluoro-1,3-dihydro-2H-isoindol-2-yl, [3-(ethylamino)azetidin-1-yl, (3S)-3-isobutylpiperazin-1-yl, 3-(methylamino)pyrrolidin-1-yl, 3,8-diazabicyclo[3.2.1]oct-3-yl, (3S)-3-isopropylpiperazin-1-yl, 3-ethylpiperazin-1-yl, 3-aminoazetidin-1-yl, [3-(aminomethyl)azetidin-1-yl, (3R)-3-aminopyrrolidin-1-yl, (2S)-2-methylpiperazin-1-yl, (3R)-3-methylpiperazin-1-yl, (3S)-3-methylpiperazin-1-yl, (³S)-3-(methylamino)pyrrolidin-1-yl, (3S)-3-aminopyrrolidin-1-yl[, 1-methylpiperidin-4-yl, 1-methylpyrrolidin-3-yl, 3-(dimethylamino)pyrrolidin-1-yl, 4-ethylpiperazin-1-yl, 4-methyl-1,4-diazepan-1-yl, hexahydropyrrolo[1,2-a]pyrazin-2(1H)-yl, (3aR*,6aS*)-hexahydropyrrolo[3,4-c]pyrrol-2(1H)-yl, (1R*,5S*)-8-azabicyclo[3.2.1]oct-8-yl, (tetrahydro-2H-pyran-4-yl, 4-(methylamino)piperidin-1-yl, 3-methyl-3,4-dihydroisoquinolin-2(1H)-yl), 6-azabicyclo[3.2.1]oct-6-yl, 4-azepan-1-yl, 4-chlorophenyl)piperidin-4-ol, 3-phenylpiperidin-1-yl, 7-azabicyclo[2.2.1]hept-7-yl, 1-azabicyclo[2.2.2]oct-3-yl.

More preferred heterocycles are methylpiperazin-1-yl, 1,3-dihydro-2H-isoindol-2-yl, piperazin-1-yl, 3-aminopyrrolidin-1-yl, (2-methylpyrrolidin-1-yl, ³-(methylamino)pyrrolidin-1-yl, (3-methylpiperazin-1-yl, 1,4-diazepan-1-yl, (4aR*,7aR*)-octahydro-6H-pyrrolo[3,4-b]pyridin-6-yl), (hexahydropyrrolo[1,2-a]pyrazin-2(1H)-yl, 3-(methylamino)azetidin-1-yl.

In one embodiment of the invention A is a group of formula II wherein usually n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups.

In another embodiment n is 1; and R¹ is hydrogen or is methyl or is cyclopropyl or is ethyl or is isopropyl; and R^{a} is hydrogen or methy; and R^{b} is hydrogen or is methyl; and R^{c} is hydrogen or is methyl.

In a preferred embodiment, n is 1; and R¹ is hydrogen or methyl or ethyl; and R^{a} is hydrogen or methyl or ethyl or iso-propyl or iso-butyl; and R^{b} is hydrogen; and R^{c} is hydrogen or methyl.

In another preferred embodiment, n is 1; and R¹ is methyl; and R^{a} is hydrogen; and R^{b} is hydrogen; and R^{c} is hydrogen. In a preferred embodiment, n is 1; and R¹ is ethyl; and R^{a} is hydrogen; and R^{b} is hydrogen; and R^{c} is hydrogen.

In another preferred embodiment, n is 1; and R¹ is hydrogen; and R^{a} is methyl; and R^{b} is hydrogen; and R^{c} is hydrogen.

In another preferred embodiment, n is 1; and R¹ is hydrogen; and R^{a} is methyl; and R^{b} is hydrogen; and R^{c} is methyl. In another preferred embodiment, n is 1; and R¹ is hydrogen; and R^{a} is ethyl; and R^{b} is hydrogen; and R^{c} is hydrogen. In another preferred embodiment, n is 1; and R¹ is hydrogen; and R^{a} is iso-butyl; and R^{b} is hydrogen; and R^{c} is hydrogen. In another preferred embodiment, n is 1; and R¹ is hydrogen; and R^{a} is iso-propyl; and R^{b} is hydrogen; and R^{c} is hydrogen.

In another preferred embodiment n is 2; and R¹ is methyl; and R^{a} is hydrogen; and R^{b} is hydrogen; and R^{c} is hydrogen.

In a more preferred embodiment, n is 2; and R¹ is hydrogen; and R^{a} is hydrogen; and R^{b} is hydrogen; and R^{c} is hydrogen. In another more preferred embodiment, n is 1; and R¹ is hydrogen; and R^{a} is hydrogen; and R^{b} is hydrogen; and R^{c} is hydrogen. In another more preferred embodiment, n is 1; and R¹ is methyl; and R^{a} is hydrogen; and R^{b} is hydrogen; and R^{c} is hydrogen. In another more preferred embodiment, n is 1; and R¹ is hydrogen; and R^{a} is methyl; and R^{b} is hydrogen; and R^{c} is hydrogen.

In another embodiment of the invention A is a group of formula III wherein usually m is 0, 1 or 2; and R² is hydrogen or is unsubstituted C₁₋₃ alkyl group; and R³ is hydrogen or is unsubstituted C₁₋₃ alkyl group; and R⁴ is hydrogen or is unsubstituted C₁₋₃ alkyl group; and R^{d} is hydrogen or is unsubstituted C₁₋₃ alkyl group; and R^{e} is hydrogen or is unsubstituted C₁₋₃ alkyl group.

In another embodiment m is 1; and R² is methyl or hydrogen; and R³ is methyl; and R⁴ is methyl; and R^{d} is hydrogen; and R^{e} is hydrogen.

In a preferred embodiment m is 0; and R² is hydrogen; and R³ is methyl; and R⁴ is methyl; and R^{d} is hydrogen; and R^{e} is hydrogen.

In another preferred embodiment m is 0; and R² is hydrogen; and R³ is hydrogen; and R⁴ is methyl; and R^{d} is hydrogen; and R^{e} is hydrogen.

In a more preferred embodiment m is 0; and R² is hydrogen; and R³ is methyl; and R⁴ is hydrogen; and R^{d} is hydrogen; and R^{e} is hydrogen.

In another embodiment of the invention A is a group of formula IV wherein usually o is 0 or 1; and r is 0 or 1 or 2; x is 0 or 1; and R⁵ is hydrogen or unsubstituted C₁₋₃ alkyl group; and R⁶ is hydrogen or unsubstituted C₁₋₃ alkyl group; R⁷ is hydrogen or unsubstituted C₁₋₃ alkyl group.

In another embodiment usually o is 0; and r is 0 or 1; x is 0 or 1; and R⁵ is hydrogen or methyl; and R⁶ is hydrogen or methyl or ethyl; R⁷ is hydrogen or ethyl.

In a preferred embodiment o is 0; and r is 1; and x is 0; and R⁵ is hydrogen; and R⁶ is methyl and R⁷ is methyl. In another preferred embodiment o is 0; and r is 1; and x is 0; and R⁵ is hydrogen; and R⁶ is hydrogen; and R⁷ is hydrogen. In another preferred embodiment o is 1; and r is 1; and x is 0; and R⁵ is hydrogen; and R⁶ is hydrogen; and R⁷ is methyl. In another preferred embodiment o is 0; and r is 1; and x is 0; and R⁵ is hydrogen; and R⁶ is methyl; and R⁷ is hydrogen. In another preferred embodiment o is 0; and r is 0; and x is 0; and R⁵ is hydrogen; and R⁶ is hydrogen; and R⁷ is hydrogen.

In another preferred embodiment o is 0; and r is 1; and x is 1; and R⁵ is hydrogen; and R⁶ is hydrogen; and R⁷ is hydrogen. In another preferred embodiment o is 0; and r is 1; and x is 0; and R⁵ is hydrogen; and R⁶ is ethyl; and R⁷ is hydrogen.

In a more preferred embodiment o is 0; and r is 1; and x is 0; and R⁵ is hydrogen; and R⁶ is methyl; and R⁷ is hydrogen. In another more preferred embodiment o is 0; and r is 1; and x is 0; and R⁵ is hydrogen; and R⁶ is hydrogen; and R⁷ is hydrogen. In another more preferred embodiment o is 0; and r is 0; and x is 0; and R⁵ is hydrogen; and R⁶ is methyl; and R⁷ is hydrogen.

In another embodiment of the invention A is a group of formula V wherein usually y is 1 or 2. In a preferred embodiment y is 1.

In another embodiment of the invention A is a group of formula VI wherein usually R⁸ is hydrogen or unsubstituted C₁₋₃ alkyl group. In a preferred embodiment R⁸ is hydrogen.

In another embodiment of the invention A is a group of formula VII wherein usually k is 0 or 1; and p is 1 or 2 or 3; and q is 0 or 1 or 2; and R⁹ is hydrogen or is unsubstituted C₁₋₃ alkyl group; R¹⁰ is hydrogen or is unsubstituted C₁₋₃ alkyl group.

In a preferred embodiment of the invention p is 1; and q is 2; and k is 0; and R⁹ is hydrogen; and R¹⁰ is hydrogen. In another preferred embodiment of the invention p is 1; and q is 2; and k is 0; and R⁹ is methyl; and R¹⁰ is methyl. In another preferred embodiment of the invention p is 2 and q is 2; and k is 0; and R⁹ is hydrogen; and R¹⁰ is methyl.

In another embodiment of the invention A is a group of formula VIII, wherein z is 0, 1, 2 or 3; and w is 0 or 1; and R^{10a} is hydrogen or unsubstituted C₁₋₃ alkyl group or is NH₂; and R^{10e} is a CH group. In another embodiment of the invention z is 0, 1, 2 or 3; and w is 0 or 1; and R^{10a} is hydrogen or unsubstituted C₁₋₃ alkyl group; and R¹⁰³ is N. In a preferred embodiment z is 1; and w is 1; and R¹⁰¹ is hydrogen; and R^{10e} is N. In a more preferred embodiment z is 3; and w is 0; and R^{10a} is hydrogen; and R^{10e} is N.

In another embodiment of the invention A is a group of formula XII wherein R^{f} is hydrogen or is unsubstituted C₁₋₃ alkyl group. In a preferred embodiment usually R^{f} is hydrogen.

In another embodiment of the invention A is a group of formula XIII wherein R^{10f} is hydrogen or is unsubstituted C₁₋₃ alkyl group; and R^{10g} is hydrogen or is unsubstituted C₁₋₃ alkyl group. In a preferred embodiment R^{10f} is hydrogen; and R^{10g} is hydrogen. In another preferred embodiment R^{10f} is methyl; and R^{10g} is hydrogen. In another embodiment of the invention A is a group of formula XIV wherein D is NH; and E is CH; and t is 1, 2 or 3; and R^{10h} is hydrogen or is unsubstituted C₁₋₃ alkyl group. In another embodiment D is NH; and E is CH; and t is 2; and R^{10h} is hydrogen. In another embodiment of the D is a direct bond; and E is CH or N; and t is 1, 2 or 3; and R^{10h} is hydrogen or is unsubstituted C₁₋₃ alkyl group. In another embodiment D is NH; and E is CH; and t is 2; and R^{10h} is methyl. In a preferred embodiment D is direct bond; and E is N; and t is 2; and R^{10h} is hydrogen.

In one embodiment of the invention usually B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C_{1'}haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl;or B is C₅₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups. In another embodiment of the invention B is cyclohexyl or 2-phenylethyl or cyclopentyl or ethyl-1,3-dioxalane or (E)-2-phenylvinyl or tert-butyl or (1E) 3,3 dimethylbuty-1-en or adamantyl or (E)-2-cyclopropylvinyl or cyclopentylmethyl or cyclohexylmethyl.

In a preferred embodiment B is cyclohexyl or adamantyl or cyclopentyl or ethyl-2-propyl or iso-propyl or 1-methyl-pentyl or 2-phenylethyl or cyclopropyl or methylcyclohexyl orcycloheptyl or 1,2,3,4 tetrahydronaphtalen-2-yl or (1E ) 3,3 dimethylbuty-1-en.

In a more preferred embodiment B is cyclohexyl or adamantyl or cyclopentyl or cyclohexylmethyl or cyclopentylmethyl or cyclohex-1-en.

In another embodiment of the invention B is a group of formula IX herein usually R¹¹ is hydrogen and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁-₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In another embodiment R¹¹ is hydrogen; and R¹² is piperidine or 1 acetylpiperidine or 1R*, 2S*, 4S* -bicyclo[2.2.1]hept-2-yl-N-benzyl or N-phenylcyclohexylcarboxamide or N -phenylcyclohexanecarboxamide or N-methylcyclohexanecarboxamide or N-cyclopropylcyclohexanecarboxamide or N-tert-butylcyclohexanecarboxamide or N-(4-methoxyphenylcyclohexane or cyclohexyl or 1-phenylpiperidine or 1-benzylpiperidin-4-yl or 2,3-dihydro-1H-inden-1-yl or 1,2,3,4-tetrahydronaphthalen-2-yl or (1R*,2R*,4S*)-bicyclo[2.2.1]hept-2-yl.

In a preferred embodiment R¹¹ is hydrogen; and R¹² is adamantyl or cyclohexyl or 1,3,3-trimethylbicyclo[2.2.1]hept-2-yl or (1R; 4R) 1,7,7-trimethylbicyclo[2.2.1]hept-2-yl or (tetrahydro-2H-pyran-4-yl or cyclopentyl or cycloheptyl or 1R,*2S*,4S*)-bicyclo[2.2.1]hept-2-yl or bicyclo[2.2.1]hept-2-yl or (1R*,2R*,4S*)-bicyclo[2.2.1]hept-2-yl.

In another preferred embodiment R¹¹ is methyl; and R¹² is cyclohexyl or cyclopentyl or methyl.

In a more preferred embodiment R¹¹ is hydrogen and R¹² is cyclohexyl or (1R*,2R*,4S*)-bicyclo[2.2.1]hept-2-yl or(1R*,2S*,4S*)-bicyclo[2.2.1]hept-2-yl or [exo-bicyclo[2.2.1]hept-2-yl.

In another embodiment of the invention B is a group of formula X wherein usually R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C_{1'}haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and

R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups; and d is 0 to 2.

In another embodiment R¹³ is hydrogen or n-propyl or tert-butyl or methyl or (2S)-2-methoxymethyl; and R¹⁴ is hydrogen or 4-trifluoromethylphenoxy or 4-chlorophenyl or 2-methoxyphenyl or together with R¹⁵ can form a 1,3-dihydro-2H-isoindol-2-yl group or a 5-fluoro-1,3-dihydro-2H-isoindol-2-yl ring; R¹⁵ is hydrogen and R¹⁶ is hydrogen or together with R¹³ an ethylene chain.

In a preferred embodiment R¹³ is hydrogen or methyl; and R¹⁴ is hydrogen or 4-chlorophenyl or 2-methoxyphenyl or together with R¹⁵ can form a 1,3-dihydro-2H-isoindol-2-yl group or a 5-fluoro-1,3-dihydro-2H-isoindol-2-yl ring; and R¹⁵ is hydrogen; and R¹⁶ is hydrogen or together with R¹³ an ethylene chain.

In a more preferred embodiment R¹³ is hydrogen or methyl; and R¹⁴ is hydrogen or together with R¹⁵ can form a 1,3-dihydro-2H-isoindol-2-yl group; and R¹⁵ is hydrogen; and R¹⁶ is hydrogen.

In another embodiment of the invention B is a group of formula XI wherein R¹⁷ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by nitrile, or by aryl optionally substituted by 1 to 3 C₁₋₄ alkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl, or can form together with R¹⁸ a benzene ring fused to the nitrogen heterocycle; and R¹⁸ is hydrogen or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1-3 C₁₋₄ alkyl groups or can form together with R²¹ a benzene ring fused to the nitrogen heterocycle, in which case R²² is not present; and R¹⁹ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups or is C₁₋₃ dialkylamide or can form together with R¹⁷ a C₁₋₃ alkylene chain; and R²⁰ is hydrogen or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups, or can form together with R¹⁷ a C₁₋₃ alkylene chain; and X is CR²¹R²², or is NR²³ or is C₂₋₃ alkylene chain; and R²¹ is hydrogen, or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or is C₁₋₇ alkyl groups (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups or is C₁₋₃ alkoxy group; and R²² is hydrogen, or is hydroxyl, or is C₁₋₃ alkoxy, or is C₁₋₃ haloalkyl groups; and R²³ is hydrogen, or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by 1 to 3 C₁₋₄ alkyl groups, or is aryl optionally substituted by a heterocycle (aromatic or nonaromatic) optionally substituted by a ketone moiety or is aryl optionally substituted by a heterocycle (aromatic or nonaromatic) optionally substituted by nitrile or is aryl optionally substituted by a heterocycle (aromatic or nonaromatic) optionally substituted by 1 to 3 C₁₋₄ alkyl groups; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups;and d is 0 to 2.

In another embodiment R¹⁷ is hydrogen or methyl or ethyl or together with R¹⁹ a methylene; and R¹⁸ is hydrogen or phenyl or 3-fluorophenyl or together with R²¹ a 3,4-dihydroisoquinoline ring; and R¹⁹ is hydrogen; and R²⁰ is hydrogen or methyl or together with R¹⁷ can form an ethylene; and X is CR²¹ R²² or NR²³ or ethylene; and R²¹ is hydrogen or benzyl or phenyl or 4-cyanophenoxy or 4-chlorophenoxy or 4-chlorobenzyl or methyl or tert-butyl or 3-fluorophenyl or 2-trifluoromethylphenyl or 2 methoxyphenyl or methoxy or 2-methylphenyl or 4-fluorobenzyl or 2-fluorophenyl or 4-chlorophenyl; and R²² is hydrogen or hydroxyl or trifluoromethyl; and R²³ is 2-methoxyphenyl or 3-trifluoromethyl phenyl or 4-fluorophenyl.

In a preferred embodiment R¹⁷ is hydrogen or methyl or ethyl or can form together with R¹⁹ a methylene group; and R¹⁸ is hydrogen or phenyl or together with R²¹ a 3,4-dihydroisoquinoline ring; and R¹⁹ is hydrogen; and R²⁰ is hydrogen or methyl or together with R¹⁷ can form an ethylene; and X is CR²¹R²² or ethylene or NR²³; and R²¹ is hydrogen or methyl or 2-methoxyphenyl or 4-chlorophenyl; and R²² is hydroxyl or methyl; and R²³ is 2-methoxyphenyl or 3-trifluoromethyl phenyl or 4-fluorophenyl.

In a preferred embodiment of the invention A is a group of formula II wherein n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

except:

4-[(4-ethyl-1-piperazinyl)-6-propyl]pyrimidin-2-amine.

In another preferred embodiment of the invention A is a group of formula II wherein n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups; and B is a group of formula X wherein R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and R¹⁻⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups;

and d is 0to 2.

In another preferred embodiment of the invention A is a group of formula II wherein n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups; and B is a group of formula XI wherein R¹⁷ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by nitrile, or by aryl optionally substituted by 1 to 3 C₁₋₄ alkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl, or can form together with R¹⁸ a benzene ring fused to the nitrogen heterocycle; and R¹⁸ is hydrogen or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1-3 C₁₋₄ alkyl groups or can form together with R²¹ a benzene ring fused to the nitrogen heterocycle, in which case R²² is not present; and R¹⁹ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups or is C₁₋₃ dialkylamide or can form together with R¹⁷ a C₁₋₃ alkylene chain; and R²⁰ is hydrogen or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups, or can form together with R¹⁷ a C₁₋₃ alkylene chain; and X is CR²¹R²², or is NR²³ or is C₂₋₃ alkylene chain; and R²¹ is hydrogen, or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or is C₁₋₇ alkyl groups (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups or is C₁₋₃ alkoxy group; and R²² is hydrogen, or is hydroxyl, or is C₁₋₃ alkoxy, or is C₁₋₃ haloalkyl groups; and R²³ is hydrogen, or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by 1 to 3 C₁₋₄ alkyl groups, or is aryl optionally substituted by a heterocycle (aromatic or nonaromatic) optionally substituted by a ketone moiety or is aryl optionally substituted by a heterocycle (aromatic or nonaromatic) optionally substituted by nitrile or is aryl optionally substituted by a heterocycle (aromatic or nonaromatic) optionally substituted by 1 to 3 C₁₋₄ alkyl groups; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups; and d is 0 to 2.

In another preferred embodiment of the invention A is a group of formula II wherein n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups; and B is a group of formula IX wherein R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In another preferred embodiment of the invention A is a group of formula V wherein y is 1 or 2; and B is a group of formula X wherein R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups;

and d is 0 to 2.

In another preferred embodiment of the invention A is a group of formula IV wherein o is 0 or 1; and r is 0, 1 or 2; and x is 0 or 1; and R⁵ is hydrogen or is unsubstituted C₁₋₃ alkyl group; and R⁶ is hydrogen or is unsubstituted C₁₋₃ alkyl group; and R⁷ is hydrogen or is unsubstituted C₁₋₃ alkyl group; and B is a group of formula XI wherein R¹⁷ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by nitrile, or by aryl optionally substituted by 1 to 3 C₁₋₄ alkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl, or can form together with R¹⁸ a benzene ring fused to the nitrogen heterocycle; and R¹⁸ is hydrogen or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1-3 C₁₋₄ alkyl groups or can form together with R²¹ a benzene ring fused to the nitrogen heterocycle, in which case R²² is not present; and R¹⁹ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups or is C₁₋₃ dialkylamide or can form together with R¹⁷ a C₁₋₃ alkylene chain; and R²⁰ is hydrogen or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups, or can form together with R¹⁷ a C₁₋₃ alkylene chain; and X is CR²¹R²², or is NR²³ or is C₂₋₃ alkylene chain; and R²¹ is hydrogen, or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or is C₁₋₇ alkyl groups (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups or is C₁₋₃ alkoxy group; and R²² is hydrogen, or is hydroxyl, or is C₁₋₃ alkoxy, or is C₁₋₃ haloalkyl groups; and R²³ is hydrogen, or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by 1 to 3 C₁₋₄ alkyl groups, or is aryl optionally substituted by a heterocycle (aromatic or nonaromatic) optionally substituted by a ketone moiety or is aryl optionally substituted by a heterocycle (aromatic or nonaromatic) optionally substituted by nitrile or is aryl optionally substituted by a heterocycle (aromatic or nonaromatic) optionally substituted by 1 to 3 C₁₋₄ alkyl groups; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups; and d is 0 to 2;

except 4-[3-(Methylamino)azetidin-1-yl]-6(4-methylpiperidin-1-yl)pyrimidin-2,4-diamine; and 4-[3-(Methylamino)azetidin-1-yl]-6-piperidin-1-yl )pyrimidin-2-diamine.

In another preferred embodiment of the invention A is a group of formula IV wherein o is 0 or 1; and r is 0, 1 or 2; and x is 0 or 1; and R⁵ is hydrogen or is unsubstituted C₁₋₃ alkyl group; and R⁶ is hydrogen or is unsubstituted C₁₋₃ alkyl group; and R⁷ is hydrogen or is unsubstituted C₁₋₃ alkyl group; and B is a group of formula X wherein R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C_{1'}alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups;

and d is 0 to 2.

In another preferred embodiment of the invention A is a group of formula V wherein wherein y is 1 or 2; and B is a group of formula IX wherein R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In another preferred embodiment of the invention A is a group of formula VI wherein R⁸ is hydrogen or is unsubstituted C₁₋₃ alkyl group; and B is a group of formula X wherein R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups; and d is 0 to 2.

In another preferred embodiment of the invention A is a group of formula VII wherein k is 0 or 1; and p is 1 or 2 or 3; and q is 0 or 1 or 2; and R⁹ is hydrogen or is unsubstituted C₁₋₃ alkyl group; and R¹⁰ is hydrogen or is unsubstituted C₁₋₃ alkyl group; and B is a group of formula X wherein R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups.

In another preferred embodiment A is a group of formula IV wherein o is 0 or 1; and

r is 0, 1 or 2;and x is 0 or 1; and R⁵ is hydrogen or is unsubstituted C₁₋₃ alkyl group; and

R⁶ is hydrogen or is unsubstituted C₁₋₃ alkyl group; and R⁷ is hydrogen or is unsubstituted C₁₋₃ alkyl group; and B is a group of formula IX wherein R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

except N⁴-Bicyclo[1.1.1]pent-1-yl-6[(3R)-3-methylamino)pyrolidin-1-yl]pyrimidine-2,4-diamine.

In another preferred embodiment A is a group of formula III wherein m is 0, 1 or 2; and R² is hydrogen or is unsubstituted C₁₋₃ alkyl group; and R³ is hydrogen or is unsubstituted C₁₋₃ alkyl group; and R⁴ is hydrogen or is unsubstituted C₁₋₃ alkyl group; and R^{d} is hydrogen or is unsubstituted C₁₋₃ alkyl group; and R^{e} is hydrogen or is unsubstituted C₁₋₃ alkyl group; and B is a group of formula IX wherein R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In another preferred embodiment A is a group of formula VIII wherein z is 0, 1, 2 or 3; and

w is 0 or 1; and R^{10a} is hydrogen or unsubstituted C₁₋₃ alkyl group; and R^{10e} is a CH group or N; and B is a group of formula IX wherein R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In a preferred embodiment of the invention A is a group of formula V wherein y is 1 or 2; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

In a preferred embodiment of the invention A is a group of formula IV wherein o is 0 or 1; and r is 0, 1 or 2; and x is 0 or 1; and R⁵ is hydrogen or is unsubstituted C₁₋₃ alkyl group; and R⁶ is hydrogen or is unsubstituted C₁₋₃ alkyl group; and R⁷ is hydrogen or is unsubstituted C₁₋₃ alkyl group; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁-₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In a preferred embodiment of the invention A is a group of formula VII wherein k is 0 or 1; and p is 1 or 2 or 3; and q is 0 or 1 or 2; and R⁹ is hydrogen or is unsubstituted C₁₋₃ alkyl group; and R¹⁰ is hydrogen or is unsubstituted C₁₋₃ alkyl group; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1

C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

In a preferred embodiment of the invention A is a group of formula III wherein m is 0, 1 or 2; and R² is hydrogen or is unsubstituted C₁₋₃ alkyl group; and R³ is hydrogen or is unsubstituted C₁₋₃ alkyl group; and R⁴ is hydrogen or is unsubstituted C₁₋₃ alkyl group; and R^{d} is hydrogen or is unsubstituted C₁₋₃ alkyl group; and R^{e} is hydrogen or is unsubstituted C₁₋₃ alkyl group; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In a preferred embodiment of the invention A is a group of formula XIV wherein D is NH and E is CH; or wherein D is direct bond and E is CH or N; and t is 1, 2 or 3; and R^{10h} is hydrogen or is unsubstituted C₁₋₃ alkyl group; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In a more preferred embodiment of the invention A is a group of formula II wherein n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups; and B is a group of formula X wherein R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups;

and d is 0 to 2.

In another more preferred embodiment of the invention A is a group of formula II wherein n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups; and B is a group of formula IX wherein

R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In a more preferred embodiment of the invention A is a group of formula II wherein n is 1 or 2; and R¹ is hydrogen or is unsubstituted C₁₋₃ alkyl groups or is C₃₋₅ unsubstituted cycloalkyl; and R^{a} is hydrogen or is unsubstituted C₁₋₄ alkyl groups; and R^{b} is hydrogen or is unsubstituted C₁₋₃ alkyl groups; and R^{c} is hydrogen or is unsubstituted C₁₋₃ alkyl groups; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁-4 alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

except: 4-[(4-ethyl-1-piperazinyl)-6-propyl]pyrimidin-2-amine.

In another more preferred embodiment of the invention A is a group of formula IV wherein o is 0 or 1; and r is 0, 1 or 2; and x is 0 or 1; and R⁵ is hydrogen or is unsubstituted C₁₋₃ alkyl group; and R⁶ is hydrogen or is unsubstituted C₁₋₃ alkyl group; and R⁷ is hydrogen or is unsubstituted C₁₋₃ alkyl group; and B is a group of formula X wherein R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C_{1'}alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen; and R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens; and R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups; and R¹⁶ is hydrogen or can form together with R¹³ a C₁₋₃ alkylene chain; and R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups;

and d is 0 to 2.

In another more preferred embodiment of the invention A is a group of formula V wherein wherein y is 1 or 2; and B is a group of formula IX wherein R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In another more preferred embodiment A is a group of formula IV wherein o is 0 or 1; and r is 0, 1 or 2;and x is 0 or 1; and R⁵ is hydrogen or is unsubstituted C₁₋₃ alkyl group; and R⁶ is hydrogen or is unsubstituted C₁₋₃ alkyl group; and R⁷ is hydrogen or is unsubstituted C₁₋₃ alkyl group; and B is a group of formula IX wherein R¹¹ is hydrogen; and R¹² is C₅₋₁₀ cycloalkyl (mono- or polycyclic) optionally substituted by 1 to 3 C₁₋₄ alkyl groups, or by aryl, or fused to an aryl;

or R¹¹ is hydrogen; and R¹² is C₅₋₁₀ nonaromatic heterocycle (mono- or polycyclic) optionally substituted by a ketone moiety, or by aryl, or by C₁₋₄ alkyl;

or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₃₋₁₀ cycloalkyl (mono- or polycyclic) optionally substituted by 1 to 3 C₁₋₄ alkyl groups;

or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups; and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;

except N⁴-Bicyclo[1.1.1]pent-1-yl-6[(3R)-3-methylamino)pyrolidin-1-yl]pyrimidine-2,4-diamine.

In another more preferred embodiment A is a group of formula III wherein m is 0, 1 or 2; and R² is hydrogen or is unsubstituted C₁₋₃ alkyl group; and R³ is hydrogen or is unsubstituted C₁₋₃ alkyl group; and R⁴ is hydrogen or is unsubstituted C₁₋₃ alkyl group; and R^{d} is hydrogen or is unsubstituted C₁₋₃ alkyl group; and R^{e} is hydrogen or is unsubstituted C₁₋₃ alkyl group; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

In another more preferred embodiment of the invention A is a group of formula IV wherein o is 0 or 1; and r is 0, 1 or 2; and x is 0 or 1; and R⁵ is hydrogen or is unsubstituted C₁₋₃ alkyl group; and R⁶ is hydrogen or is unsubstituted C₁₋₃ alkyl group; and R⁷ is hydrogen or is unsubstituted C₁₋₃ alkyl group; and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C_{1'}alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1 C₁₋₃ alkoxy group, or by a heterocycle (aromatic or non aromatic), or by C₁₋₄ alkyl, or by aryl;

or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

Preferred compounds of the invention are:
4-(4-methylpiperazin-1-yl )-6-piperidin-1-ylpyrimidin-2-amine;
4-(1,3-dihydro-2H-isoindol-2-yl)-6-(4-methylpiperazin-1-yl)pyrimidin-2-amine;
4-(4-methylpiperazin-1-yl)-6-(4-methylpiperidin-1-yl)pyrimidin-2-amine;
4-[4-(2-methoxyphenyl)piperidin-1-yl]-6-(4-methylpiperazin-1-yl)pyrimidin-2-amine;
4-(3,4-dihydroisoquinolin-2(1H)-yl)-6-(4-methylpiperazin-1-yl)pyrimidin-2-amine;

4-(5-fluoro-1,3-dihydro-2H-isoindol-2-yl)-6-(4-methylpiperazin-1-yl )pyrimidin-2-amine;
4-(3-aminopyrrolidin-1-yl)-6-(1,3-dihydro-2H-isoindol-2-yl)pyrimidin-2-amine;
4-(3-aminopyrrolidin-1-yl)-6-(1,3-dihydro-2H-isoindol-2-yl)pyrimidin-2-amine;
4-(4-methylpiperazin-1-yl)-6-(2-methylpyrrolidin-1-yl)pyrimidin-2-amine;
4-(2-ethylpiperidin-1-yl)-6-(4-methylpiperazin-1-yl )pyrimidin-2-amine;
4-[3-(2-methoxyphenyl)pyrrolidin-1-yl]-6-(4-methylpiperazin-1-yl)pyrimidin-2-amine;
4-[3-(4-chlorophenyl)pyrrolidin-1-yl]-6-(4-methylpiperazin-1-yl)pyrimidin-2-amine;
4-(2-methylpyrrolidin-1-yl)-6-piperazin-1-ylpyrimidin-2-amine;
4-(4-methylpiperazin-1-yl)-6-[(2R)-2-methylpyrrolidin-1-yl]pyrimidin-2-amine;
4-(4-methylpiperazin-1-yl)-6-[(2S)-2-methylpyrrolidin-1-yl]pyrimidin-2-amine;
4-(2,6-dimethylpiperidin-1-yl)-6-(4-methylpiperazin-1-yl)pyrimidin-2-amine;
4-(6-azabicyclo[3.2.1]oct-6-yl)-6-(4-methylpiperazin-1-yl)pyrimidin-2-amine;
4-azepan-1-yl-6-(4-methylpiperazin-1-yl)pyrimidin-2-amine;
1-[2-amino-6-(4-methylpiperazin-1-yl)pyrimidin-4-yl]-4-(4-chlorophenyl)piperidin-4-ol;
4-(4-methylpiperazin-1-yl)-6-(3-phenylpiperidin-1-yl)pyrimidin-2-amine;
N⁴-cyclohexyl-6-(hexahydropyrrolo[1,2-a]pyrazin-2(1 H)-yl)pyrimidine-2,4-diamine;
N⁴-adamantan-1-yl-6-(4-methylpiperazin-1-yl)pyrimidine-2,4-diamine;
6-(4-methylpiperazin-1-yl)-N⁴-(1,3,3-trimethylbicyclo[2.2.1]hept-2-yl)pyrimidine-2,4-diamine;
N⁴-adamantan-2-yl-6-(4-methylpiperazin-1-yl)pyrimidine-2,4-diamine;
6-(4-methylpiperazin-1-yl)-N⁴(1R; 4R)(1,7,7-trimethylbicyclo[2.2.1]hept-2-yl)pyrimidine-2,4-diamine;
N⁴-cyclohexyl-N⁴-methyl-6-(4-methylpiperazin-1-yl)pyrimidine-2,4-diamine;
4-(7-azabicyclo[2.2.1]hept-7-yl)-6-(4-methylpiperazin-1-yl)pyrimidin-2-amine;
4-(3-aminopyrrolidin-1-yl)-6-(5-fluoro-1,3-dihydro-2H-isoindol-2-yl)pyrimidin-2-amine;
4-(hexahydropyrrolo[1,2-a]pyrazin-2(1H)-yl)-6-(2-methylpyrrolidin-1-yl)pyrimidin-2-amine;
N⁴-1-azabicyclo[2.2.2]oct-3-yl-6-(1,3-dihydro-2H-isoindol-2-yl)pyrimidine-2,4-diamine;
4-(3-methyl-3,4-dihydroisoquinolin-2(1H)-yl )-6-(4-methylpiperazin-1-yl)pyrimidin-2-amine;
N⁴-1-azabicyclo[2.2.2]oct-3-yl-6-(2-methylpyrrolidin-1-yl)pyrimidine-2,4-diamine;
6-(2-methylpyrrolidin-1-yl)-N⁴-pyrrolidin-3-yl)pyrimidine-2,4-diamine;
4-[4-(methylamino)piperidin-1-yl]-6-(2-methylpyrrolidin-1-yl)pyrimidin-2-amine;
4-(1,3-dihydro-2H-isoindol-2-yl)-6-[4-(methylamino)piperidin-1-yl]pyrimidin-2-amine;
4-(1,3-dihydro-2H-isoindol-2-yl)-6-(4-methyl-1,4-diazepan-1-yl)pyrimidin-2-amine;
6-[(3R)-3-aminopyrrolidin-1-yl]-N⁴cyclohexylpyrimidine-2,4-diamine triacetate salt;
4-(3-methyl-3,4-dihydroisoquinolin-2(1H)-yl)-6-(4-methylpiperazin-1-yl)pyrimidin-2-amine;
(R)-4-(3-methyl-3,4-dihydroisoquinolin-2(1H)-yl)-6-(4-methylpiperazin-1-yl)pyrimidin-2-amine;
(S)-4-(3-methyl-3,4-dihydroisoquinolin-2(1H)-yl)-6-(4-methylpiperazin-1-yl)pyrimidin-2-amine;
N⁴-cyclohexyl-N⁶-[2-(dimethylamino)ethyl]pyrimidine-2,4,6-triamine;
N⁴-cyclohexyl-6-[4-(methylamino)piperidin-1-yl]pyrimidine-2,4-diamine;
6-[(3S)-3-aminopyrrolidin-1-yl]-N⁴-cyclohexylpyrimidine-2,4-diamine;
N⁴-cyclopentyl-6-(4-methylpiperazin-1-yl)pyrimidine-2,4-diamine;
N⁴-cyclopentyl-N⁴-methyl-6-(4-methylpiperazin-1-yl)pyrimidine-2,4-diamine;
N⁴-cycloheptyl-6-(4-methylpiperazin-1-yl)pyrimidine-2,4-diamine;
4-[(1R*,5S*)-8-azabicyclo[3.2.1]oct-8-yl]-6-(4-methylpiperazin-1-yl)pyrimidin-2-amine;
N⁴-[(1R*,2S*,4S*)-bicyclo[2.2.1]hept-2-yl]-6-(4-methylpiperazin-1-yl)pyrimidine-2,4-diamine;
N⁴-bicyclo[2.2.1]hept-2-yl-6-(4-methylpiperazin-1-yl)pyrimidine-2,4-diamine;
N⁴-[(1R*,2R*,4S*)-bicyclo[2.2.1]hept-2-yl]-N⁶-[2-(dimethylamino)ethyl]pyrimidin-2,4,6-triamine;
N⁴-[(1R*,2R*,4S*)-bicyclo[2.2.1]hept-2-yl]-6-[(3aR*,6aS*)-hexahydropyrrolo[3,4-c]pyrrol-2(1 H)-yl]pyrimidine-2,4-diamine;
4-cyclohexyl-6-(4-methylpiperazin-1-yl)pyrimidin-2-amine;
4-adamantan-2-yl-6-(4-methylpiperazin-1-yl)pyrimidin-2-amine;
4-isopropyl-6-(4-methylpiperazin-1-yl)pyrimidin-2-amine;
4-(1-methylpentyl)-6-(4-methylpiperazin-1-yl)pyrimidin-2-amine;
4-cyclopentyl-6-(4-methylpiperazin-1-yl)pyrimidin-2-amine;
4-(1-ethylpropyl)-6-(4-methylpiperazin-1-yl)pyrimidin-2-amine;
4-cyclohexyl-6-(hexahydropyrrolo[1,2-a]pyrazin-2(1H)-yl)pyrimidin-2-amine;
4-cyclohexyl-6-(4-methyl-1,4-diazepan-1-yl)pyrimidin-2-amine;
4-cyclohexyl-6-(4-ethylpiperazin-1-yl)pyrimidin-2-amine;
4-cyclohexyl-6-[3-(dimethylamino)pyrrolidin-1-yl]pyrimidin-2-amine;
6-cyclohexyl-N⁴-methyl-N⁴-(1-methylpyrrolidin-3-yl)pyrimidine-2,4-diamine;
6-cyclohexyl-N⁴-(1-methylpiperidin-4-yl)pyrimidine-2,4-diamine;
4(R)-(4-methylpiperazin-1-yl)-6-(1-phenylethyl)pyrimidin-2-amine;
4(S)-(4-methylpiperazin-1-yl)-6-(1-phenylethyl)pyrimidin-2-amine;
4-(3-aminopyrrolidin-1-yl)-6-cyclopropylpyrimidin-2-amine;
4-cyclopropyl-6-[3-(methylamino)pyrrolidin-1-yl]pyrimidin-2-amine;
4-(3-aminopyrrolidin-1-yl)-6-(4-methylcyclohex-1-en-1-yl)pyrimidin-2-amine di-trifluoroacetic acid salt;
6-cyclohex-1-en-1-yl-N⁴-[2-(dimethylamino)ethyl]pyrimidine-2,4-diamine;
4-tert-butyl-6-(4-methylpiperazin-1-yl)pyrimidin-2-amine acetate salt;
4-tert-butyl-6-[3-(methylamino)pyrrolidin-1-yl]pyrimidin-2-amine acetate salt;
4-[adamantan-2-yl]-6-[(3S)-3-aminopyrrolidin-1-yl]pyrimidin-2-amine;
4-[adamantan-2-yl]-6-[(3S)-3-(methylamino)pyrrolidin-1-yl]pyrimidin-2-amine;
4-cyclohept-1-en-1-yl-6-(4-methylpiperazin-1-yl)pyrimidin-2-amine;
4-cyclopentyl-6-(3-methylpiperazin-1-yl)pyrimidin-2-amine;
4-(3-aminoazetidin-1-yl)-6-cyclohex-1-en-1-ylpyrimidin-2-amine;
4-cyclohexyl-6-(3-methylpiperazin-1-yl)pyrimidin-2-amine;
4-(3-aminoazetidin-1-yl)-6-cyclohexylpyrimidin-2-amine;
4-(4-methylpiperazin-1-yl)-6-(1,2,3,4-tetrahydronaphthalen-2-yl)pyrimidin-2-amine;
4-[3-(methylamino)pyrrolidin-1-yl]-6-(1,2,3,4-tetrahydronaphthalen-2-yl)pyrimidin-2-amine;
4-(3-aminopyrrolidin-1-yl)-6-(1,2,3,4-tetrahydronaphthalen-2-yl)pyrimidin-2-amine;
4-cyclopentyl-6-[(3S)-3-methylpiperazin-1-yl]pyrimidin-2-amine;
4-cyclopentyl-6-[(3R)-3-methylpiperazin-1-yl]pyrimidin-2-amine;
4-cyclohexyl-6-[(2S)-2-methylpiperazin-1-yl]pyrimidin-2-amine;
4-[(3R)-3-aminopyrrolidin-1-yl]-6-[(1E)-3,3-dimethylbut-1-en-1-yl]pyrimidin-2-amine;
4-[3-(aminomethyl)azetidin-1-yl]-6-cyclohexylpyrimidin-2-amine;
4-(3-aminoazetidin-1-yl)-6-cyclopentylpyrimidin-2-amine;
4-cyclopentyl-6-(3-ethylpiperazin-1-yl)pyrimidin-2-amine;
4-cyclopentyl-6-[(3S)-3-isopropylpiperazin-1-yl]pyrimidin-2-amine;
4-cyclopentyl-6-(3,8-diazabicyclo[3.2.1]oct-3-yl)pyrimidin-2-amine;
N⁴-(2,3-dihydro-1H-inden-2-yl)-6-[3-(methylamino)pyrrolidin-1-yl]pyrimidine-2,4-diamine;
4-cyclopentyl-6-[(3S)-3-isobutylpiperazin-1-yl]pyrimidin-2-amine;
4-cyclopentyl-6-[3-(ethylamino)azetidin-1-yl]pyrimidin-2-amine;
6-(4-methylpiperazin-1-yl)-N⁴-(tetrahydro-2H-pyran-4-yl)pyrimidine-2,4-diamine;
6-(4-methylpiperazin-1-yl)-N⁴-(tetrahydro-2H-pyran-4-yl)pyrimidine-2,4-diamine acetate;
N⁴-cyclohexyl-6-(4-methylpiperazin-1-yl)pyrimidine-2,4-diamine;
4-(1,3-dihydro-2H-isoindol-2-yl)-6-piperazin-1-ylpyrimidin-2-amine;
4-(3-aminopyrrolidin-1-yl)-6-(2-methylpyrrolidin-1-yl)pyrimidin-2-amine;
4-[3-(methylamino)pyrrolidin-1-yl]-6-(2-methylpyrrolidin-1-yl)pyrimidin-2-amine;
6-(3-aminopyrrolidin-1-yl)-N⁴-[(1R*,2S*,4S*)-bicyclo[2.2.1]hept-2-yl]pyrimidine-2,4-diamine;
N⁴-[(1R,2S*,4S*)-bicyclo[2.2.]hept-2-yl]-6-[3-(methylamino)pyrrolidin-1-yl]pyrimidine-2,4-diamine;
6-[(3S)-3-aminopyrrolidin-1-yl]-N⁴-[(1R,2S*,4S*)-bicyclo[2.2.]hept-2-yl]pyrimidine-2,4-diamine;
6-[(3R)-3-aminopyrrolidin-1-yl]-N⁴-[(1R*,2S*,4S*)-bicyclo[2.2.]hept-2-yl]pyrimidine-2,4-diamine;
6-[(3S)-3-aminopyrrolidin-1-yl]-N⁴-[(1 R,2R*,4S*)-bicyclo[2.2.1]hept-2-yl]pyrimidine-2,4-diamine;
6-[(3R)-3-aminopyrrolidin-1-yl]-N⁴-[(1R*,2R*,4S*)-bicyclo[2.2.1]hept-2-yl]pyrimidine-2,4-diamine;
N⁴-[exo-bicyclo[2.2.1]hept-2-yl]-6-[(3S)-3-(methylamino)pyrrolidin-1-yl]pyrimidine-2,4-diamine;
N⁴-[(1R*,2R*,4S*)-bicyclo[2.2.1]hept-2-yl]-6-[(3R)-3-(methylamino)pyrrolidin-1-yl]pyrimidine-2,4-diamine;
N⁴-[(1R*,2S*,4S*)-bicyclo[2.2.1]hept-2-yl]-6-[(3S)-3-(methylamino)pyrrolidin-1-yl]pyrimidine-2,4-diamine;
N⁴-[(1R*,2S*,4S*)-bicyclo[2.2.1]hept-2-yl]-6-[(3R)-3-(methylamino)pyrrolidin-1-yl]pyrimidine-2,4-diamine;
N⁴-[(1R*,2R*,4S*)-bicyclo[2.2.1]hept-2-yl]-6-(3-methylpiperazin-1-yl)pyrimidine-2,4-diamine;
N⁴-[(1R*,2R*,4S*)-bicyclo[2.2.1]hept-2-yl]-6-(1,4-diazepan-1-yl)pyrimidine-2,4-diamine;
N⁴-[(1R*,2R*,4S*)-bicyclo[2.2.1]hept-2-yl]-6-((4aR*,7aR*)-octahydro-6H-pyrrolo[3,4-b]pyridin-6-yl)pyrimidine-2,4-diamine;
N⁴-[(1R*,2R*,4S*)-bicyclo[2.2.1]hept-2-yl]-6-(hexahydropyrrolo[1,2-a]pyrazin-2(1H)-yl)pyrimidine-2,4-diamine;
N⁴-[(1R*,2R*,4S*)-bicyclo[2.2.1]hept-2-yl]-6-piperazin-1-ylpyrimidine-2,4-diamine;
4-(3-aminopyrrolidin-1-yl)-6-cyclopentylpyrimidin-2-amine;
4-adamantan-2-yl-6-(3-aminopyrrolidin-1-yl)pyrimidin-2-amine;
4-cyclopentyl-6-[3-(methylamino)pyrrolidin-1-yl]pyrimidin-2-amine;
4-[(3S)-3-aminopyrrolidin-1-yl]-6-cyclohexylpyrimidin-2-amine;
4-[(3R)-3-aminopyrrolidin-1-yl]-6-cyclohexylpyrimidin-2-amine;
4-(3-aminopyrrolidin-1-yl)-6-(cyclohexylmethyl)pyrimidin-2-amine;
4-cyclopentyl-6-piperazin-1-ylpyrimidin-2-amine;
4-[adamantan-2-yl]-6-[(3R)-3-(methylamino)pyrrolidin-1-yl]pyrimidin-2-amine;
4-[adamantan-2-yl]-6-[(3R)-3-aminopyrrolidin-1-yl]pyrimidin-2-amine;
4-(cyclopentylmethyl)-6-(3-methylpiperazin-1-yl)pyrimidin-2-amine;
4-(3-aminopyrrolidin-1-yl)-6-(cyclopentylmethyl)pyrimidin-2-amine;
4-cyclohexyl-6-[3-(methylamino)azetidin-1-yl]pyrimidin-2-amine;
4-cyclopentyl-6-[(4aR*,7aR*)-octahydro-6H-pyrrolo[3,4-b]pyridin-6-yl]pyrimidin-2-amine;
4-cyclohexyl-6-(1,4-diazepan-1-yl)pyrimidin-2-amine;
4-cyclopentyl-6-(1,4-diazepan-1-yl)pyrimidin-2-amine;
6-cyclopentyl-N⁴-[2-(methylamino)ethyl]pyrimidine-2,4-diamine;
4-[(3R)-3-aminopyrrolidin-1-yl]-6-cyclohex-1-en-1-ylpyrimidin-2-amine.

More preferred compounds of the invention are:
N⁴-cyclohexyl-6-(4-methylpiperazin-1-yl)pyrimidine-2,4-diamine;
4-(1,3-dihydro-2H-isoindol-2-yl)-6-piperazin-1-ylpyrimidin-2-amine;
4-(3-aminopyrrolidin-1-yl)-6-(2-methylpyrrolidin-1-yl)pyrimidin-2-amine;
4-[3-(methylamino)pyrrolidin-1-yl]-6-(2-methylpyrrolidin-1-yl)pyrimidin-2-amine;
6-(3-aminopyrrolidin-1-yl)-N⁴-[(1R,2S*,4S*)-bicyclo[2.2.1]hept-2-yl]pyrimidine-2,4-diamine;
N⁴-[(1R*,2S*,4S*)-bicyclo[2.2.1]hept-2-yl]-6-[3-(methylamino)pyrrolidin-1-yl]pyrimidine-2,4-diamine;
6-[(3S)-3-aminopyrrolidin-1-yl]-N⁴-[(1R,2S*,4S*)-bicyclo[2.2.1]hept-2-yl]pyrimidine-2,4-diamine;
6-[(3R)-3-aminopyrrolidin-1-yl]-N⁴-[(1R*,2S*,4S*)-bicyclo[2.2.1]hept-2-yl]pyrimidine-2,4-diamine;
6-[(3S)-3-aminopyrrolidin-1-yl]-N⁴-[(1R*,2R*,4S*)-bicyclo[2.2.1]hept-2yl]pyrimidine-2,4-diamine;
6-[(3R)-3-aminopyrrolidin-1-yl]-N⁴-[(1R,2R*,4S*)-bicyclo[2.2.1]hept-2-yl]pyrimidine-2,4-diamine;
N⁴-[exo-bicyclo[2.2.1]hept-2-yl]-6-[(3S)-3-(methylamino)pyrrolidin-1-yl]pyrimidine-2,4-diamine;
N⁴-[(1R*,2R*,4S*)-bicyclo[2.2.1]hept-2-yl]-6-[(3R)-3-(methylamino)pyrrolidin-1-yl]pyrimidine-2,4-diamine;
N⁴-[(1R*,2S*,4S*)-bicyclo[2.2.1]hept-2-yl]-6-[(3S)-3-(methylamino)pyrrolidin-1-yl]pyrimidine-2,4-diamine;
N⁴-[(1R*,2S*,4S*)-bicyclo[2.2.1]hept-2-yl]-6-[(3R)-3-(methylamino)pyrrolidin-1-yl]pyrimidine-2,4-diamine;
N⁴-[(1R*,2R*,4S*)-bicyclo[2.2.1]hept-2-yl]-6-(3-methylpiperazin-1-yl)pyrimidine-2,4-diamine;
N⁴-[(1R*,2R*,4S*)-bicyclo[2.2.1]hept-2-yl]-6-(1,4-diazepan-1-yl)pyrimidine-2,4-diamine;
N⁴-[(1R*,2R*,4S*)-bicyclo[2.2.1]hept-2-yl]-6-((4aR*,7aR*)-octahydro-6H-pyrrolo[3,4-b]pyridin-6-yl)pyrimidine-2,4-diamine;
N⁴-[(1R*,2R*,4S*)-bicyclo[2.2.1]hept-2-yl]-6-(hexahydropyrrolo[1,2-a]pyrazin-2(1H)-ylpyrimidine-2,4-diamine;
N⁴-[(1R*,2R*,4S*)-bicyclo[2.2.1]hept-2-yl]-6-piperazin-1-ylpyrimidine-2,4-diamine;
4-(3-aminopyrrolidin-1-yl)-6-cyclopentylpyrimidin-2-amine;
4-adamantan-2-yl-6-(3-aminopyrrolidin-1-yl)pyrimidin-2-amine;
4-cyclopentyl-6-[3-(methylamino)pyrrolidin-1-yl]pyrimidin-2-amine;
4-[(3S)-3-aminopyrrolidin-1-yl]-6-cyclohexylpyrimidin-2-amine;
4-[(3R)-3-aminopyrrolidin-1-yl]-6-cyclohexylpyrimidin-2-amine;
4-(3-aminopyrrolidin-1-yl)-6-(cyclohexylmethyl)pyrimidin-2-amine;
4-cyclopentyl-6-piperazin-1-ylpyrimidin-2-amine;
4-[adamantan-2-yl]-6-[(3R)-3-(methylamino)pyrrolidin-1-yl]pyrimidin-2-amine;
4-[adamantan-2-yl]-6-[(3R)-3-aminopyrrolidin-1-yl]pyrimidin-2-amine;
4-(cyclopentylmethyl)-6-(3-methylpiperazin-1-yl)pyrimidin-2-amine;
4-(3-aminopyrrolidin-1-yl)-6-(cyclopentylmethyl)pyrimidin-2-amine;
4-cyclohexyl-6-[3-(methylamino)azetidin-1-yl]pyrimidin-2-amine;
4-cyclopentyl-6-[(4aR*,7aR*)-octahydro-6H-pyrrolo[3,4-b]pyridin-6-yl]pyrimidin-2-amine;
4-cyclohexyl-6-(1,4-diazepan-1-yl)pyrimidin-2-amine;
4-cyclopentyl-6-(1,4-diazepan-1-yl)pyrimidin-2-amine;
6-cyclopentyl-N⁴-[2-(methylamino)ethyl]pyrimidine-2,4-diamine;
4-[(3R)-3-aminopyrrolidin-1-yl]-6-cyclohex-1-en-1-ylpyrimidin-2-amine.

The “pharmaceutically acceptable salts” according to the invention include all therapeutically active, non-toxic acid salt forms which the compounds of formula (I) are able to form. The acid addition salt form of a compound of formula (I) that occurs in its free form as a base can be obtained by treating the free base with an appropriate acid such as an inorganic acid, for example, a hydrohalic such as hydrochloric, hydroiodic or hydrobromic, sulfuric, nitric, phosphoric and the like; or an organic acid, such as, for example, acetic, oxalic, p-bromophenylsulfonic, carbonic, benzoic, formic, propionic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, palmoic, and the like. Conversely said salt forms can be converted into the free forms by treatment with an appropriate base.

The “pharmaceutically acceptable salts” according to the invention include therapeutically active, non-toxic base salt forms which the compounds of formula I are able to form. For example the compounds of formula I containing acidic protons may be converted into their therapeutically active, non-toxic base addition salt forms, e.g. metal or amine salts, by treatment with appropriate organic and inorganic bases. Appropriate base salt forms include, for example but are not limited to, ammonium salts, alkali and alkaline earth metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like. Conversely said salt forms can be converted into the free forms by treatment with an appropriate acid.

Compounds of the formula I and their salts can be in the form of solvates, which are included within the scope of the present invention. Such solvates include for example hydrates, alcoholates and the like.

Some of the compounds of formula I and some of their intermediates have at least one stereogenic centre in their structure. This stereogenic centre may be present in a R or a S configuration, said R and S notation is used in correspondence with the rules described in Pure Appl. Chem., 45 (1976) 11-30.

The invention also relates to all stereoisomeric forms such as enantiomeric and diastereoisomeric forms of the compounds of formula I or mixtures thereof (including all possible mixtures of stereoisomers).

The invention also relates to all pure enantiomers of the racemic mixtures among which 4-(3-methyl-3,4-dihydroisoquinolin-2(1H)-yl)-6-(4-methylpiperazin-1-yl)pyrimidin-2-amine, 4-(4-methylpiperazin-1-yl)-6-(1-phenylethyl)pyrimidin-2-amine.

Some of the compounds of formula I may also exist in tautomeric forms. Such forms although not explicitly indicated in the above formula are intended to be included within the scope of the present invention.

With respect to the present invention reference to a compound or compounds is intended to encompass that compound in each of its possible isomeric forms and mixtures thereof unless the particular isomeric form is referred to specifically.

Compounds according to the present invention may exist in different polymorphic forms. Although not explicitly indicated in the above formula, such forms are intended to be included within the scope of the present invention.

The invention also includes within its scope prodrug forms of the compounds of formula I and its various sub-scopes and sub-groups.

The term “pro-drug” as used herein includes compound forms, which are rapidly transformed in vivo to the parent compound according to the invention, for example, by hydrolysis in blood. Pro-drugs are compounds bearing groups that are removed by biotransformation prior to exhibiting their pharmacological action. Pro-drugs form a class of groups well known to practitioners of the art. In the present case they include, tertbutyl carbamate groups. The compounds bearing this functional group are also used as synthetic intermediates. Pro-drug compounds have the advantage that they may exhibit improved bioavailability as a result of enhanced solubility and/or rate of absorption (T. Higuchi and V. Stella, “Pro-drugs as Novel Delivery System”, Vol. 14 of the A.C.S. Symposium Series; “Bioreversible Carriers in Drug Design”, ed. Edward B. Roche, American Pharmaceutical Association and Pergamon Press, 1987).

Potential pro-drugs of the invention are:
tert-butyl [1-(6-adamantan-2-yl-2-aminopyrimidin4-yl)pyrrolidin-3-yl]carbamate formate salt;
tert-butyl(3aR*,6aS*)-5-(2-amino-6-cyclohexylpyrimidin-4-yl)hexahydropyrrolo[3,4-c]pyrrole-2(1H)-carboxylate);
(tert-butyl(1R*,5S*,6S*)-6-[(2-amino-6-cyclohexylpyrimidin-4-yl)amino]-3-azabicyclo[3.1.0]hexane-3-carboxylate);
(tert-butyl(1R*,5S*,6S*)-6-[(2-amino-6-cyclopentylpyrimidin-4-yl)amino]-3-azabicyclo[3.1.0]hexane-3-carboxylate);
(tert-butyl(1R*,5S*,6S*)-6-{[2-amino-6-(cyclopentylmethyl)pyrimidin-4-yl]amino}-3-azabicyclo[3.1.0]hexane-3-carboxylate);
(tert-butyl 4-[2-amino-6-(tert-butylamino)pyrimidin-4-yl]-1,4-diazepane-1-carboxylate);
(tert-butyl({1-[2-amino-6-(tert-butylamino)pyrimidin-4-yl]azetidin-3-yl}methyl)carbamate);
(tert-butyl{[1-(2-amino-6-cyclohexylpyrimidin-4-yl)azetidin-3-yl]methyl}carbamate);
(tert-butyl 4-{2-amino-6-[(1R,2R*,4S*)-bicyclo[2.2.1]hept-2-ylamino]pyrimidin-4-yl}-2-methylpiperazine-1-carboxylate);
(tert-butyl 4-{2-amino-6-[(1R*,2R*,4S*)-bicyclo[2.2.1]hept-2-ylamino]pyrimidin-4-yl}-1,4-diazepane-1-carboxylate);
(tert-butyl(4aR*,7aR*)-6-{2-amino-6-[(1R*,2R*,4S*)-bicyclo[2.2.1]hept-2-ylamino]pyrimidin-4-yl}octahydro-1H-pyrrolo[3,4-b]pyridine-1-carboxylate);
(tert-butyl(3aR*,6aS*)-5-{2-amino-6-[( R*,2R*,4S)-bicyclo[2.2.1]hept-2-ylamino]pyrimidin-4-yl}hexahydropyrrolo[3,4-c]pyrrole-2(1H)-carboxylate);
(tert-butyl 4-{2-amino-6-[(1R*,2R*,4S*)-bicyclo[2.2.1]hept-2-ylamino]pyrimidin-4-yl}piperazine-1-carboxylate);
(tert-butyl(3S)-4-(2-amino-6-cyclohexylpyrimidin-4-yl)-3-methylpiperazine-1-carboxylate);
(tert-butyl(3R)-4-(2-amino-6-cyclohexylpyrimidin4-yl)-3-methylpiperazine-1-carboxylate);
(tert-butyl(1-{2-amino-6-[(1R*,2S*,4S*)-bicyclo[2.2.1]hept-2-ylamino]pyrimidin-4-yl}pyrrolidin-3-yl)methylcarbamate);
(tert-butyl[(3S)-1-{2-amino-6-[(1R*,2S*,4S*)-bicyclo[2.2.1]hept-2-ylamino]pyrimidin-4-yl}pyrrolidin-3-yl]carbamate);
(tert-butyl[(3R)-1-{2-amino-6-[(1R*,2S*,4S*)-bicyclo[2.2.1]hept-2-ylamino]pyrimidin-4-yl}pyrrolidin-3-yl]carbamate);
(tert-butyl{1-[2-amino-6-(cyclohexylmethyl)pyrimidin-4-yl]pyrrolidin-3-yl}carbamate);
(tert-butyl[1-(2-amino-6-cyclopropylpyrimidin-4-yl)pyrrolidin-3-yl]carbamate);
(tert-butyl{1-[2-amino-6-(2-phenylethyl)pyrimidin-4-yl]pyrrolidin-3-yl}carbamate);
(tert-butyl 4-(2-amino-6-cyclopentylpyrimidin-4-yl)piperazine-1-carboxylate);
(tert-butyl{1-[2-amino-6-(cyclopentylmethyl)pyrimidin-4-yl]pyrrolidin-3-yl}carbamate);
(tert-butyl 4-{6-[adamantan-2-yl]-2-aminopyrimidin-4-yl}-2-methylpiperazine-1-carboxylate);
(tert-butyl(4aR*,7aR*)-6-(2-amino-6-cyclopentylpyrimidin-4-yl)octahydro-1H-pyrrolo[3,4-b]pyridine-1-carboxylate);
(tert-butyl[1-(2-amino-6-cyclopentylpyrimidin-4-yl)azetidin-3-yl]carbamate);
(tert-butyl(1-{2-amino-6-[(1R*,2S*,4S*)-bicyclo[2.2.]hept-2-ylamino]pyrimidin-4-yl}pyrrolidin-3-yl)carbamate);
(tert-butyl 4-(2-amino-6-cyclohexylpyrimidin-4-yl)-methylpiperazine-1 carboxylate);
tert-butyl[1-(2-amino-6-cyclohexylpyrimidin-4-yl)azetidin-3-yl]carbamate
(tert-butyl{1-[2-amino-6-(1,2,3,4-tetrahydronaphthalen-2-yl)pyrimidin-4-yl]pyrrolidin-3-yl}carbamate);
(tert-butyl 3-[(2-amino-6-cyclohexylpyrimidin-4-yl)(methyl)amino]pyrrolidine-1-carboxylate);
(tert-butyl[1-(2-amino-6-cyclohexylpyrimidin-4-yl)azetidin-3-yl]carbamate);
(tert-butyl(2S)-4-(2-amino-6-cyclopentylpyrimidin-4-yl)-2-methylpiperazine-1-carboxylate);
(tert-butyl(2R)-4-(2-amino-6-cyclopentylpyrimidin-4-yl)-2-methylpiperazine-1-carboxylate).

For example N-[1-(2-amino-6-cyclopentylpyrimidin-4-yl)azetidin-3-yl]acetamide, submitted to in vivo enzymatic hydrolysis of its amide can eliberate compound 4-(3-aminoazetidin-1-yl)-6-cyclopentylpyrimidin-2-amine.

It has now been found that compounds of formula I and their pharmaceutically acceptable salts are useful in a variety of pharmaceutical indications.

For example, the compounds according to the invention including are useful for the treatment of inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

Thus, the present invention, in a further aspect, concerns the use of a compound of formula I or a pharmaceutically acceptable salt thereof for the manufacture of a medicament for the treatment of disorders such as mentioned above.

In particular, the present invention concerns the use of a compound of formula I or a pharmaceutically acceptable salt thereof, for the manufacture of a medicament for the treatment of H₄ dependent such as inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin or diseases of the gastrointestinal tract including inflammatory bowel disease, Crohn's disease, ulcerative colitis or autoimmune diseases including rheumatoid arthritis, multiple sclerosis.

The compounds of the invention are useful for treating conditions in which there is an influx of leukocytes in the tissues. These conditions include inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin or diseases of the gastrointestinal tract including inflammatory bowel disease, Crohn's disease, ulcerative colitis or autoimmune diseases including rheumatoid arthritis, multiple sclerosis.

The compounds of the invention exhibit the biological activity by inhibiting the histamine binding to the H₄ receptor or on an activated H₄ receptor. Subjects in need of treatment for a H₄ dependent inflammatory disorder or inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or dermatological diseases such as dermatitis and psoriasis and treatment of itchy skin or diseases of the gastrointestinal tract including inflammatory bowel disease, Crohn's disease, ulcerative colitis or autoimmune diseases including rheumatoid arthritis, multiple sclerosis, can be treated by administering to the patient an effective amount of one or more of the above-identified compounds or a pharmaceutically acceptable derivative or salt thereof in a pharmaceutically acceptable carrier or diluent to reduce formation of oxygen radicals. The active materials can be administered by any appropriate route, for example, orally, parenterally, intravenously, intradermally, subcutaneously, intramuscularly or topically, in liquid, cream, gel or solid form, via a buccal or nasal spray, or aerosol.

The invention further concerns the use of the compounds of formula I for the manufacture of a medicament for therapeutic application. In particular, the invention concerns the use of the compounds of formula I for the manufacture of a medicament useful for treating conditions in which there is likely to be a H₄ dependent inflammatory component.

The invention concerns the use of the compound of formula I for the manufacture of a medicament useful for treating inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal disfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

The invention further concerns the compounds of formula I for use as medicaments.

The invention concerns the compounds of formula I for use as a medicament for inflammatory disorders or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; lymphatic diseases.

The activity and properties of the active compounds, oral availability and stability in vitro or in vivo can vary significantly among the optical isomers of the disclosed compounds.

In a preferred embodiment, the active compound is administered in an enantiomerically enriched form, i.e., substantially in the form of one isomer. By the term “substantially” we understand greater or equal to 95% of the said isomer.

The present invention also concerns a method for treating H₄ dependent inflammatory conditions inflammatory disorders, or respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion or diseases of the gastrointestinal tract such as inflammatory bowel disease, Crohn's disease, ulcerative colitis or autoimmune diseases such as rheumatoid arthritis, multiple sclerosis, atherosclerosis, skin diseases where there's an influx of inflammatory cells, cardiovascular diseases, in a mammal in need of such treatment, comprising administering a therapeutic dose of at least one compound of formula I or a pharmaceutically acceptable salt thereof to a patient.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 0.01 to 1000 mg, preferably 0.05 to 500 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The activity of the compounds of formula I or their pharmaceutically acceptable salts, as H₄ antagonists can be determined in a tritiated histamine binding assay and in a H₄ GTPγS³⁵ binding assay. The objective of this test is to evaluate the anti-H₄ potential of a compound by measuring its inhibitory effect on histamine binding to the H₄ receptor or on H₄ receptor activation. Results obtained with compounds of formula I are indicative of a strong pharmacological effect.

For treating diseases, compounds of formula I or their pharmaceutically acceptable salts, may be employed at an effective daily dosage and administered in the form of a pharmaceutical composition.

Therefore, another embodiment of the present invention concerns a pharmaceutical composition comprising an effective amount of a compound of formula I or a pharmaceutically acceptable salt thereof in combination with a pharmaceutically acceptable diluent or carrier.

To prepare a pharmaceutical composition according to the invention, one or more of the compounds of formula I or a pharmaceutically acceptable salt thereof, is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, or parenteral.

Pharmaceutical compositions comprising compounds according to the invention can, for example, be administered orally or parenterally, i.e., intravenously, intramuscularly, subcutaneously, transdermally, intrathecally or by inhalation.

Pharmaceutical compositions suitable for oral administration can be solids or liquids and can, for example, be in the form of tablets, pills, dragees, gelatine capsules, solutions, syrups, suppositories, patches, inhalants, and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula I in compositions for oral administration is at least 0.5% by weight and can be up to 80% by weight with respect to the total weight of the composition.

For the preferred oral compositions, the daily dosage is in the range 0.01 to 1000 milligrams (mg) of compounds of formula I. In compositions for parenteral administration, the quantity of compound of formula I, present is at least 0.5% by weight and can be up to 33% by weight with respect to the total weight of the composition. For the preferred parenteral compositions, the dosage unit is in the range 0.01 mg to 1000 mg of compounds of formula I.

The daily dose can fall within a wide range of dosage units of compound of formula I is generally in the range 0.01 to 1000 mg. However, it should be understood that the specific doses could be adapted to particular cases depending on the individual requirements, at the physician's discretion.

The compounds of the invention may be co-administered with another therapeutic agent most likely from a different therapeutic area.

Co-administration in this context means the dosing either of components, which are formulated together as a single dosage form; or the administration of separately formulated agents at substantially the same time, or sequential dosing of a compound of the invention followed by a therapeutic agent of a different therapeutic area.

In this context suitable examples of therapeutic agents may include, but are not limited to, histamine H₁ antagonists such as cetirizine, histamine H₂ antagonists, histamine H₃ antagonists, leukotriene antagonists, PDE₄ inhibitors such as roflumilast, muscarinic M₃ antagonists, β2 agonists, theophylline, sodium cromoglycate, anti-TNF antibodies such as certolizumab pegol or adalimumab, anti-IL6 antibodies, anti-IL17 antibodies, adhesion molecule inhibitors, inhibitors of cytokine synthesis such as P38 MAP kinase inhibitors and inhibitors of PI3 kinase, methotrexate.

The present invention concerns also processes for preparing the compounds of formula I.

The compounds of formula I according to the invention can be prepared analogously to conventional methods as understood by the person skilled in the art of synthetic organic chemistry.

The following processes description sets forth certain synthesis routes in an illustrative manner. Other alternative and/or analogous methods will be readily apparent to those skilled in this art.

Compounds of formula I may be prepared according to one of the following general methods.

The synthesis of the compounds of the invention can be done by starting from a 4,6-dichloropyrimidine bearing a leaving group at the 2 position, usually 2,4,6-trichloropyrimidine or a 2 alkylthio-4,6-dichloropyrimidine or from 2-amino-4,6-dichloropyrimidine.

In the case when B is C₃₋₁₀ cycloalkyl, or is C₅₋₁₀ cycloalkenyl group, or is C₂₋₇ alkyl group, or is C₂₋₆ alkenyl, or is substituted C₁ alkyl as previously defined, the coupling reaction between B and the 4,6-dichloropyrimidine moiety can take place between the corresponding metal complex of B, B-M (M can be zinc, magnesium, copper, a boronic derivative) in the presence of a catalyst such as Pd(PPh₃)₄ or PdCl₂(dppf) in a solvent such as refluxing tetrahydrofuran in the range of temperatures of. “X” on the pyrimidine ring can be amino or a protected amino group or a leaving group such as halogen or alkylthio.

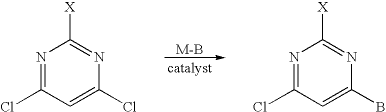

In the case when B is according to formulae IX, X or XI, as previously defined, the coupling reaction can take place in the presence of a suitable base (such as triethylamine, potassium carbonate, N,N-diisopropylethylamine etc.) in a solvent (alcohols, N,N dimethylformamide, N-methylpyrrolidinone, dimethylsulfoxide, dioxane, etc.) from room temperature to 220° C. under conventional or microwave conditions.

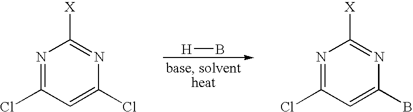

“X” on the pyrimidine ring can be amino or a protected amino group or a leaving group such as halogen or alkylthio. In H-B, B is according to formulae IX, X or XI respectively.

The coupling of the A moiety, according to formulae II, III, IV, V, VI, VII, VIII, XII, XIII, XIV as previously described, on the pyrimidine ring, which has already the B moiety coupled on, in the presence of a base (such as N-methylpyrrolidinone or triethylamine) in a solvent such as methanol, under conventional or microwave heating conditions.

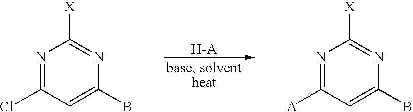

In H-A, A is according to formulae II, III, IV, V, VI, VII, VIII, XII, XIII, XIV respectively. Some of the nitrogens on the A moieties, especially in the case of 3-amino pyrrolidine or N-methylamino pyrrolidine might bear protecting groups, such as tert butoxycarbonyl (BOC). The deprotection of the amino groups takes place in the presence of trifluoroacetic acid (TFA). For more details concerning deprotection methods, see “Protective Groups in Organic Chemistry”, Chapter 2, J. F. W. Omie, Plenum Press, London and New York, 1973 and “Protective Groups in Organic Synthesis”, Chapter 7, Th. W. Greene, John Wiley & Sons, 1999.

The coupling of the A moiety, according to formulae II, III, IV, V, VI, VII, VIII, XII, XIII, XIV as previously described, on the 4,6-dichloropyrimidine ring, can take place in the presence of a base, such as or triethylamine, in a solvent, such as N-methylpyrrolidinone, at temperatures from 0° C. to 150° C. In this case “X” can be —NH₂, optionally substituted with bis-trimethylsilyl groups or can be halogen or alkylthio.

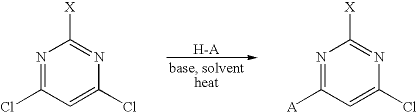

The coupling of the B moiety, in the case when B is C₃₋₁₀ cycloalkyl or is C₅₋₁₀ cycloalkenyl group, or is C₂₋₇ alkyl group, or substituted C₁ alkyl or is C₂₋₆ alkenyl, as previously defined, on the chloropyrimidine ring already having the A moiety coupled on, can take place between the corresponding metal complex of B, B-M (M can be zinc, magnesium, copper, a boronic derivative) in the presence of a catalyst such as Pd(PPh₃)₄ or PdCl₂(dppf) in solvents such as tetrahydrofuran, dioxan or toluene from room temperature to 200° C.

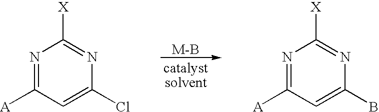

In the case when B is according to formulae IX, X or XI, as previously defined, the coupling reaction can take place between B—H and the chloropyrimidine already having the A moiety coupled on, in the presence of a suitable base (such as triethylamine, potassium carbonate, N,N-diisopropylethylamine etc.) in a solvent (alcohols, N,N dimethylformamide, N-methylpyrrolidinone, dimethylsulfoxide, dioxane, etc.) from room temperature to 220° C. under conventional or microwave conditions.

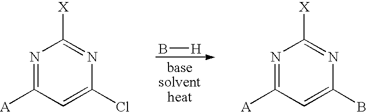

The leaving group “X” (a halogen or alkylthio) in position 2 of the pyrimidine, bearing the A and B moieties, can be displaced with ammonia or protected ammonia equivalents followed by a deprotection step.

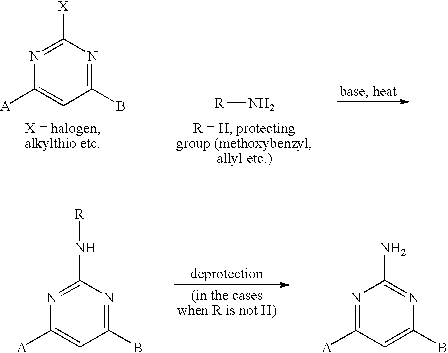

The pyrimidine ring may be constructed from the appropriate keto ester bearing the B group where B is C₃₋₁₀ cycloalkyl or is C₅₋₁₀ cycloalkenyl group, or is C₂₋₇ alkyl group, or is substituted C₁ alkyl, or is C₂₋₆ alkenyl, as previously defined, using a reagent such as guanidine in the presence of a co-reagent such as sodium acetate. The resulting hydroxypyrimidine can then be chlorinated using a reagent such as phosphorus oxychloride.

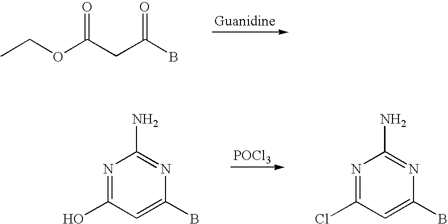

The present invention also relates to synthetic intermediates geometrical isomers, enantiomers, diastereoisomers, pharmaceutically acceptable salts and all possible mixtures thereof.

Specific synthetic intermediates are selected from the group consisting of:
4-cyclopentyl-N-(4-methoxybenzyl)-6-[3-(methylamino)pyrrolidin-1-yl]pyrimidin-2-amine;
4-cyclohexyl-6-(hexahydropyrrolo[1,2-a]pyrazin-2(1 H)-yl)-N-(4-methoxybenzyl)pyrimidin-2-amine formate salt;
4-(adamantan-2-yl)-6-chloropyrimidin-2-amine;
tert-butyl [1-(6-adamantan-2-yl-2-aminopyrimidin-4-yl)pyrrolidin-3-yl]carbamate formate salt;
4-chloro-6-(2-methylpyrrolidin-1-yl)pyrimidin-2-amine;
tert-butyl[1-(2-amino-6-cyclohexylpyrimidin-4-yl)azetidin-3-yl]carbamate;
tert-butyl(3aR*,6aS*)-5-(2-amino-6-cyclohexylpyrimidin-4-yl)hexahydropyrrolo[3,4-c]pyrrole-2(1H)-carboxylate;
4-cyclohexyl-6-[(1R*,5S*,6S*)-6-nitro-3-azabicyclo[3.1.0]hex-3-yl]pyrimidin-2-amine;
tert-butyl(1R*,5S*,6S*)-6-[(2-amino-6-cyclohexylpyrimidin-4-yl)amino]-3-azabicyclo[3.1.0]hexane-3-carboxylate;
tert-butyl(1R*,5S*,6S*)-6-[(2-amino-6-cyclopentylpyrimidin-4-yl)amino]-3-azabicyclo[3.1.0]hexane-3-carboxylate;
tert-butyl(1R*,5S*,6S*)-6-{[2-amino-6-(cyclopentylmethyl)pyrimidin-4-yl]amino}-3-azabicyclo[3.1.0]hexane-3-carboxylate;
6-chloro-N⁴-(tetrahydro-2H-pyran-4-yl)pyrimidine-2,4-diamine;
N⁴-[(1R*,2S*,4S*)-bicyclo[2.2.1]hept-2-yl]-6-chloropyrimidine-2,4-diamine;
tert-butyl[1-(2-amino-6-cyclopropylpyrimidin-4-yl)pyrrolidin-3-yl]carbamate;
6-chloro-N⁴-[2-(dimethylamino)ethyl]pyrimidine-2,4-diamine;
N⁴-(8-benzyl-8-azabicyclo[3.2.1]oct-3-yl)-6-cyclohexylpyrimidine-2,4-diamine;
4-chloro-6-(1,3-dihydro-2H-isoindol-2-yl)pyrimidin-2-amine;
6-chloro-N⁴-cyclohexyl-N⁴-methylpyrimidine-2,4-diamine;
tert-butyl 3-[(2-amino-6-cyclohexylpyrimidin-4-yl)(methyl)amino]pyrrolidine-1-carboxylate;
tert-butyl{[1-(2-amino-6-cyclohexylpyrimidin-4-yl)azetidin-3-yl]methyl}carbamate;
tert-butyl 4-{2-amino-6-[(1R*,2R*,4S*)-bicyclo[2.2.1]hept-2-ylamino]pyrimidin-4-yl}-2-methylpiperazine-1-carboxylate;
tert-butyl[1-(2-amino-6-cyclopentylpyrimidin-4-yl)pyrrolidin-3-yl]carbamate di-formate salt;
tert-butyl 4-{[2-amino-6-(4-methylpiperazin-1-yl)pyrimidin-4-yl]amino}piperidine-1-carboxylate;
4-chloro-6-[(E)-2-phenylvinyl]pyrimidin-2-amine;
tert-butyl(1-{2-amino-6-[(E)-2-phenylvinyl]pyrimidin-4-yl}pyrrolidin-3-yl)carbamate;
4-chloro-6-(4-methylcyclohex-1-en-1-yl)pyrimidin-2-amine;
tert-butyl{1-[2-amino-6-(4-methylcyclohex-1-en-1-yl)pyrimidin-4-yl]pyrrolidin-3-yl}carbamate;
2-amino-6-(1,2,3,4-tetrahydronaphthalen-2-yl)pyrimidin-4-ol;
2-amino-6-(cyclohexylmethyl)pyrimidin-4-ol;
2-amino-6-(cyclopentylmethyl)pyrimidin-4-ol;
4-cyclohexyl-6-(4-cyclopropylpiperazin-1-yl)-N-(4-methoxybenzyl)pyrimidin-2-amine bis-formate salt;
tert-butyl{1-[2-amino-6-(1,2,3,4-tetrahydronaphthalen-2-yl)pyrimidin-4-yl]pyrrolidin-3-yl}carbamate;
tert-butyl{1-[2-amino-6-(cyclohexylmethyl)pyrimidin-4-yl]pyrrolidin-3-yl}carbamate formate salt;
tert-butyl{1-[2-amino-6-(2-phenylethyl)pyrimidin-4-yl]pyrrolidin-3-yl}carbamate;
4-chloro-6-(cyclopentylmethyl)pyrimidin-2-amine;
N-[1-(2-amino-6-cyclopentylpyrimidin-4-yl)azetidin-3-yl]acetamide;
2,4-dichloro-6-cyclohexylpyrimidine;
tert-butyl[(3R)-1-(2-chloro-6-cyclohexylpyrimidin-4-yl)pyrrolidin-3-yl]carbamate;
2-chloro-4-cyclohexyl-6-(4-cyclopropylpiperazin-1-yl)pyrimidine;
tert-butyl[(3R)-1-{6-[adamantan-2-yl]-2-chloropyrimidin-4-yl}pyrrolidin-3-yl]carbamate;
tert-butyl 1(3S)-1-[6-[adamantan-2-yl]-2-chloropyrimidin-4-yl}pyrrolidin-3-yl]carbamate;
tert-butyl[(3S)-1-(2-chloro-6-cyclohexylpyrimidin-4-yl)pyrrolidin-3-yl]carbamate;
tert-butyl[1-(2-chloro-6-cyclopentylpyrimidin-4-yl)pyrrolidin-3-yl]methylcarbamate;
2-(2-chloro-6-cyclohexylpyrimidin-4-yl)octahydropyrrolo[1,2-a]pyrazine;
tert-butyl[1-(2-chloro-6-cyclopentylpyrimidin-4-yl)pyrrolidin-3-yl]carbamat;
3-[(4-fluorobenzyl)oxy]pyrrolidine;
(3S)-1-{6-[adamantan-2-yl]-2-chloropyrimidin-4-yl}-N-methylpyrrolidin-3-amine;
cis-4-amino-N-phenylcyclohexanecarboxamide;
(1S*,3R*)-3-amino-N-phenylcyclohexanecarboxamide;
trans-4-amino-N-methylcyclohexanecarboxamide;
trans-4-amino-N-cyclopropylcyclohexanecarboxamide;
trans-4-amino-N-tert-butylcyclohexanecarboxamide;
trans-4-amino-N-(4-methoxyphenyl)cyclohexanecarboxamide;
tert-butyl((1S*,3R*)-3-(anilinocarbonyl)cyclohexyl]carbamate;
tert-butyl[trans-4-(methylcarbamoyl)cyclohexyl]carbamate;
tert-butyl[trans-4-(cyclopropylcarbamoyl)cyclohexyl]carbamate;
tert-butyl[trans-4-(tert-butylcarbamoyl)cyclohexyl]carbamate;
tert-butyl{trans-4-[(4-methoxyphenyl)carbamoyl]cyclohexyl}carbamate;
methyl 3-oxo-3-(1,2,3,4-tetrahydronaphthalen-2-yl)propanoate;
4-[adamantan-2-yl]-2,6-dichloropyrimidine.

Experimental Part

The following examples are provided for illustrative purposes only. Those skilled in the art will appreciate that routine variations and modifications of the following examples can be made without exceeding the spirit or scope of the invention.

Unless specified otherwise in the examples, characterization of the compounds is performed according to (LCMS) liquid chromatography mass spectra, preparative liquid chromatography LC, NMR, and silica gel chromatography methods.

NMR spectra are recorded on Bruker AV300 or DRX 400 spectrometers at 300 or 400 MHz respectively.

Chromatographic separations are performed on DAVISIL 40-63 μM silica gel.

Various reactions took place in an Emrys Optimiser microwave reactor. The following abbreviations are used in the examples:
MeCN—Acetonitrile
HCOOH—Formic acid
LiAlH₄—Lithium aluminum hydride
NH₄OAc—Ammonium acetate
PdCl₂(dppf)—Dichloro palladium [1,1′-bis(diphenylphosphino)ferrocene]
MgSO₄—Magnesium sulfate
RT—Retention time
R_{f}—Retention factor
CD₃OD—Deuterated methanol
H₂O—Water
K₂CO₃—Potassium carbonate
Pd(PPh₃)₄—Tetrakis-(triphenylphosphine)-palladium
Na₂CO₃—Sodium carbonate
POCl₃—Phosphorus oxychloride
Na₂SO₄—Sodium sulfate
Et₂O —Diethylether
H₂—Hydrogen
Et₃N —TEA—Triethylamine
NH₃—Ammonia
NaHCO₃—Sodium hydrogencarbonate
NaBH(OAc)₃—Sodiumborohydridetriacetate
CDCl₃—Deuterated chloroform
N₂—Nitrogen
DCM—Dichloromethane
DIPEA—N,N-Diisopropylethylamine
DMSO—Dimethyl sulphoxide
DMF—N,N-Dimethylformamide
d₆₋DMSO—Dimethyl-d₆ sulphoxide
MeOH—Methanol
NMP—1-Methyl-2-pyrrolidinone
MTBE—Methyl tert-butyl ether
TFA—Trifluoroacetic acid
EtOAc—Ethyl acetate
THF—Tetrahydrofuran
ESI—Electrospray ionization
Pos—Positive
Neg—Negative
Atm—Atmosphere

The IUPAC names of compounds are generated using ACD (Labs Release: 9.00, product version: 9.04). The stereochemical nomenclature is according to the guidelines found in

“A Guide to IUPAC Nomenclature of Organic Compounds (Recommendations 1993), Blackwell Scientific Publications, Oxford, 1993”.

All the reagents, solvents, catalysts for which the synthesis is not described have been purchased from chemical vendors such as Sigma Aldrich, Fluka, Lancaster, however some known reaction intermediates, for which the registry numbers (RN) are mentioned, have been prepared in-house following known procedures.

The following analytical LCMS conditions were used to obtain the retention times (RT) as described herein:

HP1100 (Diode Array) linked to a Finnigan LC-Q Mass Spectrometer, ESI mode with Pos/Neg ionization.

| Column: | Luna C18(2) 100 × 4.6 mm, 5 μm particle | |
|---|---|---|
| | size Analytical column | |
| Column temp: | 35° C. | |
| Mobile phase: | A: Water + 0.08% formic acid | |
| | B: Acetonitrile + 0.08% formic acid | |
| Flow rate: | 3 ml/min | |
| | Time (min) | % Composition B |
| Gradient: | 0 | 5 |
| | 4.40 | 95 |
| | 5.30 | 95 |
| | 5.32 | 5 |
| | 6.50 | 5 |

| | |
|---|---|
| Run time: | 6.5 min |
| Typical injection volume: | 10 μl |
| Detector wavelength: | DAD 200-400 nm |

LCMS conditions (pH 5.8)

HP1100 (Diode Array) linked to a Finnigan LC-Q Mass Spectrometer, ESI mode with Pos/Neg ionisation.

| Column: | Luna C18(2) 100 × 4.6 mm, 5 μm particle | |
|---|---|---|
| | size Analytical column | |
| Column temp: | 35° C. | |
| Mobile phase: | A: 5 mM NH₄OAc pH 5.8 | |
| | B: 95: 5, MeCN: 100 mM NH₄OAc pH 5.8 | |
| Flow rate: | 3 ml/min | |
| | Time (min) | % Composition B |
| Gradient: | 0 | 5 |
| | 4.40 | 95 |
| | 5.30 | 95 |
| | 5.32 | 5 |
| | 6.50 | 5 |

| | |
|---|---|
| Run time: | 6.5 min |
| Typical injection volume: | 10 μl |
| Detector wavelength: | DAD 200-400 nm |

The following preparative LC conditions are used to purify compounds as described herein:

Preparative LC Conditions (pH 2.5) (Method A)

Waters autopreparative mass and UV directed: ZQ mass spectrometer, 996 PDA, 2525 pump and 2767 autosampler/fraction collector and 2757 fraction collector.

| | |
|---|---|
| Column: | Phenomenex Luna C18(2) 250 × 21.2 mm, |
| | 5 μm particle size prep column |
| Column temp: | Ambient |
| Mobile phase: | A: Water + 0.08% formic acid |
| | B: Acetonitrile + 0.08% formic acid |
| Flow rate: | 25 ml/min |
| Gradient: | Variable - depends on retention time of sample |
| | in LC-MS analysis |
| Run time: | 20 min |
| Injection volume: | 1 ml at 50 mg/ml (typically) |
| Detector wavelength: | 200 to 400 nm |

Preparative LC Conditions (pH 5.8) (Method B)

Waters autopreparative mass and UV directed: ZQ mass spectrometer, 996 PDA, 2525 pump and 2767 autosampler/fraction collector and 2757 fraction collector.

| | |
|---|---|
| Column: | Phenomenex Luna C18(2) 250 × 21.2 mm, |
| | 5 μm particle size prep column |
| Column temp: | Ambient |
| Mobile phase: | A: 10 mM ammonium acetate pH 5.8 |
| | B: 5: 95, 200 mM ammonium acetate |
| | pH 5.8: Acetonitrile |
| Flow rate: | 25 ml/min |
| Gradient: | Variable - depends on retention time of sample in |
| | LC-MS analysis |
| Run time: | 20 min |
| Injection volume: | 1 ml at 50 mg/ml (typically) |
| Detector wavelength: | 200 to 400 nm |

The following preparative LC conditions are used to purify the compounds generated in libraries:

Preparative LC Conditions (Method C) (pH 2.5):

Gilson 215 liquid handler setup.

| | |
|---|---|
| Column: | Luna C18(2) 100 × 21.2 mm, 5 μM particle |
| | size prep column. |
| Column Temp: | Ambient. |
| Gradient: | Variable- depends on retention of sample |
| Run Time: | 10 mins |
| Flow rate: | 20 ml/min |
| Typical Injection volume: | 500 μl at 30 mg/ml |
| Detector Wavelength: | Diode array |
| Mobile phase A: | Water + 0.08% formic acid |
| Mobile phase B: | MeCN + 0.08% formic acid |

Preparative LC Conditions (Method D) (pH 5.8):

Gilson 215 liquid handler setup.

| | |
|---|---|
| Column: | Luna C18(2) 100 × 21.2 mm, 5 μM particle |
| | size prep column |
| Column Temp: | Ambient |
| Gradient: | Variable - depends on retention time of sample |
| Run Time: | 10 mins |
| Flow rate: | 20 ml/min |
| Typical Injection Vol: | 500 μl at 30 mg/ml |
| Detector Wavelength: | Diode array |
| Mobile Phase: A: | 10 mM NH₄OAc in water |
| Mobile Phase B: | 10 mM NH₄OAc in MeCN |

### EXAMPLE 1

### Synthesis of 4-adamantan-2-yl-6-(4-methylpiperazin-1-yl)pyrimidin-2amine (Compound 1)

A suspension of 4-chloro-6-(4-methylpiperazin-1-yl)pyrimidin-2-amine (CAS RN 322691-38-3) (86 mg), 2-adamantylzinc bromide (0.5M solution in THF) (1.5 ml) and PdCl₂(dppf) (14 mg) in anhydrous THF (1.5 ml) is heated in the microwave for 6 mins at 110° C. Solvents are removed in vacuo and the crude material is dissolved in EtOAc (50 ml), washed with water (15 ml), dried over MgSO₄, filtered and concentrated in vacuo. Purification by preparative HPLC (Method B) affords the title compound as colorless oil (18.8 mg, 15%). LCMS 328 [M+H]⁺, RT (pH5.8) 2.24 mins. ¹H NMR 300 MHz (CD₃OD) (δ ppm): 6.20 (1H, s), 3.70 (4H, t), 2.85 (1H, s), 2.55 (4H, t), 2.45 (2H, s), 2.05-1.80 (12H, m), 1.70 (2H, d).

Compounds 2 and 3 are prepared in a similar manner to the method described for Compound 1 in Example 1. The reagents used and the results obtained are tabulated below (Table 1). The free base of the compounds is obtained.

TABLE 1

| Comp. | | | | ¹H NMR |
|---|---|---|---|---|
| No | IUPAC Name | Starting Materials | LCMS | (Solvent, δ ppm) |
| 2 | 4-cyclohexyl-6-(4- | 4-chloro-6-(4- | 276 [M + H]⁺ | CD₃OD: 6.20 (1H, s), |
| | methylpiperazin- | methylpiperazin- | RT 1.85 min | 5.55 (2H, s), 3.86 (4H, m), |
| | 1-yl) pyrimidin-2- | 1-yl)pyrimidin-2- | (pH | 2.58 (4H, m), 2.39 (3H, s), |
| | amine | amine, | 5.8). | 1.75-2.00 (5H, m), |
| | | cyclohexylzinc | | 1.30-1.59 (6H, m). |
| | | bromide | | |
| 3 | 4-[2-(1,3- | 4-chloro-6-(4- | 294 [M + H]⁺ | CD₃OD: 6.10 (1H, s), |
| | dioxolan-2- | methylpiperazin- | RT 1.53 mins | 4.90 (1H, s), 4.00 (2H, m), |
| | yl)ethyl]-6-(4- | 1-yl)pyrimidin-2- | (pH | 3.85 (2H, m), 3.70 (4H, t), |
| | methylpiperazin- | amine, | 5.8) | 2.60 (2H, m), 2.55 (4H, t), |
| | 1-yl)pyrimidin-2- | [2-(1,3-dioxolan- | | 2.35 (2H, s), 2.20 (3H, s), |
| | amine | 2-yl)ethyl]zinc | | 2.00 (2H, m). |
| | | bromide | | |

Comp. No means Compound Number
Interm. means Intermediate

### EXAMPLE 2

### Synthesis of 2,4-dichloro-6-cyclohexylpyrimidine (Intermediate 1)

2,4,6-Trichloropyrimidine (CAS RN 3764-01-0) (1.83 g) is added to a solution of cyclohexylzinc bromide (0.5M in THF, 20 ml). Nitrogen is bubbled through the solution for 10 mins, after which tetrakis(triphenylphosphine)palladium (0.05 g) is added, and the mixture is heated under N₂ at 70° C. for 18 hrs. The solution is cooled and partitioned between diethyl ether and water (30 ml each). The ether layer is dried (MgSO₄) and evaporated in vacuo. Purification of the residue by flash chromatography, eluting with petroleum ether 40:60-diethyl ether (9:1) followed by evaporation under high vacuum at 80° C. (to remove any residual starting material) affords the title compound as a colorless solid (1.40 g, 60%). LCMS 231/233 [M+H]⁺, RT 4.75 mins (pH 5.8). ¹H NMR 300 MHz (CDCl₃) (δ ppm): 7.14 (1H, s), 2.66 (1H, m), 1.81-2.02 (4H, m), 1.75 (1H, d), 1.20-1.59 (5H, m).

### EXAMPLE 3

### Synthesis of tert-butyl [(3R)-1-(2-chloro-6-cyclohexylpyrimidin-4-yl)pyrrolidin-3-yl]carbamate (Intermediate 2)

Intermediate 1 (147 mg), tert-butyl (3R)-pyrrolidin-3-ylcarbamate (CAS RN 122536-77-0) (119 mg) and triethylamine (0.089 ml) are dissolved in methanol (4 ml) and the solution stirred at room temperature for 2 hrs. The solvent is evaporated, and the residue purified by flash chromatography, eluting with EtOAc-Heptane (1:4), to afford the title compound as a colorless solid (176 mg, 72%). R_{f} (EtOAc-Heptane 3:7) 0.40. LCMS 381 [M+H]⁺, RT 4.35 mins (pH 2.5). ¹H NMR 300 MHz (CDCl₃) (δ ppm): 6.00 (1H, s), 4.64 (1H, br m), 4.32 (1H, br s), 3.15-3.88 (4H, br m), 2.48 (1H, m), 2.26 (1H, br m), 1.67-2.01 (5H, m), 1.45 (9H, s), 1.20-1.50 (6H, m).

Intermediates 3 to 9 are prepared in a similar manner to the method described for Intermediate 2 in Example 3.The reagents used and the results obtained are tabulated below (Table 2). The free base of the compounds is obtained.

TABLE 2

| Interm. | | Starting | | ¹H NMR |
|---|---|---|---|---|
| No | IUPAC Name | Materials | LCMS | (Solvent, δ ppm) |
| 3 | 2-chloro-4- | Intermediate 1, | 321 [M + H]⁺ | CDCl₃ 6.20 (1H, s), |
| | cyclohexyl-6-(4- | N- | RT 2.19 mins | 3.60 (4H, m), 2.67 (4H, m), |
| | cyclopropylpiperazin- | cyclopropylpiperazine | (pH 2.5) | 2.46 (1H, m), |
| | 1-yl)pyrimidine | | | 1.15-2.00 (10H, m), 0.89 (1H, |
| | | | | m), 0.40-0.56 (4H, m). |
| 4 | tert-butyl [(3R)-1-{6- | Intermediate 86, | 433 [M + H]⁺ | CDCl₃ 6.15 (1H, s), |
| | [adamantan-2-yl]-2- | tert-butyl (3R)- | RT 5.02 mins | 4.65 (1H, bm), 4.35 (1H, bm), |
| | chloropyrimidin-4- | pyrrolidin-3- | (pH 2.5) | 3.15-3.90 (4H, bm), |
| | yl}pyrrolidin-3- | ylcarbamate | | 2.85 (1H, s), 2.54 2H, s), |
| | yl]carbamate | (CAS RN | | 2.26 (1H, bm), |
| | | 122536-77-0) | | 1.58-2.05 (13H, m), 1.45 (9H, |
| | | | | s). |
| 5 | tert-butyl [(3S)-1-{6- | Intermediate 86, | 433 [M + H]⁺ | CDCl₃ 6.15 (1H, s), |
| | [adamantan-2-yl]-2- | tert-butyl (3S)- | RT 5.05 mins | 4.65 (1H, bm), 4.35 (1H, bm), |
| | chloropyrimidin-4- | pyrrolidin-3- | (pH 2.5) | 3.20-3.90 (4H, bm), |
| | yl}pyrrolidin-3- | ylcarbamate | | 2.85 (1H, s), 2.54 2H, s), |
| | yl]carbamate | (CAS RN | | 2.27 (1H, bm), |
| | | 122536-76-9) | | 1.58-2.03 (13H, m), 1.45 (9H, |
| | | | | s). |
| 6 | tert-butyl [(3S)-1-(2- | Intermediate 1, | 381 [M + H]⁺, | CDCl₃ |
| | chloro-6- | tert-butyl (3S)- | RT 4.35 mins | 6.00 (1H, s), 4.64 (1H, |
| | cyclohexylpyrimidin- | pyrrolidin-3- | (pH 2.5) | br m), 4.32 (1H, br s), |
| | 4-yl)pyrrolidin-3- | ylcarbamate | | 3.15-3.88 (4H, br m), |
| | yl]carbamate | | | 2.48 (1H, m), 2.27 (1H, |
| | | | | br m), 1.68-2.05 (5H, |
| | | | | m), 1.45 (9H, s), |
| | | | | 1.20-1.52 (6H, m). |
| 7 | tert-butyl [1-(2- | 2,4-dichloro-6- | 381 [M + H]⁺, | CDCl₃ 6.04 (1H, |
| | chloro-6- | cyclopentylpyrimidine | RT 4.57 mins | s), 4.88 (1H, br m), |
| | cyclopentylpyrimidin- | (CAS RN | (pH | 3.15-3.95 (4H, br m), |
| | 4-yl)pyrrolidin-3- | 199863-89-3), 3- | 2.5). | 2.93 (1H, m), 2.80 (3H, s), |
| | yl]methylcarbamate | (N-tert- | | 1.56-2.25 (10H, m), |
| | | butoxycarbonyl- | | 1.47 (9H, s). |
| | | N-methylamino)- | | |
| | | pyrrolidine | | |
| 8 | 2-(2-chloro-6- | Intermediate 1, | 321 [M + H]⁺, | CDCl₃ 6.21 (1H, s), |
| | cyclohexylpyrimidin- | octahydro- | RT 2.03 mins | 4.12-4.61 (2H, br m), |
| | 4- | pyrrolo[1,2- | (pH | 3.00-3.19 (3H, m), 2.70 (1H, |
| | yl)octahydropyrrolo[1, | a]pyrazine | 2.5). | t), 2.48 (1H, m), |
| | 2-a]pyrazine | | | 2.21-2.28 (2H, m), |
| | | | | 1.68-2.02 (9H, m), |
| | | | | 1.19-1.55 (6H, m). |
| 9 | tert-butyl [1-(2- | 2,4-dichloro-6- | 367 [M + H]⁺, | CDCl₃ |
| | chloro-6- | cyclopentylpyrimidine, | RT 4.10 mins | 6.05 (1H, s), 4.63 (1H, |
| | cyclopentylpyrimidin- | 3-(tert- | (pH | br m), 4.32 (1H, br m), |
| | 4-yl)pyrrolidin-3- | butoxycarbonylamino)pyrrolidine | 2.5) | 3.15-3.90 (4H, br m), |
| | yl]carbamate | | | 2.93 (1H, m), 2.26 (1H, |
| | | | | m), 1.50-2.10 (7H, m), |
| | | | | 1.47 (9H, s). |

Interm. No means Intermediate Number

### EXAMPLE 4

### Synthesis of 4-[(3R)-3-aminopyrrolidin-1-yl-6-cyclohexylpyrimidin-2-amine (Compound 4)

Intermediate 2 (176 mg), 4-methoxybenzylamine (0.064 ml) and triethylamine (0.071 ml) are dissolved in dry NMP (1 ml) and heated in the microwave at 180° C. for 50 mins. The solution is then diluted with brine (5 ml) and extracted with EtOAc (2×20 ml), followed by back extracting with saturated brine (2×25 ml). The combined organic layers are dried (MgSO₄) and concentrated to dryness in vacuo. The residual oil is treated with TFA (1.8 ml) and the solution heated at 75° C. for 1 hr. The excess TFA is then removed in vacuo, the residue diluted with brine (5 ml), neutralised with 48% NaOH solution and extracted with EtOAc (2×20 ml). The extracts are dried (MgSO₄) and concentrated to dryness in vacuo. After preparative HPLC of the residue (Method B), the fractions are concentrated in vacuo, re-dissolved in DCM (25 ml), washed with saturated NaHCO₃ solution (5 ml), dried (MgSO₄) and concentrated in vacuo to afford the title compound as a colorless solid (21.9 mg, 18%). LCMS 262 [M+H]⁺RT 1.51 mins (pH 5.8). ¹H NMR 300 MHz (CDCl₃) (δ ppm): 5.58 (1H, s), 5.00 (2H, br s), 3.52-3.75 (3H, m), 3.46 (1H, br s), 3.17 (1H, br s), 1.59-2.41 (10H, m), 1.15-1.50 (5H, m).

Compounds 5 to 8 are prepared in a similar manner to the method described for Compound 4 in Example 4.The reagents used and the results obtained are tabulated below (Table 3). The free base of the compounds is obtained.

TABLE 3

| Comp. | | | | ¹H NMR |
|---|---|---|---|---|
| No | IUPAC Name | Starting Materials | LCMS | (Solvent, δ ppm) |
| 5 | 4-[adamantan- | Intermediate 4, | 314 [M + H]⁺ | CD₃OD 6.06 (1H, s), |
| | 2-yl]-6-[(3R)-3- | 4- | RT | 3.67-4.13 (5H, m), |
| | aminopyrrolidin- | methoxybenzylamine, | 1.27 mins | 2.97 (1H, s), 2.50 (1H, |
| | 1- | | (pH | bm), 2.41 (2H, s), |
| | yl]pyrimidin-2- | | 2.5) | 1.70-2.32 (13H, m). |
| | amine | | | |
| 6 | 4-[adamantan- | Intermediate 5,4- | 314 [M + H]⁺ | CD₃OD 5.92 (1H, s), |
| | 2-yl]-6-[(3S)-3- | methoxybenzylamine, | RT | 3.34-3.86 (5H, m), |
| | aminopyrrolidin- | | 1.30 mins | 2.83 (1H, s), 2.44 (2H, |
| | 1- | | (pH | s), 2.30 (1H, m), |
| | yl]pyrimidin-2- | | 2.5) | 1.58-2.10 (13H, m). |
| | amine | | | |
| 7 | 4-[adamantan- | Intermediate 65, | 328 [M + H]⁺ | CD₃OD 6.09 (1H, s), |
| | 2-yl]-6-[(3S)-3- | 4- | RT | 3.65-4.10 (5H, m), |
| | (methylamino)pyrrolidin- | methoxybenzylamine, | 2.14 mins | 2.97 (1H, s), 2.75 (3H, |
| | 1- | | (pH | s), 2.38-2.56 (3H, m), |
| | yl]pyrimidin-2- | | 5.8) | 2.27 (1H, m), |
| | amine | | | 1.69-2.15 (12H, m). |
| 8 | 4-[(3S)-3- | Intermediate 6, | 262 [M + H]⁺ | CDCl₃ 5.58 (1H, s), |
| | aminopyrrolidin- | 4- | RT | 4.86 (2H, br s), |
| | 1-yl]-6- | methoxybenzylamine | 1.46 mins | 3.52-3.76 (3H, m), 3.45 (1H, |
| | cyclohexylpyrimidin- | | (pH | br s), 3.17 (1H, br s), |
| | 2-amine | | 5.8). | 2.32 (1H, m), 2.15 (1H, |
| | | | | m), 1.56-2.41 (8H, m), |
| | | | | 1.15-1.50 (5H, m). |

Comp. No means Compound Number

### EXAMPLE 5

### Synthesis of 4-cyclopentyl-N-(4-methoxybenzyl)-6-[3-(methylamino)pyrrolidin-1-yl]pyrimidin-2-amine (Intermediate 10)

Intermediate 7 (145 mg), 4-methoxybenzylamine (0.052 ml) and triethylamine (0.056 ml) are dissolved in dry NMP (1 ml) and heated under microwave irradiation at 180° C. for 30 mins. The solution is then treated at room temperature with TFA (2.75 ml), and after 4 hrs, excess TFA is removed in vacuo, and the residue diluted with brine (10 ml), saturated NaHCO₃ (10 ml) and 48% NaOH (6 ml). The aqueous phase is extracted with EtOAc (2×15 ml), dried (MgSO₄) and concentrated in vacuo. Purification by preparative HPLC (Method A) affords the title compound as a colorless glass (50 mg, 35%). LCMS 382 [M+H]⁺RT 1.39 mins (pH 2.5). ¹H NMR 300 MHz (CDCl₃) (δ ppm): 9.79 (1H, m), 7.24 (2H, d), 6.81 (2H, d), 5.60 (1H, s), 4.35-4.55 (2H, m), 3.40-4.04 (10H, m), 2.94 (1H, m), 2,62 (3H, s), 1.60-2.46 (9H, m).

### EXAMPLE 6

### Synthesis of 4-cyclopentyl-6-[3-(methylamino)pyrrolidin-1-yl]pyrimidin-2-amine (Compound 9)

Intermediate 10 (50 mg) is dissolved in TFA (1 ml) and the solution is heated at 75° C. for 1.5 hrs. The excess TFA is removed in vacuo. The residue is redissolved in DCM and azeotroped with heptane. Purification by preparative HPLC (Method B) followed by a DCM/saturated NaHCO₃ partition affords the title compound as a colorless solid (20.5 mg, 60%). LCMS 262 [M+H]⁺ RT 1.64 mins (pH 5.8). ¹H NMR 300 MHz (CDCl₃) (δ ppm): 5.62 (1H, s), 5.23 (2H, br s), 2.71-3.74 (7H, m), 2.47 (3H, s), 1.57-2.30 (10H, m).

### EXAMPLE 7

### Synthesis of 4-cyclohexyl-6-(hexahydropryrrolo[1,2-a]pyrazin-2(1H)-yl)-N-(4-methoxybenzyl)pyrimidin-2-amine formate salt (Intermediate 11)

4-Methoxybenzylamine (0.068 ml) and triethylamine (0.074 ml) are added to a solution of Intermediate 8 (80 mg) in dry NMP (1.5 ml), and the solution heated under microwave irradiation at 150° C. for 40 mins. Purification of the crude reaction mixture by preparative HPLC (Method A) affords the title compound as its di-formate salt (44 mg, 38%) LCMS 422 [M+H]⁺, RT 1.48 mins (pH 2.5). ¹H NMR 300 MHz (CDCl₃) (δ ppm): 10.65 (1H, br m), 8.45 (HCOOH), 7.28 (2H, d), 6.84 (2H, d), 5.72 (1H, s), 4.48 (2H, d), 3.78 (3H, s), 1.12-3.34 (24H, m).

### EXAMPLE 8

### Synthesis of 4-cyclohexyl-6-(hexahydropyrrolo[1,2-a]pyrazin-2(1H)-yl)pyrimidin-2-amine (Compound 10)

Intermediate 11 (52 mg) is dissolved in TFA (1.5 ml) and the solution is stirred and heated at 75° C. for 90 mins. The solution is cooled, and concentrated in vacuo. The residue is dissolved in DCM (20 ml), and washed with saturated NaHCO₃ (10 ml). The organic phase is dried (MgSO₄) and concentrated in vacuo. Purification of the residue by preparative HPLC (Method B) affords pure product which after isolation in vacuo is partitioned between DCM (20 ml) and saturated NaHCO₃ solution (10 ml); the organic phase is dried (MgSO₄) and concentrated in vacuo to afford the title compound as a pale yellow solid (30.1 mg, 100%). LCMS 302 [M+H]⁺ RT 2.06 mins (pH 5.8). ¹H NMR 300 MHz (CDCl₃) (δ ppm): 5.82 (1H, s), 4.81 (2H, br s), 4.45 (1H, br d), 4.29 (1H, br d), 2.91-3.19 (3H, m), 2.61 (1H, dd), 2.10-2.40 (3H, m), 1.65-2.01 (9H, m), 1.15-1.55 (7H, m).

### EXAMPLE 9

### Synthesis of 4-(adamantan-2-yl)-6-chloropyrimidin-2-amine (Intermediate 12)

4,6-Dichloropyrimidin-2-amine (CAS RN 56-05-3)(615 mg) and 2-adamantylzinc bromide (0.5M in THF, 9.75 ml) are dissolved in THF (5 ml), and degassed by bubbling N₂ through the solution for 15 mins. Then 1,1′-bis(diphenylphosphino)ferrocene palladium (II) (153 mg) is added, and the reaction heated under N₂ at 78° C. for 18 hrs. The reaction is quenched with water (5 ml) and the THF removed in vacuo. The residue is partitioned between DCM (60 ml) and water (30 ml), and filtered through Kieselguhr. The organic phase is separated, dried (MgSO₄) and concentrated to dryness to afford an orange oil. Purification by flash chromatography, eluting with EtOAc-Heptane 3:17, affords the title compound as a colorless solid (69 mg, 7%). R_{f} (EtOAc-Heptane 3:17) 0.32. LCMS 264 [M+H]⁺, RT 4.47 mins (pH 2.5). ¹H NMR 300 MHz (CDCl₃) (δ ppm): 6.66 (1H, s), 5.05 (2H, br s), 2.81 (1H, m), 2.50 (2H, m), 1.58-2.02 (12H, m).

### EXAMPLE 10

### Synthesis of tert-butyl[1-(6-adamantan-2-yl-2-aminopyrimidin-4-yl)pyrrolidin-3-yl]carbamate formate salt (Intermediate 13)

Intermediate 12 (42.6 mg), tert-butyl pyrrolidin-3-ylcarbamate (CAS RN 99724-19-3) (30 mg) and triethylamine (0.023 ml) are dissolved in dry NMP (1 ml) and heated under microwave irradiation at 150° C. for 30 mins. The solution is diluted with DMSO and purified by preparative HPLC (Method A) to afford the title compound as a colorless solid as its formate salt (46 mg, 57%). LCMS 414 [M+H]⁺ RT 2.48 mins (pH 2.5). ¹H NMR 300 MHz (CDCl₃) (δ ppm): 8.48 (HCOOH), 6.40 (0.5H, br s), 5.80 (1H, m), 5.52 (0.5H, br m), 4.16-4.43 (1H, m), 3.35-3.90 (4H, m), 2.95 (1H, m), 1.60-2.55 (18H, m), 1.47 (9H, s).

### EXAMPLE 11

### Synthesis of 4-adamantan-2-yl-6-(3-aminopyrrolidin-1-yl)pyrimidin-2-amine (Compound 11)

Intermediate 13 (46 mg) is dissolved in DCM (3 ml) and TFA (0.7 ml) is added. The solution is allowed to stand at room temperature for 90 mins. The solution is concentrated in vacuo, azeotroped with MeOH/Heptane, and purified by preparative HPLC (Method B). The pure fractions are concentrated in vacuo, the residue is dissolved in DCM (50 ml) and washed with saturated NaHCO₃ solution (20 ml). The organic phase is dried (MgSO₄) and concentrated in vacuo to afford the title compound as a colorless solid (31.9 mg, 92%). LCMS 314 [M+H]⁺ RT 1.27 mins (pH 2.5). ¹H NMR 300 MHz (CDCl₃) (δ ppm): 5.75 (1H, s), 5.15 (2H, br m), 3.55-3.78 (2H, m), 3.49 (1H, br m), 3.20 (1H, br m), 2.75 (1H, m), 2.09-2.60 (6H, m), 1.50-2.00 (13H, m).

Compounds 12 to 40 are prepared in a similar manner to the method described for Compound 11 in Example 11. The reagents used and the results obtained are tabulated below (Table 4). The free base of the compounds is obtained unless otherwise stated.

TABLE 4

| Comp. | | Starting | | | ¹H NMR |
|---|---|---|---|---|---|
| No | IUPAC Name | Materials | Salt | LCMS | (Solvent, δ ppm) |
| 12 | 4-cyclohexyl-6- | Interm. | | 288 [M + H]⁺ | CDCl₃ 5.65 (1H, s), |
| | [(3aR*,6aS*)- | 20 | | RT 1.66 mins | 5.49 (2H, s), |
| | hexahydropyrrolo[3,4- | | | (pH 5.8) | 3.85 (2H, br s), 3.65 (2H, |
| | c]pyrrol-2(1H)- | | | | m), 3.40 (2H, d), |
| | yl]pyrimidin-2-amine | | | | 3.15 (2H, m), |
| | | | | | 2.95 (2H, br d), 2.85 (1H, |
| | | | | | br d), 2.35 (1H, m), |
| | | | | | 1.65-1.98 (5H, m), |
| | | | | | 1.20-1.49 (5H, m) |
| 13 | N⁴-[(1R*,5S*,6S*)-3- | Interm. | | 274 [M + H]⁺ | CDCl₃ 5.85 (1H, s), |
| | azabicyclo[3.1.0]hex- | 22 | | RT 1.73 mins | 4.90 (1H, s), |
| | 6-yl]-6- | | | (pH 5.8) | 4.65 (2H, s), 3.20 (2H, d), |
| | cyclohexylpyrimidine- | | | | 3.05 (2H, d), |
| | 2,4-diamine | | | | 2.38 (1H, m), 2.35 (1H, |
| | | | | | m), 1.65-1.98 (5H, |
| | | | | | m), 1.63 (2H, s), |
| | | | | | 1.20-1.49 (5H, m) |
| 14 | N⁴-[(1R*,5S*,6S*)-3- | Interm. | | 260 [M + H]⁺ | CDCl₃ 5.85 (1H, s), |
| | azabicyclo[3.1.0]hex- | 23 | | RT 1.73 mins | 4.95 (1H, s), |
| | 6-yl]-6- | | | (pH 5.8) | 4.70 (2H, s), 3.18 (2H, d), |
| | cyclopentylpyrimidine- | | | | 3.05 (2H, d), |
| | 2,4-diamine | | | | 2.85 (1H, m), 2.30 (1H, |
| | | | | | s), 1.65-2.05 (9H, |
| | | | | | m), 1.60 (2H, s) |
| 15 | N⁴-[(1R*,5S*,6S*)-3- | Interm. | | 274 [M + H]⁺ | CDCl₃ 5.85 (1H, s), |
| | azabicyclo[3.1.0]hex- | 24 | | RT 1.92 mins | 4.95 (1H, s), |
| | 6-yl]-6- | | | (pH 5.8) | 4.70 (2H, s), 3.18 (2H, d), |
| | (cyclopentylmethyl)pyrimidine- | | | | 3.05 (2H, d), |
| | 2,4-diamine | | | | 2.50 (2H, d), 2.35 (1H, s), |
| | | | | | 2.20 (1H, m), |
| | | | | | 2.00 (1H, br s), 1.73 (2H, |
| | | | | | m), 1.65 (2H, s), |
| | | | | | 1.58 (2H, m), |
| | | | | | 1.18 (4H, m) |
| 16 | 4-[3- | Interm. | | 262 [M + H]⁺ | CD₃OD 5.55 (1H, s), |
| | (aminomethyl)azetidin- | 32 | | RT 1.60 mins | 4.10 (2H, dd), |
| | 1-yl]-6- | | | (pH 5.8) | 3.75 (2H, dd), 2.90 (2H, |
| | cyclohexylpyrimidin-2- | | | | d), 2.70-2.85 (1H, |
| | amine | | | | m), 2.25-2.35 (1H, |
| | | | | | m), 1.80-1.90 (4H, |
| | | | | | m), 1.70-1.80 (1H, |
| | | | | | m), 1.20-1.50 (5H, |
| | | | | | m) |
| 17 | N⁴-[(1R*,2R*,4S*)- | Interm. | | 303 [M + H]⁺ | CD₃OD 5.07 (1H, s), |
| | bicyclo[2.2.1]hept-2- | 33 | | RT 2.07 mins | 4.01-4.16 (2H, m), |
| | yl]-6-(3- | | | (pH 5.8) | 3.43-3.50 (1H, m), |
| | methylpiperazin-1- | | | | 3.37 (1H, m), |
| | yl)pyrimidine-2,4- | | | | 3.01 (1H, d), |
| | diamine | | | | 2.70-2.87 (3H, mm), 2.43 (1H, |
| | | | | | dd), 2.25 (2H, bd), |
| | | | | | 1.80 (1H, ddd), |
| | | | | | 1.46-1.60 (3H, mm), |
| | | | | | 1.10-1.35 (4H, |
| | | | | | mm), 1.12 (3H, d). |
| 18 | N⁴-[(1R*,2R*,4S*)- | Interm. | | 303 [M + H]⁺ | CD₃OD 4.88 (1H, |
| | bicyclo[2.2.1]hept-2- | 34 | | RT 2.01 mins | obscured by H₂O |
| | yl]-6-(1,4-diazepan-1- | | | (pH 5.8) | peak, s), |
| | yl)pyrimidine-2,4- | | | | 3.60-3.76 (4H, m), |
| | diamine | | | | 3.38-3.48 (1H, m), 2.98 (2H, t), |
| | | | | | 2.85 (2H, t), |
| | | | | | 2.27 (2H, bd), |
| | | | | | 1.86-1.98 (2H, m), 1.80 (1H, |
| | | | | | ddd), |
| | | | | | 1.47-1.60 (3H, m), |
| | | | | | 1.37-1.14 (4H, m). |
| 19 | N⁴-[(1R*,2R*,4S*)- | Interm. | | 329 [M + H]⁺ | CD₃OD 4.78 (1H, s), |
| | bicyclo[2.2.1]hept-2- | 35 | | RT 2.04 mins | 3.20-3.46 (5H, |
| | yl]-6-((4aR*,7aR*)- | | | (pH 5.8) | mm), 2.87 (1H, dt), |
| | octahydro-6H- | | | | 2.52-2.62 (1H, |
| | pyrrolo[3,4-b]pyridin- | | | | bdd), |
| | 6-yl)pyrimidine-2,4- | | | | 2.24-2.40 (1H, m), 2.16 (2H, |
| | diamine | | | | bdd), |
| | | | | | 1.64-1.75 (3H, m), |
| | | | | | 1.48-1.64 (1H, m), |
| | | | | | 1.35-1.47 (4H, m), |
| | | | | | 1.00-1.30 (5H, m). |
| 20 | N⁴-[(1R*,2R*,4S*)- | Interm. | | 315 [M + H]⁺ | CD₃OD 4.90 (1H, |
| | bicyclo[2.2.1]hept-2- | 36 | | RT 1.86 mins | obscured by H₂O |
| | yl]-6-[(3aR*,6aS*)- | | | (pH 5.8) | peak, s), |
| | hexahydropyrrolo[3,4- | | | | 3.54-3.64 (2H, m), |
| | c]pyrrol-2(1H)- | | | | 3.30-3.47 (5H, mm), |
| | yl]pyrimidine-2,4- | | | | 2.92-3.14 (4H, m), |
| | diamine | | | | 2.26 (2H, bd), |
| | | | | | 1.76-1.84 (1H, m), |
| | | | | | 1.47-1.59 (3H, mm), |
| | | | | | 1.14-1.37 (4H, mm). |
| 21 | N⁴-[(1R*,2R*,4S*)- | Interm. | | 289 [M + H]⁺ | CD₃OD 4.80 (1H, |
| | bicyclo[2.2.1]hept-2- | 37 | | RT 1.85 mins | obscured by H₂O |
| | yl]-6-piperazin-1- | | | (pH 5.8) | peak, s), |
| | ylpyrimidine-2,4- | | | | 3.19-3.24 (6H, m), 2.78 (3H, |
| | diamine | | | | bt), 2.14 (2H, bd), |
| | | | | | 1.68 (1H, ddd), |
| | | | | | 1.35-1.46 (3H, m), |
| | | | | | 1.00-1.26 (4H, mm). |
| 22 | 4-cyclohexyl-6-[(2S)- | Interm. | | 276 [M + H]⁺ | CD₃OD 5.91 (1H, s), |
| | 2-methylpiperazin-1- | 40 | | RT 1.81 mins | 4.51 (1H, bs), |
| | yl]pyrimidin-2-amine | | | (pH 5.8) | 4.13 (1H, bdd), |
| | | | | | 2.97-3.11 (2H, m), |
| | | | | | 2.85-2.96 (1H, m), |
| | | | | | 2.69 (1H, td), 2.35 (1H, |
| | | | | | td), 1.83-1.95 (4H, |
| | | | | | m), 1.73-1.82 (1H, |
| | | | | | bd), 1.27-1.56 (6H, |
| | | | | | mm), 1.23 (3H, d). |
| 23 | 4-cyclohexyl-6-[(2R)- | Interm. | | 276 [M + H]⁺ | CD₃OD 5.91 (1H, s), |
| | 2-methylpiperazin-1- | 41 | | RT 1.89 mins | 4.51 (1H, bs), |
| | yl]pyrimidin-2-amine | | | (pH 5.8) | 4.13 (1H, bdd), |
| | | | | | 2.97-3.11 (2H, m), |
| | | | | | 2.85-2.96 (1H, m), |
| | | | | | 2.69 (1H, td), 2.35 (1H, |
| | | | | | td), 1.83-1.95 (4H, |
| | | | | | m), 1.73-1.82 (1H, |
| | | | | | bd), 1.27-1.56 (6H, |
| | | | | | mm), 1.23 (3H, d). |
| 24 | N⁴-[(1R*,2S*,4S*)- | Interm. | | 303 [M + H]⁺ | CDCl₃ 5.19 (1H, s), |
| | bicyclo[2.2.1]hept-2- | 42 | | RT 1.93 mins | 4.90 (1H, m), |
| | yl]-6-[3- | | | (pH 5.8) | 4.52-4.69 (3H, m), |
| | (methylamino)pyrrolidin- | | | | 3.05-3.65 (6H, m), |
| | 1-yl]pyrimidine-2,4- | | | | 1.85-2.57 (5H, m), |
| | diamine | | | | 2.35 (3H, s), 1.70 (1H, |
| | | | | | m), 1.20-1.60 (5H, |
| | | | | | m), 0.70 (1H, m). |
| 25 | 6-[(3S)-3- | Interm. | | 289 [M + H]⁺ | CDCl₃ 4.90 (1H, s), |
| | aminopyrrolidin-1-yl]- | 43 | | RT 1.86 mins | 4.77 (2H, m), |
| | N⁴-[(1R*,2S*,4S*)- | | | (pH 5.8) | 4.62 (2H, bs), |
| | bicyclo[2.2.1]hept-2- | | | | 3.33-3.80 (5H, m), 3.15 (1H, |
| | yl]pyrimidine-2,4- | | | | m), 1.90-2.55 (6H, |
| | diamine | | | | m), 1.30-1.82 (6H, |
| | | | | | m), 0.80 (1H, m). |
| 26 | 6-[(3R)-3- | Interm. | | 289 [M + H]⁺ | CDCl₃ 4.85 (1H, d), |
| | aminopyrrolidin-1-yl]- | 44 | | RT 1.99 mins | 4.75 (1H, s), |
| | N⁴-[(1R*,2S*,4S*)- | | | (pH 5.8) | 4.60 (2H, bs), |
| | bicyclo[2.2.1]hept-2- | | | | 3.35-3.78 (5H, m), 3.15 (1H, |
| | yl]pyrimidine-2,4- | | | | m), 2.47 (1H, s), |
| | diamine | | | | 1.86-2.35 (6H, m), |
| | | | | | 1.12-1.83 (6H, m), |
| | | | | | 0.80 (1H, m). |
| 27 | 4-(3-aminopyrrolidin- | Interm. | | 276 [M + H]⁺ | CDCl₃ 6.30 (2H, bs), |
| | 1-yl)-6- | 84 | | RT 1.81 mins | 5.47 (1H, s), |
| | (cyclohexylmethyl)pyrimidin- | | | (pH 5.8) | 2.97-4.03 (10H, m), |
| | 2-amine | | | | 2.30 (2H, d), 2.10 (1H, |
| | | | | | m), 1.50-1.80 (3H, |
| | | | | | m), 0.78-1.28 (6H, |
| | | | | | m). |
| 28 | 4-(3-aminopyrrolidin- | Interm. | | 220 [M + H]⁺ | CDCl₃ 5.33 (1H, s), |
| | 1-yl)-6- | 27 | | RT 1.34 mins | 3.30-3.87 (5H, m), |
| | cyclopropylpyrimidin- | | | (pH 5.8) | 1.70-2.32 (5H, m), |
| | 2-amine | | | | 0.90-1.35 (6H, m). |
| 29 | 4-(3-aminopyrrolidin- | Interm. | | 284 [M + H]⁺ | CDCl₃ |
| | 1-yl)-6-(2- | 85 | | RT 1.74 mins | 7.12-7.35 (5H, m), 5.52 (1H, |
| | phenylethyl)pyrimidin- | | | (pH 5.8) | s), 5.30 (2H, bs), |
| | 2-amine | | | | 3.05-3.75 (5H, m), |
| | | | | | 2.95 (2H, dd), |
| | | | | | 2.75 (2H, dd), 2.55 (2H, |
| | | | | | bs), 2.15 (1H, m), |
| | | | | | 1.75 (1H, m). |
| 30 | 4-cyclopentyl-6- | Interm. | | 248 [M + H]⁺ | d₆-DMSO 5.89 (1H, |
| | piperazin-1- | 45 | | RT 1.49 mins | s), 5.86 (2H, bs), |
| | ylpyrimidin-2-amine | | | (pH 5.8) | 3.45 (4H, m), |
| | | | | | 3.32 (1H, bs), 2.75 (4H, |
| | | | | | m), 2.50 (1H, m), |
| | | | | | 1.52-1.90 (8H, m). |
| 31 | 4-cyclopentyl-6-(3- | Interm. | | 262 [M + H]⁺ | CDCl₃ 6.62 (2H, bs), |
| | methylpiperazin-1- | 46 | | RT 1.73 mins | 5.55-6.20 (1H, bs), |
| | yl)pyrimidin-2-amine | | | (pH 5.8) | 5.85 (1H, s), |
| | | | | | 4.27 (2H, m), 3.10 (1H, |
| | | | | | d), 2.75-3.05 (4H, |
| | | | | | m), 2.60 (1H, t), |
| | | | | | 1.96-2.15 (2H, m), |
| | | | | | 1.55-1.88 (6H, m), |
| | | | | | 1.15 (3H, d). |
| 32 | 4-(3-aminopyrrolidin- | Interm. | | 262 [M + H]⁺ | CDCl₃ 7.18 (2H, bs), |
| | 1-yl)-6- | 47 | | RT 1.84 mins | 5.63 (1H, s), |
| | (cyclopentylmethyl)pyrimidin- | | | (pH 5.8) | 4.63 (2H, bs), |
| | 2-amine | | | | 3.05-3.85 (5H, m), 2.54 (2H, |
| | | | | | d), 2.20 (2H, m), |
| | | | | | 1.43-1.95 (7H, m), |
| | | | | | 1.10-1.32 (2H, m). |
| 33 | 4-[adamantan-2-yl]-6- | Interm. | | 328 [M + H]⁺ | CD₃OD 6.40 (1H, s), |
| | (3-methylpiperazin-1- | 48 | | RT 1.37 mins | 5.05 (1H, m), |
| | yl)pyrimidin-2-amine | | | (pH 2.5) | 4.40 (1H, m), |
| | | | | | 3.10-3.70 (5H, m), 3.02 (1H, |
| | | | | | s), 2.45 (2H, s), |
| | | | | | 1.68-2.15 (12H, m), |
| | | | | | 1.43 (3H, d). |
| 34 | 4-cyclopentyl-6- | Interm. | | 288 [M + H]⁺ | CDCl₃ |
| | [(4aR*,7aR*)- | 50 | | RT 1.66 mins | 5.90-6.50 (2H, bm), 5.65 (1H, |
| | octahydro-6H- | | | (pH 5.8) | s), 3.15-3.75 (5H, |
| | pyrrolo[3,4-b]pyridin- | | | | m), 3.00 (1H, m), |
| | 6-yl]pyrimidin-2-amine | | | | 2.90 (1H, m), |
| | | | | | 2.65 (1H, m), |
| | | | | | 2.20-2.47 (1H, bm), 2.00 (1H, |
| | | | | | m), |
| | | | | | 1.40-1.85 (12H, m). |
| 35 | 4-(3-aminoazetidin-1- | Interm. | | 234 [M + H]⁺ | CD₃OD 5.70 (1H, s), |
| | yl)-6- | 51 | | RT 1.48 mins | 4.30 (2H, dt), |
| | cyclopentylpyrimidin- | | | (pH 5.8) | 3.93 (1H, m), 3.78 (2H, |
| | 2-amine | | | | dd), 2.84 (1H, m), |
| | | | | | 1.95-2.08 (2H, m), |
| | | | | | 1.60-1.89 (6H, m). |
| 36 | 6-(3-aminopyrrolidin- | Interm. | | 289 [M + H]⁺ | CDCl₃ 5.58 (1H, bs), |
| | 1-yl)-N⁴- | 52 | | RT 1.80 mins | 5.15 (1H, s), |
| | [(1R*,2S*,4S*)- | | | (pH 5.8) | 4.95 (2H, bs), 4.60 (1H, |
| | bicyclo[2.2.1]hept-2- | | | | s), 3.32-3.60 (6H, |
| | yl]pyrimidine-2,4- | | | | m), 3.05 (1H, m), |
| | diamine | | | | 2.33 (1H, s), |
| | | | | | 2.10 (1H, s), |
| | | | | | 1.95-2.07 (2H, m), 1.65 (1H, |
| | | | | | m), 1.10-1.58 (6H, |
| | | | | | m), 0.75 (1H, m). |
| 37 | 4-cyclohexyl-6-(3- | Interm. | | 276 [M + H]⁺ | CDCl₃ 5.80 (1H, s), |
| | methylpiperazin-1- | 53 | | RT 1.81 mins | 5.00 (2H, br s), |
| | yl)pyrimidin-2-amine | | | (pH 5.8) | 4.20 (2H, m), 3.05 (1H, |
| | | | | | m), 2.75-2.93 (3H, |
| | | | | | m), 2.50 (1H, m), |
| | | | | | 2.35 (1H, m), |
| | | | | | 1.70-1.95 (5H, m), |
| | | | | | 1.22-1.50 (6H, m), |
| | | | | | 1.13 (3H, d) |
| 38 | 4-cyolopentyl-6-[(3S)- | Interm. | | 290 [M + H]⁺ | CD₃OD 5.86 (1H, s), |
| | 3-isopropylpiperazin- | 56 | | RT 2.02 mins | 4.32 (1H, bd), |
| | 1-yl]pyrimidin-2-amine | | | (pH 5.8) | 4.09 (1H, bd), 2.94 (1H, |
| | | | | | dt), 2.45-2.85 (4H, |
| | | | | | mm), 2.24 (1H, ddd), |
| | | | | | 1.80-1.97 (2H, m), |
| | | | | | 1.49-1.77 (7H, |
| | | | | | mm), 0.92 (6H, d) |
| 39 | 4-cyclopentyl-6-(3,8- | Interm. | | 274 [M + H]⁺ | CD₃OD 5.97 (1H, s), |
| | diazabicyolo[3.2.1]oct- | 54 | | RT 1.63 mins | 4.08 (2H, m), |
| | 3-yl)pyrimidin-2-amine | | | (pH 5.8) | 3.65 (2H, m), 3.04 (2H, |
| | | | | | m), 2.84 (1H, m), |
| | | | | | 1.61-2.11 (12H, m) |
| 40 | 4-cyclopentyl-6-[(3S)- | Interm. | Diacetate | 304 [M + H]⁺ | CD₃OD 6.20 (1H, s), |
| | 3-isobutylpiperazin-1- | 57 | | RT 2.39 mins | 4.55 (1H, bd), |
| | yl]pyrimidin-2-amine | | | (pH 5.8) | 4.42 (1H, bd), |
| | | | | | 3.05-3.24 (2H, m), |
| | | | | | 2.74-2.98 (4H, m), |
| | | | | | 2.03-2.14 (3H, bs), 2.0 (6H, 2 × AcOH, |
| | | | | | s), |
| | | | | | 1.68-1.93 (7H, mm), |
| | | | | | 1.41 (1H, t), 1.00 (6H, t) |

Comp No means Compound Number
Interm. means Intermediate

### EXAMPLE 12

### Synthesis of 4-chloro-6-(2-methylpyrrolidin-1-yl)pyrimidin-2-amine (Intermediate 14)

4,6-Dichloropyrimidin-2-amine (2.0 g), 2-methylpyrrolidine (2 ml) and triethylamine (2 ml) are dissolved in dry NMP (5 ml) and heated under microwave irradiation at 110° C. for 20 mins. The solution is then added to water (20 ml) and extracted with DCM (2×20 ml). The organic phase is then washed with water (2×20 ml), dried (MgSO₄) and concentrated in vacuo. Purification by flash chromatography, eluting with EtOAc affords the title compound as a white solid (2.30 g, 89%). LCMS 262 [M+H]⁺ RT 2.20 mins (pH 2.5). ¹H NMR 300 MHz (CDCl₃) (δ ppm): 5.75 (1H, s), 4.80 (2H, br s), 3.82-4.39 (1H, br m), 3.13-3.61 (2H, m), 1.85-2.13 (4H, m), 1.20 (3H, d).

Intermediates 15 to 17 are prepared from 4,6-dichloropyrimidin-2-amine, in a similar manner to the method described for Intermediate 14 in Example 12.The reagents used and the results obtained are tabulated below (Table 5). The free base of the compounds is obtained.

TABLE 5

| Interm. | | | | ¹H NMR |
|---|---|---|---|---|
| No | IUPAC Name | Starting Materials | LCMS | (Solvent, δ ppm) |
| 15 | 6-chloro-N⁴- | 1,2,3,4- | 275/277 [M + H]⁺ | CDCl₃ 7.25-7.30 (1H, m), |
| | (1,2,3,4- | tetrahydronaphthalen- | RT 3.60 mins | 7.10-7.25 (3H, m), |
| | tetrahydronaphthalen- | 2-amine | (pH 5.8) | 5.85 (1H, bs), 4.75-5.20 (4H, |
| | 2- | (CAS RN 2954- | | m), 2.75-2.90 (2H, m), |
| | yl)pyrimidine- | 50-9) | | 1.95-2.10 (1H, m), |
| | 2,4-diamine | | | 1.80-1.95 (3H, m) |
| 16 | 6-chloro-N⁴- | indan-2-amine | 261/263 [M + H]⁺ | CDCl₃ 7.15-7.25 (4H, m), |
| | (2,3-dihydro- | (CAS RN | RT 3.37 mins | 5.85 (1H, s), |
| | 1H-inden-2- | 915232-20-1) | (pH 5.8) | 4.90-5.05 (1H, m), 4.70-4.85 (2H, |
| | yl)pyrimidine- | | | bs), 4.45-4.65 (1H, m), |
| | 2,4-diamine | | | 3.35 (2H, dd), 2.85-(2H, |
| | | | | dd) |
| 17 | N⁴- | (1R*,2R*,4S*)- | 239 [M + H]⁺ | CDCl₃ 5.76 (1H, s), |
| | [(1R*,2R*,4S*)- | bicyclo[2.2.1]heptan- | RT 2.77 mins | 4.87 (1H, bs), 4.80 (2H, bs), |
| | bicyclo[2.2.1]hept- | 2-amine | (pH 5.8) | 3.38 (1H, bs), 2.27 (2H, |
| | 2-yl]-6- | (CAS RN 7242- | | bdd), 1.84 (1H, ddd), |
| | chloropyrimidine- | 92-4) | | 1.44-1.63 (2H, m), |
| | 2,4- | | | 1.36-1.42 (1H, m), 1.10-1.28 (4H, |
| | diamine | | | m). |

Interm. No means Intermediate Number

### EXAMPLE 13

### Synthesis of 4-chloro-6-(1,3-dihydro-2H-isoindol-2-yl)pyrimidin-2-amine (Intermediate 18)

4,6-Dichloropyrimidin-2-amine (1.12 g), isoindoline (1.2 ml), and triethylamine (1.63 ml) in EtOH (33 ml) are heated with stirring at 85° C. for 6 hours. The solvent is removed in vacuo, and the residual solid suspended in DCM (40 ml) and dilute citric acid solution (30 ml). The suspended solid is filtered off, washed with DCM and water, and dried to afford the title compound as a grey solid (1.573 g, 93%). LCMS 247 [M+H]⁺, RT 3.05 mins (pH 2.5). 1H NMR 300 MHz (CDCl₃) (δ ppm): 7.25-7.48 (4H, m), 6.58 (2H, s), 5.92 (1 H, s), 4.71 (4H, br d).

Intermediates 19 to 29 are prepared in a similar manner to the method described for Intermediate 18 in Example 13.The reagents used and the results obtained are tabulated below (Table 6). The free base of the compounds is obtained unless otherwise stated.

TABLE 6

| Interm. | | | | | ¹H NMR |
|---|---|---|---|---|---|
| No | IUPAC Name | Starting Materials | Salt | LCMS | (Solvent, δ ppm) |
| 19 | tert-butyl [1-(2- | 4-chloro-6- | | 348 [M + H]⁺ | CDCl₃ 5.49 (1H, |
| | amino-6- | cyclohexylpyrimidin- | | RT 2.97 mins | s), 5.00 (2H, br s), |
| | cyclohexylpyrimidin- | 2-amine CAS | | (pH 5.8) | 4.55 (1H, br s), |
| | 4- | RN (688782-65- | | | 4.35 (2H, t), |
| | yl)azetidin-3- | 2), tert-butyl | | | 3.80 (2H, dd), 2.35 (1H, |
| | yl]carbamate | azetidin-3- | | | br t), 1.90 (2H, br |
| | | ylcarbamate (CAS | | | d), 1.80 (2H, br d), |
| | | RN 91188-13-5) | | | 1.75 (1H, br d), |
| | | | | | 1.48 (9H, s), |
| | | | | | 1.20-1.46 (5H, m) |
| 20 | tert-butyl | 4-chloro-6- | | 388 [M + H]⁺ | CDCl₃ 5.60 (1H, |
| | (3aR*,6aS*)-5- | cyclohexylpyrimidin- | | RT 3.18 mins | s), 4.85 (2H, br s), |
| | (2-amino-6- | 2-amine, tert- | | (pH 5.8) | 3.65 (4H, m), |
| | cyclohexylpyrimidin- | butyl (3aR*,6aS*)- | | | 3.35 (2H, m), 3.25 (1H, |
| | 4- | hexahydropyrrolo[3,4- | | | s), 2.95 (2H, br s), |
| | yl)- | c]pyrrole- | | | 2.75 (1H, m), |
| | hexahydropyrrolo[3,4- | 2(1H)-carboxylate | | | 2.35 (1H, m), |
| | c]pyrrole- | (CAS RN 445309- | | | 1.65-1.98 (5H, m), 1.50 (9H, |
| | 2(1H)- | 99-9) | | | s), 1.20-1.49 (5H, |
| | carboxylate | | | | m) |
| 21 | 4-cyclohexyl- | 4-chloro-6- | | 304 [M + H]⁺ | CDCl₃ 5.55 (1H, |
| | 6- | cyclohexylpyrimidin- | | RT 3.03 mins | s), 4.85 (2H, s), |
| | [(1R*,5S*,6S*)- | 2-amine, | | (pH 5.8) | 4.15 (1H, s), |
| | 6-nitro-3- | (1R*,5S*,6S*)-6- | | | 3.90 (2H, d), 3.58 (2H, |
| | azabicyclo[3.1.0]hex- | nitro-3- | | | d), 2.80 (2H, s), |
| | 3- | azabicyclo[3.1.0]hexane | | | 2.30 (1H, m), |
| | yl]pyrimidin-2- | hydrochloride | | | 1.65-1.98 (5H, m), |
| | amine | (CAS RN 171176- | | | 1.20-1.49 (5H, m) |
| | | 54-8) | | | |
| 22 | tert-butyl | 4-chloro-6- | | 374 [M + H]⁺ | CDCl₃ 5.85 (1H, |
| | (1R*,5S*,6S*)- | cyclohexylpyrimidin- | | RT 2.37 mins | s), 4.95 (1H, s), |
| | 6-[(2-amino-6- | 2-amine, tert- | | (pH 2.5) | 4.65 (2H, s), |
| | cyclohexylpyrimidin- | butyl | | | 3.75 (1H, d), 3.65 (1H, |
| | 4- | (1R*,5S*,6S*)-6- | | | d), 3.48 (2H, m), |
| | yl)amino]-3- | amino-3- | | | 2.40 (1H, m), |
| | azabicyclo[3.1.0]hexane- | azabicyclo[3.1.0]hexane- | | | 2.30 (1H, d), |
| | 3- | 3- | | | 1.65-1.98 (5H, m), 1.49 (9H, |
| | carboxylate | carboxylate (CAS | | | s), 1.20-1.49 (5H, |
| | | RN 273206-92-1) | | | m) |
| 23 | tert-butyl | 4-chloro-6- | | 360 [M + H]⁺ | CDCl₃ 5.85 (1H, |
| | (1R*,5S*,6S*)- | cyclopentylpyrimidin- | | RT 3.04 mins | s), 5.09 (1H, s), |
| | 6-[(2-amino-6- | 2-amine (CAS | | (pH 5.8) | 4.75 (2H, s), |
| | cyclopentylpyrimidin- | RN 199863-83-3), | | | 3.73 (1H, d), 3.65 (1H, |
| | 4- | tert-butyl | | | d), 3.48 (2H, m), |
| | yl)amino]-3- | (1R*,5S*,6S*)-6- | | | 2.85 (1H, t), |
| | azabicyclo[3.1.0]hexane- | amino-3- | | | 2.30 (1H, s), 2.00 (2H, |
| | 3- | azabicyclo[3.1.0]hexane- | | | m), 1.60-1.85 (8H, |
| | carboxylate | 3- | | | m), 1.49 (9H, s) |
| | | carboxylate | | | |
| 24 | tert-butyl | Intermediate 87, | | 374 [M + H]⁺ | CDCl₃ 5.85 (1H, |
| | (1R*,5S*,6S*)- | tert-butyl | | RT 3.39 mins | s), 5.05 (1H, s), |
| | 6-{[2-amino-6- | (1R*,5S*,6S*)-6- | | (pH 5.8) | 4.70 (2H, s), |
| | (cyclopentylmethyl)pyrimidin- | amino-3- | | | (1H, d), 3.63 (1H, |
| | 4-yl]amino}-3- | azabicyclo[3.1.0]hexane- | | | d), 3.48 (2H, m), |
| | azabicyclo[3.1.0]hexane- | 3- | | | 2.50 (2H, d), |
| | 3- | carboxylate | | | 2.33 (1H, s), 2.20 (2H, |
| | carboxylate | | | | m), 1.60-1.85 (6H, |
| | | | | | m), 1.49 (9H, s), |
| | | | | | 1.20 (2H, m) |
| 25 | 6-chloro-N⁴- | 4,6- | | 229 [M + H]⁺ | d₆-DMSO |
| | (tetrahydro- | dichloropyrimidin- | | RT 2.00 mins | 7.10 (1H, d), 6.37 (2H, |
| | 2H-pyran-4- | 2-amine, | | (pH 5.8) | s), 5.67 (1H, s), |
| | yl)pyrimidine- | tetrahydro-2H- | | | 3.95 (1H, br m), |
| | 2,4-diamine | pyran-4-amine | | | 3.85 (2H, d), |
| | | (CAS RN 38041- | | | 3.4 (3H, m), 1.79 (2H, |
| | | 19-9) | | | d), 1.40 (2H, m) |
| 26 | N⁴- | 4,6- | | 239 [M + H]⁺ | d₆-DMSO |
| | [(1R*,2S*,4S*)- | dichloropyrimidin- | | RT 2.74 mins | 8.65 (1H, bs), 7.60 (2H, |
| | bicyclo[2.2.1]hept- | 2-amine, | | (pH 2.5) | bs), 6.15 (1H, s), |
| | 2-yl]-6- | (1R*,2S*,4S*)- | | | 4.12 (1H, m), |
| | chloropyrimidine- | bicyclo[2.2.1]heptan- | | | 2.20 (1H, s), 2.00 (1H, |
| | 2,4- | 2-amine | | | m), |
| | diamine | hydrochloride | | | 1.23-1.65 (7H, m), 1.00 (1H, |
| | | (CAS RN 65481- | | | m). |
| | | 69-8) | | | |
| 27 | tert-butyl [1-(2- | 4-chloro-6- | formate | 320 [M + H]⁺ | CDCl₃ 8.5 (1H, |
| | amino-6- | cyclopropylpyrimidin- | | RT 1.93 mins | HCOOH), |
| | cyclopropylpyrimidin- | 2-amine (CAS | | (pH 2.5) | 6.05 (2H, bs), 5.30 (1H, |
| | 4- | RN 21573-09-1), | | | m), |
| | yl)pyrrolidin-3- | tert-butyl | | | 4.12-4.40 (1H, m), |
| | yl]carbamate | pyrrolidin-3-yl- | | | 3.23-3.87 (4H, m), |
| | | carbamate | | | 2.65 (1H, s), |
| | | | | | 2.15-2.52 (1H, m), |
| | | | | | 1.88-2.07 (2H, m), |
| | | | | | 1.45 (9H, s), |
| | | | | | 1.20 (2H, m), 0.96 (2H, |
| | | | | | m). |
| 28 | 6-chloro-N⁴- | 4,6- | | 216 [M + H]⁺ | CDCl₃ 5.80 (1H, |
| | [2- | dichloropyrimidin- | | RT 1.85 min | s), 5.45 (1H, br s), |
| | (dimethylamino)ethyl]pyrimidine- | 2-amine, N,N- | | (pH | 4.82 (2H, s), |
| | 2,4- | dimethylethylenediamine | | 5.8) | 3.30 (2H, m), 2.47 (2H, |
| | diamine | | | | t), 2.22 (6H, s) |
| 29 | N⁴-(8-benzyl- | 4-chloro-6- | | 392 [M + H]⁺, | CDCl₃ |
| | 8- | cyclohexylpyrimidin- | | RT 2.30 mins | 7.20-7.40 (5H, m), 5.55 (1H, |
| | azabicyclo[3.2.1]oct- | 2-amine, 8- | | (pH 5.8) | s), 4.65 (2H, br s), |
| | 3-yl)-6- | benzyl-8- | | | 4.40 (1H, m), |
| | cyclohexylpyrimidine- | azabicyclo[3.2.1]octan- | | | 4.00 (1H, br s), |
| | 2,4- | 3-amine | | | 3.55 (2H, s), 3.25 (2H, |
| | diamine | (CAS RN 96901- | | | s), 2.30 (1H, m), |
| | | 92-7) | | | 2.10 (2H, m), |
| | | | | | 1.65-1.90 (5H, m), |
| | | | | | 1.70 (2H, m), |
| | | | | | 1.20-1.59 (9H, m) |

Interm. No means Intermediate Number

### EXAMPLE 14

### Synthesis of 4-(1,3-dihydro-2H-isoindol-2-yl)-6-piperazin-1-ylpyrimidin-2-amine (Compound 41)

A mixture of Intermediate 18 (1 g), N-tert-butoxycarbonylpiperazine (1 g) in NMP (4 ml) and Et₃N (2 ml) is heated in the microwave at 150° C. for 30 mins, then cooled, added to H₂O (20 ml) and extracted with EtOAc (2×20 ml). The solvent is washed with H₂O, dried and evaporated. The crude residue is dissolved in DCM (20 ml) and TFA (4 ml) is added. The solution is stirred for 1 hr then evaporated in vacuo and azeotroped with heptane (2×20 ml). The residue is dissolved in H₂O and the solution washed with ether, then basified with solid K₂CO₃ and extracted with DCM (33 30 ml). The combined organic layer is dried and evaporated under reduced pressure to give the title compound as a colorless solid (700 mg). LCMS 297 [M+H]⁺, RT 2.05 mins (pH 5.8). ¹H NMR 300 MHz (d₆-DMSO) (δ ppm): 7.25-7.40 (4H, m), 5.62 (2H, s), 5.15 (1 H, s), 4.65 (4H, m), 2.75 (4H, s), 1.85 (6H, s).

Compounds 42 through 52 are prepared in a similar manner to the method described for Compound 41 in Example 14.The reagents used and the results obtained are tabulated below (Table 7). The free base of the compounds is obtained unless otherwise stated.

TABLE 7

| Comp. | | Starting | | | ¹H NMR |
|---|---|---|---|---|---|
| No | IUPAC Name | Materials | Salt | LCMS | (Solvent, δ ppm) |
| 42 | 4-(2- | Intermediate 14, | | 263 [M + H]⁺ | CDCl₃ 4.95 (1H, s), |
| | methylpyrrolidin- | N-tert- | | RT 1.72 mins | 4.49 (2H, s), 4.15 (1H, |
| | 1-yl)-6-piperazin- | butoxycarbonyl- | | (pH | m), 3.50 (5H, m), |
| | 1-ylpyrimidin-2- | piperazine | | 5.8) | 3.35 (1H, m), 2.90 (4H, m), |
| | amine | | | | 1.85-2.00 (4H, m), |
| | | | | | 1.68 (1H, m), 1.25 (3H, d) |
| 43 | 4-(3- | Intermediate 18, | | 297 [M + H]⁺ | CD₃OD 7.35 (4H, m), |
| | aminopyrrolidin-1- | 3-(tert- | | RT 1.72 mins | 5.50 (1H, s), 5.00 (2H, |
| | yl)-6-(1,3-dihydro- | butoxycarbonylamino)- | | (pH | s), 4.75 (4H, s), |
| | 2H-isoindol-2- | pyrrolidine | | 5.8) | 3.95 (1H, m), 3.80 (1H, m), |
| | yl)pyrimidin-2- | | | | 3.55-3.75 (2H, m), |
| | amine | | | | 2.45 (1H, m), 2.20 (1H, m), |
| | | | | | 2.00 (6H, s) |
| 44 | 4-(1,3-dihydro-2H- | Intermediate 18, | Diacetate | 325 [M + H]⁺ | d₆-DMSO |
| | isoindol-2-yl)-6-[4- | 4-N-(tert- | | RT | 7.25-7.40 (4H, m), 5.15 (2H, s), |
| | (methylamino)piperidin- | butoxycarbonyl- | | 2.08 mins | 5.15 (1H, s), 4.70 (4H, |
| | 1- | N- | | (pH 5.8) | s), 4.25 (2H, m), |
| | yl]pyrimidin-2- | methylamino)piperidine | | | 2.80 (2H, m), 2.60 (1H, m), |
| | amine | | | | 2.30 (3H, s), 1.85 (2H, |
| | | | | | m), 1.80 (6H, s), |
| | | | | | 1.20 (2H, m) |
| 45 | 6-(3- | 6-chloro-N⁴- | Diacetate | 311 [M + H]⁺ | CDCl₃ 7.85-8.00 (1H, |
| | aminopyrrolidin-1- | (2,3-dihydro-1H- | | RT 2.07 mins | m), 7.30-7.35 (1H, m), |
| | yl)-N⁴-(2,3- | inden-1- | | (pH 5.8) | 7.15-7.25 (3H, m), |
| | dihydro-1H-inden- | yl)pyrimidine- | | | 6.15-6.45 (2H, bs), |
| | 1-yl)pyrimidine- | 2,4-diamine | | | 5.40-5.70 (2H, m), 4.90-5.05 (1H, |
| | 2,4-diamine | (CAS RN | | | m), 4.85 (1H, s), |
| | | 147406-83-5), | | | 3.70-3.80 (1H, m), |
| | | tert-butyl | | | 3.50-3.70 (2H, m), 3.40-3.50 (1H, |
| | | pyrrolidin-3- | | | m), 3.20-3.35 (1H, m), |
| | | ylcarbamate | | | 3.00-3.10 (1H, m), |
| | | | | | 2.80-2.95 (1H, m), |
| | | | | | 2.50-2.60 (1H, m), 2.10-2.25 (2H, |
| | | | | | m), 2.00-2.10 (1H, m), |
| | | | | | 1.95 (6H, s, 2 AcOH) |
| 46 | 4-cyclopentyl-6- | 4-chloro-6- | | 276 [M + H]⁺ | CD₃OD 5.87 (1H, s), |
| | (3-ethylpiperazin- | cyclopentylpyrimidin- | | RT 1.77 mins | 4.20-4.31 (1H, m), |
| | 1-yl)pyrimidin-2- | 2-amine, | | (pH 5.8) | 4.12 (1H, bd), |
| | amine | tert-butyl 2- | | | 2.58-2.98 (4H, mm), |
| | | ethylpiperazine- | | | 2.36-2.48 (2H, m), |
| | | 1-carboxylate | | | 1.81-1.95 (2H, m), |
| | | | | | 1.50-1.78 (6H, mm), |
| | | | | | 1.32-1.44 (2H, m), 0.91 (3H, |
| | | | | | t). |
| 47 | N⁴-cyclohexyl-6- | 6-chloro-N⁴- | | 305 [M + H]⁺ | CD₃OD 5.40 (1H, s), |
| | [4- | cyclohexylpyrimidin- | | RT 1.71 mins | 4.45 (2H, bs), 3.42 (1H, |
| | (methylamino)piperidin- | 2,4-diamine | | (pH 5.8) | m), 3.25 (1H, m), |
| | 1- | (CAS RN 30182- | | | 2.90 (2H, m), 2.64 (3H, s), |
| | yl]pyrimidine-2,4- | 26-4), tert-butyl | | | 2.08 (2H, m), 1.87 (2H, |
| | diamine | methyl(piperidin- | | | m), 1.63-1.77 (2H, m), |
| | | 4-yl)carbamate | | | 1.06-1.63 (10H, m) |
| | | (CAS RN | | | |
| | | 108612-54-0) | | | |
| 48 | 4-cyclopropyl-6- | 4-chloro-6- | | 234 [M + H]⁺ | CDCl₃ 6.10 (2H, m), |
| | [3- | cyclopropylpyrimidin- | | RT 1.21 mins | 5.45 (1H, s), |
| | (methylamino)pyrrolidin- | 2-amine, | | (pH 5.8) | 3.22-3.72 (6H, m), 2.48 (3H, |
| | 1- | tert-butyl | | | s), 2.15 (1H, m), |
| | yl]pyrimidin-2- | methyl(pyrrolidin- | | | 1.76-1.95 (2H, m), |
| | amine | 3-yl)carbamate | | | 0.87-1.10 (4H, m). |
| | | (CAS RN | | | |
| | | 172478-00-1) | | | |
| 49 | 4- | Intermediate 87, | | 276 [M + H]⁺ | CDCl₃ 5.80 (1H, s), |
| | (cyclopentylmethyl)- | tert-butyl 2- | | RT 1.81 mins | 5.40 (2H, bs), 4.20 (2H, |
| | 6-(3- | methylpiperazine- | | (pH 5.8) | m), 3.48 (1H, bs), |
| | methylpiperazin- | 1-carboxylate | | | 3.08 (1H, m), |
| | 1-yl)pyrimidin-2- | (CAS RN | | | 2.75-2.95 (3H, m), 2.52 (1H, m), |
| | amine | 120737-78-2) | | | 2.45 (2H, d), 2.20 (1H, |
| | | | | | m), 1.43-1.83 (6H, m), |
| | | | | | 1.15-1.30 (2H, m), |
| | | | | | 1.12 (3H, d). |
| 50 | 4-cyclohexyl-6- | 4-chloro-6- | | 276 [M + H]⁺ | CDCl₃ 5.73 (1H, s), |
| | (1,4-diazepan-1- | cyclohexylpyrimidin- | | RT 1.92 mins | 4.60 (2H, s), |
| | yl)pyrimidin-2- | 2-amine, | | (pH 5.8) | 3.55-3.78 (4H, m), 3.00 (2H, m), |
| | amine | tert-butyl 1,4- | | | 2.84 (2H, m), 2.30 (1H, |
| | | diazepane-1- | | | m), 1.60-1.90 (8H, m), |
| | | carboxylate | | | 1.20-1.55 (5H, m) |
| | | (CAS RN | | | |
| | | 112275-50-0) | | | |
| 51 | 4-cyclopentyl-6- | 4-chloro-6- | | 262 [M + H]⁺ | CDCl₃ 5.78 (1H, s), |
| | (1,4-diazepan-1- | cyclopentylpyrimidin- | | RT 1.81 mins | 4.60 (2H, br s), |
| | yl)pyrimidin-2- | 2-amine, | | (pH 5.8) | 3.60-3.75 (4H, m), 3.00 (2H, |
| | amine | tert-butyl 1,4- | | | m), 2.83 (2H, m), |
| | | diazepane-1- | | | 2.75 (1H, m), |
| | | carboxylate | | | 1.56-2.05 (11H, m) |
| | | (CAS RN | | | |
| | | 112275-50-0) | | | |
| 52 | 6-cyclopentyl-N⁴- | (2-amino-4- | | 236 [M + H]⁺ | CD₃OD 5.78 (1H, s), |
| | [2- | chloro-6- | | RT 1.30 min | 3.45 (2H, t), 2.80 (1H, |
| | (methylamino)ethyl]pyrimidine- | cyclopentylpyrimidine, | | (pH | m), 2.77 (2H, t), |
| | 2,4- | tert-butyl | | 5.8) | 2.42 (3H, s), 2.00 (2H, m), |
| | diamine | (2- | | | 1.60-1.82 (4H, m) |
| | | aminoethyl)methylcarbamate | | | |
| | | (CAS RN | | | |
| | | 121492-06-6) | | | |

Comp. No means Compound Number

The maleate salt of compound 43 is prepared as described below.

### EXAMPLE 15

### Synthesis of 4-(3-aminopyrrolidin-1-yl)-6-(1,3-dihydro-2H-isoindol-2-yl)pyrimidin-2-amine maleate salt (Compound 53)

Compound 43 (0.22 g) is suspended in ethanol (10 ml) and a solution of maleic acid (CAS RN 110-16-7) (0.12 g) in ethanol (5 ml) is added dropwise. The mixture is stirred for 1 hr, then filtered and the crude product washed with ethanol (2×5 ml) to give the title compound as colorless solid (0.30 g, 98%). LCMS 297 [M+H]⁺, RT (pH 5.8) 2.01 mins. ¹H NMR 300 MHz (d₆-DMSO) (δ ppm): 8.05 (3H, br s), 7.35 (4H, m), 6.75 (2H, br s), 6.05 (2H, s), 5.10 (1H, s), 4.75 (4H, s), 3.98 (1H, m), 3.70 (1H, m), 3.55 (2H, m), 3.32 (1H, m), 2.36 (1H, m), 2.13 (1H, m).

Compounds 54 and 55 are prepared in a similar manner to the method described for Compound 53 in Example 15.The reagents used and the results obtained are tabulated below (Table 8). The free base of the compounds is obtained unless otherwise stated.

TABLE 8

| Comp. | IUPAC | Starting | | | ¹H NMR |
|---|---|---|---|---|---|
| No | Name | Materials | Salt | LCMS | (Solvent, δ ppm) |
| 54 | 4-(1,3- | Compound 41 | maleate | 297 [M + H]⁺ | d₆ DMSO 8.83 (2H, br |
| | dihydro- | | | RT 2.13 min | s), 7.35 (4H, m), |
| | 2H- | | | (pH | 6.25 (2H, br s), 6.05 (2H, s), |
| | isoindol-2- | | | 5.8) | 5.32 (1H, s), 4.72 (4H, |
| | yl)-6- | | | | s), 3.77 (4H, m), |
| | piperazin- | | | | 3.15 (4H, m) |
| | 1- | | | | |
| | ylpyrimidin- | | | | |
| | 2-amine | | | | |
| 55 | 6-(3- | Compound 36 | maleate | 289 [M + H]⁺ | d₆ DMSO 7.90 (2H, br |
| | aminopyrrolidin- | | | RT 1.96 min | s), 6.30 (1H, m), |
| | 1-yl)- | | | (pH | 6.02 (2H, s), 5.55 (2H, br s), |
| | N⁴- | | | 5.8) | 4.85 (1H, s), 3.92 (1H, |
| | [(1R*,2S*,4S*)- | | | | m), 3.83 (2H, m), |
| | bicyclo[2.2.1]hept- | | | | 3.55 (1H, m), 3.30 (2H, m), |
| | 2- | | | | 2.39 (1H, m), 2.22 (1H, |
| | yl]pyrimidine- | | | | m), 2.15 (1H, m), |
| | 2,4- | | | | 1.95 (2H, m), 1.20-1.62 (6H, |
| | diamine | | | | m), 0.85 (2H, m) |

Comp. No means Compound Number

### EXAMPLE 16

### Synthesis of 6-chloro-N⁴-cyclohexyl-N⁴-methylpyrimidine-2,4-diamine (Intermediate 30)

A solution of 4,6-dichloropyrimidin-2-amine (346 mg), triethylamine (0.44 ml) and N-methylcyclohexylamine (0.274 ml) in ethanol (2.5 ml) is heated in a microwave at 150° C. for 30 minutes. The solution is concentrated in vacuo and the residue is purified by column chromatography on silica, eluting with 20%-50% EtOAc in heptane to afford the title compound as a colorless solid (404 mg, 80%). LCMS 241 [M+H]⁺, RT 3.78 mins (pH 5.8). ¹H NMR 300 MHz (CDCl₃) (δ ppm): 5.9 (1H, s), 4.8 (2H, s), 4.1-4.5 (1H, m), s), 1.85 (2H, m), 1.6-1.75 (3H, m), 1.3-1.5 (4H, m), 1.2 (1H, m).

### Example 17

### Synthesis of N⁴-cyclohexyl-6-(4-methylpiperazin-1-yl)pyrimidine-2,4-diamine (Compound 56)

A mixture of 4-chloro-6-(4-methylpiperazin-1-yl)pyrimidin-2-amine (70 mg) and cyclohexylamine (1 ml) is heated in a microwave at 200° C. for 3 hours. Purification by preparative HPLC (Method B) affords the title compound as a colorless solid (38 mg, 42%). LCMS 291 [M+H]⁺, RT 2.09 mins (pH 5.8). ¹H NMR 300 MHz (CDCl₃) (δ ppm): 5.0 (1H, s), 4.5 (2H, br s), 3.6 (1H, m), 3.5, (4H, m), 2.45 (4H, m), 2.3 (3H, s), 2.2 (1H, br s), 2.0 (2H, m), 1.75(2H, m), 1.6 (1H, m), 1.2-1.5 (5H, m).

Compounds 57 through 118 and Intermediates 31 to 57 are prepared in a similar manner to the method described for Compound 56 in Example 17. Note that this method can be performed with or without NMP as the solvent and with or without the presence of a base (e.g. Et₃N). Purification of the examples is achieved by preparative HPLC using either Method C or Method D.

The reagents used and the results obtained are tabulated below (Table 9). The free base is obtained unless otherwise stated.

TABLE 9

| | | | | | ¹H NMR |
|---|---|---|---|---|---|
| Comp. No | IUPAC Name | Starting Materials | Salt | LCMS | (Solvent, δ ppm) |
| 57 | 4-(4- | 4-chloro-6-(4- | | 277 [M + H]⁺ | d₆₋DMSO |
| | methylpiperazin- | methylpiperazin- | | RT 1.95 mins | 4.95 (1H, s), |
| | 1-yl)-6-[2- | 1-yl)pyrimidin-2- | | (pH 5.8) | 4.70 (2H, br s), |
| | methylpyrrolidin- | amine, 2- | | | 4.15 (1H, m), |
| | 1-yl]pyrimidin-2- | methylpyrrolidine | | | 3.55 (4H, m), |
| | amine | | | | 3.45 (1H, m), |
| | | | | | 3.35 (1H, m), |
| | | | | | 2.50 (4H, m), |
| | | | | | 2.35 (3H, s), |
| | | | | | 1.85-2.10 (3H, m), |
| | | | | | 1.70 (1H, m), |
| | | | | | 1.20 (3H, d) |
| 58 | 4-cyclohexyl-6- | 4-chloro-6- | | 290 [M + H]⁺ | CDCl₃ 5.7 (1H, |
| | (4-methyl-1,4- | cyclohexylpyrimidin- | | RT 1.86 mins | s), 4.75 (2H, s), |
| | diazepan-1- | 2-amine, | | (pH 5.8) | 3.8 (2H, m), |
| | yl)pyrimidin-2- | N- | | | 3.6 (2H, m), |
| | amine | methylhomopiperazine | | | 2.65 (2H, m), |
| | | | | | 2.55 (2H, m), |
| | | | | | 2.4 (3H, s), |
| | | | | | 2.35 (1H, m), |
| | | | | | 1.7-2.0 (7H, m), |
| | | | | | 1.2-1.5 (5H, m) |
| 59 | 4-(4- | 4-chloro-6-(4- | | 291 [M + H]⁺ | CDCl₃ 5.20 (1H, |
| | methylpiperazin- | methylpiperazin- | | RT 2.03 mins | s), 4.40 (2H, s), |
| | 1-yl)-6-(4- | 1-yl)pyrimidin-2- | | (pH 5.8) | 4.25 (2H, m), |
| | methylpiperidin- | amine, 4- | | | 3.50 (4H, m), |
| | 1-yl)pyrimidin-2- | methylpiperidine | | | 2.75 (2H, m), |
| | amine | | | | 2.45 (4H, m), |
| | | | | | 2.23 (3H, s), |
| | | | | | 1.50-1.70 (3H, |
| | | | | | m), 1.20 (2H, |
| | | | | | m), 0.96 (3H, d) |
| 60 | N⁴-cyclohexyl- | Intermediate 30, | | 305 [M + H]⁺ | CDCl₃ 5.0 (1H, |
| | N⁴-methyl-6-(4- | N- | | RT 2.47 mins | s), 4.7 (2H, br |
| | methylpiperazin- | methylpiperazine | | (pH 5.8) | s), 4.4 (1H, br |
| | 1-yl)pyrimidine- | | | | m), 3.55 (4H, |
| | 2,4-diamine | | | | m), 2.7 (3H, s), |
| | | | | | 2.5 (4H, m), |
| | | | | | 2.35 (3H, s), |
| | | | | | 1.8 (2H, m), |
| | | | | | 1.7 (3H, m), |
| | | | | | 1.35-1.5 (4H, m), |
| | | | | | 1.1 (1H, m) |
| 61 | 4-(1,3-dihydro- | 4-chloro-6-(4- | | 311 [M + H]⁺ | CDCl₃ 7.35 (4H, |
| | 2H-isoindol-2- | methylpiperazin- | | RT 2.14 mins | m), 5.10 (1H, s), |
| | yl)-6-(4- | 1-yl)pyrimidin-2- | | (pH 5.8) | 4.76 (4H, s), |
| | methylpiperazin- | amine, | | | 4.52 (2H, s), |
| | 1-yl)pyrimidin-2- | isoindoline | | | 3.60 (4H, m), |
| | amine | | | | 2.48 (4H, m), |
| | | | | | 2.36 (3H, s) |
| 62 | N⁴-cyclohexyl- | 6-chloro-N⁴- | | 317 [M + H]⁺ | CDCl₃ 4.97 (1H, |
| | 6- | cyclohexylpyrimidine- | | RT 2.28 mins | s), 4.64 (2H, |
| | (hexahydropyrrolo[1,2- | 2,4-diamine, | | (pH 5.8) | s), 4.39 (1H, |
| | a]pyrazin- | 1,4- | | | m), 4.19 (1H, |
| | 2(1H)- | diazobicyclo[4.3.0]nonane | | | m), 3.28-3.46 (1H, |
| | yl)pyrimidine- | | | | m), |
| | 2,4-diamine | | | | 3.04-3.18 (2H, m), |
| | | | | | 2.96 (1H, td, J = 12.1, |
| | | | | | 3.4 Hz), 2.57 (, 1H, dd, |
| | | | | | J = 12.2, 10.2 Hz), |
| | | | | | 2.23 (1H, |
| | | | | | dd, J = 11.3, 3.4 Hz), |
| | | | | | 2.09-2.19 (1H, m), |
| | | | | | 1.67-2.04 (8H, m, |
| | | | | | 1.12-1.66 (7H, |
| | | | | | m) |
| 63 | 4-(4- | 4-chloro-6-(4- | | 353 [M + H]⁺ | d₆-DMSO |
| | methylpiperazin- | methylpiperazin- | | RT 1.42 mins | 7.2-7.4 (5H, m), |
| | 1-yl)-6-(3- | 1-yl)pyrimidin-2- | | (pH 2) | 5.37 (2H, s), |
| | phenylpiperidin- | amine, 3- | | | 5.27 (1H, s), |
| | 1-yl)pyrimidin-2- | phenylpiperidine | | | 4.35 (1H, m), |
| | amine | | | | 4.15 (1H, m), |
| | | | | | 3.4 (4H, m), |
| | | | | | 2.75 (2H, m), |
| | | | | | 2.3 (4H, m), |
| | | | | | 2.15 (3H, s), |
| | | | | | 1.9 (1H, m), |
| | | | | | 1.7 (2H, m), |
| | | | | | 1.45 (1H, m), |
| | | | | | 1.15 (1H, m) |
| 64 | 6-cyclohexyl- | 4-chloro-6- | | 290 [M + H]⁺ | CDCl₃ 5.6 (1H, |
| | N⁴-(1- | cyclohexylpyrimidin- | | RT 1.98 mins | s), 4.85 (2H, s), |
| | methylpiperidin- | 2-amine, | | (pH 5.8) | 4.6 (1H, s), |
| | 4-yl)pyrimidine- | 4-amino-1- | | | 3.6 (1H, m), |
| | 2,4-diamine | methylpiperidine | | | 2.7-2.9 (4H, m), |
| | | | | | 2.3 (4H, m), |
| | | | | | 1.7-2.2 (8H, m), |
| | | | | | 1.2-1.5 (6H, m) |
| 65 | 4-cyclohexyl-6- | 4-chloro-6- | | 290 [M + H]⁺ | d₆-DMSO |
| | [3- | cyclohexylpyrimidin- | | RT 1.00 mins | 5.7 (2H, s), 5.6 (1H, |
| | (dimethylamino)pyrrolidin- | 2-amine, 3- | | (pH 2) | s), 3.2-3.3 (3H, |
| | 1- | (dimethylamino)pyrrolidine | | | m), |
| | yl]pyrimidin-2- | | | | 2.95-3.05 (3H, m), |
| | amine | | | | 2.2 (6H, s), |
| | | | | | 1.65-1.8 (6H, m), |
| | | | | | 1.4 (2H, m), |
| | | | | | 1.15-1.3 (4H, m) |
| 66 | 4-[4-(2- | 4-chloro-6-(4- | | 383 [M + H]⁺ | d₆-DMSO |
| | methoxyphenyl)piperidin- | methylpiperazin- | | RT 2.95 mins | 7.20 (2H, m), |
| | 1-yl]-6- | 1-yl)pyrimidin-2- | | (pH 5.8) | 6.95 (1H, d), |
| | (4- | amine, 4-(2- | | | 6.88 (1H, t), |
| | methylpiperazin- | methoxyphenyl)piperidine | | | 5.60 (2H, s), |
| | 1-yl)pyrimidin-2- | | | | 5.33 (1H, s), |
| | amine | | | | 4.50 (2H, m), |
| | | | | | 3.70 (3H, s), |
| | | | | | 3.46 (4H, m), |
| | | | | | 3.15 (1H, m), |
| | | | | | 2.75 (2H, m), |
| | | | | | 2.32 (4H, m), |
| | | | | | 2.20 (3H, s), |
| | | | | | 1.70 (2H, m), |
| | | | | | 1.50 (2H, m) |
| 67 | 6-cyclohexyl- | 4-chloro-6- | | 264 [M + H]⁺ | |
| | N⁴-[2- | cyclohexylpyrimidin- | | RT 1.00 mins | |
| | (dimethylamino)- | 2-amine,N,N- | | (pH 2) | |
| | ethyl]pyrimidine- | dimethylethylene | | | |
| | 2,4-diamine | diamine | | | |
| 68 | 1-[2-amino-6-(4- | 4-chloro-6-(4- | Di- | 403 [M + H]⁺ | CD₃OD |
| | methylpiperazin- | methylpiperazin- | acetate | RT 2.44 mins | 7.50 (2H, d), |
| | 1-yl)pyrimidin-4- | 1-yl)pyrimidin-2- | | (pH 5.8) | 7.35 (2H, d), |
| | yl]-4-(4- | amine, 4-(4- | | | 4.90 (1H, s), |
| | chlorophenyl)piperidin- | chlorophenyl)-4- | | | 4.20 (2H, d), |
| | 4-ol | hydroxypiperidine | | | 3.65 (4H, t), |
| | di-acetate salt | | | | 3.35 (2H, m), |
| | | | | | 2.70 (4H, t), |
| | | | | | 2.45 (3H, s), |
| | | | | | 2.05 (2H, d), |
| | | | | | 1.75 (2H, d) |
| 69 | 4-(2- | 4-chloro-6-(4- | | 304 [M + H]⁺ | CDCl₃ 5.13 (1H, |
| | ethylpiperidin-1- | methylpiperazin- | | RT 2.44 mins | s), 4.75 (2H, s), |
| | yl)-6-(4- | 1-yl)pyrimidin-2- | | (pH 5.8) | 4.30 (1H, m), |
| | methylpiperazin- | amine, 2- | | | 4.20 (1H, m), |
| | 1-yl)pyrimidin-2- | ethylpiperidine | | | 3.50 (4H, m), |
| | amine | | | | 2.85 (2H, m), |
| | | | | | 2.55 (4H, m), |
| | | | | | 2.35 (3H, s), |
| | | | | | 1.35-1.70 (7H, |
| | | | | | m), 0.90 (3H, t), |
| | | | | | 0.85 (1H, m) |
| 70 | 4-[3- | Intermediate 14, | | 277 [M + H]⁺ | CDCl₃ 4.85 (2H, |
| | (methylamino)pyrrolidin- | 3-(N-tert- | | RT 1.57 mins | br s), 4.69 (1H, |
| | 1-yl]-6- | butoxycarbonyl- | | (pH 5.8) | s), 4.11 (1H, m), |
| | (2- | N-methylamino)- | | | 3.17-3.70 (7H, |
| | methylpyrrolidin- | pyrrolidine | | | m), 2.47 (3H, s), |
| | 1-yl)pyrimidin-2- | | | | 1.58-2.21 (7H, |
| | amine | | | | m), 1.18 (3H, d) |
| 71 | 4-[3-(4- | 4-chloro-6-(4- | | 373/375 [M + H]⁺ | CDCl₃ 7.3 (2H, |
| | chlorophenyl)pyrrolidin- | methylpiperazin- | | RT 2.64 mins | d), 7.15 (2H, d), |
| | 1-yl]-6- | 1-yl)pyrimidin-2- | | (pH 5.8) | 4.95 (1H, s), |
| | (4- | amine, 3-(4- | | | 4.48 (2H, s), |
| | methylpiperazin- | chlorophenyl)pyrrolidine | | | 3.90 (1H, m), |
| | 1-yl)pyrimidin-2- | | | | 3.65 (1H, m), |
| | amine | | | | 3.55 (4H, t), |
| | | | | | 3.43 (2H, m), |
| | | | | | 3.39 (2H, m), |
| | | | | | 2.48 (4H, t), |
| | | | | | 2.35 (3H, s), |
| | | | | | 2.05 (1H, m). |
| 72 | 6-cyclohexyl- | 4-chloro-6- | | 278 [M + H]⁺ | |
| | N⁴-[3- | cyclohexylpyrimidin- | | RT 1.05 mins | |
| | (dimethylamino)propyl]pyrimidine- | 2-amine, N,N- | | (pH 2) | |
| | 2,4-diamine | dimethyl-1,3- | | | |
| | | propanediamine | | | |
| 73 | 4-(4- | 4-chloro-6-(4- | | 208 [M + H]⁺ | CDCl₃ 5.19 (1H, |
| | methylpiperazin- | methylpiperazin- | | RT 1.30 mins | s), 4.80 (2H, s), |
| | 1-yl)-6-[4- | 1-yl)pyrimidin-2- | | (pH 5.8) | 4.39 (2H, m), |
| | (trifluoromethyl)piperidin- | amine, 4- | | | 3.60 (4H, m), |
| | 1- | trifluoromethylpiperidine | | | 2.78 (2H, m), |
| | yl]pyrimidin-2- | | | | 2.60 (4H, m), |
| | amine | | | | 2.35 (3H, s), |
| | | | | | 2.25 (1H, m), |
| | | | | | 1.90 (2H, m), |
| | | | | | 1.55 (2H, m) |
| 74 | 4-(6- | 4-chloro-6-(4- | | 303 [M + H]⁺ | d₆-DMSO |
| | azabicyclo[3.2.1]oct- | methylpiperazin- | | RT 0.92 mins | 5.5 (2H, s), 5.0 (1H, |
| | 6-yl)-6-(4- | 1-yl)pyrimidin-2- | | (pH 2) | s), 3.4 (4H, m), |
| | methylpiperazin- | amine, 6-aza- | | | 3.32 (1H, m), |
| | 1-yl)pyrimidin-2- | bicyclo[3.2.1]octane | | | 2.3 (4H, m), |
| | amine | hydrochloride | | | 2.17 (3H, s), 1.9 (1H, |
| | | | | | m), 1.75 (1H, m), |
| | | | | | 1.45-1.6 (4H, m), |
| | | | | | 1.2-1.35 (2H, m) |
| | | | | | partial only |
| | | | | | (other signals |
| | | | | | under DMSO) |
| 75 | 4-(3- | Intermediate 14, | | 263 [M + H]⁺ | CDCl₃ 4.90 (2H, |
| | aminopyrrolidin- | 3-(tert- | | RT 1.42 mins | br s), 4.69 (1H, |
| | 1-yl)-6-(2- | butoxycarbonylamino)pyrrolidine, | | (pH 5.8) | s), 4.12 (1H, m), |
| | methylpyrrolidin- | | | | 3.08-3.75 (7H, |
| | 1-yl)pyrimidin-2- | | | | m), |
| | amine | | | | 1.58-2.54 (8H, m), |
| | | | | | 1.20 (3H, d) |
| 76 | 4-[4-(2- | 4-chloro-6-(4- | | 384 [M + H]⁺ | CDCl₃ |
| | methoxyphenyl)piperazin- | methylpiperazin- | | RT 2.39 mins | 6.85-7.10 (4H, m), |
| | 1-yl]- | 1-yl)pyrimidin-2- | | (pH 5.8) | 5.20 (1H, s), |
| | 6-(4- | amine, 4-(2- | | | 4.95 (2H, s), |
| | methylpiperazin- | methoxyphenyl)piperazine | | | 3.90 (3H, s), |
| | 1-yl)pyrimidin-2- | | | | 3.76 (4H, m), |
| | amine | | | | 3.60 (4H, m), |
| | | | | | 3.12 (4H, m), |
| | | | | | 2.60 (4H, m), |
| | | | | | 2.38 (3H, s) |
| 77 | 4-azepan-1-yl-6- | 4-chloro-6-(4- | | 291 [M + H]⁺ | d₆-DMSO |
| | (4- | methylpiperazin- | | RT 0.9 mins | 5.35 (2H, s), |
| | methylpiperazin- | 1-yl)pyrimidin-2- | | (pH 2) | 5.07 (1H, s), |
| | 1-yl)pyrimidin-2- | amine, | | | 3.4 (4H, m), |
| | amine | Azepine | | | 2.3 (4H, m), |
| | | | | | 2.17 (3H, s), |
| | | | | | 2.16 (4H, br m), |
| | | | | | 1.55 (4H, br m), |
| | | | | | 1.45 (4H, br m) |
| 78 | 4- | Intermediate | | 303 [M + H]⁺ | CDCl₃ 4.95 (1H, |
| | (hexahydropyrrolo[1,2- | 14,octahydro- | | RT 1.89 mins | s), 4.58 (2H, br |
| | a]pyrazin- | pyrrolo[1,2- | | (pH 5.8) | s), 4.36 (1H, m), |
| | 2(1H)-yl)-6-(2- | a]pyrazine | | | 4.05-4.25 (2H, |
| | methylpyrrolidin- | | | | m), 3.48 (1H, m), |
| | 1-yl)pyrimidin-2- | | | | 3.32 (1H, m), |
| | amine | | | | 3.02-3.20 (2H, |
| | | | | | m), 2.92 (1H, dt), |
| | | | | | 2.54 (1H, t), |
| | | | | | 1.39-2.30 (11H, m), |
| | | | | | 1.19 (3H, d) |
| 79 | 4-[3-(2- | 4-chloro-6-(4- | | 369/370 [M + H]⁺ | CDCl₃ 7.19 (2H, |
| | methoxyphenyl)pyrrolidin- | methylpiperazin- | | RT 2.42 mins | m), 6.90 (2H, |
| | 1-yl]- | 1-yl)pyrimidin-2- | | (pH 5.8) | m), 4.95 (1H, s), |
| | 6-(4- | amine, | | | 4.48 (2H, s), |
| | methylpiperazin- | 3-(2- | | | 3.85 (3H, s), |
| | 1-yl)pyrimidin-2- | methoxyphenyl)pyrrolidine | | | 3.80 (1H, m), |
| | amine | | | | 3.60 (1H, m), |
| | | | | | 3.55 (4H, t), |
| | | | | | 3.43 (2H, m), |
| | | | | | 3.39 (2H, m), |
| | | | | | 2.48 (4H, t), |
| | | | | | 2.35 (3H, s), |
| | | | | | 2.05 (1H, m). |
| 80 | 4-(3,4- | 4-chloro-6-(4- | | 325 [M + H]⁺ | CDCl₃ 7.20 (4H, |
| | dihydroisoquinolin- | methylpiperazin- | | RT 2.45 mins | m), 5.22 (1H, s), |
| | 2(1H)-yl)-6- | 1-yl)pyrimidin-2- | | (pH 5.8) | 4.70 (2H, s), |
| | (4- | amine, | | | 4.50 (2H, s), |
| | methylpiperazin- | tetrahydroisoquinoline | | | 3.82 (2H, t), |
| | 1-yl)pyrimidin-2- | | | | 3.60 (4H, m), |
| | amine | | | | 2.90 (2H, t), |
| | | | | | 2.45 (4H, m), |
| | | | | | 2.33 (3H, s) |
| 81 | 4-(4- | 4-chloro-6-(4- | | 305 [M + H]⁺ | |
| | methylpiperazin- | methylpiperazin- | | RT 1.15 mins | |
| | 1-yl)-6-(2- | 1-yl)pyrimidin-2- | | (pH 5.8). | |
| | propylpyrrolidin- | amine, 2- | | | |
| | 1-yl)pyrimidin-2- | propylpyrrolidine | | | |
| | amine | | | | |
| 82 | 6-cyclohexyl- | 4-chloro-6- | | 290 [M + H]⁺ | d₆-DMSO |
| | N⁴-methyl-N⁴- | cyclohexylpyrimidin- | | RT 1.13 mins | 5.6 (3H, m), |
| | (1- | 2-amine, | | (pH 2) | 2.97 (1H, m), |
| | methylpyrrolidin- | 1-methyl-3- | | | 2.85 (2H, m), |
| | 3-yl)pyrimidine- | (methylamino)pyrrolidine | | | 2.25 (3H, s), |
| | 2,4-diamine | | | | 1.6-1.75 (6H, m), |
| | | | | | 1.15-1.3 (6H, m) |
| | | | | | partial only |
| | | | | | (other signals |
| | | | | | under DMSO) |
| 83 | 4-(4- | 4-chloro-6-(4- | | 422 [M + H]⁺ | CDCl₃ 7.36 (1H, |
| | methylpiperazin- | methylpiperazin- | | RT 3.09 mins | m), 7.10 (3H, |
| | 1-yl)-6-{4-[3- | 1-yl)pyrimidin-2- | | (pH 5.8) | m), 5.25 (1H, s), |
| | (trifluoromethyl)phenyl]piperazin- | amine, 4-(3- | | | 4.50 (2H, s), |
| | 1-yl}pyrimidin- | trifluoromethylphenyl)piperazine | | | 3.72 (4H, m), |
| | 2-amine | | | | 3.60 (4H, m), |
| | | | | | 3.29 (4H, m), |
| | | | | | 2.45 (4H, m), |
| | | | | | 2.35 (4H, m) |
| 84 | 4-(4- | 4-chloro-6-(4- | | 353 [M + H]⁺ | CDCl₃ 7.05 (5H, |
| | methylpiperazin- | methylpiperazin- | | RT 2.62 mins | m), 5.25 (1H, s), |
| | 1-yl)-6-(4- | 1-yl)pyrimidin-2- | | (pH 5.8) | 4.50 (2H, m), |
| | phenylpiperidin- | amine, 4- | | | 4.45 (2H, br s), |
| | 1-yl)pyrimidin-2- | phenylpiperidine | | | 3.55 (4H, m), |
| | amine | | | | 2.82 (2H, m), |
| | | | | | 2.75 (1H, m), |
| | | | | | 2.45 (4H, m), |
| | | | | | 2.33 (3H, s), |
| | | | | | 1.85 (2H, m), |
| | | | | | 1.70 (2H, m) |
| 85 | 4-(2-tert- | 4-chloro-6-(4- | | 319 [M + H]⁺ | |
| | butylpyrrolidin- | methylpiperazin- | | RT 1.2 mins | |
| | 1-yl)-6-(4- | 1-yl)pyrimidin-2- | | (pH 2) | |
| | methylpiperazin- | amine, 2-tert- | | | |
| | 1-yl)pyrimidin-2- | butylpyrrolidine | | | |
| | amine | | | | |
| 86 | 4-(4- | 4-chloro-6-(4- | | 277 [M + H]⁺ | CDCl₃ 5.20 (1H, |
| | methylpiperazin- | methylpiperazin- | | RT 1.86 mins | s), 4.45 (2H, s), |
| | 1-yl)-6-piperidin- | 1-yl)pyrimidin-2- | | (pH 5.8) | 3.50 (8H, m), |
| | 1-ylpyrimidin-2- | amine, piperidine | | | 2.50 (4H, m), |
| | amine | | | | 2.35 (3H, s), |
| | | | | | 1.65 (5H, m) |
| 87 | 4-[4-(4- | 4-chloro-6-(4- | | 372 [M + H]⁺ | CDCl₃ |
| | fluorophenyl)piperazin- | methylpiperazin- | | RT 2.49 mins | 6.85-7.10 (4H, m), |
| | 1-yl]-6- | 1-yl)pyrimidin-2- | | (pH 5.8) | 5.25 (2H, br s), |
| | (4- | amine, 4-(4- | | | 5.18 (1H, s), |
| | methylpiperazin- | fluorophenyl)piperazine | | | 3.67 (8H, m), |
| | 1-yl)pyrimidin-2- | | | | 3.15 (4H, m), |
| | amine | | | | 2.70 (4H, m), |
| | | | | | 2.45 (3H, s) |
| 88 | 4-(4-tert- | 4-chloro-6-(4- | | 333 [M + H]⁺ | CDCl₃ 5.18 (1H, |
| | butylpiperidin-1- | methylpiperazin- | | RT 2.34 mins | s), 4.48 (2H, s), |
| | yl)-6-(4- | 1-yl)pyrimidin-2- | | (pH 5.8) | 4.38 (2H, m), |
| | methylpiperazin- | amine, 4-tert- | | | 3.55 (4H, m), |
| | 1-yl)pyrimidin-2- | butylpiperidine | | | 2.70 (2H, m), |
| | amine | hydrochloride | | | 2.45 (4H, m), |
| | | | | | 2.32 (3H, s), |
| | | | | | 1.70 (2H, m), |
| | | | | | 1.25 (3H, m), |
| | | | | | 0.85 (9H, s) |
| 89 | 4-[4-(3- | 4-chloro-6-(4- | | 371 [M + H]⁺ | CDCl₃ 7.25 (1H, |
| | fluorophenyl)piperidin- | methylpiperazin- | | RT 2.71 mins | m), 7.00 (1H, |
| | 1-yl]-6-(4- | 1-yl)pyrimidin-2- | | (pH 5.8) | m), 6.90 (2H, |
| | methylpiperazin- | amine, 4-(3- | | | m), 5.25 (1H, s), |
| | 1-yl)pyrimidin-2- | fluorophenyl)piperidine | | | 4.50 (2H, m), |
| | amine | | | | 4.45 (2H, br s), |
| | | | | | 3.55 (4H, m), |
| | | | | | 2.82 (2H, m), |
| | | | | | 2.75 (1H, m), |
| | | | | | 2.45 (4H, m), |
| | | | | | 2.33 (3H, s), |
| | | | | | 1.85 (2H, m), |
| | | | | | 1.70 (2H, m) |
| 90 | 4-[(2S)-2- | 4-chloro-6-(4- | | 307 [M + H]⁺ | |
| | (methoxymethyl)pyrrolidin- | methylpiperazin- | | RT 0.65 mins | |
| | 1-yl]- | 1-yl)pyrimidin-2- | | (pH 2) | |
| | 6-(4- | amine, (S)-2- | | | |
| | methylpiperazin- | (methoxymethyl)pyrrolidine | | | |
| | 1-yl)pyrimidin-2- | | | | |
| | amine | | | | |
| 91 | 4-(4- | 4-chloro-6-(4- | | 307 [M + H]⁺ | CDCl₃ 5.22 (1H, |
| | methoxypiperidin- | methylpiperazin- | | RT 1.56 mins | s), 4.45 (2H, s), |
| | 1-yl)-6-(4- | 1-yl)pyrimidin-2- | | (pH 5.8) | 3.95 (2H, m), |
| | methylpiperazin- | amine, 4- | | | 3.52 (4H, m), |
| | 1-yl)pyrimidin-2- | methoxypiperidine | | | 3.40 (1H, m), |
| | amine | | | | 3.38 (3H, s), |
| | | | | | 3.19 (2H, m), |
| | | | | | 2.45 (4H, m), |
| | | | | | 2.35 (3H, s), |
| | | | | | 2.40 (2H, m), |
| | | | | | 1.55 (2H, m) |
| 92 | 6-cyclohexyl- | 4-chloro-6- | | 292 [M + H]⁺ | |
| | N⁴-[3- | cyclohexylpyrimidin- | | RT 1.17 mins | |
| | (dimethylamino)propyl]- | 2-amine, | | (pH 2) | |
| | N⁴- | N,N,N′-trimethyl- | | | |
| | methylpyrimidine- | 1,3- | | | |
| | 2,4-diamine | propanediamine | | | |
| 93 | 4-cyclohexyl-6- | 4-chloro-6- | | 290 [M + H]⁺ | d₆-DMSO |
| | (4- | cyclohexylpyrimidin- | | RT 0.99 mins | 5.9 (1H, s), |
| | ethylpiperazin- | 2-amine, 1- | | (pH 2) | 5.82 (2H, s), 3.5 (4H, |
| | 1-yl)pyrimidin-2- | ethylpiperazine | | | m), 2.75 (1H, |
| | amine | | | | m), |
| | | | | | 2.35-2.4 (6H, m), |
| | | | | | 1.7-1.75 (4H, m), |
| | | | | | 1.4 (2H, m), |
| | | | | | 1.2-1.3 (2H, m), |
| | | | | | 1.15 (2H, m), |
| | | | | | 1.0 (3H, t |
| | | | | | J = 7.4 Hz) |
| 94 | 6-(2- | Intermediate 14, | | 263 [M + H]⁺ | CDCl₃ 4.68 (3H, |
| | methylpyrrolidin- | 3-amino-1-N-tert- | | RT 1.72 mins | br s), 4.10 (1H, |
| | 1-yl)-N⁴- | butoxycarbonyl- | | (pH 5.8) | m), |
| | pyrrolidin-3- | pyrrolidine | | | 3.06-3.76 (7H, m), |
| | ylpyrimidine-2,4- | | | | 1.55-2.29 (6H, m), |
| | diamine | | | | 1.22-1.35 (2H, |
| | | | | | m), 1.19 (3H, d) |
| 95 | N⁴-1- | Intermediate 14, | | 303 [M + H]⁺ | CDCl₃ 4.93 (1H, |
| | azabicyclo[2.2.2]oct- | 3- | | RT 1.81 mins | m), |
| | 3-yl-6-(2- | aminoquinuclidine | | (pH 5.8) | 3.67-4.81 (4H, m), |
| | methylpyrrolidin- | dihydrochloride | | | 2.18-3.55 (7H, m), |
| | 1-yl)pyrimidine- | | | | 1.29-2.10 (11H, m), |
| | 2,4-diamine | | | | 1.18 (3H, d). |
| 96 | N⁴-1- | Intermediate 18 | Acetate | 337 [M + H]⁺ | CD₃OD |
| | azabicyclo[2.2.2]oct- | 3- | | RT 2.31 mins | 7.24-7.41 (4H, m), |
| | 3-yl-6-(1,3- | aminoquinuclidine | | (pH 5.8) | 5.04 (1H, m), |
| | dihydro-2H- | dihydrochloride | | | 4.75 (4H, s), |
| | isoindol-2- | | | | 4.40 (1H, m), |
| | yl)pyrimidine- | | | | 2.53-3.78 (5H, |
| | 2,4-diamine | | | | m), |
| | | | | | 1.30-2.45 (6H, m). |
| 97 | 4-[4- | Intermediate 14, | | 291 [M + H]⁺ | CDCl₃ 4.95 (1H, |
| | (methylamino)piperidin- | 4-N-tert- | | RT 1.74 mins | s), 4.45 (2H, s), |
| | 1-yl]-6- | butylcarbonyl-4- | | (pH 5.8) | 4.20 (2H, m), |
| | (2- | N- | | | 4.11 (1H, m), |
| | methylpyrrolidin- | methylaminopiperidine | | | 3.48 (1H, m), |
| | 1-yl)pyrimidin-2- | | | | 3.32 (1H, m), |
| | amine | | | | 2.82 (2H, m), |
| | | | | | 2.56 (1H, m), |
| | | | | | 2.45 (3H, s), |
| | | | | | 1.82-2.08 (6H, |
| | | | | | m), |
| | | | | | 1.20-1.38 (3H, m), |
| | | | | | 1.18 (3H, d) |
| 98 | 6-(4- | 4-chloro-6-(4- | | 345 [M + H]⁺ | |
| | methylpiperazin- | methylpiperazin- | | RT 1.54 mins | |
| | 1-yl)-N⁴- | 1-yl)pyrimidin-2- | | (pH 2) | |
| | [(1S,2S,3S,5R)- | amine, | | | |
| | 2,6,6- | (1S,2S,3S,5R)- | | | |
| | trimethylbicyclo[3.1.1]hept- | (+)- | | | |
| | 3- | isopinocampheylamine | | | |
| | yl]pyrimidine- | (CAS RN | | | |
| | 2,4-diamine | 13293-47-5) | | | |
| 99 | N⁴- | 4-chloro-6-(4- | | 303 [M + H]⁺ | CDCl₃ 4.95 (1H, |
| | [(1R*,2R*,4S*)- | methylpiperazin- | | RT 1.1 mins | s), 4.45 (3H, m) |
| | bicyclo[2.2.1]hept- | 1-yl)pyrimidin-2- | | (pH 2) | 3.55 (4H, m), |
| | 2-yl]-6-(4- | amine, | | | 3.3 (1H, m), |
| | methylpiperazin- | (1R*,2R*,4S*)- | | | 2.45 (4H, m), |
| | 1-yl)pyrimidine- | bicyclo[2.2.1]heptan- | | | 2.35 (3H, s), |
| | 2,4-diamine | 2-amine | | | 2.3 (2H, m), |
| | | | | | 1.8 (1H, m), |
| | | | | | 1.4-1.6 (3H, m), |
| | | | | | 1.1-1.25 (4H, m) |
| 100 | 4-(1,3-dihydro- | Intermediate 18, | | 325 [M + H]⁺ | CDCl₃ 7.3 (4H, |
| | 2H-isoindol-2- | N- | | RT 1.17 mins | m), 4.9 (1H, s), |
| | yl)-6-(4-methyl- | methylhomopiperazine | | (pH 2.5) | 4.75 (4H, br s), |
| | 1,4-diazepan-1- | | | | 4.6 (2H, br s), |
| | yl)pyrimidin-2- | | | | 3.8.5 (2H, m), |
| | amine | | | | 3.6 (2H, m), |
| | | | | | 2.7 (2H, m), |
| | | | | | 2.65 (2H, m), 2.4 (3H, |
| | | | | | s), 2.0 (2H, m) |
| 101 | 4-(1,3-dihydro- | Intermediate 18, | | 325 [M + H]⁺ | |
| | 2H-isoindol-2- | 3- | | RT 1.17 mins | |
| | yl)-6-[3- | (dimethylamino)pyrrolidine | | (pH 2.5) | |
| | (dimethylamino)pyrrolidin- | | | | |
| | 1- | | | | |
| | yl]pyrimidin-2- | | | | |
| | amine | | | | |
| 102 | 6-(1,3-dihydro- | Intermediate 18, | | 325 [M + H]⁺ | |
| | 2H-isoindol-2- | 4-amino-1- | | RT 1.25 mins | |
| | yl)-N⁴-(1- | methylpiperidine | | (pH 2.5) | |
| | methylpiperidin- | | | | |
| | 4-yl)pyrimidine- | | | | |
| | 2,4-diamine | | | | |
| 103 | 4-cyclohexyl-6- | 4-chloro-6- | | 304 [M + H]⁺ | d₆₋DMSO |
| | (4- | cyclohexylpyrimidin- | | RT 2.28 mins | 5.85 (1H, s), |
| | isopropylpiperazin- | 2-amine, N- | | (pH 5.8) | 5.80 (2H, bs), |
| | 1-yl)pyrimidin- | isopropylpiperazine | | | 3.50-3.60 (4H, m), |
| | 2-amine | | | | 2.60-2.70 (1H, |
| | | | | | m), |
| | | | | | 2.40-2.50 (4H, m), |
| | | | | | 2.15-2.30 (1H, m), |
| | | | | | 1.60-1.80 (5H, |
| | | | | | m), |
| | | | | | 1.35-1.45 (2H, m), |
| | | | | | 1.10-1.35 (3H, m), |
| | | | | | 0.95 (6H, d) |
| Interm. | tert-butyl 3-[(2- | 4-chloro-6- | | 376 [M + H]⁺ | CDCl₃ 5.68 (1H, |
| 31 | amino-6- | cyclohexylpyrimidin- | | RT 3.38 mins | s), |
| | cyclohexylpyrimidin- | 2-amine, tert- | | (pH 5.8) | 5.19-5.41 (1H, m), |
| | 4- | butyl 3- | | | 4.63-4.90 (2H, br s), |
| | yl)(methyl)amino]pyrrolidine- | (methylamino)pyrrolidine- | | | 3.39-3.61 (2H, |
| | 1- | 1- | | | m), |
| | carboxylate | carboxylate (CAS | | | 3.01-3.36 (2H, m), |
| | | RN 454712-26-6) | | | 2.82 (3H, s), |
| | | | | | 2.22-2.36 (1H, m), |
| | | | | | 1.60-2.03 (7H, |
| | | | | | m), 1.40 (9H, s), |
| | | | | | 1.08-1.37 (5H, |
| | | | | | m) |
| 104 | 6-cyclohexyl- | 4-chloro-6- | | 262 [M + H]⁺ | CDCl₃ 5.23 (1H, |
| | N⁴-(1- | cyclohexylpyrimidin- | | RT 2.15 mins | s), 4.20 (1H, dd), |
| | methylazetidin- | 2-amine, 1- | | (pH 5.8) | 3.90 (1H, dd), |
| | 3-yl)pyrimidine- | methylazetidin-3- | | | 3.55 (3H, m), |
| | 2,4-diamine | amine | | | 3.15 (2H, q), |
| | | | | | 3.05 (3H, s), |
| | | | | | 2.95 (1H, d), |
| | | | | | 2.25 (1H, m), |
| | | | | | 1.65-1.98 (5H, |
| | | | | | m), |
| | | | | | 1.20-1.49 (5H, m) |
| 105 | 6-(4- | Intermediate 25, | | 292 [M + H]⁺ | CDCl₃ 5.05 (1H, |
| | methylpiperazin- | N- | | RT 1.53 mins | s), 4.45 (2H, s), |
| | 1-yl)-N⁴- | methylpiperazine | | (pH 5.8) | 4.30 (1H, d), |
| | (tetrahydro-2H- | | | | 4.00 (2H, m), |
| | pyran-4- | | | | 3.75 (1H, m), |
| | yl)pyrimidine- | | | | 3.50 (6H, m), |
| | 2,4-diamine | | | | 2.45 (4H, m), |
| | | | | | 2.35 (3H, s), |
| | | | | | 2.00 (2H, d), |
| | | | | | 1.49 (2H, m) |
| 106 | 4-cyclohexyl-6- | 4-chloro-6- | | 290 [M + H]⁺ | d₆-DMSO |
| | (3,3- | cyclohexylpyrimidin- | | RT 1.90 mins | 5.82-5.90 (3H, m), |
| | dimethylpiperazin- | 2-amine, 2,2- | | (pH 5.8) | 3.45-3.55 (2H, |
| | 1-yl)pyrimidin- | dimethylpiperazine | | | m), |
| | 2-amine | (CAS RN | | | 3.25-3.42 (3H, m) (partially |
| | | 84477-72-5) | | | obscured by |
| | | | | | H₂O peak), |
| | | | | | 2.80-2.85 (2H, |
| | | | | | m), |
| | | | | | 2.15-2.30 (1H, m), |
| | | | | | 1.60-1.80 (5H, m), |
| | | | | | 1.12-1.52 (5H, |
| | | | | | m), 1.07 (6H, s) |
| 107 | N⁴-(2,3-dihydro- | 6-chloro-N⁴-(2,3- | | 325 [M + H]⁺ | CDCl₃ |
| | 1H-inden-1-yl)- | dihydro-1H-inden- | | RT 2.39 mins | 7.30-7.40 (1H, m), |
| | 6-(4- | 1-yl)pyrimidine- | | (pH 5.8) | 7.15-7.25 (3H, m), |
| | methylpiperazin- | 2,4-diamine, N- | | | 5.15-5.30 (1H, |
| | 1-yl)pyrimidine- | methylpiperazine | | | m), 5.15 (1H, s), |
| | 2,4-diamine | | | | 4.70 (1H, d), |
| | | | | | 4.45 (2H, s), |
| | | | | | 3.50-3.60 (4H, |
| | | | | | m), |
| | | | | | 2.80-3.05 (2H, m), |
| | | | | | 2.55-2.65 (1H, m), |
| | | | | | 2.40-2.50 (4H, |
| | | | | | m), 2.35 (3H, s), |
| | | | | | 1.80-1.95 (1H, |
| | | | | | m) |
| 108 | 6-(4- | Intermediate 15, | | 339 [M + H]⁺ | CDCl₃ |
| | methylpiperazin- | N- | | RT 2.55 mins | 7.30-7.40 (1H, m), |
| | 1-yl)-N⁴- | methylpiperazine | | (pH 5.8) | 7.05-7.20 (3H, m), |
| | (1,2,3,4- | | | | 5.10 (1H, s), |
| | tetrahydronaphthalen- | | | | 4.85-5.00 (1H, |
| | 2- | | | | m), |
| | yl)pyrimidine- | | | | 4.60-4.70 (1H, d), |
| | 2,4-diamine | | | | 4.40-4.55 (2H, bs), |
| | | | | | 2.95-3.10 (4H, |
| | | | | | m), |
| | | | | | 2.70-2.90 (2H, m), |
| | | | | | 2.40-2.50 (4H, m), |
| | | | | | 2.35 (3H, s), |
| | | | | | 1.95-2.10 (1H, |
| | | | | | m), |
| | | | | | 1.75-1.95 (3H, m) |
| 109 | 6-piperazin-1-yl- | Intermediate 15, | | 325 [M + H]⁺ | CDCl₃ |
| | N⁴-(1,2,3,4- | piperazine | | RT 2.31 mins | 7.30-7.40 (1H, m), |
| | tetrahydronaphthalen- | | | (pH 5.8) | 7.05-7.20 (3H, m), |
| | 2- | | | | 5.10 (1H, bs), |
| | yl)pyrimidine- | | | | 4.85-5.00 (1H, |
| | 2,4-diamine | | | | m), |
| | | | | | 4.60-4.75 (1H, d), |
| | | | | | 4.40-4.60 (2H, bs), |
| | | | | | 3.40-3.60 (4H, m), |
| | | | | | 2.85-3.00 (4H, m), |
| | | | | | 2.65-2.85 (2H, |
| | | | | | m), |
| | | | | | 1.95-2.15 (2H, m), |
| | | | | | 1.65-1.95 (3H, m) |
| 110 | N⁴-[2- | Intermediate 15, | | 327 [M + H]⁺ | CD₃OD |
| | (dimethylamino)ethyl]- | N,N- | | RT 2.28 mins | 7.20-7.30 (1H, m), |
| | N⁶- | dimethylethylenediamine | | (pH 5.8) | 7.05-7.20 (3H, |
| | (1,2,3,4- | | | | m), |
| | tetrahydronaphthalen- | | | | 4.95-5.05 (1H, bs), |
| | 2- | | | | 4.80-4.95 (1H, m), |
| | yl)pyrimidine- | | | | 3.30-3.40 (2H, |
| | 2,4,6-triamine | | | | m), |
| | | | | | 2.75-2.90 (2H, m), |
| | | | | | 2.65-2.75 (2H, m), |
| | | | | | 2.45 (6H, s), |
| | | | | | 1.80-2.10 (4H, |
| | | | | | m) |
| 111 | N⁴-(2,3-dihydro- | Intermediate 16 | | 325 [M + H]⁺ | CD₃OD |
| | 1H-inden-2-yl)- | N- | | RT 2.28 mins | 7.20-7.30 (2H, m), |
| | 6-(4- | methylpiperazine, | | (pH 5.8) | 7.10-7.20 (2H, |
| | methylpiperazin- | | | | m), 4.90 (1H, |
| | 1-yl)pyrimidine- | | | | obscured by |
| | 2,4-diamine | | | | H₂O peak, s), |
| | | | | | 4.50-4.60 (1H, |
| | | | | | m), |
| | | | | | 3.45-3.55 (4H, m), |
| | | | | | 3.30 (2H, dd), |
| | | | | | 2.85 (2H, dd), |
| | | | | | 2.50 (4H, m), |
| | | | | | 2.35 (3H, s) |
| Interm. | tert-butyl {[1-(2- | 4-chloro-6- | | 362 [M + H]⁺ | CD₃OD |
| 32 | amino-6- | cyclohexylpyrimidin- | | RT 3.09 mins | 5.55 (1H, s), |
| | cyclohexylpyrimidin- | 2-amine, tert- | | (pH 5.8) | 4.05 (2H, dd), |
| | 4-yl)azetidin- | butyl (azetidin-3- | | | 3.75 (2H, dd), |
| | 3- | ylmethyl)carbamate | | | 3.25-3.35 (2H, m), |
| | yl]methyl}carbamate | | | | 2.80-2.95 (1H, |
| | | | | | m), |
| | | | | | 2.25-2.35 (1H, m), |
| | | | | | 1.80-1.90 (4H, m), |
| | | | | | 1.70-1.80 (1H, |
| | | | | | m), 1.45 (9H, m), |
| | | | | | 1.20-1.50 (5H, |
| | | | | | m) |
| Interm | tert-butyl 4-{2- | Intermediate 17, | Diacetate | 403 [M + H]⁺ | CD₃OD |
| 33 | amino-6- | tert-butyl 2- | | RT 3.77 mins | 4.91 (1H, obscured |
| | [(1R*,2R*,4S*)- | methylpiperazine- | | (pH 5.8) | by H₂O peak, s), |
| | bicyclo[2.2.1]hept- | 1-carboxylate | | | 4.23-4.32 (1H, |
| | 2- | (CAS RN 120737- | | | m), 4.16 (1H, |
| | ylamino]pyrimidin- | 78-2) | | | bd), 4.09 (1H, |
| | 4-yl}-2- | | | | bdd), 3.88 (1H, |
| | methylpiperazine- | | | | dt), 3.43 (1H, |
| | 1-carboxylate | | | | bd), |
| | | | | | 3.19-3.37 (2H, mm), |
| | | | | | 3.07 (1H, btd), |
| | | | | | 2.30 (2H, bdd), |
| | | | | | 1.98 (6H, s, 2 × AcOH), |
| | | | | | 1.85 (1H, ddd), |
| | | | | | 1.20-1.58 (8H, mm), |
| | | | | | 1.50 (9H, s), |
| | | | | | 1.16 (3H, d). |
| Interm. | tert-butyl 4-{2- | Intermediate 17, | Diacetate | 403 [M + H]⁺ | CD₃OD |
| 34 | amino-6- | tert-butyl 1,4- | | RT 3.48 mins | 4.80 (1H, obscured |
| | [(1R*,2R*,4S*)- | diazepane-1- | | (pH 5.8) | by H₂O peak, s), |
| | bicyclo[2.2.1]hept- | carboxylate (CAS | | | 3.75-3.84 (2H, |
| | 2- | RN 112275-50-0) | | | m), |
| | ylamino]pyrimidin- | | | | 3.54-3.72 (4H, mm), |
| | 4-yl}-1,4- | | | | 3.30-3.46 (3H, mm), |
| | diazepane-1- | | | | 2.31 (2H, bdd), |
| | carboxylate | | | | 1.97 (6H, s, 2 × AcOH), |
| | | | | | 1.79-1.92 (3H, m), |
| | | | | | 1.48-1.62 (3H, |
| | | | | | m), 1.42 (3H, d), |
| | | | | | 1.16-1.46 (4H, |
| | | | | | mm). |
| Interm | tert-butyl | Intermediate 17, | Acetate | 429 [M + H]⁺ | CD₃OD |
| 35 | (4aR*,7aR*)-6- | tert-butyl | | RT 4.03 mins | 4.73-4.85 (2H, m), |
| | {2-amino-6- | (4aR*,7aR*)- | | (pH 5.8) | 4.01 (1H, bd), |
| | [(1R*,2R*,4S*)- | octahydro-1H- | | | 3.30-3.58 (5H, |
| | bicyclo[2.2.1]hept- | pyrrolo[3,4- | | | mm), 2.86 (1H, |
| | 2- | b]pyridine-1- | | | btd), 2.30 (2H, |
| | ylamino]pyrimidin- | carboxylate | | | bdd), 1.97 (3H, |
| | 4- | (CAS RN 181141- | | | s), |
| | yl}octahydro-1H- | 40-2) | | | 1.70-1.89 (3H, m), |
| | pyrrolo[3,4- | | | | 1.50 (9H, s), |
| | b]pyridine-1- | | | | 1.16-1.65 (6H, mm). |
| | carboxylate | | | | |
| Interm. | tert-butyl | Intermediate 17, | Diacetate | 415 [M + H]⁺ | CDCl₃ 7.85 (1H, |
| 36 | (3aR*,6aS*)-5- | tert-butyl | | RT 3.56 mins | bd), 5.65 (2H, |
| | {2-amino-6- | (3aR*,6aS*)- | | (pH 5.8) | bs), 4.61 (1H, s), |
| | [(1R*,2R*,4S*)- | hexahydropyrrolo[3, | | | 3.12-3.74 (9H, |
| | bicyclo[2.2.1]hept- | 4-c]pyrrole- | | | mm), 2.95 (2H, |
| | 2- | 2(1H)-carboxylate | | | bs), 2.32 (2H, |
| | ylamino]pyrimidin- | | | | bs), 2.04 (6H, s, |
| | 4- | | | | 2 × AcOH), |
| | yl}hexahydropyrrolo[3,4- | | | | 1.74 (1H, bddd), |
| | c]pyrrole- | | | | 1.40-1.58 (3H, m), |
| | 2(1H)- | | | | 1.46 (9H, s), |
| | carboxylate | | | | 1.12-1.26 (4H, |
| | | | | | mm). |
| Interm. | tert-butyl 4-{2- | Intermediate 17, | Acetate | 389 [M + H]⁺ | CD₃OD |
| 37 | amino-6- | tert-butyl | | RT 3.68 mins | 4.91 (1H, obscured |
| | [(1R*,2R*,4S*)- | piperazine-1- | | (pH 5.8) | by H₂O peak, s), |
| | bicyclo[2.2.1]hept- | carboxylate | | | 3.43-3.62 (9H, |
| | 2- | | | | mm), 2.29 (2H, |
| | ylamino]pyrimidin- | | | | bdd), 1.97 (3H, |
| | 4- | | | | s, 1 × AcOH), |
| | yl}piperazine-1- | | | | 1.84 (1H, ddd), |
| | carboxylate | | | | 1.18-1.60 (7H, |
| | | | | | mm), 1.50 (9H, |
| | | | | | s). |
| 112 | N⁴- | Intermediate 17, | | 291 [M + H]⁺ | CD₃OD |
| | [(1R*,2R*,4S*)- | N,N- | | RT 2.05 mins | 4.80 (1H, s), |
| | [bicyclo[2.2.1]hept- | dimethylethylenediamine | | (pH 5.8) | 3.29-3.43 (2H, m), |
| | 2-yl]-N⁶-[2- | | | | 2.55 (2H, t), |
| | (dimethylamino)ethyl]pyrimidine- | | | | 2.33 (6H, s), |
| | 2,4,6-triamine | | | | 2.26 (2H, bdd), |
| | | | | | 1.79 (1H, ddd), |
| | | | | | 1.46-1.62 (3H, m), |
| | | | | | 1.13-1.36 (5H, |
| | | | | | m). |
| 113 | N⁴- | Intermediate17, | Acetate | 343 [M + H]⁺ | d₆-DMSO |
| | [(1R*,2R*,4S*)- | octahydro-2H- | | RT 2.63 mins | 6.06 (1H, bd), |
| | bicyclo[2.2.1]hept- | pyrido[1,2- | | (pH 5.8) | 5.42 (2H, s), |
| | 2-yl]-6- | a]pyrazine (CAS | | | 4.97 (2H, s), |
| | (octahydro-2H- | RN 4430-75-5) | | | 3.96 (1H, t), 3.51 (1H, |
| | pyrido[1,2- | | | | bs), |
| | a]pyrazin-2- | | | | 2.80-2.66 (3H, m), |
| | yl)pyrimidine- | | | | 2.34 (1H, t), 2.13 (2H, |
| | 2,4-diamine | | | | bdd), |
| | | | | | 1.85-2.12 (1H, m), |
| | | | | | 1.90 (3H, s, 1 × AcOH), |
| | | | | | 1.34-1.82 (10H, mm), |
| | | | | | 1.01-1.30 (6H, |
| | | | | | mm). |
| 114 | N⁴- | Intermediate 17, | | 329 [M + H]⁺ | CD₃OD |
| | [(1R*,2R*,4S*)- | octahydropyrrolo[1,2- | | RT 2.38 mins | 4.90 (1H, obscured |
| | bicyclo[2.2.1]hept- | a]pyrazine | | (pH 5.8) | by H₂O peak, s), |
| | 2-yl]-6- | (CAS RN 5654- | | | 4.35 (1H, d), |
| | (hexahydropyrrolo[1,2- | 83-1) | | | 4.15 (1H, d), |
| | a]pyrazin- | | | | 3.46 (1H, bd), |
| | 2(1H)- | | | | 3.05-3.15 (2H, |
| | yl)pyrimidine- | | | | m), 2.93 (1H, td), |
| | 2,4-diamine | | | | 2.57 (1H, t), |
| | | | | | 2.18-2.31 (4H, mm), |
| | | | | | 2.02-2.14 (1H, |
| | | | | | mm), |
| | | | | | 1.75-2.00 (4H, mm), |
| | | | | | 1.40-1.59 (4H, mm), |
| | | | | | 1.14-1.35 (4H, |
| | | | | | mm). |
| Interm. | tert-butyl (2S)-4- | 4-chloro-6- | | 362 [M + H]⁺ | CDCl₃ 5.80 (1H, |
| 38 | (2-amino-6- | cyclopentylpyrimidin- | | RT 2.28 mins | s), 4.97 (1H, bs), |
| | cyclopentylpyrimidin- | 2-amine, tert- | | (pH 5.8) | 4.10-4.34 (2H, |
| | 4-yl)-2- | butyl (2S)-2- | | | mm), |
| | methylpiperazine- | methylpiperazine- | | | 3.74-4.19 (2H, ddd), |
| | 1-carboxylate | 1-carboxylate | | | 3.14-3.30 (1H, ddd), |
| | | (CAS RN 169447- | | | 2.61-3.10 (3H, |
| | | 70-5) | | | mm), |
| | | | | | 1.91-2.08 (2H, m), |
| | | | | | 1.54-1.92 (6H, mm), |
| | | | | | 1.46 & 1.48 (9H, |
| | | | | | 2s), 1.14 & |
| | | | | | 1.21 (3H, 2d). |
| Interm. | tert-butyl (2R)-4- | 4-chloro-6- | | 362 [M + H]⁺ | CDCl₃ 5.80 (1H, |
| 39 | (2-amino-6- | cyclopentylpyrimidin- | | RT 2.27 mins | s), 4.97 (1H, bs), |
| | cyclopentylpyrimidin- | 2-amine, tert- | | (pH 5.8) | 4.10-4.34 (2H, |
| | 4-yl)-2- | butyl (2R)-2- | | | mm), |
| | methylpiperazine- | methylpiperazine- | | | 3.74-4.19 (2H, ddd), |
| | 1-carboxylate | 1-carboxylate | | | 3.14-3.30 (1H, ddd), |
| | | (CAS RN 170033- | | | 2.61-3.10 (3H, |
| | | 47-3) | | | mm), |
| | | | | | 1.91-2.08 (2H, m), |
| | | | | | 1.54-1.92 (6H, mm), |
| | | | | | 1.46 & 1.48 (9H, |
| | | | | | 2s), 1.14 & |
| | | | | | 1.21 (3H, 2d). |
| Interm. | tert-butyl (3S)-4- | 4-chloro-6- | Acetate | 376 [M + H]⁺ | CD₃OD |
| 40 | (2-amino-6- | cyclohexylpyrimidin- | | RT 3.71 mins | 6.15 (1H, s), |
| | cyclohexylpyrimidin- | 2-amine, tert- | | (pH 5.8) | 4.73 (1H, bm), |
| | 4-yl)-3- | butyl (3S)-3- | | | 4.28 (1H, bd), |
| | methylpiperazine- | methylpiperazine- | | | 4.05 (1H, bd), |
| | 1-carboxylate | 1-carboxylate | | | 3.89-3.98 (1H, m), |
| | | (CAS RN 147081- | | | 2.94-3.35 (3H, |
| | | 29-6) | | | mm), 2.46 (1H, |
| | | | | | btd), |
| | | | | | 1.74-2.00 (5H, mm), |
| | | | | | 1.52 (9H, s), |
| | | | | | 1.26-1.58 (5H, mm), |
| | | | | | 1.22 (3H, d). |
| Interm. | tert-butyl (3R)-4- | 4-chloro-6- | | 376 [M + H]⁺ | CD₃OD |
| 41 | (2-amino-6- | cyclohexylpyrimidin- | | RT 3.61 mins | 6.15 (1H, s), |
| | cyclohexylpyrimidin- | 2-amine, tert- | | (pH 5.8) | 4.73 (1H, bm), |
| | 4-yl)-3- | butyl (3R)-3- | | | 4.28 (1H, bd), |
| | methylpiperazine- | methylpiperazine- | | | 4.05 (1H, bd), |
| | 1-carboxylate | 1-carboxylate | | | 3.89-3.98 (1H, m), |
| | | (CAS RN 163765- | | | 2.94-3.35 (3H, |
| | | 44-4) | | | mm), 2.46 (1H, |
| | | | | | btd), |
| | | | | | 1.74-2.00 (5H, mm), |
| | | | | | 1.52 (9H, s), |
| | | | | | 1.26-1.58 (5H, mm), |
| | | | | | 1.22 (3H, d). |
| 115 | N⁴-cyclohexyl- | 6-chloro-N⁴- | | 279 [M + H]⁺ | CDCl₃ 6.11 (1H, |
| | N⁶-[2- | cyclohexylpyrimidin- | | RT 1.92 mins | bs), 5.80 (1H, |
| | (dimethylamino)ethyl]pyrimidine- | 2,4-diamine, | | (pH 5.8) | bs), 5.35 (2H, |
| | 2,4,6-triamine | N,N- | | | bs), |
| | | dimethylethylenediamine | | | 3.20-3.35 (3H, m), |
| | | | | | 2.52 (2H, t), 2.25 (6H, |
| | | | | | s), |
| | | | | | 1.90-2.03 (2H, m), |
| | | | | | 1.68 (2H, m), |
| | | | | | 1.55-1.68 (1H, m), |
| | | | | | 1.14-1.41 (6H, |
| | | | | | m) |
| Interm. | tert-butyl (1-{2- | Intermediate 26, | formate | 403 [M + H]⁺ | CDCl₃ 8.6 (1H, |
| 42 | amino-6- | tert-butyl | | RT 2.58 mins | HCOOH), |
| | [(1R*,2S*,4S*)- | methyl(pyrrolidin- | | (pH 2.5) | 8.02 (1H, m), |
| | bicyclo[2.2.1]hept- | 3-yl)carbamate | | | 6.05 (2H, bs), |
| | 2- | (CAS RN 172478- | | | 4.82 (1H, bm), |
| | ylamino]pyrimidin- | 00-1) | | | 4.68 (1H, s), |
| | 4-yl}pyrrolidin- | | | | 3.25-3.75 (6H, m), |
| | 3- | | | | 2.80 (3H, m), |
| | yl)methylcarbamate | | | | 1.98-2.52 (6H, |
| | | | | | m), |
| | | | | | 1.25-1.75 (3H, m), |
| | | | | | 1.48 (9H, s), |
| | | | | | 1.10 (1H, m), |
| | | | | | 0.90 (1H, m). |
| Interm. | tert-butyl [(3S)- | Intermediate 26, | formate | 389 [M + H]⁺ | CDCl₃ 8.6 (1H, |
| 43 | 1-{2-amino-6- | tert-butyl (3S)- | | RT 2.27 mins | HCOOH), |
| | [(1R*,2S*,4S*)- | pyrrolidin-3- | | (pH 2.5) | 7.88 (1H, s), |
| | bicyclo[2.2.1]hept- | ylcarbamate | | | 5.95 (2H, bs), |
| | 2- | | | | 4.60-4.75 (2H, m), |
| | ylamino]pyrimidin- | | | | 4.30 (1H, m), |
| | 4-yl}pyrrolidin- | | | | 3.25-3.80 (5H, |
| | 3-yl]carbamate | | | | m), |
| | | | | | 1.22-2.90 (10H, m + HCOOH), |
| | | | | | 1.42 (9H, s), |
| | | | | | 1.05 (1H, m), |
| | | | | | 0.88 (1H, m). |
| Interm. | tert-butyl [(3R)- | Intermediate 26, | | 389 [M + H]⁺ | CDCl₃ 8.6 (1H, |
| 44 | 1-{2-amino-6- | tert-butyl (3R)- | | RT 2.28 mins | HCOOH), |
| | [(1R*,2S*,4S*)- | pyrrolidin-3- | | (pH 2.5) | 7.85 (1H, s), |
| | bicyclo[2.2.1]hept- | ylcarbamate | | | 5.95 (2H, bs), |
| | 2- | | | | 4.60-4.78 (2H, m), |
| | ylamino]pyrimidin- | | | | 4.30 (1H, m), |
| | 4-yl}pyrrolidin- | | | | 3.25-3.80 (5H, |
| | 3-yl]carbamate | | | | m), |
| | | | | | 1.22-2.90 (10H, m + HCOOH), |
| | | | | | 1.42 (9H, s), |
| | | | | | 1.05 (1H, m), |
| | | | | | 0.88 (1H, m). |
| Interm. | tert-butyl 4-(2- | 4-chloro-6- | | 348 [M + H]⁺ | CDCl₃ 5.85 (1H, |
| 45 | amino-6- | cyclopentylpyrimidin- | | RT 2.20 mins | s), 5.40 (2H, bs), |
| | cyclopentylpyrimidin- | 2-amine, tert- | | (pH 2.5) | 3.62 (4H, m), |
| | 4- | butyl piperazine- | | | 3.50 (4H, m), |
| | yl)piperazine-1- | 1-carboxylate | | | 2.87 (1H, m), |
| | carboxylate | | | | 1.97-2.10 (2H, |
| | | | | | m), |
| | | | | | 1.60-1.87 (6H, m), |
| | | | | | 1.48 (9H, s). |
| Interm. | tert-butyl 4-(2- | 4-chloro-6- | | 362 [M + H]⁺ | CDCl₃ 5.81 (1H, |
| 46 | amino-6- | cyclopentylpyrimidin- | | RT 2.21 mins | s), |
| | cyclopentylpyrimidin- | 2-amine, tert- | | (pH 2.5) | 4.89-5.18 (2H, bm), |
| | 4-yl)-2- | butyl 2- | | | 4.30 (1H, m), |
| | methylpiperazine- | methylpiperazine- | | | 4.15 (1H, m), |
| | 1-carboxylate | 1-carboxylate | | | 4.01 (1H, m), |
| | | (CAS RN 120737- | | | 3.90 (1H, m), |
| | | 78-2) | | | 3.12-3.32 (2H, m), |
| | | | | | 3.02 (1H, m), |
| | | | | | 2.85 (1H, m), |
| | | | | | 2.02 (2H, m), |
| | | | | | 1.50 (9H, s), |
| | | | | | 1.40-1.89 (6H, |
| | | | | | m), 1.15 (3H, d). |
| Interm. | tert-butyl {1-[2- | Intermediate 87, | | 362 [M + H]⁺ | CDCl₃ 5.60 (1H, |
| 47 | amino-6- | tert-butyl | | RT 2.25 mins | s), 5.55 (1H, bs), |
| | (cyclopentylmethyl)pyrimidin- | pyrrolidin-3- | | (pH 2.5) | 5.00 (1H, d), |
| | 4- | ylcarbamate | | | 4.90 (1H, bs), |
| | yl]pyrrolidin-3- | | | | 4.30 (1H, m), |
| | yl}carbamate | | | | 3.25-3.80 (4H, |
| | | | | | m), 2.48 (2H, d), |
| | | | | | 2.24 (2H, m), |
| | | | | | 1.98 (1H, m), |
| | | | | | 1.38-1.83 (6H, |
| | | | | | m), 1.45 (9H, s), |
| | | | | | 1.10-1.30 (2H, |
| | | | | | m). |
| Interm. | tert-butyl 4-{6- | Intermediate 12, | | 428 [M + H]⁺ | CDCl₃ 5.93 (1H, |
| 48 | [adamantan-2- | tert-butyl 2- | | RT 2.70 mins | s), 4.80 (2H, bs), |
| | yl]-2- | methylpiperazine- | | (pH 2.5) | 4.30 (1H, m), |
| | aminopyrimidin- | 1-carboxylate | | | 4.18 (1H, m), |
| | 4-yl}-2- | | | | 3.98 (1H, m), |
| | methylpiperazine- | | | | 3.90 (1H, m), |
| | 1-carboxylate | | | | 3.27 (1H, dd), |
| | | | | | 3.20 (1H, dt), |
| | | | | | 3.00 (1H, dt), |
| | | | | | 2.75 (1H, s), |
| | | | | | 2.50 (2H, s), |
| | | | | | 1.70-2.02 (10H, m), |
| | | | | | 1.58 (2H, m), |
| | | | | | 1.48 (9H, s), |
| | | | | | 1.15 (3H, d). |
| Interm. | 4-(1-benzyl-1,7- | 4-chloro-6- | | 378 [M + H]⁺ | CDCl₃ |
| 49 | diazaspiro[4.4]non- | cyclopentylpyrimidin- | | RT 1.30 mins | 7.20-7.35 (5H, m), |
| | 7-yl)-6- | 2-amine, 1- | | (pH 2.5) | 6.00 (2H, bs), |
| | cyclopentylpyrimidin- | benzyl-1,7- | | | 5.65 (1H, s), |
| | 2-amine | diazaspiro[4.4]nonane | | | 3.15-3.95 (6H, |
| | | (CAS RN | | | m), 2.92 (1H, m), |
| | | 128244-01-9) | | | 2.60-2.83 (2H, |
| | | | | | m), |
| | | | | | 1.50-2.45 (14H, m). |
| Interm. | tert-butyl | 4-chloro-6- | | 388 [M + H]⁺ | CDCl₃ 5.65 (1H, |
| 50 | (4aR*,7aR*)-6- | cyclopentylpyrimidin- | | RT 2.46 mins | s), 5.60 (2H, bs), |
| | (2-amino-6- | 2-amine, tert- | | (pH 2.5) | 4.80 (1H, m), |
| | cyclopentylpyrimidin- | butyl | | | 4.03 (1H, m), |
| | 4- | (4aR*,7aR*)- | | | 3.10-3.90 (4H, |
| | yl)octahydro-1H- | octahydro-1H- | | | m), 2.88 (1H, m), |
| | pyrrolo[3,4- | pyrrolo[3,4- | | | 2.75 (1H, m), |
| | b]pyridine-1- | b]pyridine-1- | | | 2.28 (1H, m), |
| | carboxylate | carboxylate | | | 2.00-2.15 (2H, |
| | | (CAS RN 181141- | | | m), |
| | | 40-2) | | | 1.60-1.92 (8H, m), |
| | | | | | 1.48 (9H, s), |
| | | | | | 1.20-1.40 (2H, m). |
| Interm. | tert-butyl [1-(2- | 4-chloro-6- | | 334 [M + H]⁺ | CDCl₃ 5.50 (1H, |
| 51 | amino-6- | cyclopentylpyrimidin- | | RT 2.07 mins | s), 5.07 (1H, m), |
| | cyclopentylpyrimidin- | 2-amine, tert- | | (pH 2.5) | 4.60 (1H, m), |
| | 4- | butyl azetidin-3- | | | 4.35 (2H, t), |
| | yl)azetidin-3- | ylcarbamate | | | 3.93 (2H, m), |
| | yl]carbamate | (CAS RN 91188- | | | 2.90 (1H, m), |
| | | 13-5) | | | 2.00-2.15 (2H, m), |
| | | | | | 1.60-1.90 (6H, |
| | | | | | m), 1.45 (9H, s). |
| Interm. | tert-butyl (1-{2- | Intermediate 26, | | 389 [M + H]⁺ | CDCl₃ 8.55 (1H, |
| 52 | amino-6- | tert-butyl | | RT 2.44 mins | HCOOH), |
| | [(1R*,2S*,4S*)- | pyrrolidin-3- | | (pH 2.5) | 7.85 (1H, bs), |
| | bicyclo[2.2.1]hept- | ylcarbamate | | | 6.25 (2H, bs), |
| | 2- | | | | 5.10 (1H, m), |
| | ylamino]pyrimidin- | | | | 4.68 (1H, s), |
| | 4-yl}pyrrolidin- | | | | 4.27 (1H, m), |
| | 3-yl)carbamate | | | | 3.15-3.90 (5H, m), |
| | | | | | 2.50 (1H, s), |
| | | | | | 1.85-2.40 (5H, |
| | | | | | m), |
| | | | | | 1.22-1.75 (5H, m), |
| | | | | | 1.45 (9H, s), |
| | | | | | 1.08 (1H, d). |
| Interm. | tert-butyl 4-(2- | 4-chloro-6- | | 376 [M + H]⁺ RT | CDCl₃ 5.76 (1H, |
| 53 | amino-6- | cyclohexylpyrimidin- | | 2.51 (pH 2) | s), 4.63 (2H, s), |
| | cyclohexylpyrimidin- | 2-amine, tert- | | | 4.29 (1H, m), |
| | 4-yl)-2- | butyl 2- | | | 4.15 (1H, m), |
| | methylpiperazine- | methylpiperazine- | | | 3.97 (1H, m), |
| | 1-carboxylate | 1-carboxylate | | | 3.87 (1H, m), |
| | | (CAS RN 120737- | | | 3.22 (1H, m), |
| | | 78-2) | | | 3.17 (1H, m), |
| | | | | | 2.99 (1H, m), |
| | | | | | 2.33 (1H, m), |
| | | | | | 1.68-1.91 (5H, |
| | | | | | m), 1.47 (9H, s), |
| | | | | | 1.23-1.45 (5H, |
| | | | | | m), 1.13 (3H, d) |
| Interm. | tert-butyl 3-(2- | 4-chloro-6- | | 374 [M + H]⁺ | CDCl₃ 5.78 (1H, |
| 54 | amino-6- | cyclopentylpyrimidin- | | RT 2.34 mins | s), 4.63 (2H, |
| | cyclopentylpyrimidin- | 2-amine, tert- | | (pH 2) | brs), |
| | 4-yl)-3,8- | butyl 3,8- | | | 4.22-4.39 (2H, m), |
| | diazabicyclo[3.2.1]octane- | diazabicyclo[3.2.1]octane- | | | 3.84-4.06 (2H, m), |
| | 8- | 8- | | | 2.97-3.14 (2H, |
| | carboxylate | carboxylate (CAS | | | m), 2.80 (1H, m), |
| | | RN 149771-44-8) | | | 1.87-2.03 (4H, |
| | | | | | m), |
| | | | | | 1.59-1.82 (8H, m), |
| | | | | | 1.48 (9H, s) |
| Interm. | tert-butyl {1-[2- | Intermediate 16, | | 411 [M + H]⁺ | CDCl₃ |
| 55 | amino-6-(2,3- | tert-butyl | | RT 3.38 mins | 7.13-7.24 (4H, m), |
| | dihydro-1H- | pyrrolidin-3- | | (pH 5.8) | 4.84 (1H, s), |
| | inden-2- | ylcarbamate | | | 4.67 (2H, m), |
| | ylamino)pyrimidin- | | | | 4.38-4.51 (3H, m), |
| | 4-yl]pyrrolidin- | | | | 4.29 (1H, m), |
| | 3-yl}carbamate | | | | 3.67 (1H, m), |
| | | | | | 3.43-3.57 (2H, |
| | | | | | m), |
| | | | | | 3.35-3.49 (3H, m), |
| | | | | | 2.85 (2H, m), |
| | | | | | 2.21 (1H, m), |
| | | | | | 1.91 (1H, m), |
| | | | | | 1.46 (9H, s) |
| Interm. | tert-butyl (2S)-4- | 4-chloro-6- | | 390 [M + H]⁺ | CD₃OD |
| 56 | (2-amino-6- | cyclopentylpyrimidin- | | RT 3.82 mins | 6.01 (1H, s), |
| | cyclopentylpyrimidin- | 2-amine, tert- | | (pH 5.8) | 4.62 (1H, bd), |
| | 4-yl)-2- | butyl (2S)-2- | | | 4.27 (1H, bd), |
| | isopropylpiperazine- | isopropylpiperazine- | | | 4.0 (1H, bd), |
| | 1-carboxylate | 1-carboxylate | | | 3.78 (1H, bd), |
| | | (CAS RN 674792- | | | 2.77-3.07 (4H, mm), |
| | | 05-3) | | | 1.63-2.07 (9H, |
| | | | | | mm), 1.51 (9H, |
| | | | | | s), 1.07 (3H, d), |
| | | | | | 0.86 (3H, d) |
| Interm. | tert-butyl (2S)-4- | 4-chloro-6- | | 404 [M + H]⁺ | CD₃OD |
| 57 | (2-amino-6- | cyclopentylpyrimidin- | | RT 3.91 mins | 6.18 (1H, s), |
| | cyclopentylpyrimidin- | 2-amine, tert- | | (pH 5.8) | 4.20-4.54 (3H, mm), |
| | 4-yl)-2- | butyl (2S)-2- | | | 3.85-4.03 (1H, |
| | isobutylpiperazine- | isobutylpiperazine- | | | m), 3.21 (1H, |
| | 1-carboxylate | 1-carboxylate | | | bdd), |
| | | (CAS RN 674792- | | | 3.02-3.13 (1H, m), |
| | | 06-4) | | | 2.93 (1H, btd), |
| | | | | | 2.82-2.86 (1H, m), |
| | | | | | 2.30-2.43 (1H, |
| | | | | | m), |
| | | | | | 1.28-2.18 (10H, mm), |
| | | | | | 1.49 (9H, s), |
| | | | | | 0.96 (6H, dd) |
| 116 | 6-cyclopentyl- | 4-chloro-6- | | 302 [M + H]⁺, | CDCl₃ 5.65 (1H, |
| | N⁴-[(3-endo)-8- | cyclopentylpyrimidin- | | RT 1.92 mins | s), 4.65 (2H, br |
| | methyl-8- | 2-amine, (3- | | (pH 5.8) | s), 4.29 (1H, br |
| | azabicyclo[3.2.1]oct- | endo)-8-methyl-8- | | | s), 3.98 (1H, br |
| | 3- | azabicyclo[3.2.1]octan- | | | s), 3.20 (2H, s), |
| | yl]pyrimidine- | 3-amine | | | 2.75 (1H, m), |
| | 2,4-diamine | (CAS RN 87571- | | | 2.30 (3H, s), |
| | | 88-8) | | | 2.10 (2H, m), |
| | | | | | 1.95 (4H, m), |
| | | | | | 1.60-1.85 (8H, |
| | | | | | m), 1.55 (2H, m) |
| 117 | 6-cyclohexyl- | 4-chloro-6- | | 304 [M + H]⁺ | CDCl₃5.65 (1H, |
| | N⁴-[(1- | cyclohexylpyrimidin- | | RT 2.04 mins | s), |
| | methylpiperidin- | 2-amine, 1-(1- | | (pH 5.8) | 5.65-5.75 (1H, br), |
| | 2- | methylpiperidin-2- | | | 4.65 (2H, br s), |
| | yl)methyl]pyrimidine- | yl)methanamine | | | 3.20-3.45 (2H, m), |
| | 2,4-diamine | (CAS RN 14613- | | | 2.85 (1H, br d), |
| | | 37-7) | | | 2.20-2.35 (1H, |
| | | | | | m), 2.25 (3H, s), |
| | | | | | 2.05-2.15 (2H, |
| | | | | | m), |
| | | | | | 1.65-1.95 (4H, m), |
| | | | | | 1.15-1.65 (12H, m) |

Comp. No means Compound Number
Interm. means Intermediate

### Example 18

### Synthesis of 4-cyclopentyl-6-(4-methylpiperazin-1-yl)pyrimidin-2-amine (Compound 118)

A suspension of 4-chloro-6-(4-methylpiperazin-1-yl)pyrimidin-2-amine (86 mg), chlorotrimethylsilane (96 μl) and DIPEA (132 μl) in dry THF is stirred at room temperature for 1 hr. To the reaction mixture is added cyclopentylzinc bromide (0.5M solution in THF) (1.5 ml) followed by PdCl₂(dppf) (14 mg). The reaction mixture is then heated under microwave irradiation for 6 mins at 110° C. Solvents are removed in vacuo and the crude material is dissolved in EtOAc (50 ml), washed with water (15 ml), dried over MgSO₄, filtered and concentrated in vacuo. Purification by preparative HPLC (Method B) affords the title compound as a colorless solid (6.2 mg, 6%). LCMS 262 [M+H]⁺, RT (pH5.8) 1.90 mins. ¹H NMR 300 MHz (CD₃OD) (δ ppm): 6.20 (1H, s), 3.70 (4H, t), 2.90 (1H, m), 2.55 (4H, t), 2.40 (3H, s), 2.10 (2H, m), 1.90 (2H, m), 1.75 (4H, m).

Compounds 119 through 121 are prepared in a similar manner to the method described for Compound 118 in Example 18. The reaction mixtures are directly purified by preparative HPLC using Method B.

The reagents used and the results obtained are tabulated in Table 10. The free base of compounds is obtained.

TABLE 10

| | | | | ¹H NMR |
|---|---|---|---|---|
| Comp. | IUPAC | Starting | | (Solvent, |
| No | Name | Materials | LCMS | δ ppm) |
| 119 | 4-(1- | 4-chloro-6- | 278 [M + H]⁺ | CD₃OD 6.00 (1H, s), |
| | methylpentyl)- | (4- | RT 2.31 mins | 3.70 (4H, t), 2.50 (5H, m), |
| | 6-(4- | methylpiperazin- | (pH | 2.35 (3H, s), 1.70 (1H, m), |
| | methylpiperazin- | 1- | 5.8). | 1.55 (1H, m), 1.35 (4H, m), |
| | 1- | yl)pyrimidin- | | 1.20 (3H, d), 0.90 (3H, t). |
| | yl)pyrimidin- | 2-amine, 1- | | |
| | 2-amine | methylpentylzinc | | |
| | | bromide | | |
| 120 | 4-(4- | 4-chloro-6- | 298 [M + H]⁺ | CD₃OD7.35 (5H, m), |
| | methylpiperazin- | (4- | RT 2.32 mins | 6.20 (1H, s), 4.00 (1H, q), |
| | 1-yl)-6-(1- | methylpiperazin- | (pH5.8). | 3.80 (4H, t), 2.60 (4H, t), 2.40 (3H, |
| | phenylethyl)pyrimidin- | 1- | | s), 1.65 (3H, d). |
| | 2- | yl)pyrimidin- | | |
| | amine | 2-amine, | | |
| | | α- | | |
| | | methylbenzylzinc | | |
| | | bromide | | |
| 121 | 4-(1- | 4-chloro-6- | 264 [M + H]⁺ | CD₃OD 6.05 (1H, s), |
| | ethylpropyl)- | (4- | RT 1.99 mins | 3.70 (4H, t), 2.55 (4H, t), 2.35 (3H, |
| | 6-(4- | methylpiperazin- | (pH | s), 2.25 (1H, m), 1.65 (4H, |
| | methylpiperazin- | 1- | 5.8). | m), 0.85 (6H, t). |
| | 1- | yl)pyrimidin- | | |
| | yl)pyrimidin- | 2-amine, 1- | | |
| | 2-amine | ethylpropylzinc | | |
| | | bromide | | |

Comp. No means Compound Number

### Example 19

### Synthesis of 3-[(4-fluorobenzyl)oxy]pyrrolidine (Intermediate 58)

To a solution of 3-hydroxypyrrolidine-1-carboxylic acid tert-butyl ester (2.67 g) in DMF (80 ml) is added potassium tert-butoxide (1.68 g). After stirring for 15 minutes 4-fluorobenzylbromide is added (1.87 ml) and the reaction mixture is stirred overnight at room temperature. The reaction mixture is diluted with EtOAc (160 ml) and washed with brine (4×150 ml), dried (MgSO₄) and concentrated under reduced pressure. The residue is dissolved in DCM (90 ml) and TFA (10 ml) is added. After stirring for 30 minutes, the mixture is concentrated in vacuo and azeotroped with toluene (4×75 ml) to give a brown oil (2.74 g, 62%). LCMS 196 [M+H]⁺, RT 1.90 mins (pH 5.8). ¹H NMR 300 MHz (d₆-DMSO) (δ ppm): 9.18-8.80 (2H, br s), 7.46-7.28 (2H, m), 7.25-7.09 (2H, m), 4.48 (2H, s), 4.32-4.22 (1H, m), 3.40-3.10 (4H, m).

### Example 20

### Synthesis of 4-(4-methylpiperazin-1-yl)-6-[(2S)-2-methylpyrrolidin-1-yl]pyrimidin-2-amine (Compound 122)

Compound 122 is prepared from 4,6-dichloropyrimidin-2-amine in two steps. A mixture of (S)-2-methylpyrrolidine hydrochloride (400 mg) and 4,6-dichloropyrimidin-2-amine (500 mg) in NMP (1.0 ml) and triethylamine (1.0 ml) is heated under microwave irradiation at 110° C. for 30 mins (step one). The vessel is cooled and N-methylpiperazine (1 ml) is added. The mixture is heated at 200° C. for 20 mins (second step). The solid mixture is added to water (20 ml) and extracted with EtOAc (30 ml). The solvent is washed with water (2×20 ml), dried and evaporated to half volume. The solution is allowed to stand for 1 hr, and then the product is collected by filtration to give the title compound as white solid (370 mg, 43%). LCMS 277 [M+H]⁺, RT 2.02 mins. ¹H NMR 300 MHz (CDCl₃) (δppm): 4.95 (1H, s), 4.45 (2H, br s), 4.15 (1H, m), 3.55 (4H, m), 3.50 (1H, m), 3.30 (1H, m), 2.50 (4H, m), 2.35 (3H, s), 1.85-2.10 (3H, m), 1.65 (1H, m), 1.20 (3H, d).

Compounds 123 through 162 are prepared from 4,6-dichloropyrimidin-2-amine in a similar manner to the method described for Compound 122 in Example 20. The first step can be carried out with either NMP or EtOH as the solvent, with either Et₃N or DIPEA as the base and at temperature ranging between 110° C. and 180° C. under microwave irradiation. The second step is performed at 200° C. under microwave irradiation. The crude reaction mixtures are directly purified by preparative HPLC using either Method C or Method D.

The reagents used and the results obtained are tabulated in Table 11. The free base of compounds is obtained unless otherwise stated.

TABLE 11

| | | | | | ¹H NMR |
|---|---|---|---|---|---|
| Comp. No | IUPAC Name | Starting Materials | Salt | LCMS | (Solvent, δ ppm) |
| 123 | 4-(4- | (R)-2- | | 353 [M + H]⁺ | CDCl₃ 4.95 (1H, s), |
| | methylpiperazin- | methylpyrrolidine | | RT 2.62 mins | 4.40 (2H, s), 4.12 (1H, |
| | 1-yl)-6- | hydrochloride, | | (pH | m), 3.50 (4H, m), |
| | [(2R)-2- | N- | | 5.8) | 3.45 (1H, m), 3.32 (1H, m), |
| | methylpyrrolidin- | methylpiperazine | | | 2.45 (4H, m), 2.32 (3H, |
| | 1- | | | | s), 1.90-2.10 (3H, m), |
| | yl]pyrimidin-2- | | | | 1.65 (1H, m), 1.19 (3H, |
| | amine | | | | d) |
| 124 | 6-(4- | (1R*,2S*,4S*)- | | 345 [M + H]⁺ | CD₃OD 4.90 (1H, s), |
| | methylpiperazin- | 1,3,3- | | RT 2.82 mins | 3.65 (4H, s), 3.35 (1H, |
| | 1-yl)-N⁴- | trimethylbicyclo[2.2.1]heptan- | | (pH | m), 2.60 (4H, t), |
| | [(1R*,2S*,4S*)- | 2- | | 5.8) | 2.40 (3H, s), 1.75 (3H, m), |
| | 1,3,3- | amine | | | 1.55 (2H, m), 1.30 (2H, |
| | trimethylbicyclo[2.2.1]hept- | hydrochloride, N- | | | m), 1.20 (3H, s), |
| | 2- | methylpiperazine | | | 1.10 (3H, s), 0.85 (3H, s) |
| | yl]pyrimidine- | | | | |
| | 2,4-diamine | | | | |
| 125 | 6-(4- | 2- | | 313 [M + H]⁺ | CD₃OD 7.25 (5H, m), |
| | methylpiperazin- | phenylethylamine, | | RT 2.23 mins | 4.90 (1H, s), 3.50 (4H, |
| | 1-yl)-N⁴- | N- | | (pH | t), 3.45 (2H, m), |
| | (2- | methylpiperazine | | 5.8) | 2.90 (2H, t), 2.50 (4H, t), |
| | phenylethyl)pyrimidine- | | | | 2.35 (3H, s) |
| | 2,4- | | | | |
| | diamine | | | | |
| 126 | 4-[(2R*,6S*)- | (2R*,6S*)- | | 305 [M + H]⁺ | CDCl₃ 5.15 (1H, s), |
| | 2,6- | dimethylpiperidine, | | RT 2.40 mins | 4.50 (2H, m), 4.43 (2H, |
| | dimethylpiperidin- | N- | | (pH | s), 3.55 (4H, m), |
| | 1-yl]-6-(4- | methylpiperazine | | 5.8) | 2.47 (4H, m), 2.35 (3H, s), |
| | methylpiperazin- | | | | 1.85 (1H, m), |
| | 1- | | | | 1.50-1.70 (5H, m), 1.20 (6H, d) |
| | yl)pyrimidin- | | | | |
| | 2-amine | | | | |
| 127 | N⁴- | (1R*,2S*,4S*)- | | 303 [M + H]⁺ | CD₃OD 4.90 (1H, s), |
| | [(1R*,2S*,4S*)- | bicyclo[2.2.1]heptan- | | RT 2.20 mins | 3.90 (1H, m), 3.65 (4H, |
| | bicyclo[2.2.1]hept- | 2-amine | | (pH | t), 2.60 (4H, t), |
| | 2-yl]-6- | hydrochloride, N- | | 5.8) | 2.55 (0.5H, m), 2.40 (3H, s), |
| | (4- | methylpiperazine | | | 2.25 (0.5H, m), |
| | methylpiperazin- | | | | 2.10 (1H, m), 2.00 (1H, m), |
| | 1- | | | | 1.65-1.30 (6H, m), |
| | yl)pyrimidin- | | | | 1.00 (1H, m) |
| | 2,4-diamine | | | | |
| 128 | 4-(7- | 7-aza- | Acetate | 289 [M + H]⁺ | CD₃OD 4.95 (1H, s), |
| | azabicyclo[2.2.1]hept- | bicyclo[2.2.1]heptane | | RT 1.86 mins | 4.48 (2H, s), |
| | 7-yl)- | hydrochloride, N- | | (pH | 3.70-3.60 (4H, m), |
| | 6-(4- | methylpiperazine | | 5.8) | 2.69-2.56 (4H, m), 2.47 (3H, |
| | methylpiperazin- | | | | s), 1.98 (3H, s), |
| | 1- | | | | 1.89-1.72 (4H, m), |
| | yl)pyrimidin- | | | | 1.62-1.46 (4H, m) |
| | 2-amine | | | | |
| | acetate salt | | | | |
| 129 | N⁴- | 2- | | 343 [M + H]⁺ | CD₃OD 4.90 (1H, s), |
| | adamantan-2- | adamantanamine | | RT 2.69 mins | 3.80 (1H, m), 3.65 (4H, |
| | yl-6-(4- | hydrochloride, N- | | (pH | t), 2.60 (4H, m), |
| | methylpiperazin- | methylpiperazine | | 5.8) | 2.40 (3H, s), 2.10 (2H, m), |
| | 1- | | | | 2.00 (6H, m), 1.80 (4H, |
| | yl)pyrimidine- | | | | m), 1.70 (2H, m) |
| | 2,4-diamine | | | | |
| 130 | 4-{3-[(4- | Intermediate 58, | | 387 [M + H]⁺ | CD₃OD |
| | fluorobenzyl)oxy]pyrrolidin- | N- | | RT 2.45 mins. | 7.42-7.31 (2H, m), |
| | 1-yl}-6-(4- | methylpiperazine | | (pH | 7.13-6.98 (2H, m), |
| | methylpiperazin- | | | 5.8) | 4.90 (1H, s), |
| | 1- | | | | 4.66-4.50 (2H, m), |
| | yl)pyrimidin- | | | | 3.64-3.41 (8H, m), |
| | 2-amine | | | | 2.58-2.46 (4H, m), |
| | | | | | 2.37 (3H, s), |
| | | | | | 2.26-2.00 (2H, m) |
| 131 | 4-(5-fluoro- | 5- | | 329 [M + H]⁺ | CDCl₃ 7.26 (1H, m), |
| | 1,3-dihydro- | fluoroisoindoline, | | RT 2.20 mins | 7.00 (2H, m), 5.04 (1H, |
| | 2H-isoindol-2- | N- | | (pH | s), 4.70 (4H, m), |
| | yl)-6-(4- | methylpiperazine | | 5.8) | 4.63 (2H, s), 3.60 (4H, m), |
| | methylpiperazin- | | | | 2.48 (4H, m), |
| | 1- | | | | |
| | yl)pyrimidin- | | | | |
| | 2-amine | | | | |
| 132 | 4-(4- | 4- | | 423 [M + H]⁺ | d₆-DMSO 7.67 (2H, d), |
| | methylpiperazin- | (trifluoromethyl)phenoxy]pyrrolidine, | | RT 2.98 mins | 7.14 (2H, d), 5.57 (2H, |
| | 1-yl)-6-{3- | N- | | (pH | s), 5.25-5.15 (1H, m), |
| | [4- | methylpiperazine | | 5.8) | 5.01 (1H, s), |
| | (trifluoromethyl)- | | | | 3.70-3.48 (3H, m), |
| | phenoxy]pyrrolidin-1- | | | | 3.47-3.36 (4H, m), |
| | yl}pyrimidin- | | | | 2.38-2.12 (7H, m), 2.20 (3H, |
| | 2-amine | | | | s) |
| 133 | N⁴- | 1- | | 343 [M + H]⁺ | CD₃OD 4.90 (1H, m), |
| | adamantan-1- | adamantanamine, | | RT 2.72 mins | 3.60 (4H, t), 2.60 (4H, |
| | yl-6-(4- | N- | | (pH | t), 2.40 (3H, s), |
| | methylpiperazin- | methylpiperazine | | 5.8) | 2.10 (9H, s), 1.80 (6H, s) |
| | 1- | | | | |
| | yl)pyrimidine- | | | | |
| | 2,4-diamine | | | | |
| 134 | 6-(4- | (1R,4R)-1,7,7- | | 345 [M + H]⁺ | CD₃OD 4.90 (1H, s), |
| | methylpiperazin- | trimethylbicyclo[2.2.1]hept- | | RT 2.88 mins | 4.00 (1H, m), 3.60 (4H, |
| | 1-yl)-N⁴- | 2- | | (pH | t), 2.60 (4H, t), |
| | ((1R,4R)- | ylamine | | 5.8) | 2.40 (3H, s), |
| | 1,7,7- | hydrochloride, N- | | | 1.90-1.65 (4H, m, br), |
| | trimethylbicyclo[2.2.1]hept- | methylpiperazine | | | 1.60-1.15 (3H, m, br), |
| | 2- | | | | 1.10-0.85 (9H, m) |
| | yl]pyrimidine- | | | | |
| | 2,4-diamine | | | | |
| 135 | 4-(4- | 4- | | 367 [M + H]⁺ | CDCl₃ 7.15-7.30 (5H, |
| | benzylpiperidin- | benzylpiperidine, | | RT 2.90 mins | m), 5.18 (1H, s), |
| | 1-yl)-6-(4- | N- | | (pH | 4.45 (2H, s), 4.25 (2H, m), |
| | methylpiperazin- | methylpiperazine | | 5.8) | 3.52 (4H, m), 2.70 (2H, |
| | 1- | | | | m), 2.55 (2H, d), |
| | yl)pyrimidin- | | | | 2.45 (4H, m), 2.32 (3H, s0, |
| | 2-amine | | | | 1.75 (1H, m), 1.70 (2H, |
| | | | | | m), 1.22 (2H, m) |
| 136 | 4-({1-[2- | 4-(4- | | 394 [M + H]⁺ | d₆-DMSO 7.78 (2H, d), |
| | amino-6-(4- | cyanophenoxy)piperidine, | | RT 2.56 mins | 7.18 (2H, d), 5.51 (2H, |
| | methylpiperazin- | N- | | (pH | s), 5.36 (1H, s), |
| | 1- | methylpiperazine | | 5.8) | 4.82-4.70 (1H, m), |
| | yl)pyrimidin- | | | | 4.02-3.39 (2H, m), |
| | 4-yl]piperidin- | | | | 3.50-3.38 (4H, m), |
| | 4- | | | | 3.30-3.17 (2H, m), |
| | yl}oxy)benzonitrile | | | | 2.38-2.23 (4H, m), 2.19 (3H, |
| | | | | | s), 2.03-1.89 (2H, m), |
| | | | | | 1.62-1.46 (2H, m) |
| 137 | 4-[4-(4- | 4- | | 403 [M + H]⁺ | d₆-DMSO 7.38 (2H, d), |
| | chlorophenoxy)piperidin- | chlorophenoxy)piperidine, | | RT 3.13 mins | 7.09 (2H, d), 5.66 (2H, |
| | 1- | N- | | (pH | s), 5.42 (1H, s), |
| | yl]-6-(4- | methylpiperazine | | 5.8) | 4.71-4.58 (1H, m), |
| | methylpiperazin- | | | | 4.08-3.94 (2H, m), |
| | 1- | | | | 3.57-3.44 (4H, m), |
| | yl)pyrimidin- | | | | 3.36-3.20 (2H, m), |
| | 2-amine | | | | 2.41-2.29 (4H, m), 2.24 (3H, |
| | | | | | s), 2.05-1.90 (2H, m), |
| | | | | | 1.65-1.48 (2H, m). |
| 138 | 4-[4-(4- | 4- | Acetate | 401 [M + H]⁺ | CD₃OD |
| | chlorobenzyl)piperidin- | chlorobenzylpiperidine, | | RT 3.26 mins | 7.38-7.13 (4H, m), 4.43 (1H, s), |
| | 1- | N- | | (pH | 4.33-4.20 (2H, m), |
| | yl]-6-(4- | methylpiperazine | | 5.8) | 3.70-3.55 (4H, m), |
| | methylpiperazin- | | | | 2.87-2.72 (2H, m), |
| | 1- | | | | 2.70-2.61 (4H, m), |
| | yl)pyrimidin- | | | | 2.60-2.50 (2H, m), |
| | 2-amine | | | | 2.43 (3H, s), 1.98 (3H, |
| | acetate salt | | | | s), 1.90-1.75 (1H, m), |
| | | | | | 1.73-1.62 (2H, m), |
| | | | | | 1.34-1.13 (2H, m) |
| 139 | 4-[4-(4- | 4- | Di- | 385 [M + H]⁺ | CD₃OD |
| | fluorobenzyl)piperidin- | fluorobenzylpiperidine, | acetate | RT 2.95 mins | 7.24-7.15 (2H, m), |
| | 1- | N- | | (pH | 7.07-6.98 (2H, m), 4.95 (1H, s), |
| | yl]-6-(4- | methylpiperazine | | 5.8) | 4.32-4.21 (2H, m), |
| | methylpiperazin- | | | | 3.66-3.55 (4H, m), |
| | 1- | | | | 2.38-2.71 (2H, m), |
| | yl)pyrimidin- | | | | 2.70-2.61 (4H, m), |
| | 2-amine | | | | 2.60-2.52 (2H, m), |
| | | | | | 2.45 (3H, s), 1.98 (6H, |
| | | | | | s), 1.88-1.76 (1H, m), |
| | | | | | 1.75-1.63 (2H, m), |
| | | | | | 1.36-1.10 (2H, m) |
| 140 | 4-(3-methyl- | 3-methyl-1,2,3,4- | | 339 [M + H]⁺ | CDCl₃ 7.25-7.14 (4H, |
| | 3,4- | tetrahydroisoquinoline, | | RT 2.71 mins | m), 5.62 (2H, br s), |
| | dihydroisoquinolin- | N- | | (pH | 5.30 (1H, s), |
| | 2(1H)- | methylpiperazine, | | 5.8) | 5.09-4.92 (1H, m), 4.78 (1H, d), |
| | yl)-6-(4- | | | | 4.21 (1H, d), |
| | methylpiperazin- | | | | 3.52-3.41 (4H, m), 3.01 (1H, |
| | 1- | | | | dd), 2.66 (1H, dd), |
| | yl)pyrimidin- | | | | 2.39-2.28 (4H, m), |
| | 2-amine | | | | 2.20 (3H, s), 0.93 (3H, d) |
| 141 | N⁴-[(3-exo)- | 8-benzyl-3-β- | | 408 [M + H]⁺ | CD₃OD 7.35-7.10 (5H, |
| | 8-benzyl-8- | amino- | | RT 1.73 mins | m), 5.00 (1H, s), |
| | azabicyclo[3.2.1]oct- | nortropane | | pH) | 4.05-3.90 (1H, m), 3.55 (2H, |
| | 3-yl]- | hemisulfate, N- | | 5.8) | s), 3.40-3.30 (4H, m), |
| | 6-(4- | methylpiperazine | | | 3.25-3.15 (2H, m) |
| | methylpiperazin- | | | | (partially obscured by |
| | 1- | | | | MeOH peak), |
| | yl)pyrimidine- | | | | 2.40-2.30 (4H, m), 2.20 (3H, |
| | 2,4-diamine | | | | s), 2.10-2.00 (2H, m), |
| | | | | | 1.80-1.70 (4H, m), |
| | | | | | 1.55-1.40 (2H, m). |
| 142 | N⁴- | cyclopentylamine, | Diacetate | 277 [M + H]⁺ | CD₃OD 4.95 (1H, |
| | cyclopentyl- | N- | | RT 2.00 mins | obscured by H₂O |
| | 6-(4- | methylpiperazine | | (pH 5.8) | peak, s), |
| | methylpiperazin- | | | | 3.90-4.00 (1H, m), 3.65-3.75 (4H, |
| | 1- | | | | m), 2.55-2.65 (4H, m), |
| | yl)pyrimidine- | | | | 2.40 (3H, s), |
| | 2,4-diamine | | | | 1.95-2.10 (2H, m), |
| | | | | | 1.95 (6H, s, 2 × AcOH), |
| | | | | | 1.50-1.85 (6H, m) |
| 143 | N⁴- | methylaminocyclopentane, | Diacetate | 291 [M + H]⁺ | CD₃OD 4.95 (1H, |
| | cyclopentyl- | N- | | RT 2.33 mins | obscured by H₂O |
| | N⁴-methyl-6- | methylpiperazine | | (pH 5.8) | peak, s), |
| | (4- | | | | 4.75-4.90 (1H, m), 3.60-3.70 (4H, |
| | methylpiperazin- | | | | m), 2.90 (3H, s), |
| | 1- | | | | 2.65-2.75 (4H, m), 2.50 (3H, |
| | yl)pyrimidine- | | | | s), 1.95 (6H, s, 2 × AcOH), |
| | 2,4-diamine | | | | 1.70-1.95 (4H, |
| | | | | | m), 1.55-1.70 (4H, m) |
| 144 | N⁴- | cycloheptylamine, | Acetate | 305 [M + H]⁺ | CD₃OD 4.95 (1H, |
| | cycloheptyl- | N- | | RT 2.43 mins | obscured by H₂O |
| | 6-(4- | methylpiperazine | | (pH 5.8) | peak, s), |
| | methylpiperazin- | | | | 3.65-3.75 (1H, m), 3.60-3.70 (4H, |
| | 1- | | | | m), 2.55-2.65 (4H, m), |
| | yl)pyrimidine- | | | | 2.40 (3H, s), |
| | 2,4-diamine | | | | 1.90-2.05 (2H, m), 1.95 (3H, s, |
| | | | | | AcOH), |
| | | | | | 1.50-1.75 (10H, m) |
| 145 | 1-[2-amino-6- | 4-(4- | Acetate | 403 [M + H]⁺ | CD₃OD 7.48 (2H, m), |
| | (4- | chlorophenyl)piperidin- | | RT 2.44 mins | 7.33 (2H, m), 4.90 (1H, |
| | methylpiperazin- | 4-ol, N- | | (pH 5.8) | s), 4.15-4.25 (2H, m), |
| | 1- | methylpiperazine | | | 3.62 (4H, m), |
| | yl)pyrimidin- | | | | 3.31-3.40 (2H, m), 2.67 (4H, m), |
| | 4-yl]-4-(4- | | | | 2.46 (3H, s), |
| | chlorophenyl)piperidin- | | | | 2.00-2.09 (2H, m), 1.98 (3H, s), |
| | 4-ol | | | | 1.71-1.80 (2H, m) |
| 146 | 4-(4- | 3- | Acetate | 353 [M + H]⁺ | CD₃OD 7.28-7.36 (3H, |
| | methylpiperazin- | phenylpiperidine, | | RT 2.99 mins | m), 7.19-7.26 (1H, m), |
| | 1-yl)-6-(3- | N- | | (pH 5.8) | 4.94 (1H, s), |
| | phenylpiperidin- | methylpiperazine | | | 4.25-4.44 (2H, m), 3.60 (4H, m), |
| | 1- | | | | 2.85-2.98 (2H, m), |
| | yl)pyrimidin- | | | | 2.60-2.80 (5H, m), |
| | 2-amine | | | | 2.43 (3H, s), 1.61-2.08 (7H) |
| 147 | trans-4-{[2- | trans-4-amino-N- | Acetate | 410 [M + H]⁺ | CD₃OD 7.56 (2H, d), |
| | amino-6-(4- | phenylcyclohexanecarboxamide | | RT | 7.31 (2H, t), 7.10 (1H, |
| | methylpiperazin- | hydrochloride | | 2.15 mins | t), 4.90 (1H, s), |
| | 1- | (CAS RN | | (pH 5.8) | 3.48-3.70 (5H, m), 2.60 (4H, |
| | yl)pyrimidin- | 412290-68-7), N- | | | m), 2.33-2.47 (4H, m), |
| | 4-yl]amino}- | methylpiperazine | | | 2.13 (2H, m), |
| | N- | | | | 1.92-2.04 (5H, m), 1.73 (2H, m), |
| | phenylcyclohexanecarboxamide | | | | 1.37 (2H, m) |
| 148 | cis-4-{[2- | Intermediate 66, | Acetate | 410 [M + H]⁺ | CD₃OD 7.56 (2H, d), |
| | amino-6-(4- | N- | | RT | 7.31 (2H, t), 7.10 (1H, |
| | methylpiperazin- | methylpiperazine | | 2.30 mins | t), 4.90 (1H, s), |
| | 1- | | | (pH 5.8) | 3.92 (1H, m), 3.58 (4H, m), |
| | yl)pyrimidin- | | | | 2.47-2.58 (5H, m), |
| | 4-yl]amino}- | | | | 2.38 (3H, s), |
| | N- | | | | 1.68-2.00 (11H, m) |
| | phenylcyclohexanecarboxamide | | | | |
| 149 | (1R*,3S*)-3- | Intermediate 67, | Acetate | 410 [M + H]⁺ | CD₃OD 7.55 (2H, d), |
| | {[2-amino-6- | N- | | RT | 7.31 (2H, t), 7.10 (1H, |
| | (4- | methylpiperazine | | 2.26 mins | t), 4.90 (1H, s), |
| | methylpiperazin- | | | (pH 5.8) | 3.54-3.76 (5H, m), |
| | 1- | | | | 2.48-2.65 (5H, m), 2.40 (3H, s), |
| | yl)pyrimidin- | | | | 1.86-2.20 (6H, m), |
| | 4-yl]amino}- | | | | 1.15-1.61 (5H, m) |
| | N- | | | | |
| | phenylcyclohexanecarboxamide | | | | |
| 150 | trans-4-{[2- | Intermediate 68, | Acetate | 348 [M + H]⁺ | CD₃OD 4.89 (1H, s), |
| | amino-6-(4- | N- | | RT | 3.48-3.67 (5H, m), |
| | methylpiperazin- | methylpiperazine | | 1.53 mins | 2.72 (3H, s), 2.58 (4H, m), |
| | 1- | | | (pH 5.8) | 2.40 (3H, s), |
| | yl)pyrimidin- | | | | 2.01-2.35 (3H, m), 1.97 (3H, s), |
| | 4-yl]amino}- | | | | 1.89 (2H, m), |
| | N- | | | | 1.55-1.71 (2H, m), 1.20-1.36 (2H, |
| | methylcyclohexanecarboxamide | | | | m) |
| 151 | trans-4-{[2- | Intermediate 69, | Acetate | 374 [M + H]⁺ | CD₃OD 4.88 (1H, s), |
| | amino-6-(4- | N- | | RT | 3.46-3.70 (5H, m), |
| | methylpiperazin- | methylpiperazine | | 1.63 mins | 2.50-2.74 (5H, m), |
| | 1- | | | (pH 5.8) | 2.39 (3H, s), 2.01-2.21 (3H, |
| | yl)pyrimidin- | | | | m), 1.97 (3H, s), |
| | 4-yl]amino}- | | | | 1.85 (2H, m), 1.55-1.71 (2H, |
| | N- | | | | m), 1.15-1.37 (2H, m), |
| | cyclopropylcyclohexane- | | | | 0.72 (2H, m), 0.49 (2H, |
| | carboxamide | | | | m) |
| 152 | trans-4-{[2- | Intermediate 70, | Acetate | 390 [M + H]⁺ | CD₃OD 4.90 (1H, s), |
| | amino-6-(4- | N- | | RT | 3.64 (4H, m), |
| | methylpiperazin- | methylpiperazine | | 1.95 mins | 3.45-3.58 (1H, m), 2.58 (4H, m), |
| | 1- | | | (pH 5.8) | 2.40 (3H, s), |
| | yl)pyrimidin- | | | | 2.01-2.21 (3H, m), 1.97 (3H, s), |
| | 4-yl]amino}- | | | | 1.83 (2H, m), |
| | N-tert- | | | | 1.51-1.69 (2H, m), |
| | butylcyclohexanecarboxamide | | | | 1.20-1.40 (11H, m) |
| 153 | trans-4-{[2- | Intermediate 71, | Acetate | | CD₃OD 7.45 (2M, d), |
| | amino-6-(4- | N- | | | 6.88 (2H, d), 4.88 (1H, |
| | methylpiperazin- | methylpiperazine | | | s), 3.79 (3H, s), |
| | 1- | | | | 3.55-3.67 (5H, m), 2.54 (4H, |
| | yl)pyrimidin- | | | | m), 2.39-2.44 (4H, m), |
| | 4-yl]amino}- | | | | 2.13 (2H, m), |
| | N-(4- | | | | 1.91-2.03 (5H, m), 1.52-1.71 (2H, |
| | methoxyphenyl)cyclohexane- | | | | m), 1.24-1.41 (2H, m) |
| | carboxamide | | | | |
| 154 | 6-(4- | 1- | | 368 [M + H]⁺ | CD₃OD 7.25 (2H, t), |
| | methylpiperazin- | phenylpiperidin- | | RT | 7.02 (2H, d), 6.85 (1H, |
| | 1-yl)-N⁴- | 4-amine (CAS | | 2.44 mins | t), 4.90 (1H, s), |
| | (1- | RN 63921-23-3), | | (pH 5.8) | 3.78 (1H, m), 3.65 (2H, m), |
| | phenylpiperidin- | N- | | | 3.54 (4H, m), 2.89 (2H, |
| | 4- | methylpiperazine | | | m), 2.53 (4H, m), |
| | yl)pyrimidine- | | | | 2.37 (3H, s), 2.08 (2H, m), |
| | 2,4-diamine | | | | 1.65 (2H, m) |
| 155 | 6-(4- | tetrahydro-2H- | Acetate | 293 [M + H]⁺ | CD₃OD 4.91 (1H, s), |
| | methylpiperazin- | pyran-4-amine | | RT | 3.97 (2H, m), 3.81 (1H, |
| | 1-yl)-N⁴- | hydrochloride | | 1.67 mins | m), 3.61 (4H, m), |
| | (tetrahydro- | (CAS RN 33024- | | (pH 5.8) | 3.54 (2H, m), 2.61 (4H, m), |
| | 2H-pyran-4- | 60-1), N- | | | 2.42 (3H, s), |
| | yl)pyrimidine- | methylpiperazine | | | 1.90-2.01 (5H, m), 1.55 (2H, m) |
| | 2,4-diamine | | | | |
| 156 | N⁴-(1- | 1- | Diacetate | 382 [M + H]⁺ | CD₃OD 7.32-7.45 (5H, |
| | benzylpiperidin- | benzylpiperidin- | | RT | m), 4.92 (1H, s), |
| | 4-yl)-6-(4- | 4-amine (CAS | | 2.05 mins | 3.83 (2H, s), 3.55-3.78 (5H, |
| | methylpiperazin- | RN 50541-93-0), | | (pH 5.8) | m), 3.10 (2H, m), |
| | 1- | N- | | | 2.48-2.65 (6H, m), 2.41 (3H, |
| | yl)pyrimidine- | methylpiperazine | | | s), 1.95-2.10 (8H, m), |
| | 2,4-diamine | | | | 1.65 (2H, m) |
| 157 | N⁴,N⁴- | dimethylamine, | Acetate | 237 [M + H]⁺ | CD₃OD 4.92 (1H, s), |
| | dimethyl-6- | N- | | RT | 3.62 (4H, m), 3.05 (6H, |
| | (4- | methylpiperazine | | 1.73 mins | s), 2.66 (4H, m), |
| | methylpiperazin- | | | (pH 5.8) | 2.45 (3H, s), 1.97 (3H, s) |
| | 1- | | | | |
| | yl)pyrimidine- | | | | |
| | 2,4-diamine | | | | |
| 158 | 4-[(1R*,5S*)- | (1R*,5S*)-8- | | 303 [M + H]⁺ | CDCl₃ 5.09 (1H, s), |
| | 8- | azabicyclo[3.2.1]octane | | RT 2.19 mins | 4.31-4.58 (4H, m), |
| | azabicyclo[3.2.1]oct- | (CAS RN | | (pH | 3.46-3.59 (4H, m), |
| | 8-yl]- | 6760-99-2), N- | | 5.8) | 2.40-2.50 (4H, m), |
| | 6-(4- | methylpiperazine | | | 2.32 (3H, s), |
| | methylpiperazine- | | | | 1.20-2.10 (10H, m) |
| | 1- | | | | |
| | yl)pyrimidin- | | | | |
| | 2-amine | | | | |
| 159 | 4-[4-(2- | 4-(2- | | 371 [M + H]⁺ | d₆ DMSO |
| | fluorophenyl)piperidin- | fluorophenyl)piperidine | | RT 2.82 min | 7.10-7.35 (4H, m), 5.10 (2H, br |
| | 1- | (CAS RN | | (pH | s), 5.35 (1H, s), |
| | yl]-6-(4- | 180161-17-5), N- | | 5.8) | 4.46 (2H, m), 3.40 (4H, m), |
| | methylpiperazin- | methylpiperazine | | | 3.05 (1H, m), 2.78 (2H, |
| | 1- | | | | m), 2.32 (4H, m), |
| | yl)pyrimidin- | | | | 2.22 (3H, s), 1.73 (2H, m), |
| | 2-amine | | | | 1.60 (2H, m) |
| 160 | 4-[3-(3- | 3-(3- | | 371 [M + H]⁺ | d₆ DMSO 7.35 (1H, |
| | fluorophenyl)piperidin- | fluorophenyl)piperidine | | RT 3.05 min | m), 7.18 (2H, m), |
| | 1- | (CAS RN | | (pH | 7.05 (1H, m), 5.60 (2H, s), |
| | yl]-6-(4- | 343856-71-3), N- | | 5.8) | 5.32 (1H, s), 4.40 (1H, |
| | methylpiperazin- | methylpiperazine | | | m), 4.30 (1H, m), |
| | 1- | | | | 3.45 (4H, m), 2.65-2.80 (3H, |
| | yl)pyrimidin- | | | | m), 2.30 (4H, m), |
| | 2-amine | | | | 2.20 (3H, s), 1.90 (1H, m), |
| | | | | | 1.70 (2H, m), 1.50 (1H, |
| | | | | | m) |
| 161 | 4-(4- | 4-[2- | | 421 [M + H]⁺ | d₆ DMSO 7.68 (1H, |
| | methylpiperazin- | (trifluoromethyl)phenyl]piperidine | | RT 3.39 min | m), 7.65 (2H, m), |
| | 1-yl)-6-{4- | (CAS RN | | (pH | 7.40 (1H, m), 5.62 (2H, s), |
| | [2- | 308823-90-7), N- | | 5.8) | 5.35 (1H, s), 4.50 (2H, |
| | (trifluoromethyl)phenyl]piperidin- | methylpiperazine | | | m), 3.45 (4H, m), |
| | 1- | | | | 3.08 (1H, m), 2.76 (2H, m), |
| | yl}pyrimidin- | | | | 2.33 (4H, m), 2.21 (3H, |
| | 2-amine | | | | s), 1.70 (4H, m) |
| 162 | 4-[4-(2- | 4-(2- | | 367 [M + H]⁺ | d₆ DMSO |
| | methylphenyl)piperidin- | methylphenyl)piperidine | | RT 3.11 min | 7.03-7.20 (4H, m), 5.58 (2H, s), |
| | 1- | (CAS RN | | (pH | 5.34 (1H, s), 4.45 (2H, |
| | yl]-6-(4- | 630116-52-8), N- | | 5.8) | m), 3.45 (4H, m), |
| | methylpiperazin- | methylpiperazine | | | 2.95 (1H, m), 2.82 (2H, m), |
| | 1- | | | | 2.33 (3H, s), 2.30 (4H, |
| | yl)pyrimidin- | | | | m), 2.20 (3H, s), |
| | 2-amine | | | | 1.70 (2H, m), 1.50 (2H, m) |

Comp. No means Compound Number

### Example 21

### Synthesis of 4-isopropyl-6-(4-methylpiperazin-1-yl)pyrimidin-2-amine (Compound 163)

A solution of 4-chloro-6-isopropylpyrimidin-2-amine (CAS RN73576-33-7) (32 mg) and N-methylpiperazine (19 mg) in ethanol (0.75 ml) is heated to 150° C. for 10 mins in a microwave. The solution is concentrated in vacuo and partitioned between dichloromethane and 10% potassium carbonate solution. The EtOAc solution is dried over MgSO₄, filtered, and concentrated in vacuo to afford the title compound as a yellow oil (45 mg 100%). LCMS 236 [M+H]⁺, RT 1.72 mins (pH 5.8). ¹H NMR 300 MHz (CDCl₃) (δ ppm): 5.8 (1H, s), 4.9 (2H, br s), 3.6 (4H, m), 2.7 (1H, m), 2.45 (4H, m), 2.35 (3H, s), 1.2 (6H, d).

### Example 22

### Synthesis of tert-butyl [1-(2-amino-6-cyclopentylpyrimidin-4-yl)pyrrolidin-3-yl]carbamate di-formate salt (Intermediate 59)

Intermediate 9 (93 mg), aqueous ammonium hydroxide (0.6 ml) and 2.0M ammonia in EtOH (1.2 ml) are combined and heated under microwave irradiation at 150° C. for 135 mins. Concentration of the solution in vacuo and purification by preparative HPLC (pH 2.5) affords the title compound as a colorless solid as its di-formate salt (11 mg, 13%). LCMS 348 [M+H]⁺, RT 2.11 mins (Method A). ¹H NMR 300 MHz (CDCl₃) (δ ppm): 8.40 (HCOOH), 5.65 (1H, d), 5.50 and 5.00 (1H, 2×br s), 4.30 (1H, m), 3.29-3.90 (4H, m), 3.00 (1H, m), 1.32-2.49 (12H, m), 1.45 (9H, s).

### Example 23

### Synthesis of 4-(3-aminopyrrolidin-1-yl)-6-cyclopentylpyrimidin-2-amine (Compound 164)

A solution of Intermediate 59 (11 mg) and TFA (0.5 ml) in DCM (3 ml) is stirred at RT for 1 hr. Purification by preparative HPLC (Method B) followed by a DCM/saturated NaHCO₃ partition affords the title compound as a colorless glass (3.7 mg, 47%). LCMS 248 [M+H]⁺, RT 1.37 mins (pH 5.8). ¹H NMR 300 MHz (CDCl₃) (δ ppm): 5.62 (1H, s), 5.21 (2H, br s), 3.40-3.76 (4H, m), 3.19 (1H, br m), 2.85 (1H, m), 1.58-2.35 (12H, m).

### Example 24

### Synthesis of 4-(3-aminopyrrolidin-1-yl)-6-(5-fluoro-1,3-dihydro-2H-isoindol-2-yl)pyrimidin-2-amine (Compound 165)

Compound 165 is prepared from 4,6-dichloropyrimidin-2-amine. A mixture of 5-fluoroisoindoline (0.70 g) and 4,6-dichloropyrimidin-2-amine (1 g) in NMP (2 ml) and triethylamine (2 ml) is heated under microwave irradiation at 100° C. A solution of 3-(tert-butoxycarbonylamino)pyrrolidine (1 g) in NMP (3 ml) is added and the mixture heated at 150° C. for 30 mins. The mixture is cooled, added to water (20 ml) and extracted with EtOAc (20 ml). The solvent is washed with water (2×20 ml), dried and evaporated and the crude product dissolved in DCM (30 ml) and TFA (10 ml). The solution is stirred for 1 hr, then evaporated in vacuo and azeotroped with heptane (2×30 ml). The crude product is dissolved in water (30 ml) and washed with EtOAc. The aqueous layer is basified with solid potassium carbonate (4 g) and extracted with EtOAc. The solvent is washed with water (20 ml), dried and evaporated to give the title compound as white solid (70 mg). LCMS 315 [M+H]⁺, RT 2.08 mins. ¹H NMR 300 MHz (DMSO) (δ ppm): 7.40 (1H, m), 7.22 (1H, m), 7.15 (1H, m), 5.51 (2H, br s), 4.75 (1H, s), 4.65 (4H, m), 3.50 (3H, m), 3.10 (1H, m), 2.20 (1H, m), 2.00 (2H, br s), 1.90 (1H, m), 1.65 (1H, m).

Compounds 166 and 167 are prepared in a similar manner to the method described for Compound 165 in example 24. The reagents used and the results obtained are tabulated below (Table 12). The free base of the compounds is obtained unless otherwise stated.

TABLE 12

| Comp. | IUPAC | | | | ¹H NMR |
|---|---|---|---|---|---|
| No | Name | Starting Materials | Salt | LCMS | (Solvent, δ ppm) |
| 166 | 6-[(3S)-3- | 4,6- | Diacetate | 289 [M + H]⁺ | CD₃OD 5.00 (1H, |
| | aminopyrrolidin- | dichloropyrimidin-2- | | RT 2.06 mins | obscured by H₂O |
| | 1-yl]- | amine, | | (pH 5.8) | peak, s), |
| | N⁴- | (1R*,2R*,4S*)- | | | 3.90-4.00 (1H, m), |
| | [(1R*,2R*,4S*)- | bicyclo[2.2.1]heptan- | | | 3.75-3.85 (1H, m), |
| | bicyclo[2.2.1]hept- | 2-amine, tert-butyl | | | 3.55-3.75 (3H, m), |
| | 2- | (3S)-pyrrolidin-3- | | | 3.35-3.40 (1H, m), |
| | yl]pyrimidine- | ylcarbamate, | | | 2.35-2.50 (1H, m), |
| | 2,4-diamine | | | | 2.25-2.35 (2H, m), |
| | | | | | 2.05-2.20 (1H, m), |
| | | | | | 1.95 (6H, s, 2 × AcOH), |
| | | | | | 1.80-1.90 (1H, m), |
| | | | | | 1.50-1.65 (3H, m), |
| | | | | | 1.35-1.50 (1H, m), |
| | | | | | 1.15-1.35 (3H, m) |
| 167 | 6-[(3R)-3- | 4,6- | Diacetate | 289 [M + H]⁺ | CD₃OD 4.95 (1H, |
| | aminopyrrolidin- | dichloropyrimidin-2- | | RT 2.06 mins | obscured by H₂O |
| | 1-yl]- | amine, | | (pH 5.8) | peak, s), |
| | N⁴- | (1R*,2R*,4S*)- | | | 3.90-4.00 (1H, m), |
| | [(1R*,2R*,4S*)- | bicyclo[2.2.1]heptan- | | | 3.75-3.85 (1H, m), |
| | bicyclo[2.2.1]hept- | 2-amine, tert-butyl | | | 3.50-3.70 (3H, m), |
| | 2- | (3R)-pyrrolidin-3- | | | 3.35-3.45 (1H, m), |
| | yl]pyrimidine- | ylcarbamate, | | | 2.35-2.45 (1H, m), |
| | 2,4-diamine | | | | 2.25-2.35 (2H, m), |
| | | | | | 2.05-2.20 (1H, m), |
| | | | | | 1.95 (6H, s, 2 × AcOH), |
| | | | | | 1.75-1.90 (1H, m), |
| | | | | | 1.50-1.65 (3H, m), |
| | | | | | 1.35-1.45 (1H, m), |
| | | | | | 1.15-1.35 (3H, m) |

Comp. No means Compound Number

### Example 25

### Synthesis of tert-butyl 4-{[2-amino-6-(4-methylpiperazin-1-yl)pyrimidin-4-yl]amino}piperidine-1-carboxylate (Intermediate 60)

4,6-Dichloropyrimidin-2-amine (500 mg), tert-butyl 4-aminopiperidin-1-ylcarbamate (611 mg) and TEA (1.27 ml) are combined in NMP (2 ml) and heated in the microwave to 160° C. for 30 mins. N-Methylpiperazine (1.01 ml) is then added and the reaction heated for a further 45 mins at 175° C. The reaction mixture is diluted with EtOAc (60 ml) and washed with brine (4×20 ml). The combined organic layers are dried over MgSO₄, filtered and evaporated. The residue is purified by column chromatography on silica, eluting with DCM-10%MeOH/DCM to afford the title compound as a cream colored solid (439 mg, 37%). LCMS 392 [M+H]⁺, RT 2.20 mins (pH 5.8). ¹H NMR 300 MHz (d₆-DMSO) (δ ppm): 6.15 (1H, d), 5.50 (2H, s), 5.00 (1H, s), 3.90-3.75 (3H, m), 3.40-3.30 (4H, m) (obscured by H₂O peak), 2.90-2.75 (2H, m), 2.35-2.25 (4H, m), 2.20 (3H, s), 1.80-1.70 (2H, m), 1.40 (9H, s), 1.30-1.15 (2H, m).

### Example 26

### Synthesis of 6-(4-methylpiperazin-1-yl)-N⁴-piperidin-4-yl pyrimidine-2,4-diamine (Compound 168)

A mixture of Intermediate 60 (300 mg) and TFA (2 ml) in DCM (10 ml) is stirred at room temperature for 3½ hrs. The reaction mixture is evaporated and taken up in H₂O (3 ml) then basified to pH12 with saturated NaHCO₃ (aq) solution and 2M NaOH(aq) and saturated with NaCl. The aqueous layer is then extracted with DCM (4×20 ml) then 5% MeOH/DCM (20 ml). The combined organic layers are dried over MgSO₄, filtered, and evaporated to afford the title compound as a cream colored solid (202 mg, 90%). LCMS 292 [M+H]⁺ (pH 5.8), RT 0.80 mins. ¹H NMR 300 MHz (d₆-DMSO) (δ ppm): 6.05 (1H, d), 5.45 (2H, s), 5.00 (1H, s), 3.75-3.60 (1H, s), 3.40-3.25 (4H, m) (obscured by H₂O peak), 2.95-2.85 (2H, m), 2.55-2.40 (2H, m) (partially obscured by DMSO peak), 2.35-2.25 (4H, m), 2.20 (3H, s), 1.80-1.70 (2H, m), 1.30-1.15 (2H, m).

### Example 27

### Synthesis of N⁴-(1-acetylpiperidin-4-yl)-6-(4-methylpiperazin-1-yl)pyrimidine-2,4-diamine (Compound 169)

Acetic anhydride (211 μl) is added to a suspension of Compound 168 (60 mg) and DIPEA (39 μl) in DCM (4 ml) under N₂ atmosphere. The mixture is stirred at room temperature for 18 hrs then diluted with DCM (10 ml) and washed with saturated NaHCO₃ (aq) solution (20 ml). The aqueous phase is extracted with DCM (2×15 ml) then the combined extracts are dried over MgSO₄, filtered, and evaporated. Purification of the residue by column chromatography on silica, eluting with DCM-10%MeOH/DCM-1%NH₄OH/10%MeOH/DCM, affords the title compound as a white solid (19.5 mg, 28%). LCMS 334 [M+H]⁺ (pH5.8), RT 1.55 mins. ¹H NMR 300 MHz (d₆-DMSO) (δ ppm): 6.15 (1H, d), 5.50 (2H, s), 5.05 (1H, s), 4.25-4.15 (1H, m), 3.95-3.70 (2H, m, br), 3.40-3.30 (4H, m) (obscured by H₂O peak), 3.15-3.05 (1H, m), 2.75-2.65 (1H. m), 2.35-2.25 (4H, m), 2.20 (3H, s), 2.00 (3H, s), 1.90-1.75 (2H, m), 1.35-1.10 (2H, m, br).

### Example 28

### Synthesis of N⁴-[1-(3-methoxybenzoyl)piperidin-4-yl]-6-(4-methylpiperazin-1-yl)pyrimidine-2,4-diamine (Compound 170)

3-Methoxybenzoyl chloride (30 μl) is added to a suspension of Compound 168 (60 mg) and DIPEA (38 μl) in DCM under N₂ atmosphere. The mixture is stirred at room temperature for 23 hrs then diluted with DCM (10 ml) and washed with saturated NaHCO₃ (aq) solution (20 ml). The aqueous phase is extracted with DCM (25 ml) and the combined organic extracts are dried over MgSO₄, filtered and evaporated. Purification of the residue by column chromatography on silica, eluting with DCM-10%MeOH/DCM-1%NH₄OH/10%MeOH/DCM, affords the title compound as a white solid (71 mg, 79%). LCMS) 426 [M+H]⁺ (pH5.8, RT 2.06 mins. ¹H NMR 300 MHz (d₆-DMSO) (δ ppm): 7.40-7.30 (1H, m), 7.00 (1H, dd), 6.95-6.85 (2H, m), 6.15 (1H, d), 5.50 (2H, s), 5.05 (1H, s), 4.40-4.25 (1H, m, br), 4.00-3.85 (1H, m, br), 3.80 (3H, s), 3.65-3.45 (1H, m, br), 3.40-3.25 (4H, m) (obscured by H₂O peak), 3.20-2.90 (2H, m, br), 2.40-2.25 (4H, m), 2.20 (3H, s), 1.95-1.70 (2H, m, br), 1.40-1.20 (2H, m, br).

### Example 29

### Synthesis of 4-chloro-6-[(E)-2-phenylvinyl]pyrimidin-2-amine (Intermediate 61)

4,6-Dichloropyrimidin-2-amine (375 mg) and (E)-phenylethenyl boronic acid (355 mg) are partially dissolved in THF (10 ml) and a solution of sodium carbonate (anhydrous, 339 mg) in water (1 ml) is added. The solution is degassed by bubbling nitrogen through it for 15 mins, then Pd(PPh₃)₄ is added (ca. 5 mg) and the solution is heated at 78° C. under N₂ for 18 hrs. The solution is then diluted with MTBE (30 ml) and washed with water (15 ml), the aqueous phase backwashed with fresh MTBE (20 ml), the combined organic layers are dried (MgSO₄) and concentrated to dryness in vacuo. Purification by flash chromatography, eluting with EtOAc-Heptane 1:4, then 1:3, affords the title compound as a colorless solid (339 mg, 64%). R_{f} (EtOAc-Heptane 1:4) 0.35. LCMS 232 [M+H]⁺, RT 3.53 mins (pH 2.5). ¹H NMR 300 MHz (CDCl₃) (δ ppm): 7.76 (1H.d), 7.56 (2H, dd), 7.38 (3H, m), 6.87 (1H, d), 6.71 (1H, s), 5.12 (2H, brs).

### Example 30

### Synthesis of tert-butyl (1-{2-amino-6-[(E)-2-phenylvinyl]pyrimidin-4-yl}pyrrolidin-3-yl)carbamate (Intermediate 62)

Intermediate 61 (339 mg), 3-(tert-butoxycarbonylamino)pyrrolidine (300 mg) and triethylamine (0.225 ml) are dissolved in dry NMP (3 ml) and heated at 120° C. for 30 mins under microwave irradiation. The solution is then diluted with MTBE (15 ml) and washed with saturated brine (3×8 ml), dried (MgSO₄) and concentrated to dryness in vacuo. Purification of the crude product by flash chromatography, eluting with DCM-MeOH 95:5 affords the title compound as a yellow foam (596 mg, quant.) R_{f} (DCM-MeOH 95:5) 0.23. LCMS 382 [M+H]⁺, RT 2.33 mins (pH 2.5). ¹H NMR 300 MHz (CDCl₃) (δ ppm): 7.66 (1H, d), 7.54 (2H, dd), 7.21-7.40 (3H, m), 6.83 (1H, d), 5.80 (1H, s), 4.69 (2H, br s), 4.32 (1H, m), 3.73 (1H, m), 3.55 (2H, m), 1.89-2.41 (4H, m), 1.43 (9H, s).

### Example 31

### Synthesis of 4-(3-aminopyrrolidin-1-yl)-6-[(E)-2-phenylvinyl]pyrimidin-2-amine (Compound 171)

Intermediate 62 (68.7 mg) in DCM (3 ml) is treated with TFA (0.6 ml) and the solution is allowed to stand at room temperature for 18 hrs. The solution is then concentrated to dryness in vacuo, redissolved in DCM (25 ml) and neutralized with saturated NaHCO₃ (2 ml). A solid separated from solution, which is filtered, washed with DCM and dried in vacuo. Purification by preparative HPLC (Method B), followed by dissolving the residue obtained in EtOAc and washing with a small amount of NaHCO₃ solution, drying (MgSO₄) and concentrating to dryness in vacuo, affords the title compound as a yellow solid (11.1 mg, 22%). LCMS 282 [M+H]⁺, RT 2.05 mins (pH 5.8). ¹H NMR 300 MHz (CDCl₃) (δ ppm): 7.65 (1H, d), 7.54 (2H, dd), 7.20-7.42 (3H, m), 6.82 (1H, d), 5.81 (1H, s), 5.32 (2H, br s), 3.08-3.80 (5H, br m), 2.50 (2H, br s), 2.20 (1H, m), 1.81 (1H, m).

### Example 32

### Synthesis of 4-chloro-6-(4-methylcyclohex-1-en-1-yl)pyrimidin-2-amine (Intermediate 63)

4,6-Dichloropyrimidin-2-amine (273 mg) and 4-methylcyclohexen-1-yl boronic acid (256 mg) are partially dissolved in THF (5 ml) and a solution of Na₂CO₃ (anhydrous, 246 mg) in H₂O (1.16 ml) is added. The solution is degassed by bubbling nitrogen through it for 15 mins, then Pd(PPh₃)₄ is added (ca. 5 mg) and the solution is heated at 80° C. under N₂ for 18 hrs. The solution is then diluted with MTBE (20 ml) and washed with H₂O (5 ml), the aqueous phase backwashed with fresh MTBE (10 ml). The combined organic layers are dried (MgSO₄) and concentrated to dryness in vacuo to afford the title compound as a yellow solid (405 mg, quant.). LCMS 224 [M+H]⁺, RT 3.98 mins (pH 2.5). ¹H NMR 300 MHz (CDCl₃) (δ ppm): 6.89 (1H, m), 6.69 (1H, s), 5.07 (2H, br s), 2.25-2.54 (3H, m), 1.64-1.94 (3H, m), 1.34 (1H, m), 1.01 (3H, d).

### Example 33

### Synthesis of tert-butyl {1-[2-amino-6-(4-methylcyclohex-1-en-1-yl)pyrimidin-4-yl]pyrrolidin-3-yl}carbamate (Intermediate 64)

Intermediate 63 (66.5 mg), 3-(tert-butoxycarbonylamino)pyrrolidine (68 mg) and triethylamine (0.049 ml) are dissolved in absolute EtOH (3 ml) and heated at 120° C. for 50 mins under microwave irradiation. The solution is then concentrated to dryness in vacuo. Purification of the crude product by flash chromatography, eluting with DCM-MeOH 97:3 then 95:5 affords the title compound as a pale orange solid (81 mg, 72%) R_{f} (DCM-MeOH 95:5) 0.42. LCMS 374 [M+H]⁺, RT 2.40 mins (pH 2.5). ¹H NMR 300 MHz (CDCl₃) (δ ppm): 6.90 (1H, m), 5.70 (1H, s), 5.31 (2H, br s), 4.91 (1H, br m), 4.30 (1H, br m), 3.70 (1H, m), 3.55 (2H, m), 3.38 (1H, m), 2.15-2.48 (4H, m), 1.62-2.01 (4H, m), 1.45 (9H, s), 1.32 (1H, m), 1.00 (3H, s).

### Example 34

### Synthesis of 4-(3-aminopyrrolidin-1-yl)-6-(4-methylcyclohex-1-en-1-yl)pyrimidin-2-amine di-trifluoroacetic acid salt (Compound 172)

Intermediate 64 (81 mg) in DCM (5 ml) is treated with TFA (1.0 ml) and the solution is allowed to stand at room temperature for 4 hrs. The solution is then concentrated to dryness in vacuo, redissolved in DCM (20 ml) and washed with saturated NaHCO₃ (3 ml). The aqueous phase is then extracted with EtOAc (10 ml), the combined organic layers are dried (MgSO₄) and concentrated to dryness in vacuo. The residual solid is then redissolved in MeOH (15 ml) and filtered through a pad of Kieselguhr. The filtrate is concentrated to dryness in vacuo to afford the title compound as a orange solid as its di-TFA salt (98 mg, 90%). LCMS 274 [M+H]⁺, RT 2.11 mins (pH 5.8). ¹H NMR 300 MHz (CD₃OD) (δ ppm): 6.69 (1H, m), 6.10 (1H, s rotamers), 3.69-4.19 (5H, m), 2.13-2.62 (5H, m), 1.66-2.00 (3H, m), 1.39 (1H, m), 1.05 (3H, d).

### Example 35

### Synthesis of 4-cyclohexyl-6-{3-[(methylamino)methyl]azetidin-1-yl}pyrimidin-2-amine (Compound 173)

LiAlH₄ (28 mg) is loaded into a round bottom flask. To this is added a solution of Intermediate 32 (46 mg) in THF (2.5 ml). The mixture is stirred and heated under reflux and under N₂ for 2 hrs and stirred overnight at room temperature. The mixture is cooled (ice-bath) and quenched with H₂O (100 μl), followed by 15% NaOH solution (100 μl) and more water (300 μl). The residue obtained is suspended in THF and filtered through Kieselguhr (washed with THF 2×20 ml). The filtrate is removed in vacuo to give a pale yellow residue. Purification by preparative HPLC (pH 5.8) affords the title compound as a colorless gum (11.2 mg, 32%). LCMS 276 [M+H]⁺, RT 1.84 mins (pH 5.8). ¹H NMR 300 MHz (MeOD) (δ ppm): 5.55 (1H, s), 4.15 (2H, dd), 3.70 (2H, dd), 2.80-2.95 (3H, m), 2.40 (3H, s), 2.25-2.35 (1H, m), 1.80-1.90 (4H, m), 1.70-1.80 (1H, m), 1.25-1.50 (5H, m).

Compounds 174 to 185 and Intermediate 65 are prepared in a similar manner to the method described for Compound 173 in Example 35.The reagents used and the results obtained are tabulated below (Table 13). The free base of the compounds is obtained unless otherwise stated.

TABLE 13

| Comp. | | Starting | | | ¹H NMR |
|---|---|---|---|---|---|
| No | IUPAC Name | Materials | Salt | LCMS | (Solvent, δ ppm) |
| 174 | 4-[3- | Interm. 83 | | 324 [M + H]⁺ | CD₃OD 6.98 (4H, |
| | (methylamino)pyrrolidin- | | | RT 2.09 mins | brs), 5.68 (1H, s), |
| | 1-yl]- | | | (pH 5.8) | 3.37-3.67 (5H, m), |
| | 6-(1,2,3,4- | | | | 2.77-2.91 (4H, m), |
| | tetrahydronaphthalen- | | | | 2.60-2.71 (1H, m), |
| | 2- | | | | 2.32 (3H, s), |
| | yl)pyrimidin-2- | | | | 1.96-2.18 (2H, m), |
| | amine | | | | 1.72-1.90 (2H, m) |
| 175 | 4-cyclohexyl- | Interm.19 | | 262 [M + H]⁺ | CDCl₃5.50 (1H, s), |
| | 6-[3- | | | RT 1.89 mins | 4.78 (2H, br s), |
| | (methylamino)azetidin- | | | (pH 5.8) | 4.25 (2H, m), |
| | 1- | | | | 3.70 (3H, m), 2.45 (3H, |
| | yl]pyrimidin-2- | | | | s), 2.30 (1H, m), |
| | amine | | | | 1.65-1.98 (5H, m), |
| | | | | | 1.20-1.49 (5H, m) |
| 176 | 4-cyclohexyl- | Interm.20 | | 302 [M + H]⁺ | CDCl₃ 5.65 (1H, s), |
| | 6- | | | RT 1.74 mins | 4.65 (2H, s), |
| | [(3aR*,6aS*)- | | | (pH 5.8) | 3.65 (2H, m), 3.40 (2H, |
| | 5- | | | | d), 2.95 (2H, br m), |
| | methylhexahydropyrrolo[3, | | | | 2.68 (2H, m), |
| | 4- | | | | 2.45 (2H, dd), 2.30 (3H, |
| | c]pyrrol-2(1H)- | | | | s), 2.29 (1H, m), |
| | yl]pyrimidin-2- | | | | 1.65-1.98 (5H, m), |
| | amine | | | | 1.20-1.49 (5H, m) |
| 177 | 6-cyclohexyl- | Interm. 22 | | 288 [M + H]⁺ | CDCl₃ 6.35 (1H, br |
| | N⁴- | | | RT 1.78 mins | s), 5.85 (1H, s), |
| | [(1R*,5S*,6S*)- | | | (pH 5.8) | 3.65 (2H, br s), |
| | 3-methyl-3- | | | | 3.23 (2H, d), |
| | azabicyclo[3.1.0]hex- | | | | 2.85 (1H, s), 2.50 (3H, |
| | 6- | | | | m), 2.35 (3H, s), |
| | yl]pyrimidine- | | | | 1.70-1.95 (5H, m), |
| | 2,4-diamine | | | | 1.63 (2H, s), |
| | | | | | 1.20-1.49 (5H, m) |
| 178 | 6-(4- | Interm. 60 | | 306 [M + H]⁺ | d₆ -DMSO |
| | methylpiperazin- | | | RT 1.42 mins | 6.05 (1H, d), 5.45 (2H, |
| | 1-yl)-N⁴-(1- | | | (pH 5.8) | s), 5.00 (1H, s), |
| | methylpiperidin- | | | | 3.55-3.70 (1H, br |
| | 4- | | | | s), 3.30-3.40 (4H, |
| | yl)pyrimidine- | | | | m) (obscured by |
| | 2,4-diamine | | | | H₂O peak), |
| | | | | | 2.65-2.75 (2H, m), |
| | | | | | 2.25-2.32 (4H, m), |
| | | | | | 2.20 (3H, s), 2.15 (3H, |
| | | | | | s), 1.87-2.00 (2H, |
| | | | | | m), 1.70-1.80 (2H, |
| | | | | | m), 1.30-1.45 (2H, |
| | | | | | m) |
| 179 | 4-[3- | Interm. 85 | | 298 [M + H]⁺ | CDCl₃ |
| | (methylamino)pyrrolidin- | | | RT 1.82 mins | 7.15-7.33 (5H, m), 5.55 (1H, |
| | 1-yl]- | | | (pH 5.8) | s), 4.85 (2H, bs), |
| | 6-(2- | | | | 3.08-3.87 (6H, |
| | phenylethyl)pyrimidin- | | | | m), 2.95 (2H, dd), |
| | 2- | | | | 2.75 (2H, dd), |
| | amine | | | | 2.45 (3H, s), 2.15 (1H, |
| | | | | | m), 1.82 (1H, m). |
| Interm. | (3S)-1-{6- | Interm. 5 | | 347 [M + H]⁺ | CDCl₃ 6.14 (1H, s), |
| 65 | [adamantan-2- | | | RT 2.31 mins | 3.30-3.65 (4H, |
| | yl]-2- | | | (pH 2.5) | bm), 2.83 (1H, s), |
| | chloropyrimidin- | | | | 2.53 (2H, s), |
| | 4-yl}-N- | | | | 2.48 (3H, s), 2.20 (1H, |
| | methylpyrrolidin- | | | | m), |
| | 3-amine | | | | 1.71-2.04 (14H, m). |
| 180 | 4-cyclohexyl- | Interm. 53 | | 290 [M + H]⁺ | CDCl₃ 5.80 (1H, s), |
| | 6-(3,4- | | | RT 2.23 mins | 4.83 (2H, s), |
| | dimethylpiperazin- | | | (pH 5.8) | 4.10 (2H, m), 3.05 (1H, |
| | 1- | | | | m), 2.83 (1H, m), |
| | yl)pyrimidin-2- | | | | 2.63 (1H, m), |
| | amine | | | | 2.30 (3H, s), |
| | | | | | 2.20-2.30 (2H, m), 2.10 (1H, |
| | | | | | m), 1.70-1.90 (5H, |
| | | | | | m), 1.20-1.45 (5H, |
| | | | | | m), 1.12 (3H, d) |
| 181 | N⁴-(2,3- | Interm. 55 | Triacetate | 325 [M + H]⁺ | CD₃OD |
| | dihydro-1H- | | | RT 2.14 mins | 7.13-7.28 (4H, m), 4.99 (1H, |
| | inden-2-yl)-6- | | | (pH 5.8) | s), 4.50 (1H, m), |
| | [3- | | | | 3.49-3.88 (5H, m), |
| | (methylamino)pyrrolidin- | | | | 3.38 (2H, m), |
| | 1- | | | | 2.94 (2H, m), 2.70 (3H, |
| | yl]pyrimidine- | | | | s), 2.42 (1H, m), |
| | 2,4-diamine | | | | 2.20 (1H, m), |
| | | | | | 1.95 (9H, s, 3 × AcOH) |

Comp. No means Compound Number
Interm. means Intermediate

### Example 36

### Synthesis of 4-(3-aminoazetidin-1-yl)-6-cyclohexyl]pyprimidin-2-amine (Compound 182)

Intermediate 19 (0.1 g) is added to a solution of hydrochloric acid (0.2 mM in methanol, 20 ml). The solution is stirred overnight at room temperature. The solvent is evaporated in vacuo. The residue is partitioned between dichloromethane and sodium hydroxide solution (2.0 M, 30 ml each). The dichloromethane layer is dried (MgSO₄) and evaporated in vacuo. Purification of the residue by crystallization from ethyl acetate affords the title compound as a colorless solid (0.65 g, 91%). LCMS 248 [M+H]⁺, RT 1.64 mins (pH 5.8). ¹H NMR 300 MHz (CDCl₃) 5.50 (1H, s), 4.68 (2H, br s), 4.25 (2H, t), 3.95 (1H, m), 3.65 (2H, dd), 2.35 (1H, m), 1.55-1.98 (5H, m), 1.15-1.49 (5H, m).

Intermediates 66 to 71 and Compounds 183 to 186 are prepared in a similar manner to the method described for Compound 182 in Example 36.The reagents used and the results obtained are tabulated below (Table 14). The free base of the compounds is obtained unless otherwise stated.

TABLE 14

| Comp. | | Starting | | | ¹H NMR |
|---|---|---|---|---|---|
| No | IUPAC Name | Materials | Salt | LCMS | (Solvent, δ ppm) |
| Interm. | cis-4-amino-N- | Interm. | Hydrochloride | | CDCl₃ 7.53 (1.4H, d), |
| 66 | phenylcyclohexanecarboxamide | 72 | | | 7.31 (1.4H, t), 7.05-7.20 (1.3H, |
| | | | | | m), 6.66-6.80 (0.9H, m), |
| | | | | | 3.07 (0.7H, m), |
| | | | | | 2.78-2.89 (0.3H, m), 2.49 (0.3H, m), |
| | | | | | 2.38 (0.7H, m), |
| | | | | | 1.95-2.10 (2H, m), 1.53-1.80 (5.3H, |
| | | | | | m), 1.30-1.45 (0.7H, m) |
| Interm. | (1R*,3S*)-3- | Interm. | Hydrochloride | | CDCl₃ 7.52 (1.4H, d), |
| 67 | amino-N- | 73 | | | 7.31 (1.4H, t), 7.05-7.20 (1.3H, |
| | phenylcyclohexanecarboxamide | | | | m), 6.67-6.80 (0.9H, m), |
| | | | | | 2.59-2.79 (1H, m), |
| | | | | | 2.24-2.41 (1H, m), |
| | | | | | 2.04-2.17 (1H, m), 1.79-1.99 (1H, m), |
| | | | | | 0.99-1.62 (6H, m) |
| Interm. | trans-4-amino-N- | Interm. | Hydrochloride | | CDCl₃ 2.60-2.85 (4H, m), |
| 68 | methylcyclohexanecarboxamide | 74 | | | 1.82-2.08 (3H, m), |
| | | | | | 1.02-1.68 (6H, m) |
| Interm. | trans-4-amino-N- | Interm. | Hydrochloride | | CDCl₃ 2.58-2.75 (2H, m), |
| 69 | cyclopropylcyclohexanecarboxamide | 75 | | | 1.80-2.01 (5H, m), |
| | | | | | 1.42-1.60 (2H, m), |
| | | | | | 0.99-1.19 (2H, m), 0.76 (2H, m), |
| | | | | | 0.45 (2H, m) |
| Interm. | trans-4-amino-N- | Interm. | Hydrochloride | | CDCl₃ 2.65 (1H, m), |
| 70 | tert- | 76 | | | 1.76-1.99 (5H, m), 1.50 (2H, m), |
| | butylcyclohexanecarboxamide | | | | 1.33 (9H, s), 1.09 (2H, m) |
| Interm. | trans-4-amino-N- | Interm. | Hydrochloride | | CDCl₃ 7.40 (2H, d), |
| 71 | (4- | 77 | | | 6.85 (2H, d), 3.78 (3H, s), |
| | methoxyphenyl)cyclohexanecarboxamide | | | | 2.71 (1H, m), 2.15 (1H, m), |
| | | | | | 1.91-2.05 (4H, m), |
| | | | | | 1.54-1.75 (2H, m), |
| | | | | | 1.07-1.25 (2H, m) |
| 183 | 4-(3- | Interm. | | 310 [M + H]⁺ | CD₃OD 7.09 (4H, brs), |
| | aminopyrrolidin- | 83 | | RT | 5.78 (1H, s), |
| | 1-yl)-6-(1,2,3,4- | | | 2.03 mins | 3.38-3.74 (4H, m), 3.20 (1H, br- |
| | tetrahydronaphthalen- | | | (pH 5.8) | hump), 2.87-3.02 (4H, m), |
| | 2- | | | | 2.70-2.82 (1H, m), |
| | yl)pyrimidin-2- | | | | 2.08-2.27 (2H, m), |
| | amine | | | | 1.78-2.00 (2H, m) |
| 184 | 6-cyclohexyl-N⁴- | Interm. | | 276 [M + H]⁺ | CD₃OD 6.26 (1H, br s), |
| | methyl-N⁴- | 31 | | RT 1.98 mins | 5.25-5.42 (1H, m), |
| | pyrrolidin-3- | | | (pH 5.8) | 3.51-3.75 (2H, m), |
| | ylpyrimidine-2,4- | | | | 3.30-3.51 (2H, m, underlying MeOH |
| | diamine | | | | peak), 3.21 (3H, s), |
| | | | | | 2.50-2.66 (1H, m), |
| | | | | | 2.21-2.46 (2H, m), 1.85-2.03 (4H, m), |
| | | | | | 1.80 (1H, d), |
| | | | | | 1.26-1.66 (5H, m) |
| 185 | 4-cyclopentyl-6- | Interm. | hydrochloride | 262 [M + H]⁺ | d₆-DMSO 12.75 (1H, bs) |
| | [(3S)-3- | 38 | | RT 1.94 mins | 9.65 (3H, bs), 7.8 (2H, bs), |
| | methylpiperazin- | | | (pH 5.8) | 6.5 (1H, s), |
| | 1-yl]pyrimidin-2- | | | | 4.58-4.82 (1H, m), 4.20-4.48 (1H, |
| | amine | | | | m), 2.86-3.60 (6H, mm), |
| | | | | | 1.92-2.10 (2H, m), |
| | | | | | 1.58-1.85 (6H, mm), 1.30 (3H, |
| | | | | | d). |
| 186 | 4-cyclopentyl-6- | Interm. | hydrochloride | 262 [M + H]⁺ | d₆-DMSO 12.80 (1H, bs) |
| | [(3R)-3- | 39 | | RT 1.86 mins | 9.62 (3H, bs), 7.82 (2H, |
| | methylpiperazin- | | | (pH 5.8) | bs), 6.5 (1H, s), 4.75 (1H, |
| | 1-yl]pyrimidin-2- | | | | bs), 4.35 (1H, bs), |
| | amine | | | | 2.83-3.62 (6H, mm), |
| | | | | | 1.95-2.12 (2H, m), 1.60-1.90 (6H, |
| | | | | | mm), 1.32 (3H, d). |

Comp. No means Compound Number
Interm. means Intermediate

### Example 37

### Synthesis of 6-cyclohex-1-en-1-yl-N⁴-[2-(dimethylamino)ethyl]pyrimidine-2,4-diamine (Compound 187)

A solution of Intermediate 28 (200 mg) and cyclohexen-1-yl boronic acid (CAS RN 89490-05-1) (200 mg) in DME (2 ml) is degassed by passage of nitrogen gas. Sodium carbonate (2M aq, 1 ml) is added, followed by chloro(di-norbornylphosphino)-(2′-dimethylamino-1,1′-biphenyl-2-yl)palladium (II) (CAS RN 3599803-53-5) (5 mg). The vessel is sealed and the reaction mixture heated by microwave irradiation at 140° C. for 30 min. The mixture is cooled, added to water (10 ml) and extracted with EtOAc (2×10 ml). The solvent is washed with water (10 ml), dried and evaporated and the residue purified by preparative HPLC (method B). The product is dissolved in water, basified with sodium hydroxide solution (2M aq) and the mixture extracted with EtOAc. The solvent is dried and evaporated to give the title compound as colorless solid (35 mg). LCMS 262 [M+H]⁺, RT 1.64 mins (pH 5.8). ¹H NMR 300 MHz (CDCl₃) (δ ppm): 6.79 (1H, m), 5.78 (1H, s), 5.25 (1H, m), 4.69 (2H, br s), 3.33 (2H, m), 2.50 (2H, t), 2.32 (2H, m), 2.23 (6H, s), 2.20 (2H, m), 1.74 (2H, m), 1.62 (2H, m).

Compounds 188 and 189 are prepared in a similar manner to the method described for Compound 187 in Example 37.The reagents used and the results obtained are tabulated below (Table 15). The free base of the compounds is obtained.

TABLE 15

| Comp. | | Starting | | ¹H NMR |
|---|---|---|---|---|
| No | IUPAC Name | Materials | LCMS | (Solvent, δ ppm) |
| 188 | 6-[(E)-2- | Intermediate | 248 [M + H]⁺ | CDCl₃ 6.30 (1H, dd), |
| | cyclopropylvinyl]- | 28, | RT 1.57 mins | 6.25 (1H, d), |
| | N⁴-[2- | (E)- | (pH 5.8) | 5.65 (1H, s), 5.23 (1H, br |
| | (dimethylamino)ethyl]pyrimidine- | cyclopropylvinylboronic | | t), 4.65 (2H, br s), |
| | 2,4- | acid | | 3.32 (2H, dt), |
| | diamine | | | 2.48 (2H, t), 2.24 (6H, s), |
| | | | | 1.57 (1H, m), |
| | | | | 0.83 (2H, m), 0.61 (2H, m) |
| 189 | N⁴-[2- | Intermediate | 264 [M + H]⁺ | CDCl₃ 6.75 (1H, d), |
| | (dimethylamino)ethyl]- | 28, | RT 2.15 mins | 6.05 (1H, d), |
| | 6- | (E)-tert- | (pH 5.8) | 5.70 (1H, s), 5.30 (1H, br |
| | [(1E)-3,3- | butylvinylboronic | | t), 4.83 (2H, br s), |
| | dimethylbut- | acid | | 3.32 (2H, dt), |
| | 1-en-1- | | | 2.48 (2H, t), 2.24 (6H, s), |
| | yl]pyrimidine- | | | 1.10 (9H, s) |
| | 2,4-diamine | | | |

Comp. No means Compound Number

### Example 38

### 4-(3-aminoazetidin-1-yl)-6-cyclohex-1-en-1-ylpyrimidin-2-amine (Compound 190)

A solution of tert-butyl [1-(2-amino-6-chloropyrimidin-4-yl)azetidin-3-yl]carbamate (CAS RN 854038-89-4) (100 mg) and cyclohexen-1-yl boronic acid (200 mg) in DME (2 ml) is degassed by passage of nitrogen gas. Sodium carbonate (2M aq, 1 ml) is added, followed by chloro(di-norbornylphosphino)-(2′-dimethylamino-1,1′-biphenyl-2-yl)palladium (II) (CAS RN 3599803-53-5) (5 mg). The vessel is sealed and the reaction mixture heated by microwave irradiation at 140° C. for 30 min. The mixture is cooled, added to water (10 ml) and extracted with EtOAc (2×10 ml). The solvent is washed with water (10 ml), dried and evaporated and the residue filtered through a silica plug eluting with 5% MeOH/DCM. The product is dissolved in DCM (10 ml) and TFA (3 ml) added. The mixture is stirred for 2 h then evaporated in vacuo and the residue dissolved in water (10 ml) and washed with ether (2×10 ml). The aqueous layer is basified with sodium hydroxide (2M aq) and extracted with EtOAc (2×10 ml). The solvent is dried and evaporated to give the title compound as beige solid (30 mg). LCMS 246 [M+H]⁺, RT 1.94 min (pH 5.8). ¹H NMR 300 MHz (CDCl₃) (δ ppm): 6.75 (1H, m), 5.60 (1H, s), 5.58 (2H, br s), 4.33 (2H, m), 3.95 (1H, m), 3.69 (2H, m), 2.80 (2H, br s), 2.20-2.35 (4H, m), 1.60-1.79 (4H, m).

Compounds 191 to 193 are prepared in a similar manner to the method described for Compound 190 in Example 38.The reagents used and the results obtained are tabulated below (Table 16). The free base of the compounds is obtained unless otherwise stated.

TABLE 16

| | | | | | ¹H NMR |
|---|---|---|---|---|---|
| Comp. | IUPAC | Starting | | | (Solvent, |
| No | Name | Materials | Salt | LCMS | δ ppm) |
| 191 | 4-[(3R)-3- | tert-butyl | | 262 [M + H]⁺ | CDCl₃ 6.81 (1H, d), |
| | aminopyrrolidin- | [(3R)-1-(2- | | RT 1.27 min | 6.12 (1H, d), 5.70 (1H, s), |
| | 1-yl]-6- | amino-6- | | (pH | 4.67 (2H, br s), 3.60-3.73 (3H, |
| | [(1E)-3,3- | chloropyrimidin- | | 5.8) | m), 3.50 (1H, m), 3.18 (1H, |
| | dimethylbut- | 4- | | | m), 2.17 (1H, m), 1.77 (1H, |
| | 1-en-1- | yl)pyrrolidin-3- | | | m), 1.13 (9H, s) |
| | yl]pyrimidin- | yl]carbamate | | | |
| | 2-amine | (CAS RN | | | |
| | | 929716-72-3), | | | |
| | | (E)-tert- | | | |
| | | butylvinylboronic | | | |
| | | acid | | | |
| 192 | 4-[(3R)-3- | tert-butyl | | 260 [M + H]⁺ | CDCl₃ 6.79 (1H, m), |
| | aminopyrrolidin- | [(3R)-1-(2- | | RT 1.19 min | 5.72 (1H, s), 4.65 (2H, s), |
| | 1-yl]-6- | amino-6- | | (pH | 3.60-3.72 (3H, m), 3.50 (1H, m), |
| | cyclohex-1- | chloropyrimidin- | | 2) | 3.18 (1H, m), 2.35 (1H, m), |
| | en-1- | 4- | | | 2.10-2.20 (3H, m), |
| | ylpyrimidin- | yl)pyrrolidin-3- | | | 1.60-1.85 (5H, m) |
| | 2-amine | yl]carbamate, | | | |
| | | cyclohexen-1- | | | |
| | | ylboronic acid | | | |
| 193 | 4-(3- | tert-butyl [1-(2- | Acetate | 248 [M + H]⁺ | CD₃OD 6.82 (1H, d), |
| | aminoazetidin- | amino-6- | | RT 2.15 mins | 6.15 (1H, d), 4.93 (1H, s), |
| | 1-yl)-6- | chloropyrimidin- | | (pH 5.8) | 4.40 (2H, m), 4.02 (1H, m), |
| | [(1E)-3,3- | 4-yl)azetidin- | | | 3.95 (2H, m), 1.95 (6H, s), |
| | dimethylbut- | 3-yl]carbamate | | | 1.15 (9H, s) |
| | 1-en-1- | (CAS RN | | | |
| | yl]pyrimidin- | 854038-89-4), | | | |
| | 2-amine | (E)-tert- | | | |
| | | butylvinylboronic | | | |
| | | acid | | | |

Comp. No means Compound Number

### Example 39

### Synthesis of tert-butyl [cis-4-(anilinocarbonyl)cyclohexyl]carbamate (Intermediate 72)

To a solution of cis-4-[(tert-butoxycarbonyl)amino]cyclohexanecarboxylic acid (CAS No 53292-90-3) (100 mg) in DMF (3 ml) is added O-benzotriazole-N,N,N′,N′-tetramethyluroniumhexafluorophosphate (HBTU) (CAS RN 94790-37-1) (156 mg) and DIPEA (143 μl). The reaction mixture is stirred at room temperature for 15 mins. Aniline (39 μl) is added and the mixture is stirred at room temperature overnight. The solution obtained is dissolved in EtOAc, washed with 1 M HCl, saturated NaHCO₃ and saturated brine and dried over MgSO₄. After filtration, the organic layer is evaporated in vacuo to give a pale brown solid (151 mg). LCMS 219 [MH]⁺-Boc, RT 3.46 mins (pH 2). ¹H NMR 300 MHz (CDCl₃) (δ ppm): 7.50 (2H, d), 7.30 (2H, t), 7.05-7.20 (2H, m), 4.65-4.80 (1H, m), 3.70-3.85 (1H, m), 2.30-2.40 (1H, m), 1.75-1.90 (6H, m), 1.60-1.75 (2H, m), 1.45 (9H, s).

Intermediates 73 to 77 are prepared in a similar manner to the method described for Intermediate 72 in Example 39.The reagents used and the results obtained are tabulated below (Table 17). The free base of the compounds is obtained.

TABLE 17

| | | | | ¹H NMR |
|---|---|---|---|---|
| Interm. | IUPAC | | | (Solvent, |
| No | Name | Starting Materials | LCMS | δ ppm) |
| 73 | terl-butyl | (1R*,3S*)-3-[(tert- | 219 | CDCl₃ 7.51 (2H, d), |
| | ((1R*,3S*)- | butoxycarbonyl)amino]cyclohexanecarboxylic | [M + H⁺ − | 7.31 (2H, t), 7.18 (1H, brs), |
| | 3- | acid (CAS | BOC] RT | 7.10 (1H, t), 4.49 (1H, m), |
| | (anilinocarbonyl)cyclohexyl]carbamate | RN 222530-33-8), | 3.52 mins | 3.51 (1H, m), |
| | | aniline | (pH 2) | 2.20-2.38 (2H, m), 1.84-2.02 (3H, |
| | | | | m), 1.04-1.62 (13H, m) |
| 74 | tert-butyl | trans-4-[(tert- | | CDCl₃ 5.57 (1H, brs), |
| | [trans-4- | butoxycarbonyl)amino]cyclohexanecarboxylic | | 4.38 (1H, br m), 3.40 (1H, |
| | (methylcarbamoyl)cyclohexyl]carbamate | acid (CAS | | br m), 1.87-2.39 (5H, m), |
| | | RN 53292-89-0), | | 1.35-1.56 (11H, m), |
| | | methylamine | | 1.02-1.20 (2H, m) |
| 75 | tert-butyl | trans-4-[(tert- | | CDCl₃ 5.52 (1H, brs), |
| | [trans-4- | butoxycarbonyl)amino]cyclohexanecarboxylic | | 4.35 (1H, br m), 3.40 (1H, |
| | (cyclopropylcarbamoyl)cyclohexyl]- | acid, | | br m), 2.60 (1H, m), |
| | carbamate | cyclopropylamine | | 1.84-2.15 (5H, m), |
| | | | | 1.39-1.52 (11H, m), 1.08 (2H, m), |
| | | | | 0.77 (2H, m), 0.46 (2H, m) |
| 76 | tert-butyl | trans-4-[(tert- | | CDCl₃ 5.61 (1H, brs), |
| | [trans-4- | butoxycarbonyl)amino]cyclohexanecarboxylic | | 4.36 (1H, br m), 3.41 (1H, |
| | (tert- | acid, tert- | | br m), 2.08 (2H, m), |
| | butylcarbamoyl)cyclohexyl]carbamate | butylamine | | 1.82-1.95 (3H, m), |
| | | | | 1.40-1.52 (11H, m), 1.32 (9H, s), |
| | | | | 1.08 (2H, m) |
| 77 | tert-butyl | trans-4-[(tert- | | CDCl₃ 7.41 (2H, d), |
| | {trans-4- | butoxycarbonyl)amino]cyclohexanecarboxylic | | 7.10 (1H, brs), 6.85 (2H, d), |
| | [(4- | acid, 4- | | 4.40 (1H, br m), 3.56 (1H, |
| | methoxyphenyl)carbamoyl]- | methoxyaniline | | br m), 1.97-2.21 (5H, m), |
| | cyclohexyl}carbamate | | | 1.55-1.76 (2H, m), |
| | | | | 1.45 (9H, s), 1.15 (2H, m) |

Interm. No means Intermediate Number

### Example 40

### Synthesis of 6-[(3R)-3-aminopyrrolidin-1-yl]-N⁴-cyclohexylpyrimidine-2,4-diamine triacetate salt (Compound 194)

A mixture of 6-chloro-N⁴-cyclohexylpyrimidin-2,4-diamine (68 mg), tert-butyl (3R)-pyrrolidin-3-ylcarbamate (56 mg) and Et₃N (84 μl) in EtOH (1 ml) is heated at 180° C. for 20 mins under microwave irradiation. After cooling the solution is evaporated under reduced pressure. The residue obtained (140 mg) is dissolved in MeOH (2 ml) and 2M HCl in Et₂O is added. The mixture is stirred at room temperature overnight. The solvent is evaporated in vacuo to give a brown oil (133 mg). Purification by preparative HPLC (Method B) affords the title compound as a yellow oil (29 mg, 21 %). LCMS 277 [M+H]⁺, RT 1.92 mins (pH 5.8). ¹H NMR 300 MHz (CD₃OD) (δ ppm):4.95 (1H, obscured by H₂O peak, s), 3.85-3.95 (1H, m), 3.75 (1H, dd), 3.45-3.70 (4H, m), 2.30-2.45 (1H, m), 2.05-2.15 (1H, m), 1.90-2.05 (2H, m), 1.95 (9H, s, 3×AcOH), 1.60-1.85 (2H, m), 1.20-1.50 (6H, m).

Compounds 195 and 196 are prepared in a similar manner to the method described for Compound 194 in Example 40.The reagents used and the results obtained are tabulated below (Table 18). The free base of the compounds is obtained unless otherwise stated.

TABLE 18

| Comp. | | Starting | | | ¹H NMR |
|---|---|---|---|---|---|
| No | IUPAC Name | Materials | Salt | LCMS | (Solvent, δ ppm) |
| 195 | N⁴-cyclohexyl- | 6-chloro-N⁴- | Acetate | 291 [M + H]⁺ | CD₃OD |
| | 6-[3- | cyclohexylpyrimidin- | | RT | 4.92 (1H, s), |
| | (methylamino)pyrrolidin- | 2,4- | | 1.86 mins | 3.44-3.81 (6H, m), |
| | 1- | diamine, tert- | | (pH 5.8) | 2.53 (3H, s), |
| | yl]pyrimidine- | butyl | | | 2.37 (1H, m), |
| | 2,4-diamine | methyl(pyrrolidin- | | | 2.13 (1H, m), |
| | | 3- | | | 1.89-2.03 (5H, m), |
| | | yl)carbamate | | | 1.61-1.86 (3H, |
| | | (CAS RN | | | m), |
| | | 172478-00-1) | | | 1.21-1.51 (5H, m) |
| 196 | 6-[(3S)-3- | 6-chloro-N⁴- | Acetate | 277 [M + H]⁺ | CD₃OD |
| | aminopyrrolidin- | cyclohexylpyrimidin- | | RT | 4.92 (1H, s), 3.89 (1H, |
| | 1-yl]-N⁴- | 2,4- | | 1.95 mins | m), |
| | cyclohexylpyrimidine- | diamine, tert- | | (pH 5.8) | 3.44-3.80 (5H, m), |
| | 2,4- | butyl (3S)- | | | 2.38 (1H, m), |
| | diamine | pyrrolidin-3- | | | 1.92-2.15 (6H, m), |
| | | ylcarbamate | | | 1.61-1.87 (3H, |
| | | | | | m), |
| | | | | | 1.21-1.51 (5H, m) |

Comp. No means Compound Number

### Example 41

### Synthesis of N⁴-[(1R*,2R*,4S*)-bicyclo[2.2.1]hept-2-yl]-6-[(3S)-3-(methylamino)pyrrolidin-1-yl]pyrimidine-2,4-diamine (Compound 197)

4,6-Dichloropyrimidin-2-amine (0.082 g), (1S*,2S*,4R*)-bicyclo[2.2.1]heptan-2-amine (0.059 ml) and Et₃N (0.348 ml) are dissolved in EtOH (1.5 ml) and the solution is heated in the microwave at 170° C. for 30 mins. Then tert-butyl (3S)-pyrrolidin-3-ylcarbamate (0.139 g) is added to the reaction and further heated at 160° C. for 25 mins. The reaction mixture is evaporated in vacuo, dissolved in DCM (20 ml), washed with 1M HCl (10 ml), brine (10 ml), dried (MgSO₄) and evaporated in vacuo. Prep-HPLC (pH 5.8) affords a colorless glass (111 mg), which is dissolved in DCM (20 ml), washed with NaHCO₃ solution (2 ml), dried (MgSO₄) and evaporated in vacuo to give a solid (104 mg). This is dissolved in dry THF (5 ml), LiAlH₄ solution (1.0 M in THF, 1.26 ml) added, and the solution heated at 75° C. for 4 hrs. The reaction is cooled, quenched successively with water (0.035 ml), 15% aqueous NaOH (0.035 ml) and then water (0.105 ml). After 30 mins, the solids are filtered off, washed with THF (4×5 ml), and the organics concentrated in vacuo. Flash chromatography of the residue (DCM-MeOH 97:3 rising to 93:7+1% 7N NH₃ in MeOH) affords the title compound as a colorless foam (45 mg, 55%). R_{f} (DCM-MeOH 93:7+1% 7N NH₃ in MeOH) 0.40. LCMS 303 [M+H]⁺, RT 2.05 mins (pH 5.8). ¹H NMR 300 MHz (CDCl₃) (δ ppm): 4.72 (1H, s), 4.52 (2H, s), 4.47 (1H, d), 3.15-3.70 (6H, m), 2.47 (3H, s), 2.22-2.34 (2H, m), 2.15 (1H, m), 1.80 (2H, m), 1.40-1.60 (3H, m), 1.08-1.30 (5H, m).

Compounds 198 to 200 are prepared in a similar manner to the method described for Compound 197 in Example 41.The reagents used and the results obtained are tabulated below (Table 19). The free base of the compounds is obtained.

TABLE 19

| Comp. | | | | ¹H NMR |
|---|---|---|---|---|
| No | IUPAC Name | Starting Materials | LCMS | (Solvent, δ ppm) |
| 198 | N⁴- | 4,6- | 303 [M + H]⁺ | CDCl₃ |
| | [(1R*,2R*,4S*)- | dichloropyrimidin-2- | RT 2.02 mins | 4.75 (1H, s), |
| | bicyclo[2.2.1]hept- | amine, | (pH 5.8) | 4.42-4.60 (3H, m), |
| | 2-yl]-6-[(3R)- | (1R*,2R*,4S*)- | | 3.17-3.73 (6H, |
| | 3- | bicyclo[2.2.1]heptan- | | m), 2.46 (3H, |
| | (methylamino)pyrrolidin- | 2-amine, tert-butyl | | s), |
| | 1- | (3R)-pyrrolidin-3- | | 2.00-2.33 (5H, m), |
| | yl]pyrimidine- | ylcarbamate | | 1.80 (2H, m), |
| | 2,4-diamine | | | 1.08-1.60 (6H, m). |
| 199 | N⁴- | 4,6- | 303 [M + H]⁺ | CDCl₃ |
| | [(1R*,2S*,4S*)- | dichloropyrimidin-2- | RT 2.05 mins | 5.30 (1H, m), |
| | bicyclo[2.2.1]hept- | amine, | (pH 5.8) | 4.68-4.90 (3H, m), |
| | 2-yl]-6-[(3S)- | (1R*,2S*,4S*)- | | 2.90-3.78 (8H, m), |
| | 3- | bicyclo[2.2.1]heptan- | | 2.46 (3H, s), |
| | (methylamino)pyrrolidin- | 2-amine | | 2.00-2.30 (3H, m), |
| | 1- | hydrochloride, tert- | | 1.82 (1H, m), |
| | yl]pyrimidine- | butyl (3S)-pyrrolidin- | | 1.20-1.71 (6H, |
| | 2,4-diamine | 3-ylcarbamate | | m), 0.85 (1H, |
| | | | | m). |
| 200 | N⁴- | 4,6- | 303 [M + H]⁺ | CDCl₃ |
| | [(1R*,2S*,4S*)- | dichloropyrimidin-2- | RT 2.13 mins | 5.95 (1H, m), |
| | bicyclo[2.2.1]hept- | amine, | (pH 5.8) | 5.08 (2H, bs), |
| | 2-yl]-6-[(3R)- | (1R*,2S*,4S*)- | | 4.70 (1H, m), |
| | 3- | bicyclo[2.2.1]heptan- | | 3.15-3.80 (7H, m), |
| | (methylamino)pyrrolidin- | 2-amine | | 2.46 (3H, s), |
| | 1- | hydrochloride, tert- | | 1.13-2.33 (11H, m), |
| | yl]pyrimidine- | butyl (3R)-pyrrolidin- | | 0.87 (1H, m). |
| | 2,4-diamine | 3-ylcarbamate | | |

Comp. No means Coumpound Number

### Example 42

### Synthesis of methyl 3-oxo-3-(1,2,3,4-tetrahydronaphthalen-2-yl)propanoate (Intermediate 78)

Anhydrous DMF (5 drops) is added to an ice-cold solution of 1,2,3,4-tetrahydronaphthalene-2-carboxylic acid (CAS No 53440-12-3) (3.89 g) in anhydrous DCM (50 ml) under N₂, followed by the dropwise addition of oxalyl chloride (5.78 ml). The reaction mixture is stirred under N₂, warming to room temperature. After 3 hrs the reaction mixture is evaporated in vacuo, and the residue azeotroped in vacuo with 1:1 DCM/heptane (3×10 ml). The resultant amber oil is redissolved in anhydrous DCM (20 ml), and this solution is added dropwise to a stirred, ice-cold solution of 2,2-dimethyl-1,3-dioxane-4,6-dione (CAS No 131376-78-8) (3.18 g) and pyridine (5.36 ml) in anhydrous DCM (30 ml), under N₂. The reaction mixture is stirred under N₂, warming to room temperature. After 18 hrs the now dark reaction mixture is washed with water (30 ml), 1 M hydrochloric acid (2×50 ml), water (30 ml), dried (Na₂SO₄) and evaporated in vacuo. The resulting dark red oil is dissolved in anhydrous methanol (50 ml) and heated to reflux under N₂. After 3 hrs, the reaction is cooled and evaporated under reduced pressure. The residue is redissolved in EtOAc (80 ml) and washed with saturated NaHCO₃ solution (2×40 ml), water (30 ml), saturated brine (30 ml), dried (Na₂SO₄) and evaporated in vacuo to give the title compound as a red oil (4.41 g, 86%). ¹H NMR 300 MHz (CDCl₃) (δ ppm): 7.05-7.16 (4H, m), 3.75 (3H, s), 3.61 (1H, s), 2.69-3.04 (5H, m), 2.15-2.26 (1H, m), 1.70-1.91 (1H, m).

### Example 43

### Synthesis of 2-amino-6-(1,2,3,4-tetrahydronaphthalen-2-yl)pyrimidin-4-ol (Intermediate 79)

Intermediate 78 (4.40 g) and guanidine carbonate (1.54 g) is combined in ethanol (50 ml) and heated to reflux. After 18 hrs the reaction is cooled and the resultant precipitate is filtered and washed with ethanol (2×10 ml), water (2×10 ml), acetone (2×10 ml) and dried in vacuo at 50° C. for 18 hrs to provide the title compound as a white solid (2.53 g, 55%). A further crop of the title compound precipitated from the combined filtrate after standing, and this is removed by filtration, washed well with water and dried at 50° C. for 18 hrs (0.23 g, 5%). LCMS 242 [M+H]⁺, RT 1.99 mins (pH 2). ¹H NMR 300 MHz (d₆-DMSO) (δ ppm): 7.09 (4H, brs), 6.49 (2H, brs), 5.47 (1H, s), 2.75-2.92 (4H, m), 2.50-2.67 (1H, m), 1.94-2.05 (1H, m), 1.69-1.84 (1H, m).

Intermediates 80 and 81 are prepared in a similar manner to the method described for Intermediate 79 in Example 43.The reagents used and the results obtained are tabulated below (Table 20). The free base of the compounds is obtained.

TABLE 20

| Interm. | IUPAC | | | ¹H NMR |
|---|---|---|---|---|
| No | Name | Starting Materials | LCMS | (Solvent, δ ppm) |
| 80 | 2-amino-6- | ethyl 4-cyclohexyl-3- | 208 [M + H]⁺ | CD₃OD 5.57 (1H, s), |
| | (cyclohexylmethyl)pyrimidin- | oxobutanoate (CAS | RT 1.73 mins | 2.26 (2H, d), 0.90-1.83 |
| | 4-ol | RN 64127-44-2) | (pH 2.5) | 11H, m). |
| 81 | 2-amino-6- | ethyl 4-cyclopentyl- | 194 [M + H]⁺ | d₆-DMSO 11.00 (1H, |
| | (cyclopentylmethyl)pyrimidin- | 3-oxo-butanoate | RT 1.51 mins | bs), 6.55 (2H, bs), |
| | 4-ol | (CAS RN 24922-00-7) | (pH 2.5) | 5.32 (1H, s), 2.05-2.30 (3H, |
| | | | | m), 1.38-1.74 (6H, m), |
| | | | | 1.02-1.23 (2H, m). |

Interm. No means Intermadiate Number

### Example 44

### Synthesis of 4-tert-butyl-6-[3-(methylamino)pyrrolidin-1-yl]pyrimidin-2-amine acetate salt (Compound 201)

A solution of 2-amino-6-tert-butylpyrimidin-4-ol (CAS RN 139541-35-8) (0.5 g) and POCl₃ (5 ml) is heated under reflux for 2 hrs. After cooling the solution is evaporated under reduced pressure and the residue obtained is partitioned between EtOAc and water. The EtOAc layer is dried (MgSO₄) and evaporated in vacuo to give a white solid (0.77 g). The material (150 mg) is dissolved in NMP (1.2 ml) and tert-butyl methyl(pyrrolidin-3-yl)carbamate (CAS No 172478-00-1) (162 mg) and Et₃N (225 μl) are added. The solution is then heated under microwave irradiation at 180° C. for 40 mins. The crude reaction mixture is evaporated under high vacuum (Genevac) to give a brown oil (614 mg). The material (283 mg) is dissolved in MeOH (5 ml) and 2M HCl in Et₂O (5 ml) is added and the mixture is stirred at room temperature overnight. The excess solvent is removed in vacuo. Purification by preparative HPLC (Method B) affords the title compound as a yellow oil (162 mg, 65%). LCMS 250 [M+H]⁺, RT 1.78 mins (pH 5.8). ¹H NMR 300 MHz (CD₃OD) (δ ppm): 6.00 (1H, s), 3.85-3.95 (1H, m), 3.60-3.85 (4H, m), 2.65 (3H, s), 2.35-2.50 (1H, m), 2.15-2.25 (1H, m), 1.95 (3H, s, AcOH), 1.35 (9H, s).

### Example 45

### Synthesis of 4-(3-aminopyrrolidin-1-yl)-6-tert-butylpyrimidin-2-amine bis acetate salt (Compound 202)

Compound 202 is prepared according to the method described in Example 44, from 2-amino-6-tert-butylpyrimidin-4-ol (CAS RN 139541-35-8) (97.4 mg) and tert-butyl pyrrolidin-3-ylcarbamate (150 mg). Purification by preparative HPLC (Method B) affords the title compound as a yellow solid (129 mg, 68%). LCMS 236 [M+H]⁺, RT 1.71 mins (pH 5.8). ¹H NMR 300 MHz (CD₃OD) (δ ppm):) 5.95 (1H, s), 3.90-4.00 (1H, m), 3.60-3.90 (4H, m), 2.35-2.50 (1H, m), 2.05-2.20 (1H, m), 1.95 (6H, s, 2×AcOH), 1.35 (9H, s).

### Example 46

### Synthesis of 4-cyclohexyl-6-[(2S)-2,4-dimethylpiperazin-1-yl]pyrimidin-2-amine (Compound 203)

Compound 22 (30 mg), phenyl silane (24 mg), dibutyl tin hydride (3.3 mg) and paraformaldehyde (2.9 mg) is combined in THF (3 ml) and heated to 100° C. for 30 mins in the microwave. The reaction mixture is concentrated in vacuo and the residue is taken up in DMSO and purified by prep HPLC (pH 5.8) to afford the acetic acid salt. The salt is taken up in DCM (100 ml) and washed with K₂CO₃ aq (100 ml), brine (100 ml), dried (MgSO₄) and evaporated in vacuo to afford the title compound as a white solid (10.6 mg, 33%). LCMS 290.3 [M+H]⁺, RT 2.23 mins (pH 5.8). ¹H NMR 300 MHz (CD₃OD) (δ ppm): 5.92 (1H,s), 4.90 (1H, bt), 4.18 (1H, bd), 3.11(1H, td), 2.93-2.84 (1H, m), 2.80 (1H, bdt), 2.4-2.25 (1H, m), 2.30 (3H, s), 2.21 (1H, dd), 2.00 (1H, td), 1.92-1.72 (5H, mm), 1.57-1.22 (5H, mm), 1.26 (3H, d).

### Example 47

### Synthesis of 4-cyclohexyl-6-[(2R)-2,4-dimethylpiperazin-1-yl]pyrimidin-2-amine (Compound 204)

Compound 204 is prepared according to the method described in Example 46, from Compound 23 (70 mg). Prep HPLC (pH 5.8) followed by a similar work up to Example 46 affords the title compound as a white solid (42.2 mg, 57%). LCMS 290.3 [M+H]⁺, RT 2.23 mins (pH 5.8). ¹H NMR 300 MHz (CD₃OD) (δ ppm): 5.92 (1H,s), 4.90 (1H, bt), 4.18 (1H, bd), 3.11 (1H, td), 2.93-2.84 (1H, m), 2.80 (1H, bdt), 2.4-2.25 (1H, m), 2.30 (3H, s), 2.21 (1H, dd), 2.00 (1H, td), 1.92-1.72 (5H, mm), 1.57-1.22 (5H, mm), 1.26 (3H, d).

### Example 48

### Synthesis of 4-cyclohexyl-6-(4-cyclopropylpiperazin-1-yl)-N-(4-methoxybenzyl)pyrimidin-2-amine bis-formate salt (Intermediate 82)

Intermediate 82 is prepared from Intermediate 3 (0.089 g), 4-methoxybenzylamine (0.055 ml) and Et₃N (0.058 ml) in the same manner as described for Intermediate 11 in Example 7. Prep-HPLC (pH 2.5) affords the title compound as a colorless glass (58 mg, 49%). LCMS 422 [M+H]⁺, RT 1.73 mins (pH 2.5). ¹H NMR 300 MHz (CDCl₃) (δ ppm): 10.42 (1H, m), 8.30 (2H, HCOOH), 7.29 (2H, d), 6.85 (2H, d), 5.70 (1H, s), 4.48 (2H, d), 3.40-3.97 (4H, m), 3.80 (3H, s), 2.64-2.76 (4H, m), 2.55 (1H, m), 1.64-2.00 (6H, m), 1.15-1.50 (5H, m), 0.52 (4H, m).

### Example 49

### Synthesis of 4-cyclohexyl-6-(4-cyclopropylpiperazin-1-yl)pyrimidin-2-amine (Compound 205)

Compound 205 is prepared from Intermediate 82 (0.058 g) and trifluoroacetic acid (1.5 ml) in the same manner as described for Compound 9 in Example 6. Prep-HPLC (pH 5.8) followed by a DCM/saturated NaHCO₃ partition affords the title compound as colorless crystals (25 mg, 53%). LCMS 302 [M+H]⁺, RT 2.75 mins (pH 5.8). ¹H NMR 300 MHz (CDCl₃) (δ ppm): 5.80 (1H, s), 4.82 (2H, bs), 3.55 (4H, m), 3.00 (1H, m), 2.65 (4H, m), 2.35 (1H, m), 1.15-2.00 (10H, m), 0.38-0.60 (4H, m).

### Example 50

### Synthesis of 4-tert-butyl-6-(4-methylpiperazin-1-yl)pyrimidin-2-amine acetate salt (Compound 206)

A solution of 2-amino-6-tert-butylpyrimidin-4-ol (CAS RN 139541-35-8) (0.5 g) and POCl₃ (5 ml) is heated under reflux for 2 hrs. After cooling the solution is evaporated under reduced pressure. The residue obtained is partitioned between EtOAc and water. The EtOAc layer is dried (MgSO₄) and evaporated in vacuo to give a white solid (0.77 g). The crude material (150 mg) is dissolved in NMP (1.2 ml) and N-methylpiperazine (90 μl) and Et₃N (225 μl) are added. The solution is then heated under microwave irradiation at 200° C. for 40 mins. Purification by preparative HPLC (Method B) affords the title compound as a yellow oil (43.7 mg, 17%). LCMS 250 [M+H]⁺, RT 1.77 mins (pH 5.8). ¹H NMR 300 MHz (CD₃OD) (δ ppm): 6.20 (1H, s), 3.75-3.85 (4H, m), 2.50-2.60 (4H, m), 2.40 (3H, s), 1.95 (3H, s, AcOH), 1.35 (9H, s).

### Example 51

### Synthesis of 4-(4-methylpiperazin-1-yl)-6-(1,2,3,4-tetrahydronaphthalen-2-yl)pyrimidin-2-amine (Compound 207)

Intermediate 79 (493 mg) is suspended in POCl₃ (5 ml) and heated to reflux, under a calcium chloride guard tube for 2.5hrs. The POCl₃ is removed in vacuo, and the residue poured into ice-water (10 ml). Any solid lumps are broken up with a glass rod, and the suspension is filtered. The solids are washed well with water and dried under vacuum at 50° C. for 18 hrs to yield the crude 4-chloropyrimidine intermediate as a beige solid. Without purification, this crude material is suspended in MeOH (15 ml), treated with N-methylpiperazine (5 ml), and heated to reflux. After 30 mins the reaction is cooled, evaporated in vacuo, and the residue is partitioned between EtOAc (50 ml) and water (20 ml). The phases are separated and the organic phase is further washed with water (20 ml), 0.5M NaOH (20 ml), water (10 ml), saturated NaHCO₃ solution (10 ml), saturated brine (20 ml), dried (Na₂SO₄) and evaporated in vacuo to yield the title compound as an orange gum (329 mg, 50%). LCMS 324 [M+H]⁺, RT 2.46 mins (pH 5.8). ¹H NMR 300 MHz (d₆-DMSO) (δ ppm): 7.08 (4H, brs), 6.00 (1H, s), 5.94 (2H, brs), 3.50 (4H, m), 2.78-3.04 (4H, m), 2.66 (1H, m), 2.32 (4H, m), 2.20 (3H, s), 1.96-2.05 (1H, m), 1.72-1.89 (1H, m).

### Example 52

### Synthesis of tert-butyl {1-[2-amino-6-(1,2,3,4-tetrahydronaphthalen-2-yl)pyrimidin-4-yl]pyrrolidin-3-yl}carbamate (intermediate 83)

Hydrogen chloride (0.93 ml, 2.0M solution in diethyl ether) is added via syringe to Intermediate 79 (224 mg). After 5 mins, POCl₃ (5 ml) is added and the reaction heated to reflux, under a calcium chloride guard tube. After 2 hrs at reflux, anhydrous 1,4-dioxane (10 ml) is added to aid solubility and the reaction is returned to reflux for a further 2 hrs. The reaction mixture is then cooled, evaporated in vacuo, and the residue treated with ice-water (100 ml). Saturated NaHCO₃ solution is added to neutralize the mixture, which is then extracted with DCM (50 ml, then 25 ml). The combined organic extracts are washed with water (20 ml), dried (Na₂SO₄) and evaporated in vacuo. The resultant crude 4-chloropyrimidine intermediate is dissolved in MeOH (15 ml), treated with tert-butyl pyrrolidin-3-ylcarbamate (720 mg) and heated to reflux. After 18 hrs the reaction is cooled and the solvents removed under reduced pressure. The residue is dissolved in EtOAc (50 ml) and washed with water (2×20 ml), 0.5M NaOH (2×20 ml), water (20 ml), saturated brine (20 ml), dried (Na₂SO₄) and evaporated in vacuo. Purification by silica gel column chromatography, with a gradient of 20-70% (DCM/MeOH/conc. ammonium hydroxide—90:10:1) in DCM as eluent, provided the title compound as a colorless glass (118 mg, 37%). LCMS 410 [M+H]⁺, RT 2.46 mins (pH 2). ¹H NMR 300 MHz (CDCl₃) (δ ppm): 7.10 (4H, m), 5.65 (1H, s), 4.63-4.87 (3H, m), 4.30 (1H, brs), 3.24-3.78 (4H, m), 2.72-3.06 (5H, m), 2.08-2.30 (3H, m), 1.81-1.99 (2H, m), 1.46 (9H, s).

### Example 53

### Compound 208 (Isomer 1) & Compound 209 (Isomer 2). Chiral Separation of Racemic 4-(3-methyl-3,4-dihydroisoquinolin-2(1H)-yl)-6-(4-methylpiperazin-1-yl)pyrimidin-2-amine (Compound 140)

Compound 140 (76 mg) is separated by chromatography (Chiralpak AD 250*420 mm eluting with 15% EtOH in Heptane) to give the title compounds as colorless solids. Compound 208 (Isomer 1), 12.7 mg. RT 9.1 min (ChiralPak AD 250* 4.6 mm column eluting with 15% EtOH in Heptane). LCMS 339 [M+H]⁺, RT 1.28 mins (pH 2.5). ¹H NMR 300 MHz (CDCl₃) (δ ppm): 7.05-7.15 (4H, m), 5.12 (1H, s), 4.83-4.97 (1H, m), 4.69 (1H, d), 4.90 (2H, bs), 4.28 (1H, d), 3.42-3.57 (4H, m), 3.09 (1H, dd), 2.59 (1H, dd), 2.36-2.44 (4H, m), 2.26 (3H, s), 0.98 (3H, d). Compound 209 (Isomer 2), 11.7 mg. RT 12.1 min (ChiralPak AD 250* 4.6 mm column eluting with 15% EtOH in Heptane). LCMS 339 [M+H]⁺, RT 1.28 mins (pH 2.5). ¹H NMR 300 MHz (CDCl₃) (δ ppm): 7.05-7.15 (4H, m), 5.12 (1H, s), 4.83-4.97 (1H, m), 4.69 (1H, d), 4.90 (2H, bs), 4.28 (1H, d), 3.42-3.57 (4H, m), 3.09 (1H, dd), 2.59 (1H, dd), 2.36-2.44 (4H, m), 2.26 (3H, s), 0.98 (3H, d).

### Example 54

### Compound 210 (Isomer 1) & Compound 211 (Isomer 2). Chiral separation of racemic 4-(4-methylpiperazin-1-yl)-6-(1-phenylethyl)pyrimidin-2-amine (Compound 120)

Compound 120 (10 mg) is separated by chromatography (ChiralPak AD 250* 4.6 mm column eluting with 1:3 IPA/heptane) to give the title compounds as colorless solids. Compound 210 (Isomer 1), 2.6 mg. RT 6.49 min (ChiralPak AD 250* 4.6 mm column eluting with 1:3 IPA/heptane). LCMS 298 [M+H]⁺, RT 2.15 mins (pH 5.8). ¹H NMR 300 MHz (CD₃OD) (δ ppm): 7.10 (3H, m), 6.95 (2H, m), 5.79 (1H, s), 3.68 (1H, q), 3.40 (4H, m), 2.28 (4H, m), 2.12 (3H, s), 1.35 (3H, d). Compound 211(Isomer 2), 2.60 mg. RT 6.49 min (ChiralPak AD 250* 4.6 mm column eluting with 1:3 IPA/heptane). LCMS 298 [M+H]⁺, RT 2.15 mins (pH 5.8). ¹H NMR 300 MHz (CD₃OD) (δ ppm): 7.10 (3H, m), 6.95 (2H, m), 5.81 (1H, s), 3.70 (1H, q), 3.40 (4H, m), 2.28 (4H, m), 2.12 (3H, s), 1.35 (3H, d).

### Example 55

### Synthesis of 4-(3-aminopyrrolidin-1-yl)-6-[(1R*,5S*)-8-azabicyclo[3.2.1]oct-8-yl]pyrimidin-2-amine (Compound 212)

A mixture of 80 mg of (1R*,5S*)-8-azabicyclo[3.2.1]octane hydrogen chloride(CAS RN 6760-99-2) and 82 mg of 4,6-dichloropyrimidin-2-amine and DIPEA (0.5 ml) in NMP (4 ml) is heated in a microwave for 1 hour at 200° C. After cooling the reaction mixture to room temperature 100 mg of tert-butyl pyrrolidin-3-ylcarbamate is added and the mixture is again heated in a microwave for 1 hour at 200° C. The crude mixture is purified by preparative chromatography, dissolved in MeOH (0.5 ml) and added dropwise to a 2 N solution of HCl in diethyl ether. After 5 hours the mixture is concentrated and purified by preparative chromatography to give the title compound as a colorless solid (36 mg, 25%). LCMS 289 [M+H]⁺, RT 1.72 mins (pH 5.8). ¹H NMR 300 MHz (d₆-DMSO) (δ ppm): 5.49 (2H, s), 4.84 (1H, s), 4.32 (2H, bs), 3.0-3.6 (7H, m), 1.15-2.05 (12H, m).

### Example 56

### Synthesis of 4-cyclohept-1-en-1-yl-6-(4-methylpiperazin-1-yl)primidin-2-amine (Compound 213)

A mixture of 4,6-dichloropyrimidin-2-amine (507 mg, 3.09 mmol) and cyclohept-1-en-1-ylboronic acid (519 mg, 3.70 mmol) is suspended in THF (15 ml) and a solution of Na₂CO₃ (458 mg, 0.5 ml H₂O) is added. This mixture is degassed with N₂ and Pd(Ph₃)₄ (5 mg) added and heated at 78° C. for 18 hours. The reaction mixture is concentrated, redissolved in DCM (150 ml) and washed with a concentrated solution of NaHCO₃ (2×100 ml) and brine (2×100 ml). The product is further purified by silica chromatography (2.5% MeOH/DCM) to give 4-chloro-6-cyclohept-1-en-1-ylpyrimidin-2-amine (515 mg, 70% purity). This crude intermediate (140 mg) is dissolved in N-methylpiperazine (3 ml) and heated in a microwave for 30 minutes at 180° C. Purification by preparative chromatography gives the title compound as a colorless solid (65.4 mg, 52%). LCMS 288 [M+H]⁺, RT 2.45 mins (pH 5.8). ¹H NMR 300 MHz (CDCl₃) (δ ppm): 6.63 (1H, t), 5.98 (1H, s), 4.88 (2H, bs), 3.58-3.70 (4H, m), 2.52-2.64 (2H, m), 2.40-2.51 (4H, m), 2.35 (3H, s), 2.27-2.39 (2H, m), 1.74-1.90 (2H, m), 1.48-1.71 (4H, m).

### Example 57

### Synthesis of [(1R*,5S*,6S*)-3-(2-amino-6-cyclohexylpyrimidin-4-yl)-3-azabicyclo[3.1.0]hexan-6-amine (Compound 214)

10% palladium on charcoal (0.05 g) is added to a solution of Intermediate 21 (0.065 g) in methanol (20.0 ml). The mixture is stirred under H₂ at room temperature for 4 hrs. The solution is filtered and the solvent is evaporated in vacuo. Purification of the residue by flash chromatography, eluting with dichloromethane-methanol (95:5) followed by evaporation under vacuum at 40° C. affords the title compound as colorless oil (0.008 g, 14%). LCMS 274 [M+H]⁺, RT 2.01 mins (pH 5.8). ¹H NMR 300 MHz (CDCl₃) (δ ppm): 5.55 (1H, s), 4.65 (2H, s), 3.65 (2H, brs), 3.39 (2H,d), 2.25 (1H, m), 2.10 (1H, s), 1.70-1.92 (5H, m), 1.60 (4H, s), 1.20-1.49 (5H, m).

### Example 58

### Synthesis of tert-butyl {1-[2-amino-6-(cyclohexylmethyl)pyrimidin-4-yl]pyrrolidin-3-yl}carbamate formate salt (Intermediate 84)

Intermediate 80 (0.209 g) is suspended in POCl₃ (2 ml) and heated under N₂ at 110° C. for 90 mins. Excess POCl₃ is evaporated in vacuo, the residue dissolved in DCM (25 ml), washed with saturated NaHCO₃ (20 ml), dried (MgSO₄) and concentrated in vacuo. Flash chromatography of the residue (EtOAc-Heptane 1:2) affords a yellow oil (0.113 g) which is dissolved in dry NMP (2 ml), tert-butyl pyrrolidin-3-ylcarbamate (0.094 g) and Et₃N (0.07 ml) are added and heated in the microwave at 120° C. for 30 mins. Prep-HPLC of the solution (pH 2.5) affords the title compound as a colorless solid (94 mg, 45%). LCMS 376 [M+H]⁺, RT 2.37 mins (pH 2.5). ¹H NMR 300 MHz (CDCl₃) (δ ppm): 8.60 (1H, HCOOH), 6.92 (0.5H, bs), 5.59 (1H, s), 5.56 (0.5H, bs), 4.34 (0.5H, m), 4.15 (0.5H, m), 3.30-3.87 (4H, m), 2.44 (2H, d), 1.86-2.32 (3H, m), 1.60-1.78 (6H, m), 1.45 (9H, s), 0.90-1.32 (6H, m).

### Example 59

### Synthesis of tert-butyl {1-[2-amino-6-(2-phenylethyl)pyrimidin-4-yl]pyrrolidin-3-yl}carbamate (Intermediate 85)

Intermediate 62 (0.335 mg) is dissolved in absolute EtOH and degassed. 10 wt % Palladium on carbon (0.088 g) is added and the reaction stirred rapidly under 1 atm H₂ for 18 hrs. The catalyst is filtered off, washed with MeOH and the combined filtrates concentrated in vacuo to afford the title compound as nearly colorless oil (397 mg, quant.). LCMS 384 [M+H]⁺, RT 2.35 mins (pH 2.5). ¹H NMR 300 MHz (CDCl₃) (δ ppm): 7.15-7.32 (5H, m), 5.54 (1H, s), 5.13 (2H, bs), 4.67 (1H, m), 4.30 (1H, m), 3.22-3.70 (4H, m), 3.00 (2H, dd), 2.79 (2H, dd), 2.22 (1H, m), 2.02 (1H, m), 1.45 (9H, s).

### Example 60

### Synthesis of 4-[adamantan-2-yl]-2.6-dichloropyrimidine (Intermediate 86)

2,4,6-Trichloropyrimidine (2.009 g) and PdCl₂(dppf) (CAS RN 72287-26-4) (0.232 g) is added to a solution of adamantan-2-yl(bromo)zinc (CAS RN 171860-65-4) (0.5M in THF, 24.1 ml) and heated under N₂ at 75° C. for 20 hrs. The solvent is removed in vacuo, the residue partitioned between DCM (70 ml) and water (40 ml), filtered through a Celite pad, the organic phase separated, dried (MgSO₄) and concentrated in vacuo. Flash chromatography of the residue (DCM-Heptane 3:7) affords the title compound as a colorless crystalline solid (1.47 g, 48%). LCMS 283 [M+H]⁺, RT 5.12 mins (pH 2.5). ¹H NMR 300 MHz (d₆-DMSO) (δ ppm): 7.75 (1H, s), 3.05 (1H, s), 2.55 (2H, s), 1.50-2.00 (12H, m).

### Example 61

### Synthesis of 4-[adamantan-2-yl]-6-[(3R)-3-(methylamino)pyrrolidin-1-yl]pyrimidin-2-amine (Compound 215)

Intermediate 4 (0.202 g) is dissolved in dry THF (4 ml), LiAlH₄ powder (0.06 g) carefully added, and the mixture heated at 73° C. for 2 hrs. The reaction is cooled in ice, water (0.06 ml) added dropwise, followed by 15% aq NaOH solution (0.06 ml), stirred for 15 mins, then more water (0.18 ml) added. After a further 15 mins, the white solid is filtered off through Celite, washed with THF (4×1 ml), and the filtrate concentrated in vacuo. The residue (177 mg) is dissolved in dry NMP (1.4 ml), 4-methoxybenzylamine (CAS RN 2393-23-9) (0.073 ml) and Et₃N (0.072 ml) added and the solution heated in the microwave at 180° C. for 90 mins. The solution is diluted with MTBE (10 ml), washed with brine (10 ml), dried (MgSO₄) and concentrated in vacuo. Flash chromatography of the residue (DCM-MeOH 95:5 rising to 93:6+1% 7N NH₃ in MeOH) affords the benzylamine as a pale yellow glass (133 mg, 63%). Rf (DCM-MeOH 95:5) 0.18. LCMS 448 [M+H]⁺, RT 3.03 mins (pH 5.8). This is dissolved in TFA (2 ml) and heated at 75° C. for 60 mins. The TFA is removed in vacuo and the residue partitioned between EtOAc and saturated NaHCO₃, the organic phase dried (MgSO₄) and concentrated in vacuo. Prep-HPLC (pH 2.5) followed by a DCM/MeOH/Heptane azeotrope affords the title compound as a white solid (69 mg, 70%). LCMS 328 [M+H]⁺, RT 1.33 mins (pH 2.5). ¹H NMR 300 MHz (CD₃OD) (δ ppm): 6.10 (1H, s), 3.70-4.10 (5H, m), 2.98 (1H, s), 2.80 (3H, s), 2.54 (1H, m), 2.41 (2H, s), 2.30 (1H, m), 1.70-2.15 (12H, m).

### Example 62

### Synthesis of 4-chloro-6-(cyclopentylmethyl)pyrimidin-2-amine (Intermediate 87)

Intermediate 81 (0.486 g) is suspended in POCl₃ (4 ml) and heated at 110° C. for 25 mins. The resulting solution is cooled in ice, carefully quenched dropwise into water and brine (35 ml), extracted with DCM (2×30 ml), the extracts dried (MgSO₄) and concentrated in vacuo to afford the title compound as a yellow crystalline solid (491 mg, 92%). LCMS 212 [M+H]⁺, RT 3.55 mins (pH 2.5). ¹H NMR 300 MHz (CDCl₃) (δ ppm): 6.53 (1H, s), 5.10 (2h, bs), 2.55 (2H, d), 2.20 (1H, m), 1.05-1.80 (8H, m).

### Example 63

### Synthesis of 4-(3-amino-3-methylpyrrolidin-1-yl)-6-cyclopentylpyrimidin-2-amine (Compound 216)

4-Chloro-6-cyclopentylpyrimidin-2-amine (0.065 g) and N-(3-methylpyrrolidin-3-yl)acetamide (CAS RN 96567-95-2) (0.062 g) are dissolved in absolute EtOH (1.6 ml), DIPEA (0.063 ml) is added and the solution is heated in the microwave at 160° C. for 30 mins. The solvent is removed in vacuo, the residue dissolved in 1,4-dioxane (3 ml), 6N HCl solution (1.5 ml) added, and the solution heated at 95° C. for 36 hrs. The dioxane is removed in vacuo, Brine (5 ml) and 48% NaOH are added and the solution is extracted with EtOAc (3×15 ml). The combined organic layer is dried (MgSO₄), and concentrated to dryness in vacuo. Flash chromatography of the residue (DCM-MeOH 94:6+1% 7N NH₃ in MeOH) affords the title compound as a colorless oil (31 mg, 36%). Rf (DCM-MeOH 95:5+1% 7N NH₃ in MeOH) 0.19. LCMS 262 [M+H]⁺, RT 1.75 mins (pH 5.8). ¹H NMR 300 MHz (CDCl₃) (δ ppm): 5.62 (1H, s), 4.80 (2H, bs), 3.15-3.67 (4H, m), 2.80 (1H, m), 1.53-2.05 (12H, m), 1.35 (3H, s).

### Example 64

### Synthesis of 4-cyclopentyl-6-(1,7-diazaspiro[4.4]non-7-yl)pyrimidin-2-amine (Compound 217)

Intermediate 49 (0.173 g) and HCl 2.OM in diethyl ether (0.23 ml) are dissolved in absolute EtOH (15 ml), the solution degassed, 10 wt % palladium on carbon (0.08 g) added and hydrogenated under 1 atm H₂ for 4 hrs. The catalyst is filtered off through Celite, washed with EtOH, the filtrate is evaporated in vacuo, the residue dissolved in DCM (30 ml) plus EtOAc (5 ml), washed with saturated NaHCO₃ (3 ml) plus 1N NaOH (3 ml) and brine (3 ml), dried (MgSO₄) and concentrated in vacuo to afford the title compound as a pale yellow glass (115 mg, 87%). LCMS 288 [M+H]⁺, RT 1.49 mins (pH 5.8). ¹H NMR 300 MHz (CDCl₃) (δ ppm): 5.62 (1H, s), 5.00 (2H, bs), 3.20-3.70 (4H, m), 3.03 (2H, m), 2.80 (1H, m), 2.55-2.90 (1H, m), 1.53-2.03 (14H, m).

### Example 65

### Synthesis of 4-cyclopentyl-6-[3-(diethylamino)azetidin-1-yl]pyrimidin-2-amine acetate salt (Compound 218)

Compound 35 (0.065 g) is dissolved in DCM (3 ml) and THF (1 ml), trimethyl orthoformate (1.5 ml) added, and the solution cooled in an ice bath. Acetaldehyde (approx. 0.05 ml) is added, and after 5 mins, NaBH(OAc)₃ (0.065 g). The solution is stirred at room temperature for 18 hrs. DCM (15 ml) is added and the solution is washed with saturated NaHCO₃ (10 ml), dried (MgSO₄) and concentrated in vacuo. Prep-HPLC of the residue (pH 5.8) affords the title compound as a colorless solid (8.7 mg, 11%). LCMS 290 [M+H]⁺, RT 2.22 mins (pH 5.8). ¹H NMR 300 MHz (CDCl₃) (δ ppm): 6.85 (2H, bs), 5.45 (1H, s), 4.10 (2H, t), 3.95 (2H, t), 3.70 (1H, m), 2.95 (1H, m), 2.58 (4H, q), 2.05 (3H, s, AcOH), 1.50-1.88 (8H, m), 1.02 (6H, t).

### Example 66

### Synthesis of N⁴-8-azabicyclo[3.2.1]oct-3-yl-6-cyclohexylpyrimidine-2,4-diamine (Compound 219)

10% Palladium on charcoal (0.05 g) is added to a solution of Intermediate 29 (0.097 g) in methanol (20 ml). The mixture is stirred under atmospheric pressure H₂ at room temperature for 4 hrs. The solution is filtered and the solvent evaporated in vacuo. Purification of the residue by preparative HPLC (pH 5.8) followed by evaporation under vacuum at 40° C. affords the title compound as a colorless oil (0.002 g, 2.7%). LCMS 302 [M+H]⁺, RT 1.77 mins (pH 5.8). ¹H NMR 300 MHz (CDCl₃) (δ ppm): 5.55 (1H, s), 5.49 (1H, s), 4.35 (1H, br s),3.95 (2H,s), 3.80 (2H, m), 3.65 (1H, s), 2.45 (1H, m), 1.90-2.25 (4H, m), 1.60-1.90 (9H, m), 1.20-1.49 (5H, m).

### Example 67

### Synthesis of N-[1-(2-amino-6-cyclopentylpyrimidin-4-yl)azetidin-3-yl]acetamide (Intermediate 88)

Compound 35 (0.092 g) is suspended in dry DCM (6 ml), DMAP (catalytic), DIPEA (0.076 ml) and acetic anhydride (0.041 ml) are added and the resulting solution is stirred at room temperature for 3 hrs. The reaction is diluted with DCM (25 ml), quenched with aq. NH₄Cl (20 ml), the aqueous phase saturated with NaCl, the layers separated, and the aqueous phase further extracted with DCM (2×15 ml) and EtOAc (20 ml). The combined organics are dried (MgSO₄) and evaporated in vacuo. Flash chromatography (DCM-MeOH 95:5 rising to 9:1) affords the title compound as a colorless glass (45 mg, 41%). R_{f} (DCM-MeOH 95:5) 0.11. LCMS 276 [M+H]⁺, RT 1.42 mins (pH 2.5). ¹H NMR 300 MHz (CDCl₃) (δ ppm): 6.87 (1H, bd), 5.50 (1H, s), 5.32 (2H, bs), 4.80 (1H, m), 4.32 (2H, dd), 3.88 (2H, dd), 3.41 (1H, m), 2.02 (3H, s), 1.55-2.10 (8H, m).

### Example 68

### Synthesis of 4-cyclopentyl-6-[3-(ethylamino)azetidin-1-yl]pyrimidin-2-amine (Compound 220)

Intermediate 88 (0.033 g) is dissolved in anhydrous THF (5 ml), LiAlH₄ (0.020 g) is added and the resulting mixture is stirred and heated at 75° C. for 5 hrs. The reaction is cooled, quenched with water (0.020 ml), then 15% NaOH solution (0.020 ml), and finally water (0.060 ml). The resulting solids are filtered-off through Celite, washed with THF (4×3 ml), and the combined filtrate concentrated in vacuo. Prep-HPLC (pH 5.8) followed by three DCM-MeOH-Heptane azeotropes affords the title compound as a colorless solid (8.1 mg, 26%). LCMS 262 [M+H]⁺, RT 1.99 mins (pH 5.8). ¹H NMR 300 MHz (CDCl₃) (δ ppm): 7.00 (AcOH), 5.44 (1H, s), 4.82 (3H, bs), 4.25 (2H, m), 3.80 (3H, m), 2.95 (1H, m), 2.65 (2H, q), 2.00-2.15 (5H, m), 1.50-1.82 (6H, m), 1.14 (3H, t).

### Biological Examples

### Example 69

### Human H₄R³Histamine Binding Assay

Cf. The Journal of Pharmacology and Experimental Therapeutics 2001, 299(1); 121-130.

³Histamine dihydrochloride (Amersham) binding to the human H₄ receptor is determined using CHO-hH₄R membranes (350 μg/ml; Euroscreen), SPA beads (GE Healthcare; 15 mg/ml) and histamine (20 μM) in assay buffer [Tris HCl (50 mM), EDTA (5 mM, pH 7.4), 0.1% fatty acid free BSA]. The test compounds (0.5% DMSO final) are incubated with the assay mix in 96-well Optiplates (Perkin Elmer) for 15 mins at room temperature prior to addition of ³H-histamine solution (10 nM); the final assay volume is 200 μl per well. The plates are sealed and incubated for 16 h at room temperature prior detection of membrane bound radioligand on Topcount (Perkin Elmer). Unless noted, all reagents are purchased from Sigma. Affinity (pKᵢ) measurements are determined by assessing the concentration of compound necessary to displace 50% of the specifically bound ³H-histamine.

The compounds of the invention are tested in this assay their Kᵢ/EC50 measurements are of less than 10 μM. The preferred compounds of the invention give Kᵢ/EC₅₀ measurements less than 1 μM. Most preferred compounds have activities less than 100 nM.

Compound 43, 4-(3-aminopyrrolidin-1-yl)-6-(1,3-dihydro-2H-isoindol-2-yl)pyrimidin-2-amine, gives a Kᵢ/EC₅₀ between 200 and 450 nM.

### Example 70

### Human H₄ GTPγS³⁵ Assay

Cf. The Journal of Pharmacology and Experimental Therapeutics 2000, 296(3); 1058-1066.

GTPγS³⁵ (Amersham) binding is determined using CHO-hH₄R membranes (Euroscreen; 50 μg/ml), SPA beads (GE Healthcare; 10 mg/ml), GDP (15 μM) and saponin (30 μg/ml) in assay buffer [20 mM Hepes, 100 mM NaCl, 10 mM MgCl, 1 mM EDTA (pH 7.4), 0.1% BSA) in 96-well Optiplates (Perkin Elmer). Test compounds (0.5% DMSO final) are added and plates are incubated for 1 h at room temperature. GTPγS³⁵ (300 pM) is added (final assay volume 200 μl/well) and plates are incubated for a further 90 mins at room temperature prior to centrifugation of plates and detection using Topcount (Perkin Elmer). Unless noted, all reagents are purchased from Sigma. Affinity/efficacy measurements (pKᵢ/pEC₅₀) are determined by assessing the concentration of compound necessary to inhibit 50% of the functional response to a fixed concentration of histamine (GTPγS³⁵ binding), or the concentration of compound to cause a 50% increase in GTPγS³⁵ binding. The compounds of the invention are tested in this assay their Kᵢ/EC₅₀ measurements are of less than 10 μM. The preferred compounds of the invention give Kᵢ/EC₅₀ measurements less than 1 μM. Most preferred compounds have activities less than 100 nM.

Compound 43, 4-(3-aminopyrrolidin-1-yl)-6-(1,3-dihydro-2H-isoindol-2-yl)pyrimidin-2-amine, gives a Kᵢ/EC₅₀ between 75 and 250 nM.

1. A compound having formula I or pharmaceutically acceptable salts thereof or stereoisomeric forms thereof, and the geometrical isomers, enantiomers, diastereoisomers, and pharmaceutically acceptable salts thereof 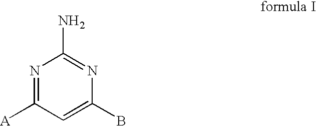
represents the point of attachment to the rest of the molecule wherein A is a group of formula II 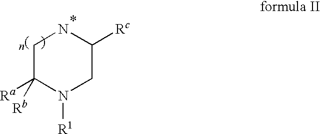
wherein
n is 1 or 2;
R¹ is hydrogen or is unsubstituted C₁₋₃ alkyl groups or is C₃₋₅ unsubstituted cycloalkyl;
R^{a} is hydrogen or is unsubstituted C₁₋₄ alkyl groups;
R^{b} is hydrogen or is unsubstituted C₁₋₃ alkyl groups;
R^{c} is hydrogen or is unsubstituted C₁₋₃ alkyl groups;
or A is a group of formula III 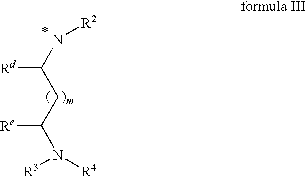
wherein
m is 0, 1 or 2;
R² is hydrogen or is unsubstituted C₁₋₃ alkyl group;
R³ is hydrogen or is unsubstituted C₁₋₃ alkyl group;
R⁴ is hydrogen or is unsubstituted C₁₋₃ alkyl group;
R^{d} is hydrogen or is unsubstituted C₁₋₃ alkyl group;
R^{e} is hydrogen or is unsubstituted C₁₋₃ alkyl group;
or A is a group of formula IV 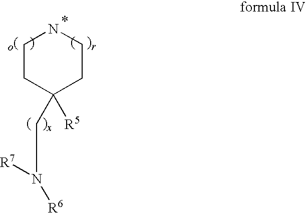
wherein
o is 0 or 1;
r is 0, 1 or 2;
x is 0 or 1;
R⁵ is hydrogen or is unsubstituted C₁₋₃ alkyl group;
R⁶ is hydrogen or is unsubstituted C₁₋₃ alkyl group;
R⁷ is hydrogen or is unsubstituted C₁₋₃ alkyl group;
or A is a group of formula V 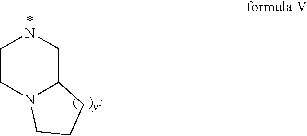
wherein
y is 1 or 2;
or A is a group of formula VI 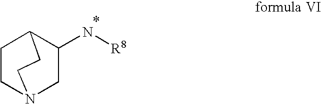
wherein
R⁸ is hydrogen or is unsubstituted C₁₋₃ alkyl group;
or A is a group of formula VII 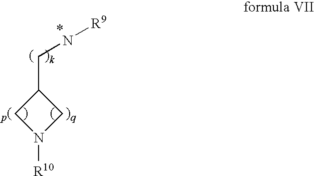
wherein
k is 0 or 1;
p is 1 or 2 or 3;
q is 0 or 1 or 2;
R⁹ is hydrogen or is unsubstituted C₁₋₃ alkyl group;
R¹⁰ is hydrogen or is unsubstituted C₁₋₃ alkyl group;
or A is a group of formula VIII 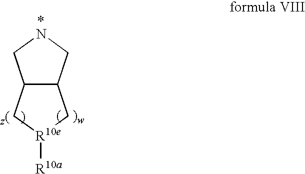
wherein
z is 0, 1, 2 or 3;
w is 0 or 1;
R^{10a} is hydrogen or is unsubstituted C₁₋₃ alkyl group or is NH₂; and R^{10e} is a CH group; or
R^{10a} is hydrogen or is unsubstituted C₁₋₃ alkyl group; and R^{10e} is N;
or A is a group of formula XII 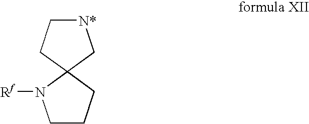
wherein
R^{f} is hydrogen or is unsubstituted C₁₋₃ alkyl group;
or A is a group of formula XIII 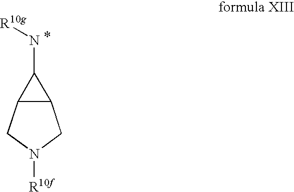
wherein
R^{10f} is hydrogen or is unsubstituted C₁₋₃ alkyl group;
R^{10g} is hydrogen or is unsubstituted C₁₋₃ alkyl group;
or A is a group of formula XIV 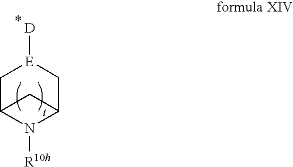
wherein
D is NH; and E is CH; or
D is direct bond; and E is CH or N;
t is 1, 2 or 3;
R^{10h} is hydrogen or is unsubstituted C₁₋₃ alkyl group;
and
wherein
B is defined as C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl;
or B is defined as C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups;
or B is defined as C2-7 alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups;
or B is defined as C₁ alkyl substituted by C₃₋₁₀ cycloalkyl (mono or polycyclic), or by 1
C₁₋₃ alkoxy group, or by a heterocycle (aromatic or non aromatic), or by C₁₋₄ alkyl, or by aryl;
or B is defined as C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;
or B is a group of formula IX 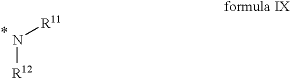
wherein
R¹¹ is hydrogen and R¹² is C₅₋₁₀ cycloalkyl (mono- or polycyclic) optionally substituted by 1 to 3 C₁₋₄ alkyl groups, or by aryl, or by an amide moiety, or fused to an aryl;
or R¹¹ is hydrogen and R¹² is C₅₋₁₀ nonaromatic heterocycle (mono- or polycyclic) optionally substituted by a ketone moiety, or by aryl, or by C₁₋₄ alkyl;
or R¹¹ is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups and R¹² is C₃₋₁₀ cycloalkyl (mono- or polycyclic) optionally substituted by 1 to 3 C₁₋₄ alkyl groups;
or R¹¹ is C₁₋₇ alkyl (linear or branched) optionally substituted by C₃₋₁₀ cycloalkyl (mono- or polycyclic), or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups and R¹² is C₁₋₇ alkyl (linear or branched) optionally substituted by C₃₋₁₀ cycloalkyl (mono- or polycyclic)or by aryl optionally substituted by 1-3 halogens, or by aryl optionally substituted by 1 or 2 C₁₋₃ alkoxy groups, or by aryl optionally substituted by C₁₋₃ haloalkoxy groups, or by aryl optionally substituted by C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;
or B is a group of formula X 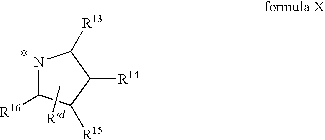
wherein
R¹³ is hydrogen or is C₁₋₇ alkyl group (linear or branched) optionally substituted by C₃₋₁₀ cycloalkyl (mono- or polycyclic) or by aryl optionally substituted by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or non-aromatic) optionally substituted by a ketone moiety, or by a heterocycle (aromatic or non-aromatic) optionally substituted by 1 to 3 C₁₋₄ alkyl groups, or one of the methylene groups can be replaced by an oxygen;
R¹⁴ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or can form together with R¹⁵ a benzene ring fused to the nitrogen heterocycle which can optionally be substituted by 1 to 3 halogens;
R¹⁵ is hydrogen or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups;
R¹⁶ is hydrogen or can form together with R¹³ a C₁₋₃ alkylene chain;
R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a nitrile, or by aryl optionally substituted by a heterocycle (aromatic or nonaromatic) optionally substituted by 1 to 3 C₁₋₄ alkyl groups;
d is 0 to 2;
or B is a group of formula XI 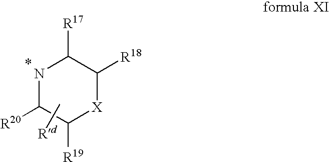
wherein
R¹⁷ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by nitrile, or by aryl optionally substituted by 1 to 3 C₁₋₄ alkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl, or can form together with R¹⁸ a benzene ring fused to the nitrogen heterocycle;
R¹⁸ is hydrogen or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1-3 C₁₋₄ alkyl groups or can form together with R²¹ a benzene ring fused to the nitrogen heterocycle, in which case R²² is not present;
R¹⁹ is hydrogen, or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups or or can form together with R¹⁷ a C₁₋₃ alkylene chain;
R²⁰ is hydrogen or is C₁₋₃ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by an heterocycle (aromatic or nonaromatic) optionally substituted by 1 to 3 C₁₋₄ alkyl groups, or can form together with R¹⁷ a C₁₋₃ alkylene chain;
X is CR²¹R²², or is NR²³ or is C₂₋₃ alkylene chain;
R²¹ is hydrogen, or is aryl optionally substituted by 1 to 3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by nitrile, or is aryl optionally substituted by 1 to 3 C₁₋₄ alkyl groups or is C₁₋₇ alkyl groups (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups or is C₁₋₃ alkoxy group;
R²² is hydrogen, or is hydroxyl, or is C₁₋₃ alkoxy, or is C₁₋₃ haloalkyl groups;
R²³ is hydrogen, or is aryl optionally substituted by 1-3 halogens, or is aryl optionally substituted by 1 or 2 C₁₋₃ alkoxy groups, or is aryl optionally substituted by 1 or 2 C₁₋₃ haloalkyl groups, or is aryl optionally substituted by 1 or 2 C₁₋₃ haloalkoxy groups, or is aryl optionally substituted by 1 to 3 C₁₋₄ alkyl groups, or is aryl optionally substituted by a heterocycle (aromatic or nonaromatic) optionally substituted by a ketone moiety or is aryl optionally substituted by a heterocycle (aromatic or nonaromatic) optionally substituted by nitrile or is aryl optionally substituted by a heterocycle (aromatic or nonaromatic) optionally substituted by 1 to 3 C₁₋₄ alkyl groups;
R′ is C₁₋₇ alkyl (linear or branched) optionally substituted by C₃₋₁₀ cycloalkyl (mono- or polycyclic) or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by nitrile, or by aryl optionally substituted by a heterocycle (aromatic or nonaromatic) optionally substituted by 1 to 3 C₁₋₄ alkyl groups;
d is 0 to 2;
except:
4-[3-(Methylamino)azetidin-1-yl]-6(4-methylpiperidin-1-yl)pyrimidin-2,4-diamine;
N⁴-Bicyclo[1.1.1]pent-1-yl-6[(3R)-3-methylamino)pyrolidin-1-yl]pyrimidine-2,4-diamine;
4-[3-(Methylamino)azetidin-1-yl]-6-piperidin-1-yl)pyrimidin-2-diamine;
4-[(4-ethyl-1-piperazinyl)-6-propyl]pyrimidin-2-amine.

2. A compound according to claim 1 wherein
when n is 1 then R¹ is hydrogen or is methyl or is cyclopropyl or is ethyl or is isopropyl and R^{a} is hydrogen or methyl and R^{b} is hydrogen or is methyl and R^{c} is hydrogen or is methyl; or
when n is 1 then R¹ is hydrogen or methyl or ethyl and R^{a} is hydrogen or methyl or ethyl or iso-propyl or iso-butyl and R^{b} is hydrogen and R^{c} is hydrogen or methyl; or
when n is 2 then R¹ is methyl and R^{a} is hydrogen and R^{b} is hydrogen and R^{c} is hydrogen; or
when n is 2 then R¹ is hydrogen and R^{a} is hydrogen and R^{b} is hydrogen and R^{c} is hydrogen;
when m is 1 then R² is methyl or hydrogen and R³ is methyl and R⁴ is methyl and R^{d} is hydrogen and R^{e} is hydrogen; or
when m is 0 then R² is hydrogen and R³ is methyl and R⁴ is methyl and R^{d} is hydrogen and R^{e} is hydrogen; or
when m is 0 then R² is hydrogen and R³ is hydrogen and R⁴ is methyl and R^{d} is hydrogen and R^{e} is hydrogen; or
when m is 0 then R² is hydrogen and R³ is methyl and R⁴ is hydrogen and R^{d} is hydrogen and R^{e} is hydrogen;
when o is 0 then r is 0 or 1 and x is 0 or 1 and R⁵ is hydrogen or methyl and R⁶ is hydrogen or methyl or ethyl and R⁷ is hydrogen or ethyl; or
when o is 1 then r is 1 and x is 0 and R⁵is hydrogen and R⁶ is hydrogen and R⁷ is methyl;
when p is 1 then q is 2 and k is 0 and R⁹ is hydrogen and R¹⁰ is hydrogen; or
when p is 1 then q is 2 and k is 0 and R⁹ is methyl and R¹⁰ is methyl; or
when p is 2 then q is 2 and k is 0 and R⁹ is hydrogen and R¹⁰ is methyl;
when z is 1 then w is 1 and R^{10a} is hydrogen and R^{10e} is N; or
when z is 3 then w is 0 and R^{10a} is hydrogen and R^{10e} is N;
when R^{10f} is hydrogen then R^{10g} is hydrogen; or
when R^{10f} is methyl then R^{10g} is hydrogen;
when D is NH then E is CH and t is 2 and R^{10h} is hydrogen; or
when D is NH then E is CH and t is 2 and R^{10h} is methyl; or
when D is direct bond then E is N and t is 2 and R^{10h} is hydrogen; or
B is cyclohexyl or adamantyl or cyclopentyl or ethyl-2-propyl or iso-propyl or 1-methyl-pentyl or 2-phenylethyl or cyclopropyl or methylcyclohexyl orcycloheptyl or 1,2,3,4 tetrahydronaphtalen-2-yl or (1E) 3,3 dimethylbuty-1-en; or
B is cyclohexyl or adamantyl or cyclopentyl or cyclohexylmethyl or cyclopentylmethyl or cyclohex-1-en; or
when R¹¹ is hydrogen then R¹² is piperidine or 1 acetylpiperidine or 1R*, 2S*, 4S*-bicyclo[2.2.1]hept-2-yl-N-benzyl or N-phenylcyclohexylcarboxamide or N-phenylcyclohexanecarboxamide or N-methylcyclohexanecarboxamide or N-cyclopropylcyclohexanecarboxamide or N-tert-butylcyclohexanecarboxamide or N-(4-methoxyphenylcyclohexane or cyclohexyl or 1-phenylpiperidine or 1-benzylpiperidin-4-yl or 2,3-dihydro-1H-inden-1-yl or 1,2,3,4-tetrahydronaphthalen-2-yl or (1R*,2R*,4S*)-bicyclo[2.2.1]hept-2-yl; or
when R¹¹ is hydrogen then R¹² is adamantyl or cyclohexyl or 1,3,3-trimethylbicyclo[2.2.1]hept-2-yl or (1R; 4R) 1,7,7-trimethylbicyclo[2.2.1]hept-2-yl or (tetrahydro-2H-pyran-4-yl or cyclopentyl or cycloheptyl or (1R*,2S*,4S*)-bicyclo[2.2.1]hept-2-yl or bicyclo[2.2.1]hept-2-yl or (1R*,2R*,4S*)-bicyclo[2.2.1]hept-2-yl; or
when R¹¹ is methyl then R¹² is cyclohexyl or cyclopentyl or methyl; or
when R¹¹ is hydrogen then R¹² is cyclohexyl or (1R*,2R*,4S*)-bicyclo[2.2.1]hept-2-yl or(1R*,2S*,4S*)-bicyclo[2.2.1]hept-2-yl or [exo-bicyclo[2.2.1]hept-2-yl;
when R¹³ is hydrogen or n-propyl or tert-butyl or methyl or (2S)-2-methoxymethyl then R¹⁴ is hydrogen or 4-trifluoromethylphenoxy or 4-chlorophenyl or 2-methoxyphenyl or together with R¹⁵ can form a 1,3-dihydro-2H-isoindol-2-yl group or a 5-fluoro-1,3-dihydro-2H-isoindol-2-yl ring and R¹⁵ is hydrogen and R¹⁶ is hydrogen or together with R¹³ an ethylene chain; or
when R¹³ is hydrogen or methyl then R¹⁴ is hydrogen or 4-chlorophenyl or 2-methoxyphenyl or together with R¹⁵ can form a 1,3-dihydro-2H-isoindol-2-yl group or a 5-fluoro-1,3-dihydro-2H-isoindol-2-yl ring and R¹⁵ is hydrogen and R¹⁶ is hydrogen or together with R¹³ an ethylene chain; or
when R¹³ is hydrogen or methyl then R¹⁴ is hydrogen or together with R¹⁵ can form a 1,3-dihydro-2H-isoindol-2-yl group and R¹⁵ is hydrogen and R¹⁶ is hydrogen;
when R¹⁷ is hydrogen or methyl or ethyl or together with R¹⁹ a methylene then R¹⁸ is hydrogen or phenyl or 3-fluorophenyl or together with R²¹ a 3,4-dihydroisoquinoline ring and R¹⁹ is hydrogen and R²⁰ is hydrogen or methyl or together with R¹⁷ can form an ethylene and X is CR²¹R²² or NR²³ or ethylene and R²¹ is hydrogen or benzyl or phenyl or 4-cyanophenoxy or 4-chlorophenoxy or 4-chlorobenzyl or methyl or tert-butyl or 3-fluorophenyl or 2-trifluoromethylphenyl or 2 methoxyphenyl or methoxy or 2-methylphenyl or 4-fluorobenzyl or 2-fluorophenyl or 4-chlorophenyl and R²² is hydrogen or hydroxyl or trifluoromethyl and R²³ is 2-methoxyphenyl or 3-trifluoromethyl phenyl or 4-fluorophenyl; or
when R¹⁷ is hydrogen or methyl or ethyl or can form together with R¹⁹ a methylene group then R¹⁸ is hydrogen or phenyl or together with R²¹ a 3,4-dihydroisoquinoline ring and R¹⁹ is hydrogen and R²⁰ is hydrogen or methyl or together with R¹⁷ can form an ethylene and X is CR²¹R²² or ethylene or NR²³ and R²¹ is hydrogen or methyl or 2-methoxyphenyl or 4-chlorophenyl and R²² is hydroxyl or methyl and R²³ is 2-methoxyphenyl or 3-trifluoromethyl phenyl or 4-fluorophenyl.

3. A compound according to claim 1 wherein A is according to formula II and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1
C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups;
except:
4-[(4-ethyl-1-piperazinyl)-6-propyl]pyrimidin-2-amine.

4. A compound according to claim 1 wherein A is a group of formula II and B is a group of formula X.

5. A compound according to claim 1 wherein A is a group of formula II and B is a group of formula XI.

6. A compound according to claim 1 wherein A is a group of formula II and B is a group of formula IX.

7. A compound according to claim 1 wherein A is a group of formula V and B is a group of formula X.

8. A compound according to claim 1 wherein A is a group of formula IV and B is a group of formula XI
except 4-[3-(Methylamino)azetidin-1-yl]-6(4-methylpiperidin-1-yl)pyrimidin-2,4-diamine; and 4-[3-(Methylamino)azetidin-1-yl]-6-piperidin-1-yl)pyrimidin-2-diamine.

9. A compound according to claim 1 wherein A is a group of formula IV and B is a group of formula X.

10. A compound according to claim 1 wherein A is a group of formula V and B is a group of formula IX.

11. A compound according to claim 1 wherein A is a group of formula VI and B is a group of formula X.

12. A compound according to claim 1 wherein A is a group of formula VII and B is a group of formula X.

13. A compound according to claim 1 wherein A is a group of formula IV and B is a group of formula IX
except N⁴-Bicyclo[1.1.1]pent-1-yl-6[(3R)-3-methylamino)pyrolidin-1-yl]pyrimidine-2,4-diamine.

14. A compound according to claim 1 wherein A is a group of formula III and B is a group of formula IX.

15. A compound according to claim 1 wherein A is a group of formula VIII and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1
C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

16. A compound according to claim 1 wherein A is a group of formula IV and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1
C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

17. A compound according to claim 1 wherein A is a group of formula VII and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1
C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

18. A compound according to claim 1 wherein A is a group of formula III and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1
C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

19. A compound according to claim 1 wherein A is a group of formula XIV and B is C₃₋₁₀ cycloalkyl group (mono- or polycyclic) optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by a heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups, or fused to an aryl; or B is C₅₋₁₀ cycloalkenyl group (mono- or polycyclic), optionally substituted by 1 to 3 C₁₋₄ alkyl groups, or by 1-3 halogens, or by 1 or 2 C₁₋₃ alkoxy groups, or by 1 or 2 C₁₋₃ haloalkoxy groups, or by 1 or 2 C₁₋₃ haloalkyl groups, or by an heterocycle (aromatic or nonaromatic) optionally substituted by a ketone moiety, or by a heterocycle (aromatic or nonaromatic) optionally substituted by 1 to 3 C₁₋₄ alkyl groups, or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, by aryl optionally substituted by 1 to 3 C₁₋₄ alkyl groups; or B is C₂₋₁₂ alkyl group (linear or branched) optionally substituted by C₃₋₁₀ cycloalkyl (mono- or polycyclic), or by 1 or 2 C₁₋₃ alkoxy groups, or by a heterocycle (aromatic or nonaromatic), or by aryl optionally substituted by 1-3 halogens, or by aryl optionally substituted by 1 or 2 C₁₋₃ alkoxy groups, or by aryl optionally substituted by 1 or 2 C₁₋₃ haloalkoxy groups, or by aryl optionally substituted by 1 or 2 C₁₋₃ haloalkyl groups, or by aryl optionally substituted by an heterocycle (aromatic or nonaromatic) optionally substituted by a ketone moiety, or by aryl optionally substituted by a heterocycle (aromatic or nonaromatic) optionally substituted by a 1 to 3 C₁₋₄ alkyl groups; or B is C₁ alkyl substituted by C₃₋₁₀ cycloalkyl (mono or polycyclic), or by 1
C₁₋₃ alkoxy group, or by a heterocycle (aromatic or non aromatic), or by C₁₋₄ alkyl, or by aryl; or B is C₂₋₆ alkenyl (linear or branched) optionally substituted by C₃₋₁₀ cycloalkyl (mono- or polycyclic), or by a heterocycle (aromatic or nonaromatic), or by aryl optionally substituted by 1 to 3 C₁₋₄ alkyl groups, or by aryl optionally substituted by 1-3 halogens, or by aryl optionally substituted by 1 or 2 C₁₋₃ haloalkoxy groups, or by aryl optionally substituted by 1 or 2 C₁₋₃ haloalkyl groups, or by aryl optionally substituted by a heterocycle (aromatic or nonaromatic) optionally substituted by a ketone moiety, or by aryl optionally substituted by a heterocycle (aromatic or nonaromatic) optionally substituted by 1 to 3 C₁₋₄ alkyl groups.

20. A compound according to claim 1 selected from the group consisting of
4-(4-methylpiperazin-1-yl)-6-piperidin-1-ylpyrimidin-2-amine;
4-(1,3-dihydro-2H-isoindol-2-yl)-6-(4-methylpiperazin-1-yl)pyrimidin-2-amine;
4-(4-methylpiperazin-1-yl)-6-(4-methylpiperidin-1-yl)pyrimidin-2-amine;
4-[4-(2-methoxyphenyl)piperidin-1-yl]-6-(4-methylpiperazin-1-yl)pyrimidin-2-amine;
4-(3,4-dihydroisoquinolin-2(1H)-yl)-6-(4-methylpiperazin-1-yl)pyrimidin-2-amine;
4-(5-fluoro-1,3-dihydro-2H-isoindol-2-yl)-6-(4-methylpiperazin-1-yl)pyrimidin-2-amine;
4-(3-aminopyrrolidin-1-yl)-6-(1,3-dihydro-2H-isoindol-2-yl)pyrimidin-2-amine;
4-(4-methylpiperazin-1-yl)-6-(2-methylpyrrolidin-1-yl)pyrimidin-2-amine;
4-(2-ethylpiperidin-1-yl)-6-(4-methylpiperazin-1-yl)pyrimidin-2-amine;
4-[3-(2-methoxyphenyl)pyrrolidin-1-yl]-6-(4-methylpiperazin-1-yl)pyrimidin-2-amine;
4-[3-(4-chlorophenyl)pyrrolidin-1-yl]-6-(4-methylpiperazin-1-yl)pyrimidin-2-amine;
4-(2-methylpyrrolidin-1-yl)-6-piperazin-1-ylpyrimidin-2-amine;
4-(4-methylpiperazin-1-yl)-6-[(2R)-2-methylpyrrolidin-1-yl]pyrimidin-2-amine;
4-(4-methylpiperazin-1-yl)-6-[(2S)-2-methylpyrrolidin-1-yl]pyrimidin-2-amine;
4-(2,6-dimethylpiperidin-1-yl)-6-(4-methylpiperazin-1-yl)pyrimidin-2-amine;
4-(6-azabicyclo[3.2.1]oct-6-yl)-6-(4-methylpiperazin-1-yl)pyrimidin-2-amine;
4-azepan-1-yl-6-(4-methylpiperazin-1-yl)pyrimidin-2-amine;
1-[2-amino-6-(4-methylpiperazin-1-yl)pyrimidin-4-yl]-4-(4-chlorophenyl)piperidin-4-ol;
4-(4-methylpiperazin-1-yl)-6-(3-phenylpiperidin-1-yl)pyrimidin-2-amine;
N⁴-cyclohexyl-6-(hexahydropyrrolo[1,2-a]pyrazin-2(1H)-yl)pyrimidine-2,4-diamine;
N⁴-adamantan-1-yl-6-(4-methylpiperazin-1-yl)pyrimidine-2,4-diamine;
6-(4-methylpiperazin-1-yl)-N⁴-(1,3,3-trimethylbicyclo[2.2.1]hept-2-yl)pyrimidine-2,4-diamine;
N⁴-adamantan-2-yl-6-(4-methylpiperazin-1-yl)pyrimidine-2,4-diamine;
6-(4-methylpiperazin-1-yl)-N⁴(1R; 4R) (1,7,7-trimethylbicyclo[2.2.1]hept-2-yl)pyrimidine-2,4-diamine;
N⁴-cyclohexyl-N⁴-methyl-6-(4-methylpiperazin-1-yl)pyrimidine-2,4-diamine;
4-(7-azabicyclo[2.2.1]hept-7-yl)-6-(4-methylpiperazin-1-yl)pyrimidin-2-amine;
4-(3-aminopyrrolidin-1-yl)-6-(5-fluoro-1,3-dihydro-2H-isoindol-2-yl)pyrimidin-2-amine;
4-(hexahydropyrrolo[1,2-a]pyrazin-2(1H)-yl)-6-(2-methylpyrrolidin-1-yl)pyrimidin-2-amine;
N⁴-1-azabicyclo[2.2.2]oct-3-yl-6-(1,3-dihydro-2H-isoindol-2-yl)pyrimidine-2,4-diamine;
4-(3-methyl-3,4-dihydroisoquinolin-2(1H)-yl)-6-(4-methylpiperazin-1-yl)pyrimidin-2-amine;
N⁴-1-azabicyclo[2.2.2]oct-3-yl-6-(2-methylpyrrolidin-1-yl)pyrimidine-2,4-diamine;
6-(2-methylpyrrolidin-1-yl)-N⁴-pyrrolidin-3-ylpyrimidine-2,4-diamine;
4-[4-(methylamino)piperidin-1-yl]-6-(2-methylpyrrolidin-1-yl)pyrimidin-2-amine;
4-(1,3-dihydro-2H-isoindol-2-yl)-6-[4-(methylamino)piperidin-1-yl]pyrimidin-2-amine;
4-(1,3-dihydro-2H-isoindol-2-yl)-6-(4-methyl-1,4-diazepan-1-yl)pyrimidin-2-amine;
6-[(3R)-3-aminopyrrolidin-1-yl]-N⁴-cyclohexylpyrimidine-2,4-diamine triacetate salt;
4(R) -(3-methyl-3,4-dihydroisoquinolin-2(1H)-yl)-6-(4-methylpiperazin-1-yl)pyrimidin-2-amine;
4(S) -(3-methyl-3,4-dihydroisoquinolin-2(1H)-yl)-6-(4-methylpiperazin-1-yl)pyrimidin-2-amine;
N⁴-cyclohexyl-N⁶-[2-(dimethylamino)ethyl]pyrimidine-2,4,6-triamine;
N⁴-cyclohexyl-6-[4-(methylamino)piperidin-1-yl]pyrimidine-2,4-diamine;
6-[(3S)-3-aminopyrrolidin-1-yl]-N⁴-cyclohexylpyrimidine-2,4-diamine;
N⁴-cyclopentyl-6-(4-methylpiperazin-1-yl)pyrimidine-2,4-diamine;
N⁴-cyclopentyl-N⁴-methyl-6-(4-methylpiperazin-1-yl)pyrimidine-2,4-diamine;
N⁴-cycloheptyl-6-(4-methylpiperazin-1-yl)pyrimidine-2,4-diamine;
4-[(1R*,5S*)-8-azabicyclo[3.2.1]oct-8-yl]-6-(4-methylpiperazin-1-yl)pyrimidin-2-amine;
N⁴-[(1R*,2S*,4S*)-bicyclo[2.2.1]hept-2-yl]-6-(4-methylpiperazin-1-yl)pyrimidine-2,4-diamine;
N⁴-bicyclo[2.2.1]hept-2-yl-6-(4-methylpiperazin-1-yl)pyrimidine-2,4-diamine;
N⁴-[(1R*,2R*,4S*)-bicyclo[2.2.1]hept-2-yl]-N⁶-[2-(dimethylamino)ethyl]pyrimidine-2,4,6-triamine;
N⁴-[(1R*,2R*,4S*)-bicyclo[2.2.1]hept-2-yl]-6-[(3aR*,6aS*)-hexahydropyrrolo[3,4-c]pyrrol-2(1H)-yl]pyrimidine-2,4-diamine;
4-cyclohexyl-6-(4-methylpiperazin-1-yl)pyrimidin-2-amine;
4-adamantan-2-yl-6-(4-methylpiperazin-1-yl)pyrimidin-2-amine;
4-isopropyl-6-(4-methylpiperazin-1-yl)pyrimidin-2-amine;
4-(1-methylpentyl)-6-(4-methylpiperazin-1-yl)pyrimidin-2-amine;
4-cyclopentyl-6-(4-methylpiperazin-1-yl)pyrimidin-2-amine;
4-(1-ethylpropyl)-6-(4-methylpiperazin-1-yl)pyrimidin-2-amine;
4-cyclohexyl-6-(hexahydropyrrolo[1,2-a]pyrazin-2(1H)-yl)pyrimidin-2-amine;
4-cyclohexyl-6-(4-methyl-1,4-diazepan-1-yl)pyrimidin-2-amine;
4-cyclohexyl-6-(4-ethylpiperazin-1-yl)pyrimidin-2-amine;
4-cyclohexyl-6-[3-(dimethylamino)pyrrolidin-1-yl]pyrimidin-2-amine;
6-cyclohexyl-N⁴-methyl-N⁴-(1-methylpyrrolidin-3-yl)pyrimidine-2,4-diamine;
6-cyclohexyl-N⁴-(1-methylpiperidin-4-yl)pyrimidine-2,4-diamine;
4(R)-(4-methylpiperazin-1-yl)-6-(1-phenylethyl)pyrimidin-2-amine;
4(S)-(4-methylpiperazin-1-yl)-6-(1-phenylethyl)pyrimidin-2-amine;
4-(3-aminopyrrolidin-1-yl)-6-cyclopropylpyrimidin-2-amine;
4-cyclopropyl-6-[3-(methylamino)pyrrolidin-1-yl]pyrimidin-2-amine;
4-(3-aminopyrrolidin-1-yl)-6-(4-methylcyclohex-1-en-1-yl)pyrimidin-2-amine ditrifluoroacetic acid salt;
6-cyclohex-1-en-1-yl-N⁴-[2-(dimethylamino)ethyl]pyrimidine-2,4-diamine;
4-tert-butyl-6-(4-methylpiperazin-1-yl)pyrimidin-2-amine acetate salt;
4-tert-butyl-6-[3-(methylamino)pyrrolidin-1-yl]pyrimidin-2-amine acetate salt;
4-[adamantan-2-yl]-6-[(3S)-3-aminopyrrolidin-1-yl]pyrimidin-2-amine;
4-[adamantan-2-yl]-6-[(3S)-3-(methylamino)pyrrolidin-1-yl]pyrimidin-2-amine;
4-cyclohept-1-en-1-yl-6-(4-methylpiperazin-1-yl)pyrimidin-2-amine;
4-cyclopentyl-6-(3-methylpiperazin-1-yl)pyrimidin-2-amine;
4-(3-aminoazetidin-1-yl)-6-cyclohex-1-en-1-ylpyrimidin-2-amine;
4-cyclohexyl-6-(3-methylpiperazin-1-yl)pyrimidin-2-amine;
4-(3-aminoazetidin-1-yl)-6-cyclohexylpyrimidin-2-amine;
4-(4-methylpiperazin-1-yl)-6-(1,2,3,4-tetrahydronaphthalen-2-yl)pyrimidin-2-amine;
4-[3-(methylamino)pyrrolidin-1-yl]-6-(1,2,3,4-tetrahydronaphthalen-2-yl)pyrimidin-2-amine;
4-(3-aminopyrrolidin-1-yl)-6-(1,2,3,4-tetrahydronaphthalen-2-yl)pyrimidin-2-amine;
4-cyclopentyl-6-[(3S)-3-methylpiperazin-1-yl]pyrimidin-2-amine;
4-cyclopentyl-6-[(3R)-3-methylpiperazin-1-yl]pyrimidin-2-amine;
4-cyclohexyl-6-[(2S)-2-methylpiperazin-1-yl]pyrimidin-2-amine;
4-[(3R)-3-aminopyrrolidin-1-yl]-6-[(1E)-3,3-dimethylbut-1-en-1-yl]pyrimidin-2-amine;
4-[3-(aminomethyl)azetidin-1-yl]-6-cyclohexylpyrimidin-2-amine;
4-(3-aminoazetidin-1-yl)-6-cyclopentylpyrimidin-2-amine;
4-cyclopentyl-6-(3-ethylpiperazin-1-yl)pyrimidin-2-amine;
4-cyclopentyl-6-[(3S)-3-isopropylpiperazin-1-yl]pyrimidin-2-amine;
4-cyclopentyl-6-(3,8-diazabicyclo[3.2.1]oct-3-yl)pyrimidin-2-amine;
N⁴-(2,3-dihydro-1H-inden-2-yl)-6-[3-(methylamino)pyrrolidin-1-yl]pyrimidine-2,4-diamine;
4-cyclopentyl-6-[(3S)-3-isobutylpiperazin-1-yl]pyrimidin-2-amine;
4-cyclopentyl-6-[3-(ethylamino)azetidin-1-yl]pyrimidin-2-amine;
6-(4-methylpiperazin-1-yl)-N⁴-(tetrahydro-2H-pyran-4-yl)pyrimidine-2,4-diamine;
6-(4-methylpiperazin-1-yl)-N⁴-(tetrahydro-2H-pyran-4-yl)pyrimidine-2,4-diamine acetate;
N⁴-cyclohexyl-6-(4-methylpiperazin-1-yl)pyrimidine-2,4-diamine;
4-(1,3-dihydro-2H-isoindol-2-yl)-6-piperazin-1-ylpyrimidin-2-amine;
4-(3-aminopyrrolidin-1-yl)-6-(2-methylpyrrolidin-1-yl)pyrimidin-2-amine;
4-[3-(methylamino)pyrrolidin-1-yl]-6-(2-methylpyrrolidin-1-yl)pyrimidin-2-amine;
6-(3-aminopyrrolidin-1-yl)-N⁴-[(1R*,2S*,4S*)-bicyclo[2.2.1]hept-2-yl]pyrimidine-2,4-diamine;
N⁴-[(1R*,2S*,4S*)-bicyclo[2.2.1]hept-2-yl]-6-[3-(methylamino)pyrrolidin-1-yl]pyrimidine-2,4-diamine;
6-[(3S)-3-aminopyrrolidin-1-yl]-N⁴-[(1R*,2S*,4S*)-bicyclo[2.2.1]hept-2-yl]pyrimidine-2,4-diamine;
6-[(3R)-3-aminopyrrolidin-1-yl]-N⁴-[(1R*,2S*,4S*)-bicyclo[2.2.1]hept-2-yl]pyrimidine-2,4-diamine;
6-[(3S)-3-aminopyrrolidin-1-yl]-N⁴-[(1R*,2R*,4S*)-bicyclo[2.2.1]hept-2-yl]pyrimidine-2,4-diamine;
6-[(3R)-3-aminopyrrolidin-1-yl]-N⁴-[(1R*,2R*,4S*)-bicyclo[2.2.1]hept-2-yl]pyrimidine-2,4-diamine;
N⁴-[exo-bicyclo[2.2.1]hept-2-yl]-6-[(3S)-3-(methylamino)pyrrolidin-1-yl]pyrimidine-2,4-diamine;
N⁴-[(1R*,2R*,4S*)-bicyclo[2.2.1]hept-2-yl]-6-[(3R)-3-(methylamino)pyrrolidin-1-yl]pyrimidine-2,4-diamine;
N⁴-[( 1R*,2S*,4S*)-bicyclo[2.2.1]hept-2-yl]-6-[(3S)-3-(methylamino)pyrrolidin-1-yl]pyrimidine-2,4-diamine;
N⁴-[(1R*,2S*,4S*)-bicyclo[2.2.1]hept-2-yl]-6-[(3R)-3-(methylamino)pyrrolidin-1-yl]pyrimidine-2,4-diamine;
N⁴-[(1R*,2R*,4S*)-bicyclo[2.2.1]hept-2-yl]-6-(3-methylpiperazin-1-yl)pyrimidine-2,4-diamine;
N⁴-[(1R*,2R*,4S*)-bicyclo[2.2.1]hept-2-yl]-6-(1,4-diazepan-1-yl)pyrimidine-2,4-diamine;
N⁴-[(1R*,2R*,4S*)-bicyclo[2.2.1]hept-2-yl]-6-((4aR*,7aR*)-octahydro-6H-pyrrolo[3,4-b]pyridin-6-yl)pyrimidine-2,4-diamine;
N⁴-[(1R*,2R*,4S*)-bicyclo[2.2.1]hept-2-yl]-6-(hexahydropyrrolo[1,2-a]pyrazin-2(1H)-yl)pyrimidine-2,4-diamine;
N⁴-[(1R*,2R*,4S*)-bicyclo[2.2.1]hept-2-yl]-6-piperazin-1-ylpyrimidine-2,4-diamine;
4-(3-aminopyrrolidin-1-yl)-6-cyclopentylpyrimidin-2-amine;
4-adamantan-2-yl-6-(3-aminopyrrolidin-1-yl)pyrimidin-2-amine;
4-cyclopentyl-6-[3-(methylamino)pyrrolidin-1-yl]pyrimidin-2-amine;
4-[(3S)-3-aminopyrrolidin-1-yl]-6-cyclohexylpyrimidin-2-amine;
4-[(3R)-3-aminopyrrolidin-1-yl]-6-cyclohexylpyrimidin-2-amine;
4-(3-aminopyrrolidin-1-yl)-6-(cyclohexylmethyl)pyrimidin-2-amine;
4-cyclopentyl-6-piperazin-1-ylpyrimidin-2-amine;
4-[adamantan-2-yl]-6-[(3R)-3-(methylamino)pyrrolidin-1-yl]pyrimidin-2-amine;
4-[adamantan-2-yl]-6-[(3R)-3-aminopyrrolidin-1-yl]pyrimidin-2-amine;
4-(cyclopentylmethyl)-6-(3-methylpiperazin-1-yl)pyrimidin-2-amine;
4-(3-aminopyrrolidin-1-yl)-6-(cyclopentylmethyl)pyrimidin-2-amine;
4-cyclohexyl-6-[3-(methylamino)azetidin-1-yl]pyrimidin-2-amine;
4-cyclopentyl-6-[(4aR*,7aR*)-octahydro-6H-pyrrolo[3,4-b]pyridin-6-yl]pyrimidin-2-amine;
4-cyclohexyl-6-(1,4-diazepan-1-yl)pyrimidin-2-amine;
4-cyclopentyl-6-(1,4-diazepan-1-yl)pyrimidin-2-amine;
6-cyclopentyl-N⁴-[2-(methylamino)ethyl]pyrimidine-2,4-diamine;
4-[(3R)-3-aminopyrrolidin-1-yl]-6-cyclohex-1-en-1-ylpyrimidin-2-amine.

21. A compound selected from the group consisting of
N⁴-cyclohexyl-6-(4-methylpiperazin-1-yl)pyrimidine-2,4-diamine;
4-(1,3-dihydro-2H-isoindol-2-yl)-6-piperazin-1-ylpyrimidin-2-amine;
4-(3-aminopyrrolidin-1-yl)-6-(2-methylpyrrolidin-1-yl)pyrimidin-2-amine;
4-[3-(methylamino)pyrrolidin-1-yl]-6-(2-methylpyrrolidin-1-yl)pyrimidin-2-amine;
6-(3-aminopyrrolidin-1-yl)-N⁴-[(1R*,2S*,4S*)-bicyclo[2.2.1]hept-2-yl]pyrimidine-2,4-diamine;
N⁴-[(1R*,2S*,4S*)-bicyclo[2.2.1]hept-2-yl]-6-[3-(methylamino)pyrrolidin-1-yl]pyrimidine-2,4-diamine;
6-[(3S)-3-aminopyrrolidin-1-yl]-N⁴-[(1R*,2S*,4S*)-bicyclo[2.2.1]hept-2-yl]pyrimidine-2,4-diamine;
6-[(3R)-3-aminopyrrolidin-1-yl]-N⁴-[(1R*,2S*,4S*)-bicyclo[2.2.1]hept-2-yl]pyrimidine-2,4-diamine;
6-[(3S)-3-aminopyrrolidin-1-yl]-N⁴-[(1R*,2R*,4S*)-bicyclo[2.2.1]hept-2-yl]pyrimidine-2,4-diamine;
6-[(3R)-3-aminopyrrolidin-1-yl]-N⁴-[(1R*,2R*,4S*)-bicyclo[2.2.1]hept-2-yl]pyrimidine-2,4-diamine;
N⁴-[exo-bicyclo[2.2.1]hept-2-yl]-6-[(3S)-3-(methylamino)pyrrolidin-1-yl]pyrimidine-2,4-diamine;
N⁴-[(1R*,2R*,4S*)-bicyclo[2.2.1]hept-2-yl]-6-[(3R)-3-(methylamino)pyrrolidin-1-yl]pyrimidine-2,4-diamine;
N⁴-[(1R*,2S*,4S*)-bicyclo[2.2.1]hept-2-yl]-6-[(3S)-3-(methylamino)pyrrolidin-1-yl]pyrimidine-2,4-diamine;
N⁴-[(1R*,2S*,4S*)-bicyclo[2.2.1]hept-2-yl]-6-[(3R)-3-(methylamino)pyrrolidin-1-yl]pyrimidine-2,4-diamine;
N⁴-[(1R*,2R*,4S*)-bicyclo[2.2.1]hept-2-yl]-6-(3-methylpiperazin-1-yl)pyrimidine-2,4-diamine;
N⁴-[(1R*,2R*,4S*)-bicyclo[2.2.1]hept-2-yl]-6-(1,4-diazepan-1-yl)pyrimidine-2,4-diamine;
N⁴-[(1R*,2R*,4S*)-bicyclo[2.2.1]hept-2-yl]-6-((4aR*,7aR*)-octahydro-6H-pyrrolo[3,4-b]pyridin-6-yl)pyrimidine-2,4-diamine;
N⁴-[(1R*,2R*,4S*)-bicyclo[2.2.1]hept-2-yl]-6-(hexahydropyrrolo[1,2-a]pyrazin-2(1H)-yl)pyrimidine-2,4-diamine;
N⁴-[(1R*,2R*,4S*)-bicyclo[2.2.1]hept-2-yl]-6-piperazin-1-ylpyrimidine-2,4-diamine;
4-(3-aminopyrrolidin-1-yl)-6-cyclopentylpyrimidin-2-amine;
4-adamantan-2-yl-6-(3-aminopyrrolidin-1-yl)pyrimidin-2-amine;
4-cyclopentyl-6-[3-(methylamino)pyrrolidin-1-yl]pyrimidin-2-amine;
4-[(3S)-3-aminopyrrolidin-1-yl]-6-cyclohexylpyrimidin-2-amine;
4-[(3R)-3-aminopyrrolidin-1-yl]-6-cyclohexylpyrimidin-2-amine;
4-(3-aminopyrrolidin-1-yl)-6-(cyclohexylmethyl)pyrimidin-2-amine;
4-cyclopentyl-6-piperazin-1-ylpyrimidin-2-amine;
4-[adamantan-2-yl]-6-[(3R)-3-(methylamino)pyrrolidin-1-yl]pyrimidin-2-amine;
4-[adamantan-2-yl]-6-[(3R)-3-aminopyrrolidin-1-yl]pyrimidin-2-amine;
4-(cyclopentylmethyl)-6-(3-methylpiperazin-1-yl)pyrimidin-2-amine;
4-(3-aminopyrrolidin-1-yl)-6-(cyclopentylmethyl)pyrimidin-2-amine;
4-cyclohexyl-6-[3-(methylamino)azetidin-1-yl]pyrimidin-2-amine;
4-cyclopentyl-6-[(4aR*,7aR*)-octahydro-6H-pyrrolo[3,4-b]pyridin-6-yl]pyrimidin-2-amine;
4-cyclohexyl-6-(1,4-diazepan-1-yl)pyrimidin-2-amine;
4-cyclopentyl-6-(1,4-diazepan-1-yl)pyrimidin-2-amine;
6-cyclopentyl-N⁴-[2-(methylamino)ethyl]pyrimidine-2,4-diamine;
4-[(3R)-3-aminopyrrolidin-1-yl]-6-cyclohex-1-en-1-ylpyrimidin-2-amine.

22. Synthesis intermediates selected from a group consisting of
4-cyclopentyl-N-(4-methoxybenzyl)-6-[3-(methylamino)pyrrolidin-1-yl]pyrimidin-2-amine;
4-cyclohexyl-6-(hexahydropyrrolo[1,2-a]pyrazin-2(1H)-yl)-N-(4-methoxybenzyl)pyrimidin-2-amine formate salt;
4-(adamantan-2-yl)-6-chloropyrimidin-2-amine;
tert-butyl [1-(6-adamantan-2-yl-2-aminopyrimidin-4-yl)pyrrolidin-3-yl]carbamate formate salt;
4-chloro-6-(2-methylpyrrolidin-1-yl)pyrimidin-2-amine;
tert-butyl [1-(2-amino-6-cyclohexylpyrimidin-4-yl)azetidin-3-yl]carbamate;
tert-butyl (3aR*,6aS*)-5-(2-amino-6-cyclohexylpyrimidin-4-yl)hexahydropyrrolo[3,4-c]pyrrole-2(1H)-carboxylate;
4-cyclohexyl-6-[(1R*,5S*,6S*)-6-nitro-3-azabicyclo[3.1.0]hex-3-yl]pyrimidin-2-amine;
tert-butyl (1R*,5S*,6S*)-6-[(2-amino-6-cyclohexylpyrimidin-4-yl)amino]-3-azabicyclo[3.1.0]hexane-3-carboxylate;
tert-butyl (1R*,5S*,6S*)-6-[(2-amino-6-cyclopentylpyrimidin-4-yl)amino]-3-azabicyclo[3.1.0]hexane-3-carboxylate;
tert-butyl (1R*,5S*,6S*)-6-{[2-amino-6-(cyclopentylmethyl)pyrimidin-4-yl]amino}-3-azabicyclo[3.1.0]hexane-3-carboxylate;
6-chloro-N⁴-(tetrahydro-2H-pyran-4-yl)pyrimidine-2,4-diamine;
N⁴-[(1R*,2S*,4S*)-bicyclo[2.2.1]hept-2-yl]-6-chloropyrimidine-2,4-diamine;
tert-butyl [1-(2-amino-6-cyclopropylpyrimidin-4-yl)pyrrolidin-3-yl]carbamate;
6-chloro-N⁴-[2-(dimethylamino)ethyl]pyrimidine-2,4-diamine;
N⁴-(8-benzyl-8-azabicyclo[3.2.1]oct-3-yl)-6-cyclohexylpyrimidine-2,4-diamine;
4-chloro-6-(1,3-dihydro-2H-isoindol-2-yl)pyrimidin-2-amine;
6-chloro-N⁴-cyclohexyl-N⁴-methylpyrimidine-2,4-diamine;
tert-butyl 3-[(2-amino-6-cyclohexylpyrimidin-4-yl)(methyl)amino]pyrrolidine-1-carboxylate;
tert-butyl {[1-(2-amino-6-cyclohexylpyrimidin-4-yl)azetidin-3-yl]methyl}carbamate;
tert-butyl 4-{2-amino-6-[(1R*,2R*,4S*)-bicyclo[2.2.1]hept-2-ylamino]pyrimidin-4-yl}-2-methylpiperazine-1-carboxylate;
tert-butyl [1-(2-amino-6-cyclopentylpyrimidin-4-yl)pyrrolidin-3-yl]carbamate di-formate salt;
tert-butyl 4-{[2-amino-6-(4-methylpiperazin-1-yl)pyrimidin-4-yl]amino}piperidine-1-carboxylate;
4-chloro-6-[(E)-2-phenylvinyl]pyrimidin-2-amine;
tert-butyl (1-{2-amino-6-[(E)-2-phenylvinyl]pyrimidin-4-yl}pyrrolidin-3-yl)carbamate;
4-chloro-6-(4-methylcyclohex-1-en-1-yl)pyrimidin-2-amine;
tert-butyl {1-[2-amino-6-(4-methylcyclohex-1-en-1-yl)pyrimidin-4-yl]pyrrolidin-3-yl}carbamate;
2-amino-6-(1,2,3,4-tetrahydronaphthalen-2-yl)pyrimidin-4-ol;
2-amino-6-(cyclohexylmethyl)pyrimidin-4-ol;
2-amino-6-(cyclopentylmethyl)pyrimidin-4-ol;
4-cyclohexyl-6-(4-cyclopropylpiperazin-1-yl)-N-(4-methoxybenzyl)pyrimidin-2-amine bis-formate salt;
tert-butyl {1-[2-amino-6-(1,2,3,4-tetrahydronaphthalen-2-yl)pyrimidin-4-yl]pyrrolidin-3-yl}carbamate;
tert-butyl {1-[2-amino-6-(cyclohexylmethyl)pyrimidin-4-yl]pyrrolidin-3-yl}carbamate formate salt;
tert-butyl {1-[2-amino-6-(2-phenylethyl)pyrimidin-4-yl]pyrrolidin-3-yl}carbamate;
4-chloro-6-(cyclopentylmethyl)pyrimidin-2-amine;
N-[1-(2-amino-6-cyclopentylpyrimidin-4-yl)azetidin-3-yl]acetamide.

23. Synthesis intermediates selected from a group consisting of
2,4-dichloro-6-cyclohexylpyrimidine;
tert-butyl [(3R)-1-(2-chloro-6-cyclohexylpyrimidin-4-yl)pyrrolidin-3-yl]carbamate;
2-chloro-4-cyclohexyl-6-(4-cyclopropylpiperazin-1-yl)pyrimidine;
tert-butyl [(3R)-1-{6-[adamantan-2-yl]-2-chloropyrimidin-4-yl}pyrrolidin-3-yl]carbamate;
tert-butyl [(3S)-1-{6-[adamantan-2-yl]-2-chloropyrimidin-4-yl}pyrrolidin-3-yl]carbamate;
tert-butyl [(3S)-1-(2-chloro-6-cyclohexylpyrimidin-4-yl)pyrrolidin-3-yl]carbamate;
tert-butyl [1-(2-chloro-6-cyclopentylpyrimidin-4-yl)pyrrolidin-3-yl]methylcarbamate;
2-(2-chloro-6-cyclohexylpyrimidin-4-yl)octahydropyrrolo[1,2-a]pyrazine;
tert-butyl [1-(2-chloro-6-cyclopentylpyrimidin-4-yl)pyrrolidin-3-yl]carbamat;
3-[(4-fluorobenzyl)oxy]pyrrolidine;
(3S)-1-{6-[adamantan-2-yl]-2-chloropyrimidin-4-yl}-N-methylpyrrolidin-3-amine;
cis-4-amino-N-phenylcyclohexanecarboxamide;
(1S*,3R*)-3-amino-N-phenylcyclohexanecarboxamide;
trans-4-amino-N-methylcyclohexanecarboxamide;
trans-4-amino-N-cyclopropylcyclohexanecarboxamide;
trans-4-amino-N-tert-butylcyclohexanecarboxamide;
trans-4-amino-N-(4-methoxyphenyl)cyclohexanecarboxamide;
tert-butyl ((1S*,3R*)-3-(anilinocarbonyl)cyclohexyl]carbamate;
tert-butyl [trans-4-(methylcarbamoyl)cyclohexyl]carbamate;
tert-butyl [trans-4-(cyclopropylcarbamoyl)cyclohexyl]carbamate;
tert-butyl [trans-4-(tert-butylcarbamoyl)cyclohexyl]carbamate;
tert-butyl {trans-4-[(4-methoxyphenyl)carbamoyl]cyclohexyl}carbamate;
methyl 3-oxo-3-(1,2,3,4-tetrahydronaphthalen-2-yl)propanoate;
4-[adamantan-2-yl]-2,6-dichloropyrimidine.

24. A pharmaceutical composition comprising as active ingredient a therapeutically effective amount of a compound according to claim 1 and a pharmaceutically acceptable adjuvant, diluent or carrier.

25. (canceled)

26. (canceled)

27. A method of treating a mammal having an H₄-dependent disease, comprising administering to the mammal an therapeutically effect amount of a compound according to any claim 1.

28. The method according to claim 27, wherein the disease is adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, allergic congestion; disorders of the genito-urinary tract such as female and male sexual dysfunction, overactive bladder conditions, urinary incontinence, neurogenic detrusor overactivity, idiopathic detrusor overactivity, benign prostate hyperplasia and lower urinary tract symptoms; dermatological diseases such as dermatitis and psoriasis and treatment of itchy skin; diseases of the cardiovascular system including thromboembolic diseases, atherosclerosis, myocardial infarction, angina pectoris (including unstable angina) myocardial ischaemia and arrhythmia, reocclusions and restenosis following angioplasty or coronary bypass, stroke, transitory ischaemic attacks, peripheral arterial occlusive diseases, pulmonary embolisms or deep venous thromboses, hypotension, pulmonary hypertension, malignant hypertension, cardiac insufficiency, heart or kidney failure, stroke and renal dysfunction; diseases of the gastrointestinal tract including inflammatory bowel disease, Crohn's disease, ulcerative colitis; autoimmune diseases including rheumatoid arthritis, multiple sclerosis; cancer; pain; or a lymphatic diseases.

29. The method according to claim 27 wherein the disease is an inflammatory disorder, a respiratory diseases selected from adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy induced airway responses, allergic rhinitis, viral rhinitis, non-allergic rhinitis, perennial and seasonal rhinitis, nasal congestion, and allergic congestion a dermatological disease selected from dermatitis and psoriasis and treatment of itchy skin, diseases of the gastrointestinal tract selected from inflammatory bowel disease, Crohn's disease, ulcerative colitis, or an autoimmune disease selected from rheumatoid arthritis and, multiple sclerosis.
